SEQUENCE LISTING (72 sequences)

SEQ ID NO: 1 - DNA, 1350 bp, synthetic construct
  source 1..1350: mol_type = other DNA
gaggtgcaggtgctggagagcggaggaggactggtgcagcccggagggtcactgagactgtcatgtgccgtgtcaggattttcactgagctcccacgctatgggctgggtgcggcaggcaccaggcaagggcctggagtggatcggcatcatcggctctagcgaccggacctactatgccagctgggccaagggcagattcaccatctccaaggataactctaagaatacagtgtacctgcagatcaactcccccagggccgaggacacagccgtgtactattgcgtgcgcgatccttatgattcttatgatgagggctaccgactgtgggggcaggggacactggtgaccgtctcctcagctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 2 - protein, 450 aa, synthetic construct
EVQVLESGGGLVQPGGSLRLSCAVSGFSLSSHAMGWVRQAPGKGLEWIGIIGSSDRTYYASWAKGRFTISKDNSKNTVYLQINSPRAEDTAVYYCVRDPYDSYDEGYRLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 651 bp, synthetic construct
  source 1..651: mol_type = other DNA
gatattcagatgacccagagccccagcacactgagcgcaagcgtcggagatagagtgaccattacctgccaggccagccagagcatttacagatacctgtcttggtatcagcagaagcccggcaaggcccctaagttcctgatctatgacgcctctaagctggccagcggcgtgccatccagattctctggcagcggctccggcaccgagtttaccctgacaatcagctccgtgcagccagacgatttcgcaacatactattgccagcaggcttacagcagcggcagcattgacaacgcattcggaggagggactaaagtggagatcaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 4 - protein, 217 aa, synthetic construct
DIQMTQSPSTLSASVGDRVTITCQASQSIYRYLSWYQQKPGKAPKFLIYDASKLASGVPSRFSGSGSGTEFTLTISSVQPDDFATYYCQQAYSSGSIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1368 bp, synthetic construct
  source 1..1368: mol_type = other DNA
gaggtgcagctggtggagagcggcggcggcctggtgcagcccggcggcagcctgagactgagctgcgccgccagcggcttcaccttcagcagctacagcatgaactgggtgagacaggcccccggcaagggcctggagtgggtgagctacatcagcagcagcagcagcaccatctactacgccgacagcgtgaagggcagattcaccatcagcagagacaacgccaagaacagcctgtacctgcagatgaacagcctgagagccgaggacaccgccgtgtactactgcgccagatacagactgcccaacttctggagcggctaccccaactacggcaccgacgtgtggggccagggcaccaccgtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 6 - protein, 456 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARYRLPNFWSGYPNYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 654 bp, synthetic construct
  source 1..654: mol_type = other DNA
cagtctgtgctgacacagccacccagcgtgagcggagcccctggacagagagtgaccatcagctgcaccggaagcagcagcaatatcggagccggatatgacgtgcattggtaccagcagctgcccggcaccgccccaaaactgctgatctacggaaacagcaacagacccagcggcgtgcccgacagattcagcggcagcaagagcggcacaagcgccagtctggctatcaccggcctgcaggccgaggacgaagccgactactactgccagagctacgacagcagcctgagcggccacgtggtgtttggaggaggaaccaagctgaccgtgctgggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 8 - protein, 218 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGHVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 9 - DNA, 1368 bp, synthetic construct
  source 1..1368: mol_type = other DNA
caggtgcagctgcaggagagcggcggcggactggtgcagccaggaggaagcctgagactgtcttgcgctgcttccggcttcacattcagcagctacagcatgaactgggtgagacaggcccccggaaaaggactggagtgggtgagctacatcagcagcagtagtagcaccatctattacgctgattccgtgaaaggcagattcacaatctcccgggacaacgccaagaacagcctgtacctgcagatgaacagcctgagagccgaggacaccgccgtgtactactgcgccaggtaccggctgcccgatttctggtccggctaccccaactacggcatggatgtgtggggccagggcaccaccgtgacagtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 10 - protein, 456 aa, synthetic construct
QVQLQESGGGLVQPGGSLRLSCAASGFTFSSYSMNWVRQAPGKGLEWVSYISSSSSTIYYADSVKGRFTISRDNAKNSLYLQMNSLRAEDTAVYYCARYRLPDFWSGYPNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 654 bp, synthetic construct; identical to SEQ ID NO: 7
  source 1..654: mol_type = other DNA

SEQ ID NO: 12 - protein, 218 aa, synthetic construct; identical to SEQ ID NO: 8

SEQ ID NO: 13 - DNA, 1350 bp, synthetic construct
  source 1..1350: mol_type = other DNA
gaggtgcagctgctggagagcggggggggactggtgcagccaggaggaagcctgagactgagctgtgccgccagcgggtttagcctgagcagccacgccatgggatgggtgaggcaggcccctggcaaaggcctggagtgggtgagcattattggcagcagtgacagaacctactacgccgacagcgtgaaagggcggttcaccatcagcagagataacagcaagaacacactgtacctgcagatgaacagcctgagagccgaggacaccgctgtgtactactgcgccaaggacccatacgatagttacgacgaaggatacagactgtgggggcagggaaccctggtgaccgtgagcagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcacctgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 14 - protein, 450 aa, synthetic construct
EVQLLESGGGLVQPGGSLRLSCAASGFSLSSHAMGWVRQAPGKGLEWVSIIGSSDRTYYADSVKGRFTISRDNSKNTLYLQMNSLRAEDTAVYYCAKDPYDSYDEGYRLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 651 bp, synthetic construct
  source 1..651: mol_type = other DNA
gacattcagatgacccagagccccagcaccctgagcgccagcgtgggagacagagtgaccatcacatgccaggctagccagagcatctacagatatttgagctggtaccagcagaaacccggcaaagcccccaaactgctgatctacgacgccagtaagctggccagcggagtgcccagcagattcagcggcagcggcagcggaaccgagttcaccctgaccatcagtagcctgcagcccgacgacttcgccacctactactgccagcaggcctactcatccggaagcattgacaacgccttcggcggcgggacaaaggtggagattaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 16 - protein, 217 aa, synthetic construct
DIQMTQSPSTLSASVGDRVTITCQASQSIYRYLSWYQQKPGKAPKLLIYDASKLASGVPSRFSGSGSGTEFTLTISSLQPDDFATYYCQQAYSSGSIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 1368 bp, synthetic construct
  source 1..1368: mol_type = other DNA
gaggtgcagctgcaggagagcggggggggactggtgcagccaggaggaagcctgagactgagctgtgccgccagcggatttacatttagtagttatagtatgaactgggtgaggcaggcccccggaaaaggcctggagtgggtgagctacattagcagcagtagcagcaccatctattacgctgactccgtgaagggcagattcaccatctccagagacaacgccaaaaacagcctgtacctgcagatgaacagcctgagagccgaggacacagccgtgtactactgcgccagatacagactgcccgatttctggagcggctaccccaactacggaatggatgtgtggggccagggcaccaccgtgacagtgagcagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccccgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 18 - protein, 456 aa, synthetic construct
EVQLQESGGGLVQPGGSLRLSCAASGFTFSSYSMNWVRQAPGKGLEWVSYISSSSSTIYYADSVKGRFTISRDNAKNSLYLQMNSLRAEDTAVYYCARYRLPDFWSGYPNYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 654 bp, synthetic construct
  source 1..654: mol_type = other DNA
cagagcgtgctgacccagccccccagcgtgagcggagctccaggacagagagtgacaatcagctgcaccggcagcagcagcaacatcggcgccggctacgacgtgcactggtaccagcagctgcctggcacagcccccaaactgctgatctacggccagagcaacagacccagcggcgtgcccgacaggttcagcggaagcaagtcaggaaccagcgcctccctggccatcaccggactgcaggccgaagatgaagctgactactactgccagagctacgacagcagcctgagtggccacgtggtgtttggcggaggaaccaagctgaccgtgctgggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 20 - protein, 218 aa, synthetic construct
QSVLTQPPSVSGAPGQRVTISCTGSSSNIGAGYDVHWYQQLPGTAPKLLIYGQSNRPSGVPDRFSGSKSGTSASLAITGLQAEDEADYYCQSYDSSLSGHVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 21 - DNA, 1368 bp, synthetic construct; identical to SEQ ID NO: 17
  source 1..1368: mol_type = other DNA

SEQ ID NO: 22 - protein, 456 aa, synthetic construct; identical to SEQ ID NO: 18

SEQ ID NO: 23 - DNA, 654 bp, synthetic construct
  source 1..654: mol_type = other DNA
cagtccgtcctcacccagcccccatccgtctccggcgcaccaggacagcgggtcaccatctcctgcaccggctcctcctccaacatcggcgccggctacgacgtccactggtaccagcagctcccaggcaccgcccccaaactcctgatctacggccagagcaacagaccctccggcgtccccgacagattctccggctccaagtctggaacatccgcctccctcgctatcaccggcctccaggcagaagatgaagctgactactactgccagtcctactcctcctccctcagcggtcacgtggtgttcggcgggggcaccaagctgacagtgctgggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 24 - protein, 218 aa, synthetic construct
QSVLTQPPSVSGAPGQRVTISCTGSSSNIGAGYDVHWYQQLPGTAPKLLIYGQSNRPSGVPDRFSGSKSGTSASLAITGLQAEDEADYYCQSYSSSLSGHVVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 25 - protein, 5 aa, synthetic construct
SHAMG

SEQ ID NO: 26 - protein, 16 aa, synthetic construct
IIGSSDRTYYASWAKG

SEQ ID NO: 27 - protein, 12 aa, synthetic construct
DPYDSYDEGYRL

SEQ ID NO: 28 - protein, 5 aa, synthetic construct
SHAMG

SEQ ID NO: 29 - protein, 16 aa, synthetic construct
IIGSSDRTYYADSVKG

SEQ ID NO: 30 - protein, 12 aa, synthetic construct
DPYDSYDEGYRL

SEQ ID NO: 31 - protein, 5 aa, synthetic construct
SYSMN

SEQ ID NO: 32 - protein, 17 aa, synthetic construct
YISSSSSTIYYADSVKG

SEQ ID NO: 33 - protein, 17 aa, synthetic construct
YRLPNFWSGYPNYGTDV

SEQ ID NO: 34 - protein, 5 aa, synthetic construct
SYSMN

SEQ ID NO: 35 - protein, 17 aa, synthetic construct
YISSSSSTIYYADSVKG

SEQ ID NO: 36 - protein, 17 aa, synthetic construct
YRLPDFWSGYPNYGMDV

SEQ ID NO: 37 - protein, 5 aa, synthetic construct
SYSMN

SEQ ID NO: 38 - protein, 17 aa, synthetic construct
YISSSSSTIYYADSVKG

SEQ ID NO: 39 - protein, 17 aa, synthetic construct
YRLPDFWSGYPNYGMDV

SEQ ID NO: 40 - protein, 5 aa, synthetic construct
SYSMN

SEQ ID NO: 41 - protein, 17 aa, synthetic construct
YISSSSSTIYYADSVKG

SEQ ID NO: 42 - protein, 17 aa, synthetic construct
YRLPDFWSGYPNYGMDV

SEQ ID NO: 43 - protein, 11 aa, synthetic construct
QASQSIYRYLS

SEQ ID NO: 44 - protein, 7 aa, synthetic construct
DASKLAS

SEQ ID NO: 45 - protein, 12 aa, synthetic construct
QQAYSSGSIDNA

SEQ ID NO: 46 - protein, 11 aa, synthetic construct
QASQSIYRYLS

SEQ ID NO: 47 - protein, 7 aa, synthetic construct
DASKLAS

SEQ ID NO: 48 - protein, 12 aa, synthetic construct
QQAYSSGSIDNA

SEQ ID NO: 49 - protein, 14 aa, synthetic construct
TGSSSNIGAGYDVH

SEQ ID NO: 50 - protein, 7 aa, synthetic construct
GNSNRPS

SEQ ID NO: 51 - protein, 12 aa, synthetic construct
QSYDSSLSGHVV

SEQ ID NO: 52 - protein, 14 aa, synthetic construct
TGSSSNIGAGYDVH

SEQ ID NO: 53 - protein, 7 aa, synthetic construct
GNSNRPS

SEQ ID NO: 54 - protein, 12 aa, synthetic construct
QSYDSSLSGHVV

SEQ ID NO: 55 - protein, 14 aa, synthetic construct
TGSSSNIGAGYDVH

SEQ ID NO: 56 - protein, 7 aa, synthetic construct
GQSNRPS

SEQ ID NO: 57 - protein, 12 aa, synthetic construct
QSYDSSLSGHVV

SEQ ID NO: 58 - protein, 14 aa, synthetic construct
TGSSSNIGAGYDVH

SEQ ID NO: 59 - protein, 7 aa, synthetic construct
GQSNRPS

SEQ ID NO: 60 - protein, 12 aa, synthetic construct
QSYSSSLSGHVV

SEQ ID NO: 61 - protein, 120 aa, synthetic construct
EVQVLESGGGLVQPGGSLRLSCAVSGFSLSSHAMGWVRQAPGKGLEWIGIIGSSDRTYYASWAKGRFTISKDNSKNTVYLQINSPRAEDTAVYYCVRDPYDSYDEGYRLWGQGTLVTVSS

SEQ ID NO: 62 - protein, 120 aa, synthetic construct
EVQLLESGGGLVQPGGSLRLSCAASGFSLSSHAMGWVRQAPGKGLEWVSIIGSSDRTYYADSVKGRFTISRDNSKNTLYLQMNSLRAEDTAVYYCAKDPYDSYDEGYRLWGQGTLVTVSS

SEQ ID NO: 63 - protein, 126 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCARYRLPNFWSGYPNYGTDVWGQGTTVTVSS

SEQ ID NO: 64 - protein, 126 aa, synthetic construct
QVQLQESGGGLVQPGGSLRLSCAASGFTFSSYSMNWVRQAPGKGLEWVSYISSSSSTIYYADSVKGRFTISRDNAKNSLYLQMNSLRAEDTAVYYCARYRLPDFWSGYPNYGMDVWGQGTTVTVSS

SEQ ID NO: 65 - protein, 126 aa, synthetic construct
EVQLQESGGGLVQPGGSLRLSCAASGFTFSSYSMNWVRQAPGKGLEWVSYISSSSSTIYYADSVKGRFTISRDNAKNSLYLQMNSLRAEDTAVYYCARYRLPDFWSGYPNYGMDVWGQGTTVTVSS

SEQ ID NO: 66 - protein, 126 aa, synthetic construct; identical to SEQ ID NO: 65

SEQ ID NO: 67 - protein, 110 aa, synthetic construct
DIQMTQSPSTLSASVGDRVTITCQASQSIYRYLSWYQQKPGKAPKLLIYDASKLASGVPSRFSGSGSGTEFTLTISSLQPDDFATYYCQQAYSSGSIDNAFGGGTKVEIK

SEQ ID NO: 68 - protein, 110 aa, synthetic construct; identical to SEQ ID NO: 67

SEQ ID NO: 69 - protein, 112 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGHVVFGGGTKLTVL

SEQ ID NO: 70 - protein, 112 aa, synthetic construct
QSVLTQPPSVSGAPGQRVTISCTGSSSNIGAGYDVHWYQQLPGTAPKLLIYGQSNRPSGVPDRFSGSKSGTSASLAITGLQAEDEADYYCQSYDSSLSGHVVFGGGTKLTVL

SEQ ID NO: 71 - protein, 112 aa, synthetic construct
QSVLTQPPSVSGAPGQRVTISCTGSSSNIGAGYDVHWYQQLPGTAPKLLIYGQSNRPSGVPDRFSGSKSGTSASLAITGLQAEDEADYYCQSYSSSLSGHVVFGGGTKLTVL

SEQ ID NO: 72 - protein, 112 aa, synthetic construct; identical to SEQ ID NO: 71

(19) United States
(12) Patent Application Publication
(10) Pub. No.: US 2026/0256937 A1
(43) Pub. Date: Sep. 3, 2026

(54) NOVEL CONJUGATES, COMPOSITIONS AND METHODS OF USE

(71) Applicant: Virtuoso BINco, Inc., San Mateo, CA (US)

(72) Inventors: David Y. JACKSON, Belmont, CA (US); Oi Kwan WONG, Belmont, CA (US); Lei SHI, Shanghai (CN); Qi FEI, Shanghai (CN)

(21) Appl. No.: 18/726,957

(22) PCT Filed: Jan. 5, 2023
(86) PCT No.: PCT/CN2023/070618

§ 371 Date: Jul. 5, 2024

(30) Foreign Application Priority Data
Jan. 6, 2022 (WO) PCT/CN2022/070521

(51) Int. Cl. A61K 47/68; A61P 35/00

(52) U.S. Cl. CPC A61K 47/6849; A61K 47/68037; A61K 47/6889; A61P 35/00

(57) ABSTRACT

Described herein are methods and compositions for the targeted delivery of therapeutic agents. Provided herein are compounds and conjugates of the formulas described herein, including antibody-linker conjugates, comprising comprising one or more moieties derived from therapeutic agents (a topoisomerase inhibitor or a poly adenosine diphosphate-ribose polymerase (PARP) inhibitor), and wherein the conjugate further comprises a polypeptide, such as an antibody. The present disclosure also provides compositions comprising such conjugates and uses and methods for treating diseases and disorders, such as cancer, using these conjugates.

### CROSS REFERENCE

This application claims priority to PCT application no. PCT/CN2022/070521, filed on Jan. 6, 2022, the entirety of which is hereby incorporated by reference herein.

### SEQUENCE LISTING

The instant application contains a Sequence Listing which has been submitted electronically in ASCII format and is hereby incorporated by reference in its entirety. Said ASCII copy, created on Dec. 21, 2022, is named txt_55429-737_602_SL.xml and is XX kilobytes in size.

### FIELD

Provided herein are compounds and conjugates of the formulas described herein, including antibody-drug conjugates, comprising one or more moieties derived from therapeutic agents (a topoisomerase inhibitor or a poly adenosine diphosphate-ribose polymerase (PARP) inhibitor), and wherein the conjugates further comprise a polypeptide, such as an antibody, that binds a target of interest (e.g., antibodies targeting ICAM1 or EphA2). Also provided herein are compositions comprising such compounds or conjugates, and methods of their making. Said antibody-drug conjugates are useful for the treatment of diseases or disorder, for example, a proliferative disease such as a cancer. Also provided herein are uses and methods for treating diseases and disorders using these conjugates.

### SUMMARY

In one aspect, provided herein are compounds of Formula (I):

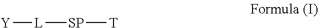

wherein,
T is a moiety derived from a compound capable of inhibiting topoisomerase or poly (ADP-ribose) polymerase (PARP);
SP is absent, a di- or tri-peptide linking moiety having L bonded to the N-terminus and T bonded to the C-terminus, or a linking moiety capable of self-immolation at pH less than 8;
L is a di- or tri-peptide linking moiety having Y bonded to the N-terminus and SP bonded to the C-terminus; and
Y is a conjugation moiety capable of forming a covalent bond with a nitrogen atom of a lysine residue or a sulfur atom of a cysteine residue;
or a salt or a hydrate thereof.

In another aspect, provided herein are conjugates of Formula (II):

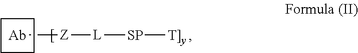

wherein:
T is a moiety derived from a compound capable of inhibiting topoisomerase enzyme or poly (ADP-ribose) polymerase (PARP);
SP is absent, a di- or tri-peptide linking moiety having L bonded to the N-terminus and T bonded to the C-terminus, or a linking moiety capable of self-immolation at pH less than 8;
L is a di- or tri-peptide linking moiety having Y bonded to the N-terminus and SP bonded to the C-terminus;
[symbol] is an antibody;
Z is a residual moiety resulting from the covalent linkage of Y to [symbol] ; and Y is a conjugation moiety capable of forming a covalent bond with a nitrogen atom of a lysine residue or a sulfur atom of a cysteine reside; and
wherein y is an integer from 1 to 20.

In certain embodiments, the conjugate is a conjugate of Formula (IIa):

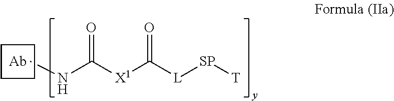

wherein,
   X¹ is selected from bond, —(CH₂)ₙ—, —(CH₂)ₙ—(OCH₂CH₂)ₘ—, —(CH₂)ₙ—O—(CH₂)ₘ—, —(CH₂)ₙ—NH—(CH₂)ₘ—, wherein n is 1 to 5, and m is 1 to 8;
   T is a moiety derived from a compound capable of inhibiting topoisomerase or poly (ADP-ribose) polymerase (PARP);
   SP is absent, a di- or tri-peptide linking moiety having L bonded to the N-terminus and T bonded to the C-terminus, or a linking moiety capable of self-immolation at pH less than 8;
   L is a di- or tri-peptide linking moiety having SP bonded to the C-terminus;
   [symbol] is an anti-ICAM1 or anti-EphA2 antibody; and
wherein y is an integer from 1 to 20.

In certain embodiments, the conjugate is a conjugate of Formula (IIb):

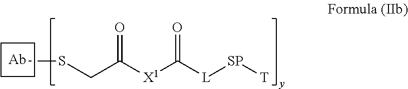

wherein,
   X¹ is selected from bond, —(CH₂)ₙ—, —(CH₂)ₙ—(OCH₂CH₂)ₘ—, —(CH₂)ₙ—O—(CH₂)ₘ—, —(CH₂)ₙ—NH—(CH₂)ₘ—, wherein n is 1 to 5, and m is 1 to 8;
   T is a moiety derived from a compound capable of inhibiting topoisomerase or poly (ADP-ribose) polymerase (PARP);
   SP is absent, a di- or tri-peptide linking moiety having L bonded to the N-terminus and T bonded to the C-terminus, or a linking moiety capable of self-immolation at pH less than 8;
   L is a di- or tripeptide linking moiety having SP bonded to the C-terminus;
   [symbol] is an anti-ICAM1 or anti-EphA2 antibody;
wherein y is an integer from 1 to 8.

In certain embodiments, the conjugate is a conjugate of Formula (IIc),

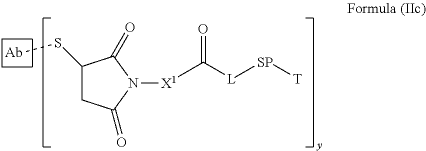

wherein,
   X¹ is selected from bond, —(CH₂)ₙ—, —(CH₂)ₙ—(OCH₂CH₂)ₘ—, —(CH₂)ₙ—O—(CH₂)ₘ—, —(CH₂)ₙ—NH—(CH₂)ₘ—, wherein n is 1 to 5, and m is 1 to 8;
   T is a moiety derived from a compound capable of inhibiting topoisomerase or poly (ADP-ribose) polymerase (PARP);
   SP is absent, a di- or tri-peptide linking moiety having L bonded to the N-terminus and T bonded to the C-terminus, or a linking moiety capable of self-immolation at pH less than 8;
   L is a di- or tripeptide linking moiety SP bonded to the C-terminus;
   Ab is an anti-ICAM1 or anti-EphA2 antibody; and
wherein y is an integer from 1 to 8.

In certain embodiments, the conjugate is a conjugate of Formula (IId):

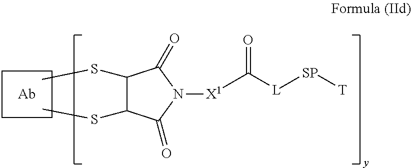

wherein,
   X¹ is selected from bond, —(CH₂)ₙ—, —(CH₂)ₙ—(OCH₂CH₂)ₘ—, —(CH₂)ₘ—O—(CH₂)ₘ—, —(CH₂)ₙ—NH—(CH₂)ₘ—, wherein n is 1 to 5, and m is 1 to 8;
   T is a moiety derived from a compound capable of inhibiting topoisomerase or poly (ADP-ribose) polymerase (PARP);
   SP is absent, a di- or tri-peptide linking moiety having L bonded to the N-terminus and T bonded to the C-terminus, or a linking moiety capable of self-immolation at pH less than 8;
   L is a di- or tri-peptide linking moiety having SP bonded to the C-terminus;
   Ab is an anti-ICAM1 or anti-EphA2 antibody; and
wherein y is an integer from 1 to 4.

In another aspect, provided herein are pharmaceutical compositions comprising a conjugate described herein (e.g. an antibody-drug conjugate), and at least one pharmaceutically acceptable carrier.

In another aspect, provided herein are methods of treating a disease or disorder in a patient in need thereof, comprising administering to the patient the conjugate or the pharmaceutical composition described herein (e.g. an antibody-drug conjugate).

In certain embodiments, the disease or disorder is cancer. In certain embodiments, the cancer is a solid tumor. In certain embodiments, the cancer is a hematological malignancy.

In certain embodiments, the solid tumor is selected from the group consisting of ovarian cancer, head and neck cancer, thyroid cancer, gastric cancer, bladder cancer, cholangiocarcinoma, endometrial cancer, hepatocellular carcinoma, kidney cancer, melanoma, lung cancer (e.g., non-small cell lung cancer), colorectal cancer, prostate cancer, pancreatic cancer, and Ewing's sarcoma. In certain embodiments, the cancer is lung cancer (e.g., non-small cell lung cancer). In certain embodiments, the the cancer is colorectal cancer (CRC). In certain embodiments, the cancer is prostate cancer. In certain embodiments, the cancer is pancreatic cancer. In some embodiments, the cancer is hepatocellular carcinoma. In certain embodiments, the hematological malignancy is multiple myeloma (MM). In certain embodiments, the hematological malignancy is non-hodgkin lymphoma (NHL).

### BRIEF DESCRIPTION OF THE DRAWINGS

FIGS. 1A-1B show characterizations of Conjugate IC-1 (DAR8) by LC/MS (FIG. 1A) and SEC-HPLC (FIG. 1i).

FIGS. 2A-2B show characterizations of Conjugate IC-1 (DAR5) by LC/MS (FIG. 2A) and SEC-HPLC (FIG. 2B).

FIGS. 3A-3B show characterizations of Conjugate IC-3 by LC/MS (FIG. 3A) and SEC-HPLC (FIG. 3B).

FIGS. 4A-4B show characterizations of Conjugate IC-4 by LC/MS (FIG. 4A) and SEC-HPLC (FIG. 4B).

FIGS. 5A-5B show characterizations of Conjugate IC-7 by LC/MS (FIG. 5A) and SEC-HPLC (FIG. 5B).

FIGS. 6A-6B show characterizations of Conjugate IC-9 by LC/MS (FIG. 6A) and SEC-HPLC (FIG. 6B).

FIGS. 7A-7B show characterizations of Conjugate IC-12 by LC/MS (FIG. 7A) and SEC-HPLC (FIG. 7B).

FIGS. 8A-8B show characterizations of Conjugate AC-1 (DAR8) by LC/MS (FIG. 8A) and SEC-HPLC (FIG. 8B).

FIGS. 9A-9B show characterizations of Conjugate AC-1 (DAR5) by LC/MS (FIG. 9A) and SEC-HPLC (FIG. 9B).

FIGS. 10A-10B show characterizations of Conjugate AC-3 (DAR5) by LC/MS (FIG. 10A) and SEC-HPLC (FIG. 10B).

FIGS. 11A-11B show characterizations of Conjugate AC-4 (DAR4) by LC/MS (FIG. 11A) and SEC-HPLC (FIG. 11B).

FIGS. 12A-12B show characterizations of Conjugate AC-7 (DAR4) by LC/MS (FIG. 12A) and SEC-HPLC (FIG. 12B).

FIGS. 13A-13B show characterizations of Conjugate AC-9 (DAR4) by LC/MS (FIG. 13A) and SEC-HPLC (FIG. 13B).

FIGS. 14A-14B show characterizations of Conjugate AC-12 (DAR4) by LC/MS (FIG. 14A) and SEC-HPLC (FIG. 14B).

FIGS. 15A-15B illustrate in vitro activities of anti-ICAM1 antibody VP0270 Conjugates IC-1, IC-3, IC-7, IC-9, and IC-12 (FIG. 15A) and in vitro activities of anti-EphA2 antibody VP0633 Conjugates AC-1 (DAR8), AC-1 (DAR5), AC-3, AC-7, AC-9, and AC-12 (FIG. 15B) in inhibiting growth of MDA-MB436 (breast cancer) cells.

FIG. 16 illustrates that both Conjugates IC-1 (DAR5) and IC-1 (DAR8), which comprises anti-ICAM1 antibody VP0270, effectively inhibited HCC827 tumor growth in vivo in a NSCLC xenograft model.

FIGS. 17A and 17B illustrate that antibody conjugates AC-40 (VP0633-Compound 40) and AC-105 (VP0633-Compound 105) have higher inhibitory activity in PC3 cell line (FIG. 17A) than in the EphA2 negative Raji cell line (FIG. 17B), and that antibody conjugate AC-40 has comparable activity when compared to the antibody conjugate with a reference payload of DXd (conjugate AC-105).

FIGS. 18A and 18B illustrate the in vitro activities of three anti-EphA2 antibody conjugates AC-C1 (VP0253-Compound 1), AC-A1 (VP1127-Compound 1) and AC-B1 (VP1342-Compound 1) in OVCAR3 ovarian cell line (FIG. 18A) and in vitro activities of two anti-ICAM1 antibody conjugates IC-40 (VP0270-Compound 40) and IC-A40 (VP1157-Compound 40) compared to isotype antibody conjugate (Isotype-Compound 40) in Raji lymphoma cell line (FIG. 18B).

FIG. 19 illustrates the in vivo anti-tumor activities of six anti-EphA2 antibody conjugates AC-1 (VP0633-Compound 1), AC-40 (VP0633-Compound 40), AC-105 (VP0633-Compound 105), AC-A1 (VP1127-Compound 1), AC-B1 (VP1342-Compound 1), and AC-C1 (VP0253-Compound 1) in OVCAR3 ovarian xenograft model after a single injection of the ADCs.

FIG. 20 compares the in vivo anti-tumor activities of two anti-ICAM1 antibody conjugates IC-106 (VP0270-Compound 106) and IC-105 (VP0270-Compound 105) in NCI-H441 non-small cell lung cancer xenograft model after a single injection of the ADCs.

FIG. 21 illustrates the in vivo anti-tumor activities of two anti-ICAM1 antibody conjugates IC-1 (VP0270-Compound 1) and IC-A1 (VP1157-Compound 1) compared to isotype antibody conjugate (Isotype-Compound 1) in Hep3B2.1-7 hepatocellular carcinoma xenograft model after two doses of the ADCs.

FIG. 22 illustrates the in vivo anti-tumor activities of five anti-ICAM1 antibody conjugates IC-1 (VP0270-Compound 1), IC-40 (VP0270-Compound 40), IC-105 (VP0270-Compound 105), IC-A40 (VP1157-Compound 40), and IC-A105 (VP1157-Compound 105) in NCI-H2444 non-small cell lung cancer xenograft model after a single injection of the ADCs.

### DETAILED DESCRIPTION OF THE INVENTION

Improving the delivery of therapeutic agents to the target cells, tissues and tumors to achieve maximal efficacy and minimal toxicity has been the focus of considerable research for many years. Though many attempts have been made to develop effective methods for importing biologically active molecules into cells, both in vivo and in vitro, none has proved to be entirely satisfactory. Optimizing the association of the drug with its intracellular target, while minimizing intercellular redistribution of the drug, e.g., to neighboring cells, is often difficult or inefficient.

Most agents currently administered to a patient parenterally are not targeted, resulting in systemic delivery of the agent to cells and tissues of the body where it is unnecessary, and often undesirable. This may result in adverse drug side effects, and often limits the dose of a drug (e.g., chemotherapeutic (anti-cancer), cytotoxic, enzyme inhibitor agents and antiviral or antimicrobial drugs) that can be administered. By comparison, although oral administration of drugs is considered to be a convenient and economical mode of administration, it shares the same concerns of non-specific toxicity to unaffected cells once the drug has been absorbed into the systemic circulation. Further complications involve problems with oral bioavailability and residence of drug in the gut leading to additional exposure of gut to the drug and hence risk of gut toxicities.

Accordingly, a major goal has been to develop methods for specifically targeting therapeutic agents to cells and tissues. The benefits of such treatment include avoiding the general physiological effects of inappropriate delivery of such agents to other cells and tissues. Intracellular targeting may be achieved by methods, compounds and formulations which allow accumulation or retention of biologically active agents, i.e. active metabolites, inside cells. There is a clear unmet need in the art for therapeutic chemotherapeutic agents having significantly lower toxicity, yet useful therapeutic efficiency. These and other limitations and problems of the past are addressed by the disclosure herein.

### Certain Terminology

Unless defined otherwise, all technical and scientific terms used herein have the same meaning as is commonly understood by one of skill in the art to which the claimed subject matter belongs. All patents, patent applications, published applications and publications, GENBANK sequences, websites and other published materials referred to throughout the entire disclosure herein, unless noted otherwise, are incorporated by reference in their entirety. Generally, the procedures for cell culture, cell infection, antibody production and molecular biology methods are methods commonly used in the art. Such standard techniques can be found, for example, in reference manual, such as, for example, Sambrook et al. (2000) and Ausubel et al. (1994).

As used herein, the singular forms “a,” “an” and “the” include plural referents unless the context clearly dictates otherwise. In this application, the use of the singular includes the plural unless specifically stated otherwise. As used herein, the use of “or” means “and/or” unless stated otherwise. Furthermore, use of the term “including” as well as other forms (e.g., “include”, “includes”, and “included”) is not limiting.

The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention.

As used herein, ranges and amounts can be expressed as “about” a particular value or range. About also includes the exact amount. Hence “about 40 mg” means “about 40 mg” and also “40 mg.” Generally, the terms “about” and “approximately” include an amount that would be expected to be within experimental error.

The terms “individual,” “patient,” or “subject” are used interchangeably. As used herein, they mean any mammal (i.e. species of any orders, families, and genus within the taxonomic classification animalia: chordata: vertebrata: mammalia). In some embodiments, the mammal is a human. None of the terms require or are limited to situation characterized by the supervision (e.g. constant or intermittent) of a health care worker (e.g. a doctor, a registered nurse, a nurse practitioner, a physician's assistant, an orderly, or a hospice worker).

The terms “polypeptide,” “peptide” and “protein” are used interchangeably herein to refer to a polymer of amino acid residues. The terms apply to naturally occurring amino acid polymers as well as amino acid polymers in which one or more amino acid residues is a non-naturally occurring amino acid (e.g., an amino acid analog). The terms encompass amino acid chains of any length, including full length proteins (i.e., antigens), wherein the amino acid residues are linked by covalent peptide bonds.

Where an amino acid sequence is provided herein, L-, D-, or beta amino acid versions of the sequence are also contemplated as well as retro, inversion, and retro-inversion isoforms. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other modified linkages (e.g., where the peptide bond is replaced by an α-ester, a β-ester, a thioamide, phosphonamide, carbamate, hydroxylate, and the like (see, e.g., Spatola, (1983) Chem. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like)).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acids are grouped as hydrophobic amino acids, polar amino acids, non-polar amino acids, and charged amino acids. Hydrophobic amino acids include small hydrophobic amino acids and large hydrophobic amino acids. Small hydrophobic amino acid can be glycine, alanine, proline, and analogs thereof. Large hydrophobic amino acids can be valine, leucine, isoleucine, phenylalanine, methionine, tryptophan, and analogs thereof. Polar amino acids can be serine, threonine, asparagine, glutamine, cysteine, tyrosine, and analogs thereof. Non-polar amino acids can be glycine, alanine, valine, leucine, isoleucine, methionine, phenylalanine, tryptophan, proline, and analogs thereof. Charged amino acids can be lysine, arginine, histidine, aspartate, glutamate, and analogs thereof. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids are either D amino acids or L amino acids.

As used in the specification and appended claims, unless specified to the contrary, the following terms have the meaning indicated below.

“Amino” refers to the —NH₂ radical.

“Cyano” refers to the —CN radical.

“Halo” means a fluoro, chloro, bromo, or iodo group.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)R^{a} (where t is 1 or 2), —S(O)ₜOR^{a}(where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans). Thus, the compounds provided herein may be enantiomerically pure, or be stereoisomeric or diastereomeric mixtures. The compounds provided herein may contain chiral centers. Such chiral centers may be of either the (R) or (S) configurations, or may be a mixture thereof. The chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form. Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

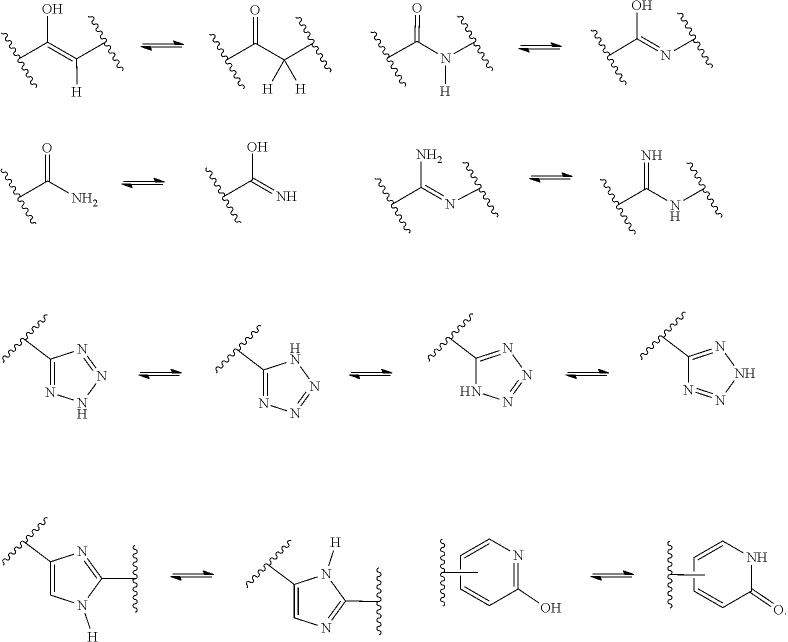

“Pharmaceutically acceptable salt” includes both acid and base addition salts. A pharmaceutically acceptable salt of any one of the compounds or conjugates described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

As used herein, the terms “antibody” and “immunoglobulin” are terms of art and can be used interchangeably herein, and refer to a molecule with an antigen binding site that specifically binds an antigen. In certain embodiments, an isolated antibody (e.g., monoclonal antibody) described herein, or an antigen-binding fragment thereof, which specifically binds to a protein of interest.

Antibodies can include, for example, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain/antibody heavy chain pair, an antibody with two light chain/heavy chain pairs (e.g., identical pairs), intrabodies, heteroconjugate antibodies, single domain antibodies, monovalent antibodies, bivalent antibodies (including monospecific or bispecific bivalent antibodies), single chain antibodies, or single-chain Fvs (scFv), camelized antibodies, affybodies, Fab fragments, F(ab′) fragments, F(ab′)₂ fragments, disulfide-linked Fvs (sdFv), anti-idiotypic (anti-Id) antibodies (including, e.g., anti-anti-Id antibodies), and epitope-binding fragments of any of the above.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies (e.g., human IgG), or a class (e.g., human IgG1, IgG2, IgG3 or IgG4) or subclass thereof.

The CDR sequence(s) for the antibodies disclosed herein, or the anti-CD47 or anti-ICAM1 binding domain sequences disclosed herein, may be defined or determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; A1-Lazikani et al., 1997, J. Mol. Biol., 273:927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

The term “chimeric” antibody refers to an antibody in which a portion of the heavy and/or light chain is derived from a particular source or species, while the remainder of the heavy and/or light chain is derived from a different source or species.

The term “human antibody” or “humanized antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). In some instances, human antibodies are also produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al, Proc. Natl. Acad. Sci. USA 90 (1993) 2551-2555; Jakobovits, A., et al, Nature 362 (1993) 255-258; Bruggemann, M., et al, Year Immunol. 7 (1993) 33-40). In additional instances, human antibodies are also produced in phage display libraries (Hoogenboom, H. R., and Winter, G., J. Mol. Biol. 227 (1992) 381-388; Marks, J. D., et al, J. Mol. Biol. 222 (1991) 581-597). The techniques of Cole et al. and Boerner et al. are also available for the preparation of human monoclonal antibodies (Cole, et al., Monoclonal Antibodies and Cancer Therapy, Alan R. Liss, p. 77 (1985); and Boerner, P., et al, J. Immunol. 147 (1991) 86-95).

As used herein, an “antigen” is a moiety or molecule that contains an epitope to which an antibody can specifically bind. As such, an antigen is also specifically bound by an antibody. In a specific embodiment, the antigen, to which an antibody described herein binds, is a protein of interest, for example, ICAM1, EphA2, or a fragment thereof.

As used herein, the term “heavy chain” when used in reference to an antibody can refer to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

As used herein, the term “light chain” when used in reference to an antibody can refer to any distinct types, e.g., kappa (κ) of lambda (λ) based on the amino acid sequence of the constant domains. Light chain amino acid sequences are well known in the art. In specific embodiments, the light chain is a human light chain.

As used herein, the term “percent (%) amino acid sequence identity” or “sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “full length antibody,” “intact antibody” and “whole antibody” are used herein interchangeably to refer to an antibody in its substantially intact form, and are not antibody fragments as defined below. The terms particularly refer to an antibody with heavy chains that contain the Fc region.

“Antibody fragments” comprise only a portion of an intact antibody, wherein the portion retains at least one, two, three and as many as most or all of the functions normally associated with that portion when present in an intact antibody. In one aspect, an antibody fragment comprises an antigen binding site of the intact antibody and thus retains the ability to bind antigen.

As used herein, an “epitope” is a term known in the art and refers to a localized region of an antigen to which an antibody can specifically bind. An epitope can be a linear epitope of contiguous amino acids or can comprise amino acids from two or more non-contiguous regions of the antigen.

As used herein, the terms “binds,” “binds to,” “specifically binds” or “specifically binds to” in the context of antibody binding refer to antibody binding to an antigen (e.g., epitope) as such binding is understood by one skilled in the art. In a specific embodiment, molecules that specifically bind to an antigen bind to the antigen with an affinity (K_{d}) that is at least 2 logs, 2.5 logs, 3 logs, 4 logs lower (higher affinity) than the K_{d} when the molecules bind to another antigen. In another specific embodiment, molecules that specifically bind to an antigen do not cross react with other proteins.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

A “linking moiety” or “linker” (e.g., noted as L) is a molecule with two reactive termini, one for conjugation to a polypeptide (e.g., an antibody) through conjugation moiety Y and the other for conjugation to a linking moiety (noted as SP) or a moiety of T when SP is absent. The polypeptide conjugation reactive terminus of the linker is typically a site that is capable of conjugation to the polypeptide (e.g., an antibody) through a cysteine thiol or lysine amine group on the polypeptide (e.g., an antibody), and so is typically a thiol-reactive group such as a maleimide or a dibromomaleimide, or as defined herein, or an amine-reactive group such as a tetrafluorophenyl acetate or perfluorophenyl acetate, or as defined herein.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### Payloads

Antibody drug conjugates (ADCs) combine the target specificity of an antibody (e.g., a monoclonal antibody) with the potency of a small molecule drug (known as payload) by connecting them into a single ADC molecule that retain the properties of both. The improved selectivity and potency of ADCs leads to superior safety and efficacy resulting in broader therapeutic windows compared to conventional chemotherapeutic drugs.

Over the past two decades a total of nine ADCs have been approved by the FDA; including: gemtuzumab ozogamicin (Mylotarg™), brentuximab vedotin (Adcetris™), ado-trastuzumab emtansine (Kadcyla™), inotuzumab ozogamicin (Besponsa™), polatuzumab vedotin (Polivy™), enfortumab vedotin (Padcev™), trastuzumab deruxtecan (Enhertu™) sacituzumab govitecan (Trodelvy™) and belantamab mafodotin (Blenrep™).

In certain embodiments, the payload is a topoisomerase inhibitor. In certain embodiment, the topoisomerase inhibitor is selected from the group consisting of 10-Hydroxycamptothecin Aclarubicin hydrochloride, AEZS-112 (ZEN012), Afeletecan, Amonafide dihydrochloride, Amonafide L-malate, Amrubicin (SM-5887), Amsacrine, Asulacrine (CI-921, NSC-343499, or SN-21407), Annamycin, ARQ-501, Becatecarin (BMY-27557, XL119 or BMS-181176), Belotecan, Berubicin hydrochloride, Betulinic acid, Camptothecin, Carubicin, Celastrol, Celiptium, CHIR-124, Dactinomycin, Daunorubicin hydrochloride, Detorubici, Dexrazoxane, Doxorubici, Edotecarin, Ellipticine, Elomotecan, Elsamitrucin, Epirubici, Epirubicin, Esorubicin, Etoposide, Exatecan, GENZ-644282, Gimatecan, GPX100, Idarubicin, Idronoxil, Indimitecan, Indotecan, Irinotecan HCl trihydrate, Karenitecin, KU59403, Ledoxantrone, Lucanthone, Lurtotecan, Mitoxantrone, MK-2206, Mureletecan, Nemorubicin, NK314, Pirarubicin, Piroxantrone, Pixantrone, Rubitecan, Sabarubicin, Silatecan, Simmitecan, SN38, TAS-103 (BMS-247615), Teloxantrone, Tenifatecan, Teniposid, Thiocolchicine, Thioguanine, Topotecan, UNBS3157, Valrubicin, Voreloxin, XK469, and Zorubicin. In certain embodiments, the payload is a topoisomerase I inhibitor. In one embodiment, the topoisomerase 1 inhibitor is Exatecan or SN38. In a specific embodiment, the topoisomerase 1 inhibitor is Exatecan. In another specific embodiment, the topoisomerase 1 inhibitor is SN38.

In certain embodiments, the payload is a poly adenosine diphosphate-ribose polymerase (PARP) inhibitor. In certain embodiments, the payload is selected from the group consisting of A-966492, ABT-737, ABT-767, AG14361, ARQ-761, AZD-2461, CEP-6800, CEP-9722, CGP74514A, Chlorin E6, CWP232204, DAT-230, E7016 (GPI21016), E7449 (2X-121), Etalocib, GPI-15427, Iniparib, INO-1001, JW55, KU-0058684, L-2286, Mahanine, Niraparib, NU1025, NU1064, NU1085, NU6027, Olaparib (AZD-2281 or KU-59436), PJ-34, Rucaparib (AG-14699 or PF-01367338), Talazoparib, Tubacin, Veliparib, VMY-1-103, WP-1034, and YK-3-237. In one embodiment, the PARP inhibitor is talazoparib or niraparib. In a specific embodiment, the PARP inhibitor is Talazoparib. In another specific embodiment, the PARP inhibitor is Niraparib.

In other embodiments, the payload is an auristatin, for example an MMAE or MMAF. In other embodiments, the payload is a maytansine, for example, DM1 or DM4. In other embodiments, the payload is a pyrrolobenzodiazepine dimer (PBD).

### Compounds

In one aspect, provided herein are compounds of Formula (I):

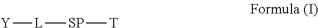

wherein,
T is a moiety derived from a compound capable of inhibiting topoisomerase enzyme or poly (ADP-ribose) polymerase (PARP);
SP is absent, a di- or tri-peptide linking moiety having L bonded to the N-terminus and T bonded to the C-terminus, or a linking moiety capable of self-immolation at pH less than 8;
L is a di- or tri-peptide linking moiety having Y bonded to the N-terminus and SP bonded to the C-terminus; and
Y is a conjugation moiety capable of forming a covalent bond with a nitrogen atom of a lysine residue or a sulfur atom of a cysteine residue;
or a salt or a hydrate thereof.

In certain embodiments, the linker -SP-L- comprises one or more straight or branched-chain carbon moieties and polyether (e.g., PEG) moieties, and combinations thereof. In certain embodiments, these linkers optionally have amide linkages, sulfhydryl linkages, or hetero functional linkages. In certain embodiments, the linker -SP-L- comprises one or more of carbon atoms, nitrogen atoms, sulfur atoms, oxygen atoms, and combinations thereof. In certain embodiments, the linker -SP-L- comprises one or more of an ether bond, thioether bond, amine bond, amide bond, carbon-carbon bond, carbon-nitrogen bond, carbon-oxygen bond, carbon-sulfur bond, and combinations thereof. In certain embodiments, the linker -SP-L- comprises a linear structure. In certain embodiments, the linker -SP-L- comprises a branched structure. In certain embodiments, the linker comprises a cyclic structure.

In certain embodiments, the linker -SP-L- is between about 10 Å and about 20 Å in length. In certain embodiments, the linker -SP-L- is between about 15 Å and about 20 Å in length. In certain embodiments, the linker -SP-L- is about 15 Å in length. In certain embodiments, L is about 16 Å in length. In certain embodiments, the linker -SP-L- is about 17 Ain length.

In certain embodiments, the linker -SP-L- is a linker between about 5 Å and about 500 Å. In certain embodiments, the linker -SP-L- is between about 10 Å and about 100 Å. In certain embodiments, the linker -SP-L- is between about 10 Å and about 20 Å, between about 20 Å and about 30 Å, between about 30 Å and about 40 Å, between about 40 Å and about 50 Å, between about 50 Å and about 60 Å, between about 60 Å and about 70 Å, between about 70 Å and about 80 Å, between about 80 Å and about 90 Å, or between about 90 Å and about 100 Å.

In certain embodiments, -SP-L- separates T and Y by a chain of 4 or 5 consecutive atoms, by a chain of 6 to 10 consecutive atoms, by a chain of 11 to 15 consecutive atoms, by a chain of 16 to 20 consecutive atoms, by a chain of 21 to 25 consecutive atoms, by a chain of 26 to 30 consecutive atoms, by a chain of 31 to 35 consecutive atoms, by a chain of 36 to 40 consecutive atoms, by a chain of 41 to 45 consecutive atoms, or by a chain of 46 to 50 consecutive atoms.

In certain embodiments, Y is a conjugation moiety capable of forming a covalent bond with an amino acid of a polypeptide.

In certain embodiments, Y is a conjugation moiety capable of forming a covalent bond with a nitrogen atom, e.g., a nitrogen atom from an amino acid of a polypeptide (e.g., a nitrogen atom from an amino group).

In certain embodiments, the conjugation moiety comprises an activated carbonyl group. In certain embodiments, the conjugation moiety comprises an activated carbonyl group of

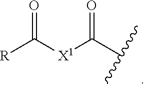

In some embodiments, X¹ is selected from bond, —(CH₂)ₙ—, —(CH₂)ₙ—(OCH₂CH₂)ₘ—, —(CH₂)ₙ—O—(CH₂)ₘ—, and —(CH₂)ₙ—NH—(CH₂)ₘ—, wherein n is an integer from 1 to 5, and m is an integer from 1 to 8. In some embodiments, X¹ is selected from —(CH₂)ₙ—(OCH₂CH₂)ₘ—, —(CH₂)ₙ—O—(CH₂)ₘ—, and —(CH₂)ₙ—NH—(CH₂)ₘ—. In some embodiments, X¹ is selected from —(CH₂)ₙ—, —(CH₂)ₘ—O—(CH₂)ₘ—, and —(CH₂)ₙ—NH—(CH₂)ₘ—. In some embodiments, X¹ is selected from —(CH₂)ₙ—, —(CH₂)ₙ—(OCH₂CH₂)ₘ—, and —(CH₂)ₙ—NH—(CH₂)ₘ—. In some embodiments, X¹ is selected from —(CH₂)ₙ—, —(CH₂)ₙ—(OCH₂CH₂)ₘ—, and —(CH₂)ₙ—O—(CH₂)ₘ—. In one embodiment, X¹ is —(CH₂)ₙ—. In another embodiment, X¹ is —(CH₂)ₙ—(OCH₂CH₂)ₘ—. In yet another embodiment, X¹ is —(CH₂)ₙ—O—(CH₂)ₘ—. In yet another embodiment, X¹ is —(CH₂)ₙ—NH—(CH₂)ₘ—.

In yet another embodiment, X¹ is bond.

In some embodiments, R is selected from the group consisting of:

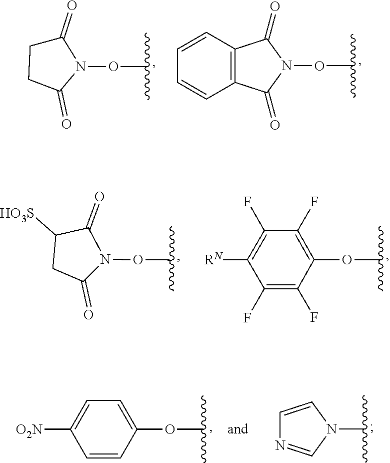

wherein R^{N} is hydrogen or fluoro.

In certain embodiments, the conjugation moiety Y is selected from

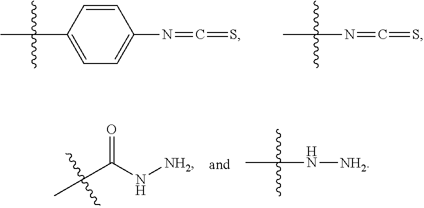

In certain embodiments, the conjugation moiety comprises an acyl halide group or a Michael acceptor group.

In certain embodiments, the conjugation moiety Y is an acyl halide group. In certain embodiments, the acyl halide is:

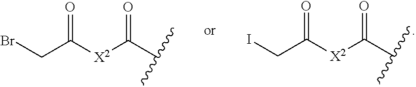

In certain embodiments, wherein X² is selected from bond, —(CH₂)ₙ—, —(CH₂)ₙ—(OCH₂CH₂)ₚ—, —(CH₂)ₙ—O—(CH₂)ₚ—, and —(CH₂)ₙ—NH—(CH₂)ₚ—, wherein n is an integer from 1 to 5, and p is an integer from 1 to 5. In some embodiments, X² is selected from —(CH₂)ₙ—(OCH₂CH₂)ₚ—, —(CH₂)ₙ—O—(CH₂)ₚ—, and —(CH₂)ₙ—NH—(CH₂)ₚ—. In some embodiments, X² is selected from —(CH₂)ₙ—, —(CH₂)ₙ—O—(CH₂)ₚ—, and —(CH₂)ₙ—NH—(CH₂)ₚ—. In some embodiments, X² is selected from —(CH₂)ₙ—, —(CH₂)ₙ—(OCH₂CH₂)ₚ—, and —(CH₂)ₙ—NH—(CH₂)ₚ—. In some embodiments, X² is selected from —(CH₂)ₙ—, —(CH₂)ₙ—(OCH₂CH₂)ₚ—, and —(CH₂)ₙ—O—(CH₂)ₚ—. In one embodiment, X² is —(CH₂)ₙ—. In another embodiment, X² is —(CH₂)ₙ—(OCH₂CH₂)ₚ—. In yet another embodiment, X² is —(CH₂)ₙ—O—(CH₂)ₚ—. In yet another embodiment, X² is —(CH₂)ₙ—NH—(CH₂)ₚ—. In yet another embodiment, X² is bond.

In certain embodiments, Y is a conjugation moiety capable of forming a covalent bond with a sulfur atom from an amino acid of a polypeptide (e.g., a sulfur atom from a thiol group). In certain embodiments, the conjugation moiety comprises a Michael acceptor group. In certain embodiments, the Michael acceptor group is

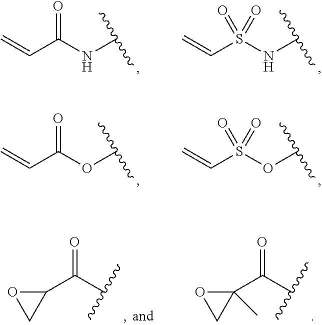

In some embodiments, the Michael acceptor group is

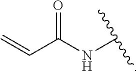 [SMILES: *NC(=O)C=C]

In some embodiments, the Michael acceptor group is

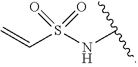 [SMILES: *NS(=O)(=O)C=C]

In some embodiments, the Michael acceptor group is

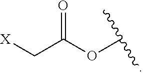

In some embodiments, the Michael acceptor group is

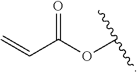 [SMILES: *OC(=O)C=C]

In some embodiments, the Michael acceptor group is

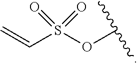 [SMILES: *OS(=O)(=O)C=C]

In some embodiments, the Michael acceptor group is

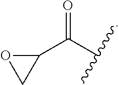 [SMILES: *C(=O)C1CO1]

In some embodiments, the Michael acceptor group is

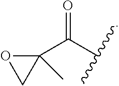 [SMILES: *C(=O)C1(C)CO1]

In certain embodiments, the Michael acceptor group is:

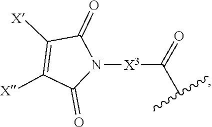

wherein X³ is selected from bond, —(CH₂)ₙ—, —(CH₂)ₙ—(OCH₂CH₂)ₘ—, —(CH₂)ₙ—O—(CH₂)ₚ—, —(CH₂)ₙ—NH—(CH₂)ₚ—, wherein n is an integer from 1 to 5, and p is an integer from 1 to 8; wherein x′ is H, Cl, Br, I, 2-thiopyridyl, or 4-cyanophenoxy; and x″ is H, Cl, Br, I, 2-thiopyridyl, or 4-cyanphenoxy.

In certain embodiments, wherein X³ is selected from bond, —(CH₂)ₙ—, —(CH₂)ₙ—(OCH₂CH₂)ₚ—, —(CH₂)ₙ—O—(CH₂)ₚ—, and —(CH₂)ₙ—NH—(CH₂)ₚ—, wherein n is an integer from 1 to 5, and p is an integer from 1 to 8. In some embodiments, X³ is selected from —(CH₂)ₙ—(OCH₂CH₂)ₚ—, —(CH₂)ₙ—O—(CH₂)ₚ—, and —(CH₂)ₙ—NH—(CH₂)ₚ—. In some embodiments, X³ is selected from —(CH₂)ₙ—, —(CH₂)ₙ—O—(CH₂)ₚ—, and —(CH₂)ₙ—NH—(CH₂)ₚ—. In some embodiments, X³ is selected from —(CH₂)ₙ—, —(CH₂)ₙ—(OCH₂CH₂)ₚ—, and —(CH₂)ₙ—NH—(CH₂)ₚ—. In some embodiments, X³ is selected from —(CH₂)ₙ—, —(CH₂)ₙ—(OCH₂CH₂)ₚ—, and —(CH₂)ₙ—O—(CH₂)ₚ—. In one embodiment, X³ is —(CH₂)ₙ—. In another embodiment, X³ is —(CH₂)ₙ—(OCH₂CH₂)ₚ—. In yet another embodiment, X³ is —(CH₂)ₙ—O—(CH₂)ₚ—. In yet another embodiment, X³ is —(CH₂)ₙ—NH—(CH₂)ₚ—. In yet another embodiment, X³ is bond.

In certain embodiments, x′ and x″ are each independently H, Cl, Br, or I. In certain embodiments, x′ and x″ are the same. In certain embodiments, x′ and x″ are both H or both Br. In one embodiment, x′ and x″ are both H. In certain embodiments, x′ and x″ are both Br.

In certain embodiments, L is a di-peptide linking moiety having the structure of:

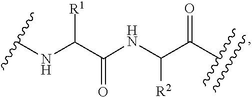

wherein R¹ and R² are independently selected from H, —CH₂CH₂CH₂NHCONH₂, or the side chain of a naturally occurring amino acid, wherein [symbol] represents the point of attachment to Y and [symbol] represents the point of attachment to SP.

In certain embodiments, R¹ and R² are independently selected from the group consisting of hydrogen, —CH₃, —CH(CH₃)₂, —CH₂CH₂CH₂CH₂NH₂, —CH₂C₆H₅, —CH₂C₆H₄OH, —CH₂CH₂CH₂NH(NH)NH₂, —CH₂CH₂CH₂NHCONH₂, and —CH₂CH₂CO₂H. In certain embodiments, R¹ is selected from the group consisting of —CH₃, —CH(CH₃)₂, —CH₂CH₂CH₂CH₂NH₂, and —CH₂C₆H₅. In certain embodiments, R² is selected from the group consisting of —CH₃, —CH₂CH₂CH₂CH₂NH₂, —CH₂C₆H₅, and —CH₂CH₂CH₂NHCONH₂. In certain embodiments, R¹ is —CH₃ and R² is —CH₂CH₂CH₂CH₂NH₂. In certain embodiments, R¹ is —CH₃ and R² is —CH₃. In certain embodiments, R¹ is —CH(CH₃)₂ and R² is —CH₂CH₂CH₂NHCONH₂. In certain embodiments, R¹ is —CH₂C₆H₅ and R² is —CH₂CH₂CH₂CH₂NH₂. In certain embodiments, R¹ is —CH₃ and R² is —CH₂C₆H₅. In certain embodiments, R¹ is —CH₂CH₂CH₂CH₂NH₂ and R² is —CH₃.

In certain embodiments, L is a tri-peptide linking moiety having the structure of.

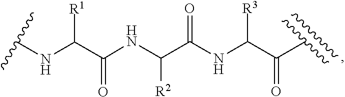

wherein R¹, R² and R³ are independently H, —CH₂CH₂CH₂NHCONH₂, or the side chain of a naturally occurring amino acid, and wherein [symbol] represents the point of attachment to Y and [symbol] represents the point of attachment to SP. In certain embodiments, R¹, R², and R³ are independently selected from the group consisting of hydrogen, CH₃, —CH(CH₃)₂, —CH₂CH₂CH₂CH₂NH₂, —CH₂C₆H₅, CH₂C₆H₄OH, —CH₂CH₂CH₂NH(NH)NH₂, —CH₂CH₂CH₂NHCONH₂, and —CH₂CH₂CO₂H. In certain embodiments, R¹ is H or CH₃. In certain embodiments, R² is —CH₂CH₂CH₂CH₂NH₂, —CH₂C₆H₅, or CH₃. In certain embodiments, R³ is H or CH₃. In certain embodiments, R¹ is H, R² is —CH₂C₆H5, and R³ is H. In certain embodiments, R¹ is H, R² is —CH₂CH₂CH₂CH₂NH₂, and R³ is H. In certain embodiments, R¹ is CH₃, R² is CH₃, and R³ is CH₃. In certain embodiments, R² is —CH₂CH₂CH₂CH₂NH₂. In certain embodiments, R¹ is CH₃, R² is —CH₂CH₂CH₂CH₂NH₂, and R³ is H. In certain embodiments, R² is —CH₂CH₂CH₂CH₂NH₂, and R³ is H or CH₃.

L is a peptide linking moiety having the structure of -(AA¹)ₐ(AA²)_{b}(AA³)_{c}(AA⁴)_{d}-, wherein AA¹, AA², AA³, and AA⁴ are each independently a natural or unnatural amino acid, and wherein a, b, c, and d are each independently 0 or 1. L is a peptide linking moiety having the structure of: -(AA¹)ₐ(AA²)_{b}(AA³)_{c}-, wherein AA¹, AA², and AA³ are each independently a natural or unnatural amino acid, and wherein a, b, c, and d are each independently 0 or 1.

In certain embodiments, SP is a linking moiety.

In certain embodiments, SP is absent. In certain embodiments, SP is a di- or tri-peptide linking moiety having L bonded to the N-terminus, and T bonded to the C-terminus. In certain embodiments, SP is a di-peptide linking moiety. In certain embodiments, the di-peptide linking moiety of SP is

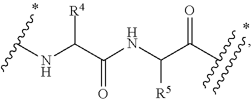

wherein R⁴ and R⁵ are each independently H or optionally substituted C₁₋₆ alkyl, wherein [symbol] represents the point of attachment to L and [symbol] represents the point of attachment to T. In certain embodiments, R⁴ and R⁵ are each independently H or —CH₃. In certain embodiments, R⁴ is H, and R⁵ is H. In certain embodiments, SP is a tri-peptide linking moiety. In certain embodiments, the tri-peptide linking moiety of SP is

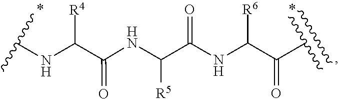

wherein R⁴, R⁵ and R⁶ are each independently H or optionally substituted C₁₋₆ alkyl, wherein [symbol] represents the point of attachment to L and [symbol] represents the point of attachment to T. In certain embodiments, R⁴, R⁵ and R⁶ are each independently H or —CH₃. In certain embodiments, R⁴ is H; R⁵ is H; and R⁶ is H.

In certain embodiments, SP is a linking moiety capable of self-immolation at pH less than 8.

In certain embodiments, SP is

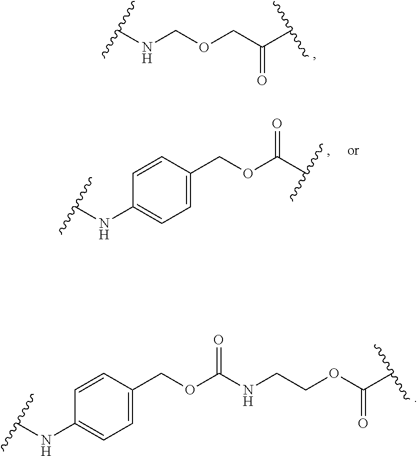

In certain embodiments, SP is

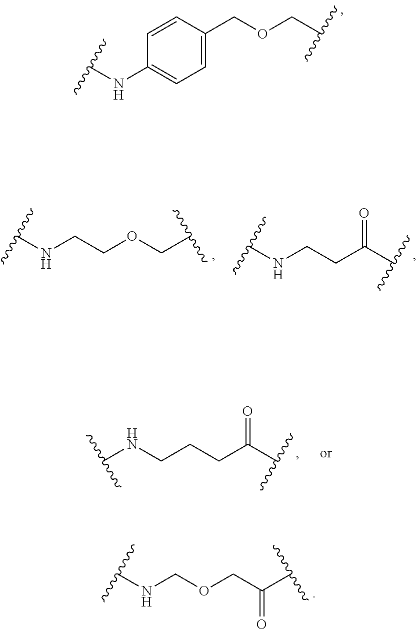 [SMILES: *COCc1ccc(N*)cc1 ; *COCCN* ; *NCCC(*)=O ; *NCCCC(*)=O ; *NCOCC(*)=O]

In certain embodiments, SP is selected from:

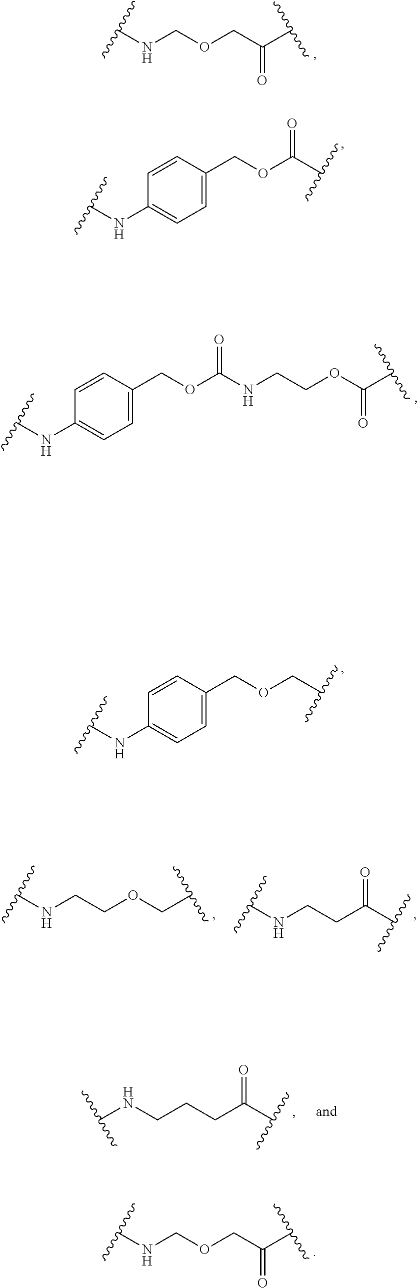

In certain embodiments, T is a moiety derived from a compound capable of inhibiting topoisomerase (e.g., a topoisomerase I).

In certain embodiments, T is selected from:

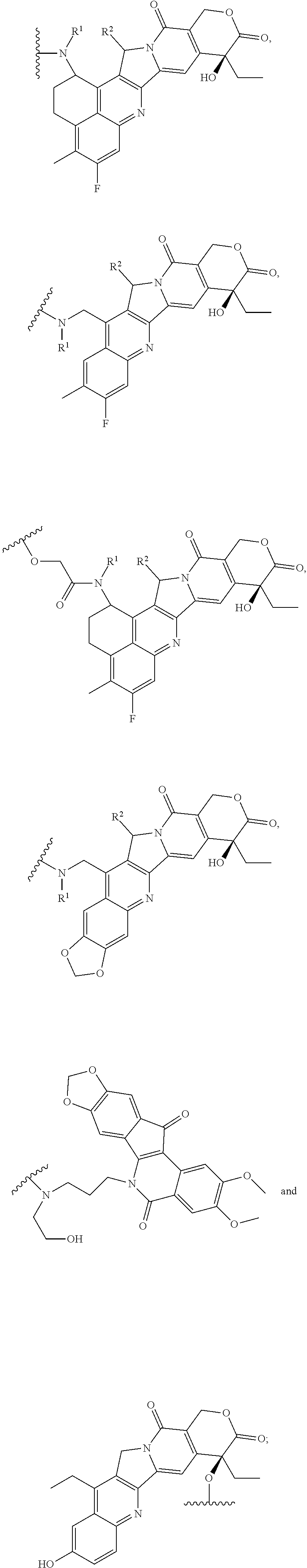

wherein R¹ is H, CH₃ or SO₂CH₃; and R² is H, or CH₂OH.

In certain embodiments, T is a moiety derived from a compound capable of inhibiting poly (ADP-ribose) polymerase (PARP).

In certain embodiments, T is selected from:

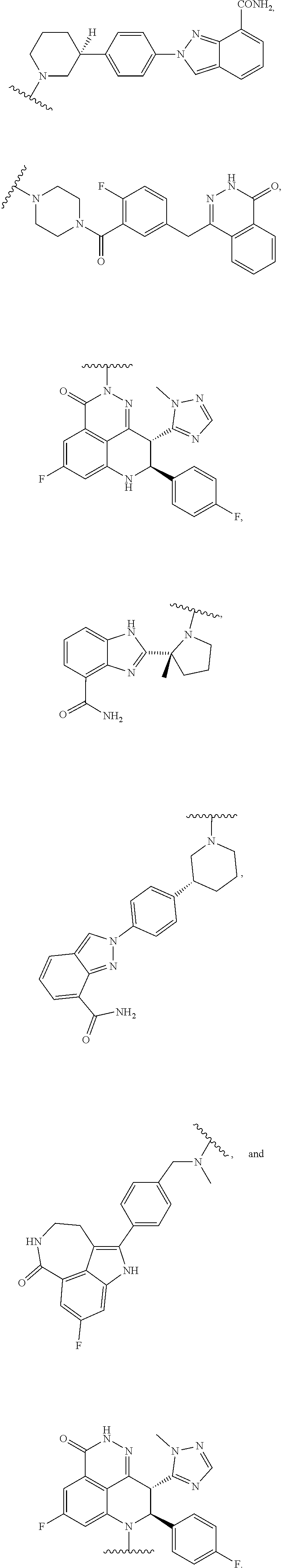

In certain embodiments, T is selected from:

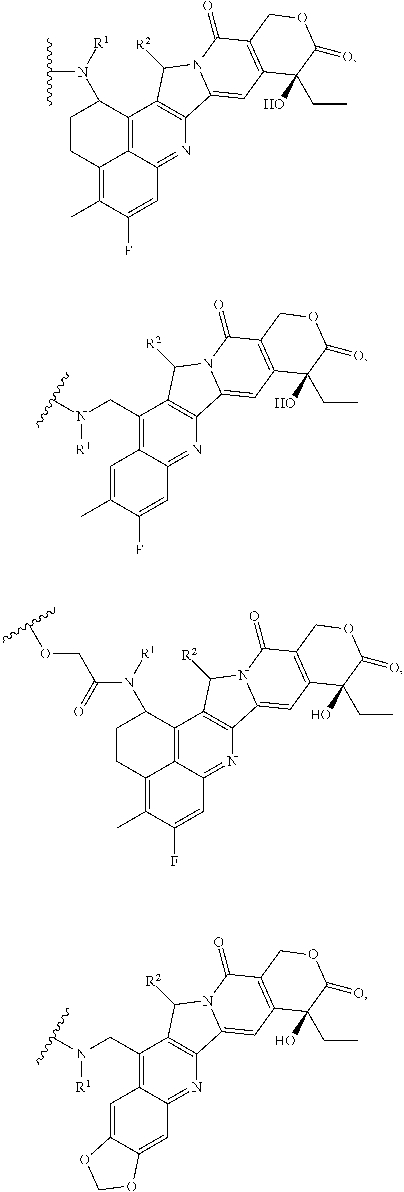

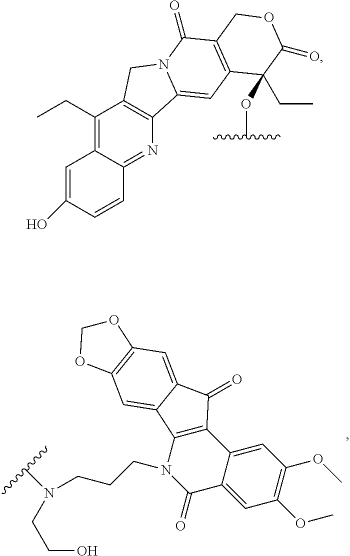

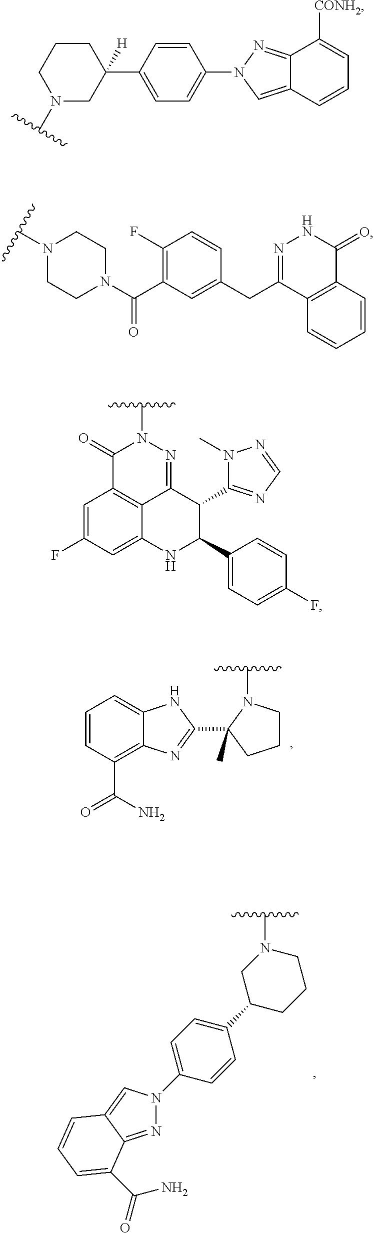

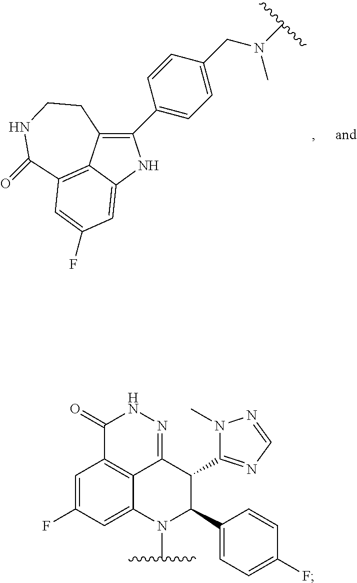

wherein R¹ is H, CH₃ or SO₂CH₃; an R² is H, or CH₂OH.

In one embodiment, T is

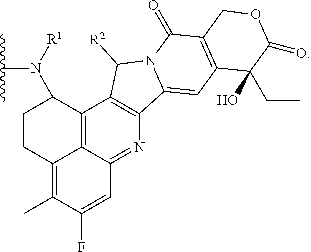

In another embodiment, T is

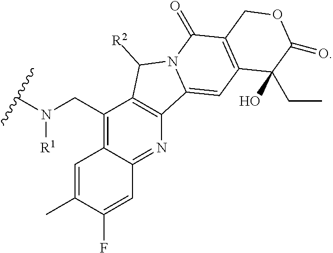

In yet another embodiment, T is

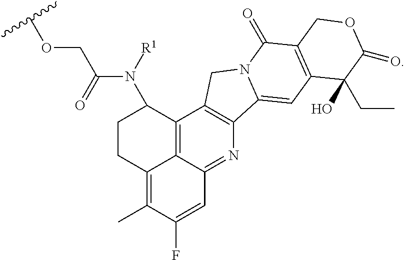

In yet another embodiment, T is

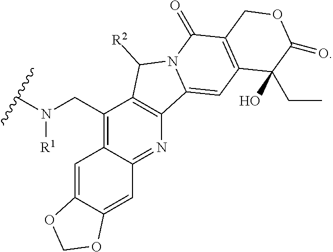

In yet another embodiment, T is

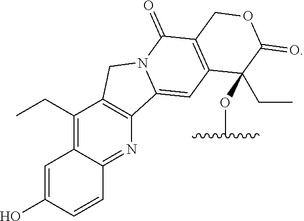 [SMILES: *O[C@]1(CC)C(=O)OCc2c1cc1n(c2=O)Cc2c-1nc1ccc(O)cc1c2CC]

In yet another embodiment, T is

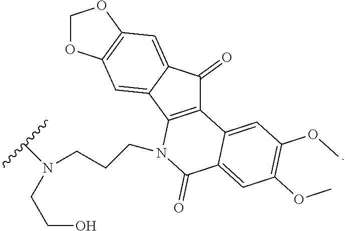 [SMILES: *N(CCO)CCCn1c2c(c3cc(OC)c(OC)cc3c1=O)C(=O)c1cc3c(cc1-2)OCO3]

In yet another embodiment, T is

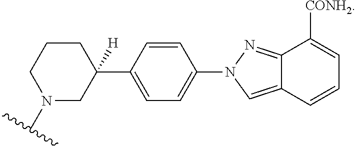 [SMILES: *N1CCC[C@@H](c2ccc(-n3cc4cccc(C(N)=O)c4n3)cc2)C1]

In yet another embodiment, T is

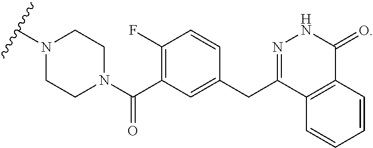 [SMILES: *N1CCN(C(=O)c2cc(Cc3n[nH]c(=O)c4ccccc34)ccc2F)CC1]

In yet another embodiment, T is

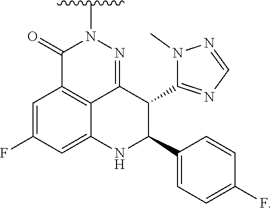 [SMILES: *n1nc2c3c(cc(F)cc3c1=O)N[C@H](c1ccc(F)cc1)[C@H]2c1ncnn1C]

In yet another embodiment, T is

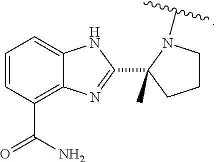

In yet another embodiment, T is

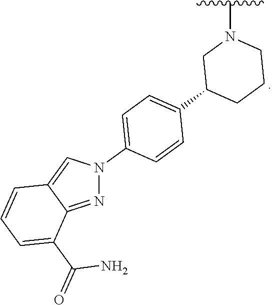 [SMILES: *N1CCC[C@H](c2ccc(-n3cc4cccc(C(N)=O)c4n3)cc2)C1]

In yet another embodiment, T is

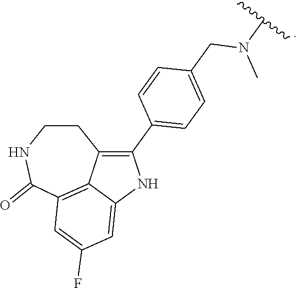 [SMILES: *N(C)Cc1ccc(-c2[nH]c3cc(F)cc4c3c2CCNC4=O)cc1]

In yet another embodiment, T is

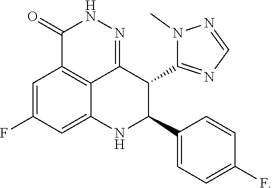 [SMILES: Cn1ncnc1[C@H]1c2n[nH]c(=O)c3cc(F)cc(c23)N[C@@H]1c1ccc(F)cc1]

### EXEMPLARY COMPOUNDS

In some embodiments, the compounds described herein has a structure provided in Table 1.

TABLE 1

| Compound | |
|---|---|
| # | Chemical Structure |
| 1 | 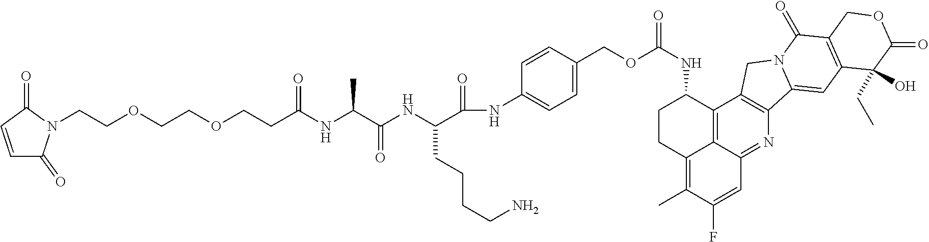 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCCN)NC(=O)[C@H](C)NC(=O)CCOCCOCCN2C(=O)C=CC2=O)cc1)CC3] |
| 2 | 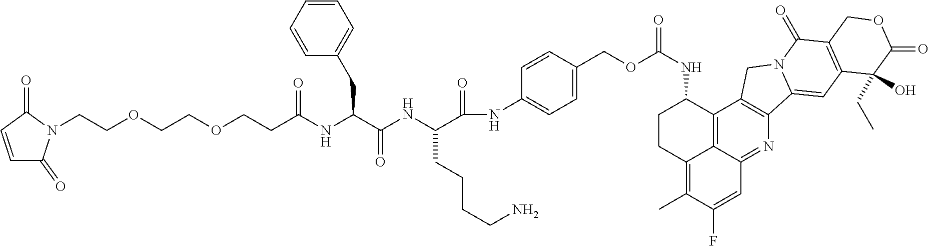 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCCN)NC(=O)[C@H](Cc2ccccc2)NC(=O)CCOCCOCCN2C(=O)C=CC2=O)cc1)CC3] |
| 3 | 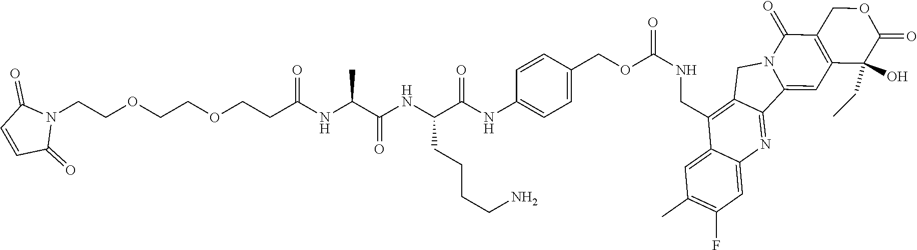 [SMILES: CC[C@@]1(O)C(=O)OCc2c1cc1n(c2=O)Cc2c-1nc1cc(F)c(C)cc1c2CNC(=O)OCc1ccc(NC(=O)[C@H](CCCCN)NC(=O)[C@H](C)NC(=O)CCOCCOCCN2C(=O)C=CC2=O)cc1] |
| 4 | 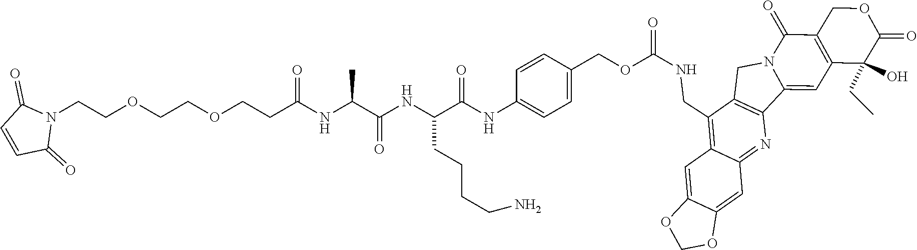 [SMILES: CC[C@@]1(O)C(=O)OCc2c1cc1n(c2=O)Cc2c-1nc1cc3c(cc1c2CNC(=O)OCc1ccc(NC(=O)[C@H](CCCCN)NC(=O)[C@H](C)NC(=O)CCOCCOCCN2C(=O)C=CC2=O)cc1)OCO3] |
| 5 | 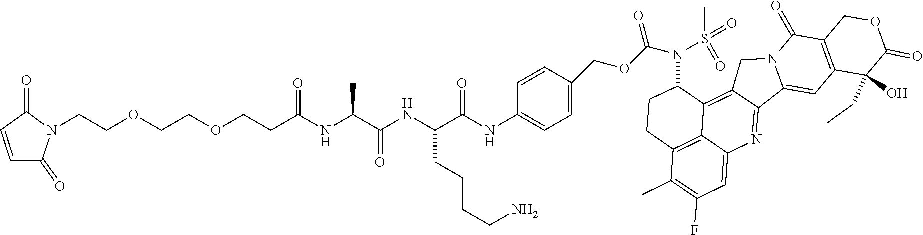 [SMILES: CC[C@@]1(O)C(=O)OCc2c1cc1n(c2=O)Cc2c-1nc1cc(F)c(C)c3c1c2[C@@H](N(C(=O)OCc1ccc(NC(=O)[C@H](CCCCN)NC(=O)[C@H](C)NC(=O)CCOCCOCCN2C(=O)C=CC2=O)cc1)S(C)(=O)=O)CC3] |
| 6 | 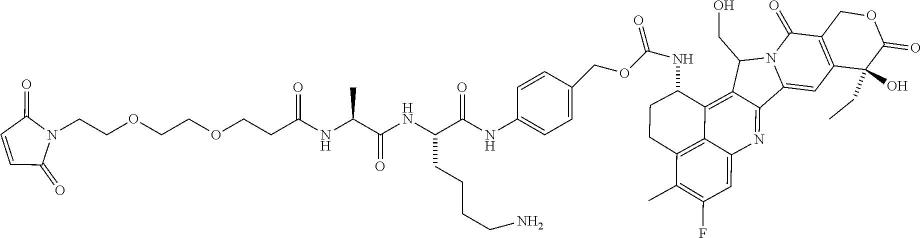 [SMILES: CC[C@@]1(O)C(=O)OCc2c1cc1n(c2=O)C(CO)c2c-1nc1cc(F)c(C)c3c1c2[C@@H](NC(=O)OCc1ccc(NC(=O)[C@H](CCCCN)NC(=O)[C@H](C)NC(=O)CCOCCOCCN2C(=O)C=CC2=O)cc1)CC3] |
| 7 | 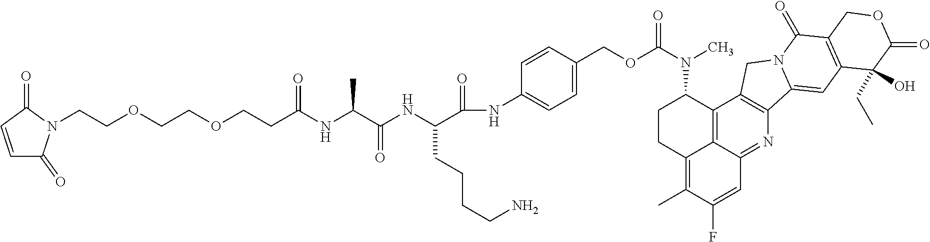 [SMILES: CC[C@@]1(O)C(=O)OCc2c1cc1n(c2=O)Cc2c-1nc1cc(F)c(C)c3c1c2[C@@H](N(C)C(=O)OCc1ccc(NC(=O)[C@H](CCCCN)NC(=O)[C@H](C)NC(=O)CCOCCOCCN2C(=O)C=CC2=O)cc1)CC3] |
| 8 | 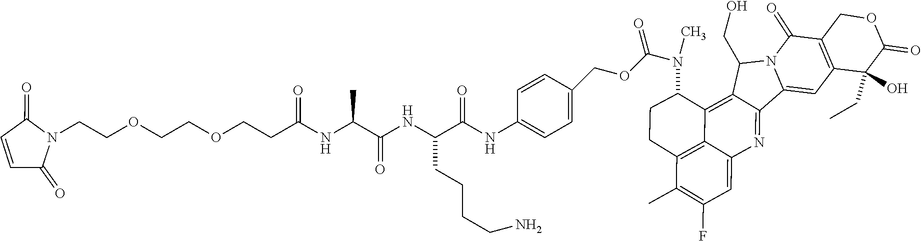 [SMILES: CC[C@@]1(O)C(=O)OCc2c1cc1n(c2=O)C(CO)c2c-1nc1cc(F)c(C)c3c1c2[C@@H](N(C)C(=O)OCc1ccc(NC(=O)[C@H](CCCCN)NC(=O)[C@H](C)NC(=O)CCOCCOCCN2C(=O)C=CC2=O)cc1)CC3] |
| 9 | 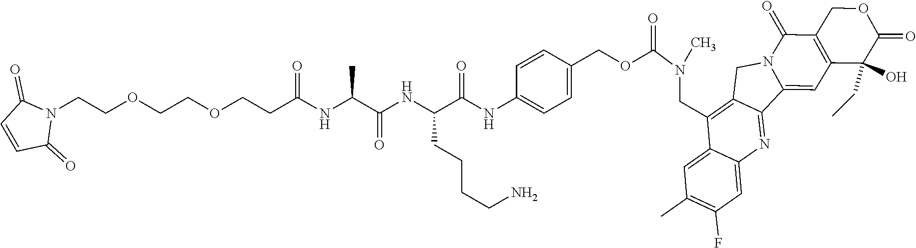 [SMILES: CC[C@@]1(O)C(=O)OCc2c1cc1n(c2=O)Cc2c-1nc1cc(F)c(C)cc1c2CN(C)C(=O)OCc1ccc(NC(=O)[C@H](CCCCN)NC(=O)[C@H](C)NC(=O)CCOCCOCCN2C(=O)C=CC2=O)cc1] |
| 10 | 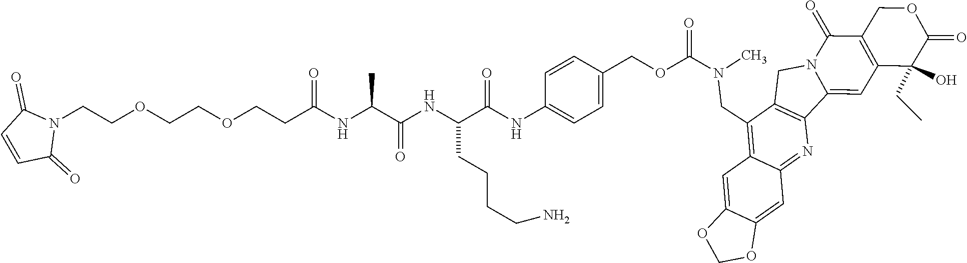 [SMILES: CC[C@@]1(O)C(=O)OCc2c1cc1n(c2=O)Cc2c-1nc1cc3c(cc1c2CN(C)C(=O)OCc1ccc(NC(=O)[C@H](CCCCN)NC(=O)[C@H](C)NC(=O)CCOCCOCCN2C(=O)C=CC2=O)cc1)OCO3] |
| 11 | 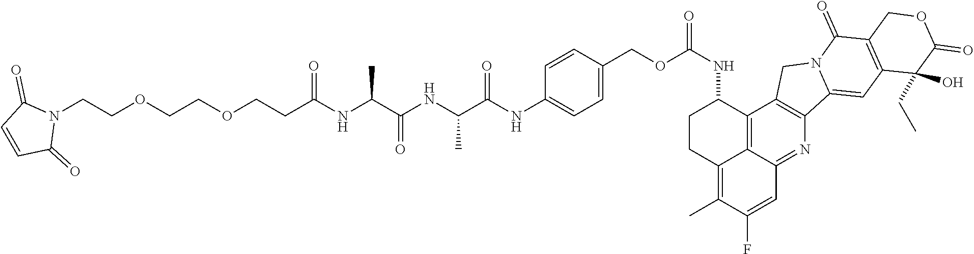 |
| 12 | 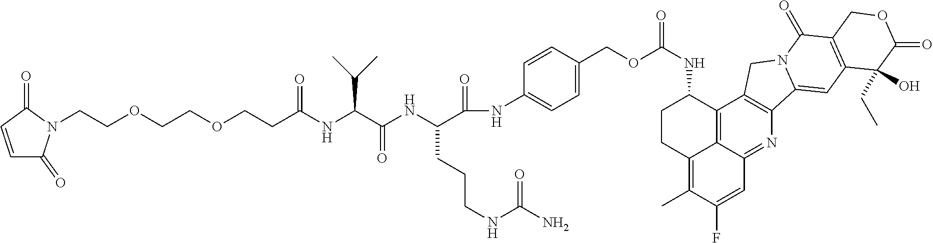 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)CCOCCOCCN2C(=O)C=CC2=O)C(C)C)cc1)CC3] |
| 13 | 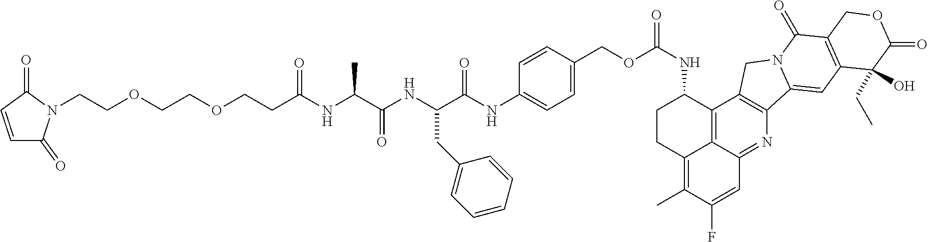 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](Cc2ccccc2)NC(=O)[C@H](C)NC(=O)CCOCCOCCN2C(=O)C=CC2=O)cc1)CC3] |
| 14 | 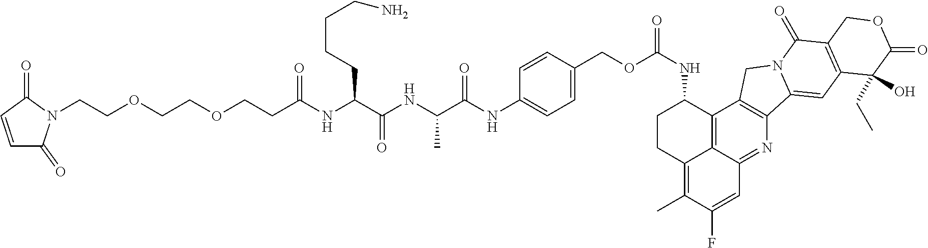 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@H](CCCCN)NC(=O)CCOCCOCCN2C(=O)C=CC2=O)cc1)CC3] |
| 15 | 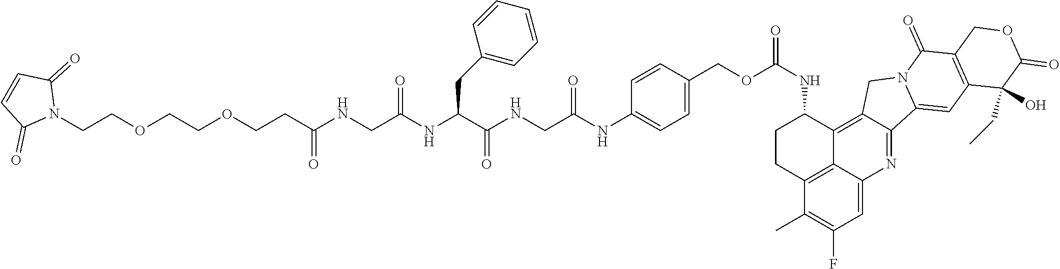 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)CNC(=O)[C@H](Cc2ccccc2)NC(=O)CNC(=O)CCOCCOCCN2C(=O)C=CC2=O)cc1)CC3] |
| 16 | 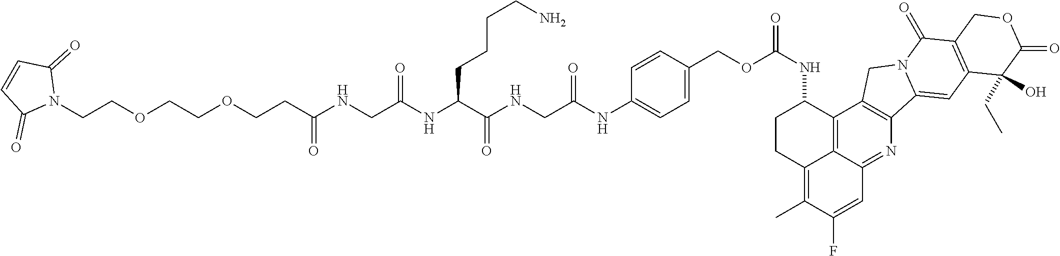 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)CNC(=O)[C@H](CCCCN)NC(=O)CNC(=O)CCOCCOCCN2C(=O)C=CC2=O)cc1)CC3] |
| 17 | 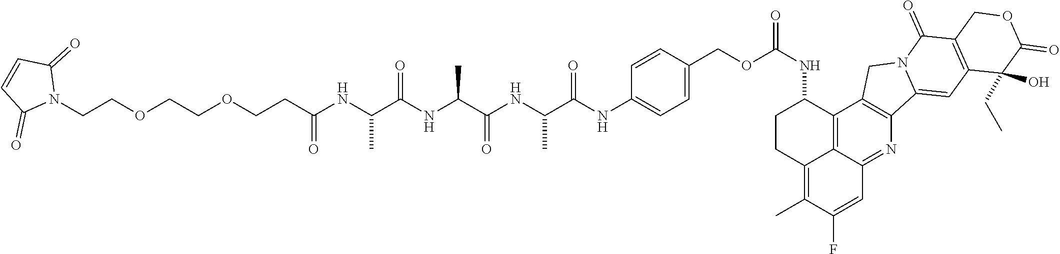 |
| 18 | 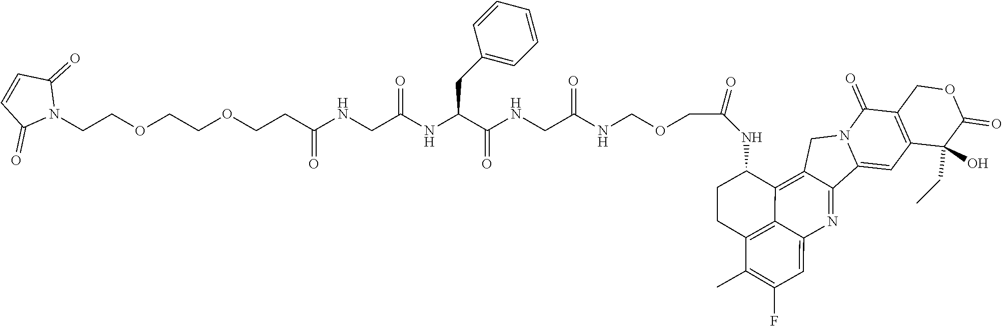 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CCOCCOCCN1C(=O)C=CC1=O)CC3] |
| 19 | 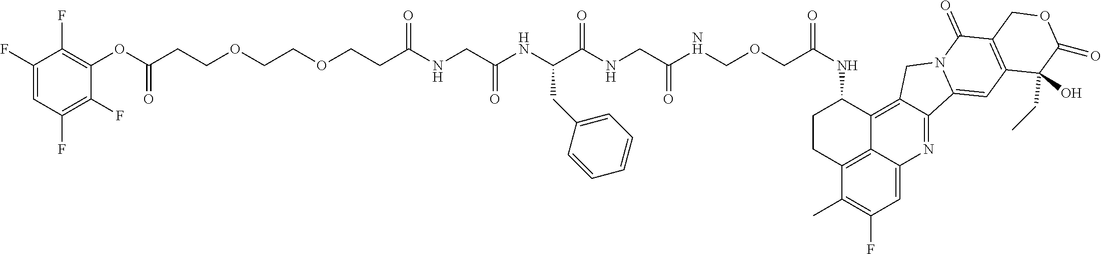 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CCOCCOCCC(=O)Oc1c(F)c(F)cc(F)c1F)CC3] |
| 20 | 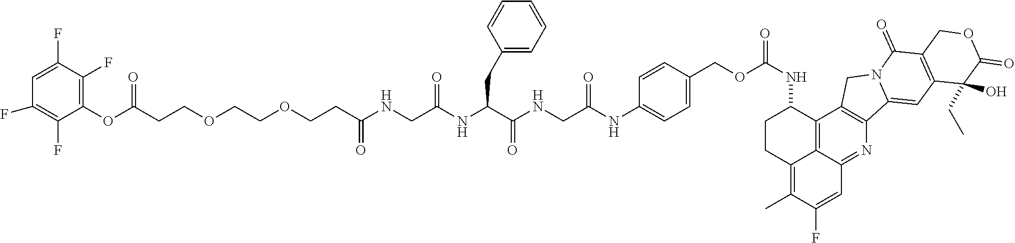 |
| 21 | 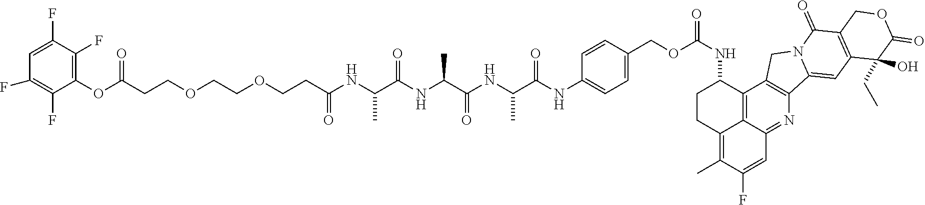 |
| 22 | 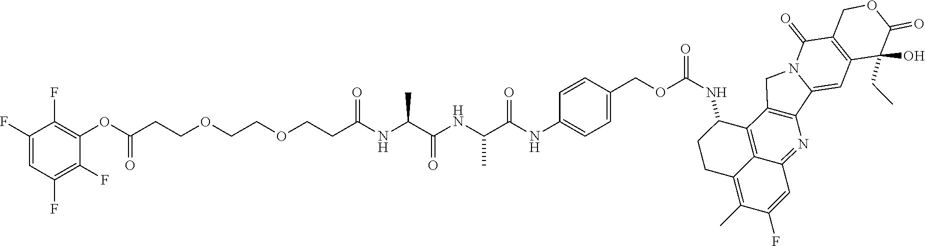 |
| 33 | 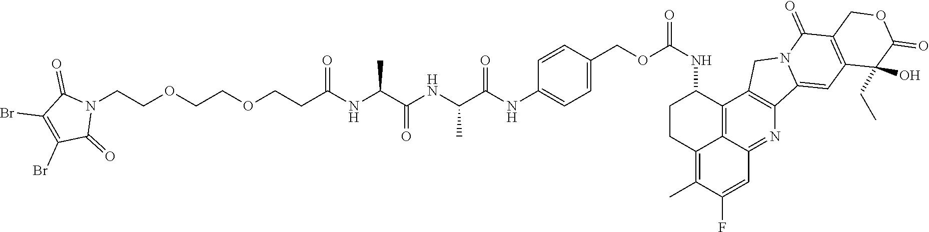 |
| 34 | 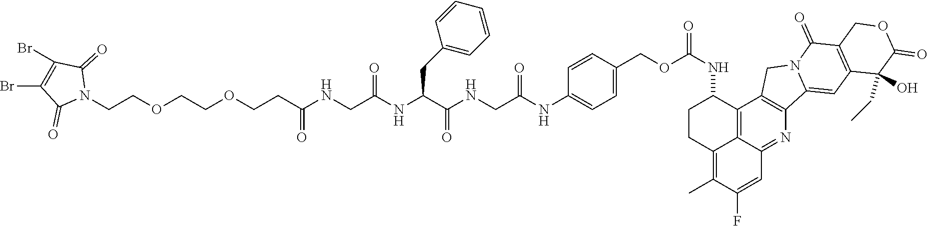 |
| 37 | 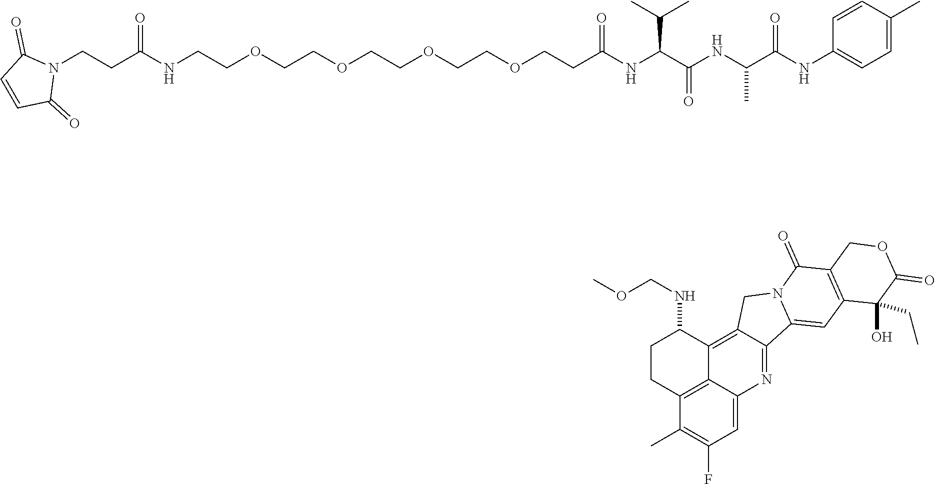 |
| 38 | 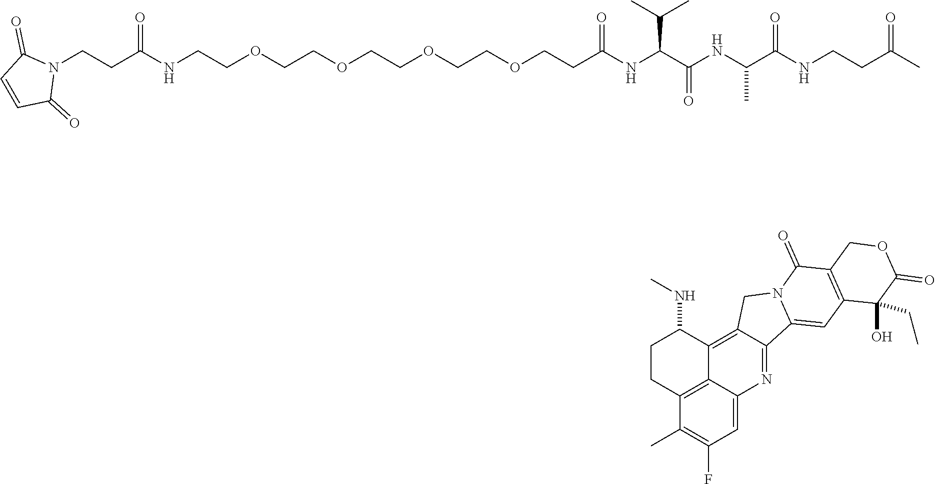 |
| 39 | 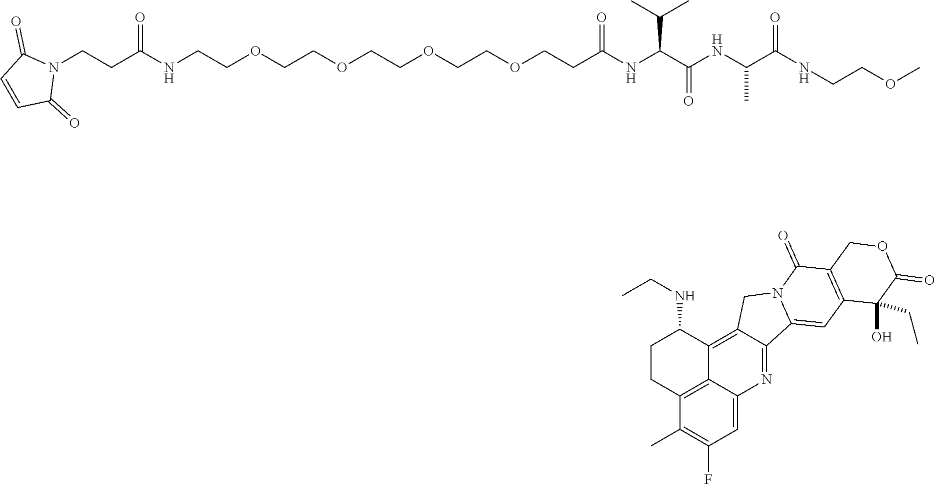 |
| 40 | 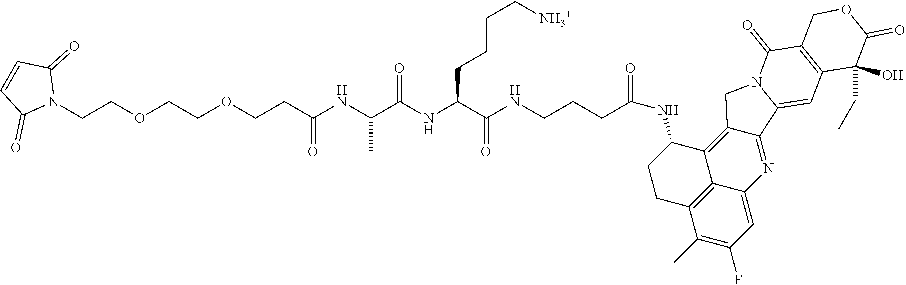 [SMILES: CC[C@@]1(O)C(=O)OCc2c1cc1n(c2=O)Cc2c-1nc1cc(F)c(C)c3c1c2[C@@H](NC(=O)CCCNC(=O)[C@H](CCCC[NH3+])NC(=O)[C@H](C)NC(=O)CCOCCOCCN1C(=O)C=CC1=O)CC3] |
| 41 | 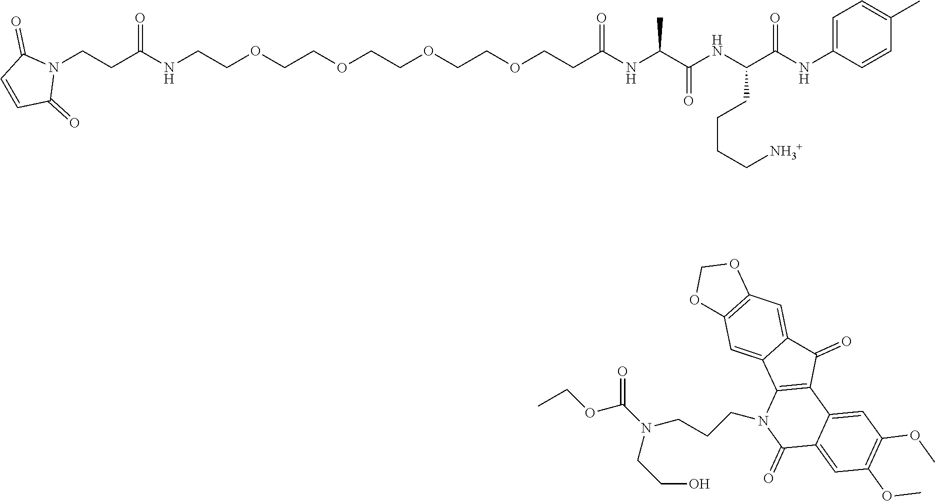 |
| 42 | 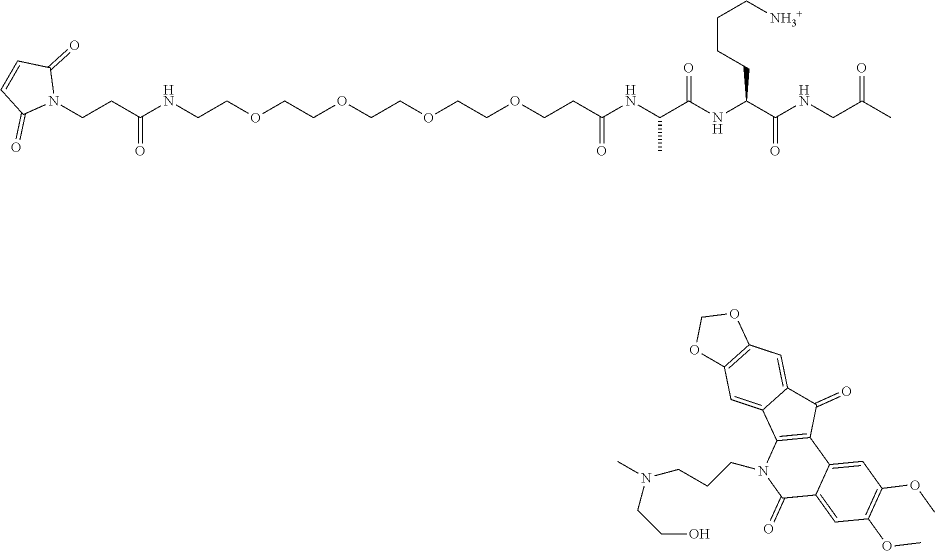 |
| 43 | 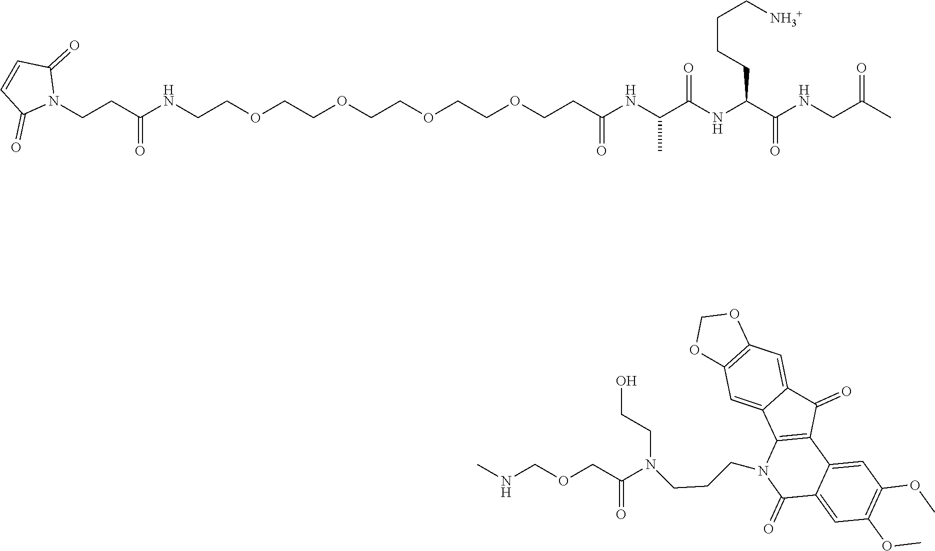 |
| 44 | 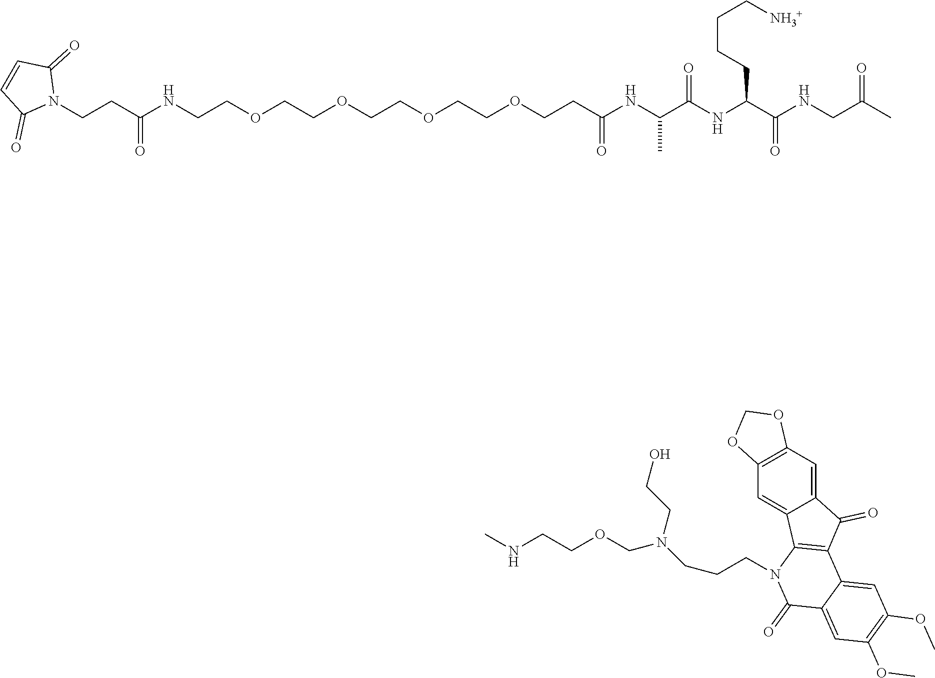 |
| 58 | 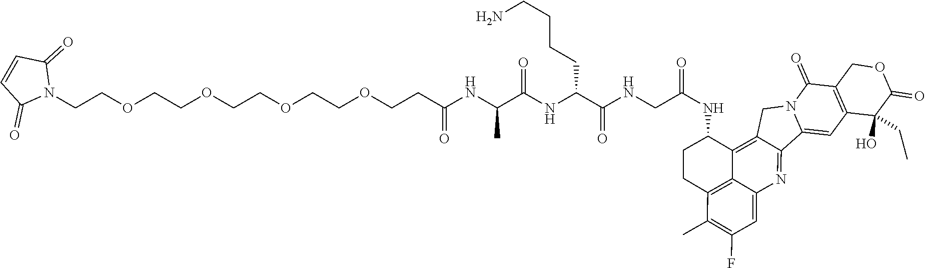 [SMILES: CC[C@@]1(O)C(=O)OCc2c1cc1n(c2=O)Cc2c-1nc1cc(F)c(C)c3c1c2[C@@H](NC(=O)CNC(=O)[C@@H](CCCCN)NC(=O)[C@@H](C)NC(=O)CCOCCOCCOCCOCCN1C(=O)C=CC1=O)CC3] |
| 59 | 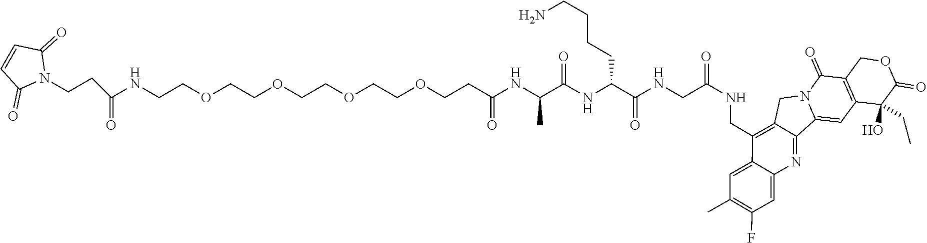 [SMILES: CC[C@@]1(O)C(=O)OCc2c1cc1n(c2=O)Cc2c-1nc1cc(F)c(C)cc1c2CNC(=O)CNC(=O)[C@@H](CCCCN)NC(=O)[C@@H](C)NC(=O)CCOCCOCCOCCOCCNC(=O)CCN1C(=O)C=CC1=O] |
| 60 | 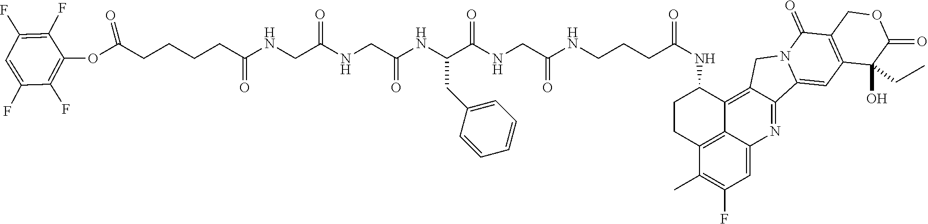 [SMILES: CC[C@@]1(O)C(=O)OCc2c1cc1n(c2=O)Cc2c-1nc1cc(F)c(C)c3c1c2[C@@H](NC(=O)CCCNC(=O)CNC(=O)[C@H](Cc1ccccc1)NC(=O)CNC(=O)CNC(=O)CCCCC(=O)Oc1c(F)c(F)cc(F)c1F)CC3] |
| 61 | 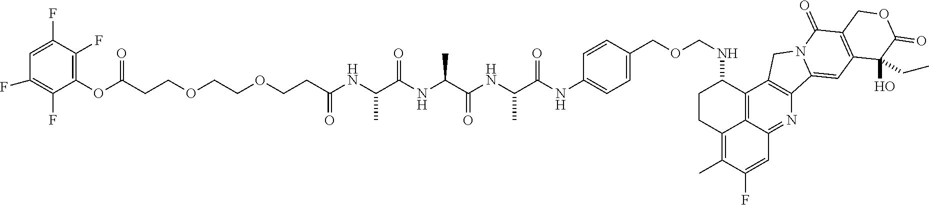 |
| 62 | 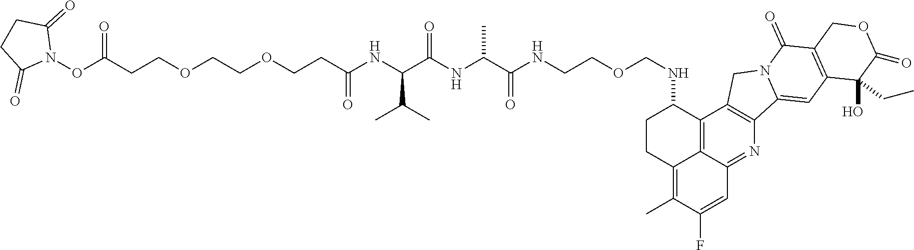 [SMILES: CC[C@@]1(O)C(=O)OCc2c1cc1n(c2=O)Cc2c-1nc1cc(F)c(C)c3c1c2[C@@H](NCOCCNC(=O)[C@@H](C)NC(=O)[C@H](NC(=O)CCOCCOCCC(=O)ON1C(=O)CCC1=O)C(C)C)CC3] |
| 63 | 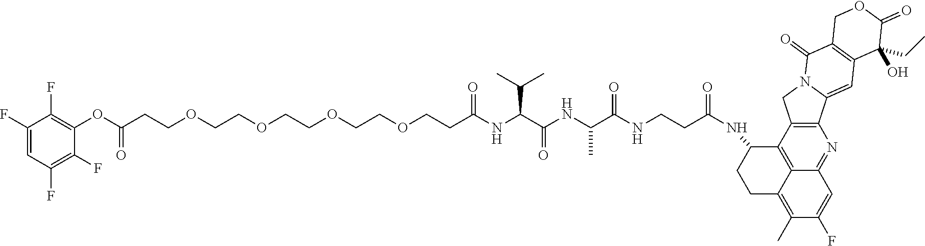 |
| 64 | 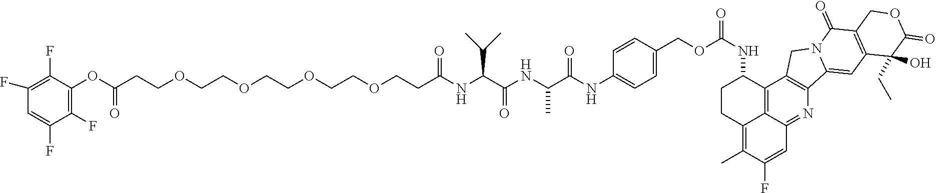 |
| 69 | 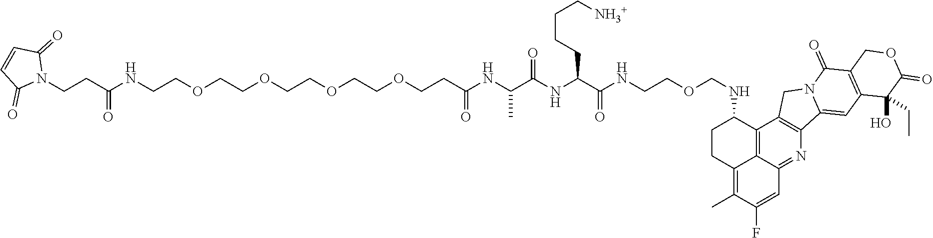 [SMILES: CC[C@@]1(O)C(=O)OCc2c1cc1n(c2=O)Cc2c-1nc1cc(F)c(C)c3c1c2[C@@H](NCOCCNC(=O)[C@H](CCCC[NH3+])NC(=O)[C@H](C)NC(=O)CCOCCOCCOCCOCCNC(=O)CCN1C(=O)C=CC1=O)CC3] |
| 70 | 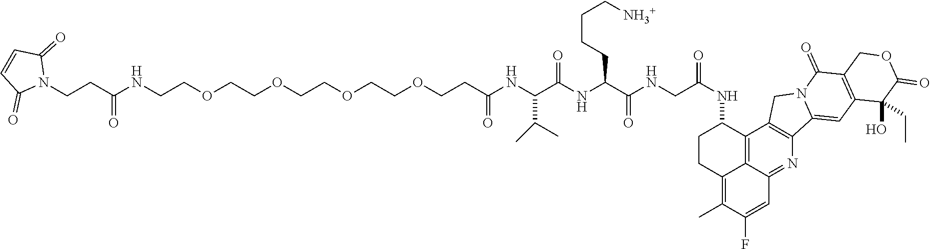 |
| 71 | 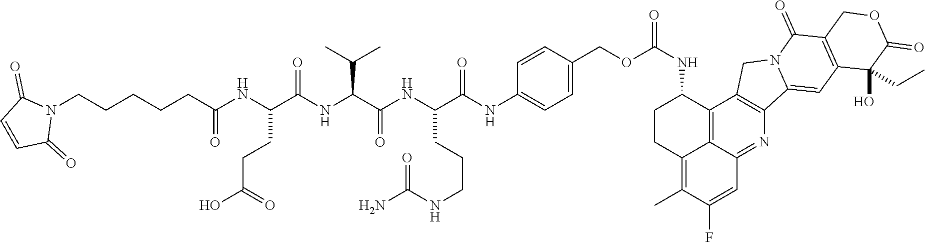 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)[C@H](CCC(=O)O)NC(=O)CCCCCN2C(=O)C=CC2=O)C(C)C)cc1)CC3] |
| 72 | 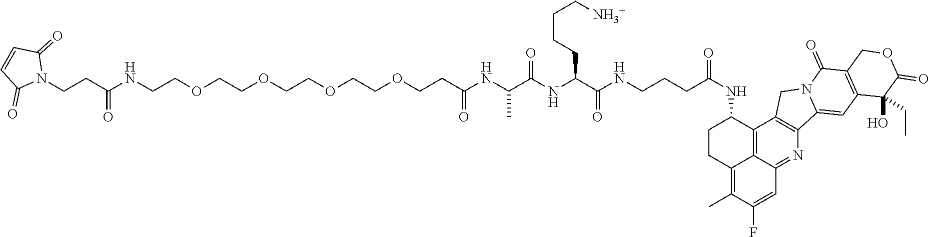 [SMILES: CC[C@@]1(O)C(=O)OCc2c1cc1n(c2=O)Cc2c-1nc1cc(F)c(C)c3c1c2[C@@H](NC(=O)CCCNC(=O)[C@H](CCCC[NH3+])NC(=O)[C@H](C)NC(=O)CCOCCOCCOCCOCCNC(=O)CCN1C(=O)C=CC1=O)CC3] |
| 73 | 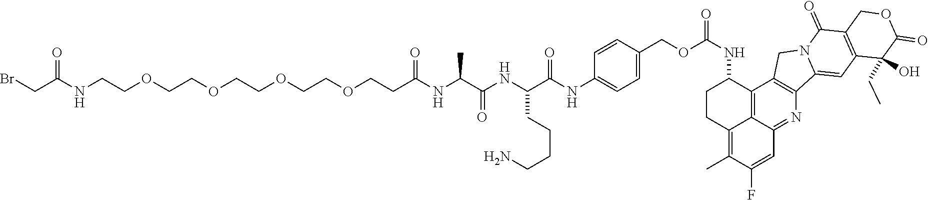 |
| 74 | 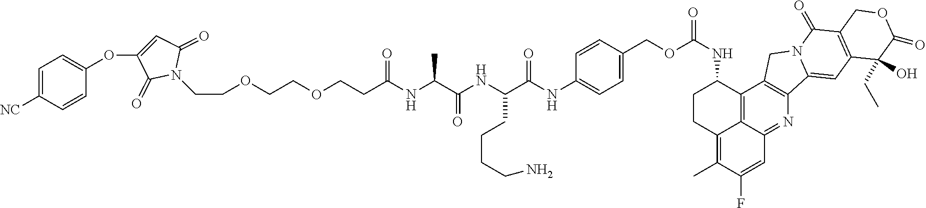 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCCN)NC(=O)[C@H](C)NC(=O)CCOCCOCCN2C(=O)C=C(Oc4ccc(C#N)cc4)C2=O)cc1)CC3] |
| 92 | 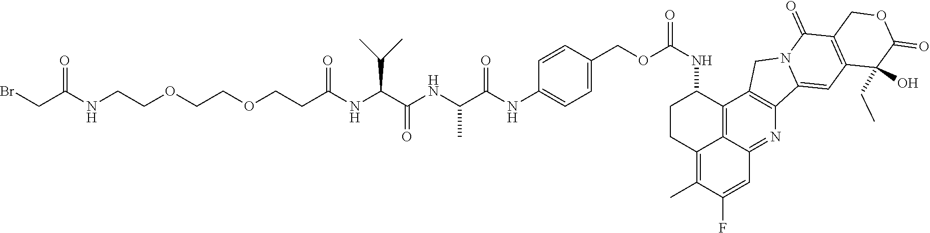 |
| 94 | 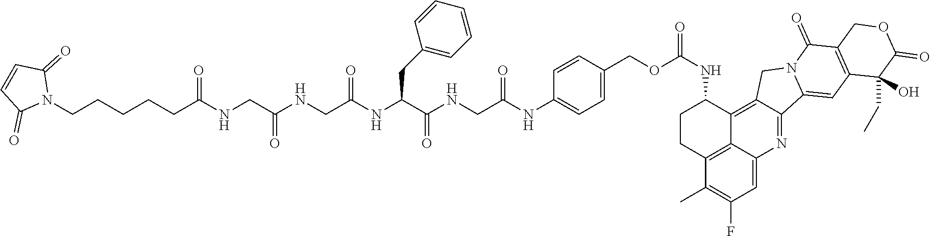 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)CNC(=O)[C@H](Cc2ccccc2)NC(=O)CNC(=O)CNC(=O)CCCCCN2C(=O)C=CC2=O)cc1)CC3] |
| 95 | 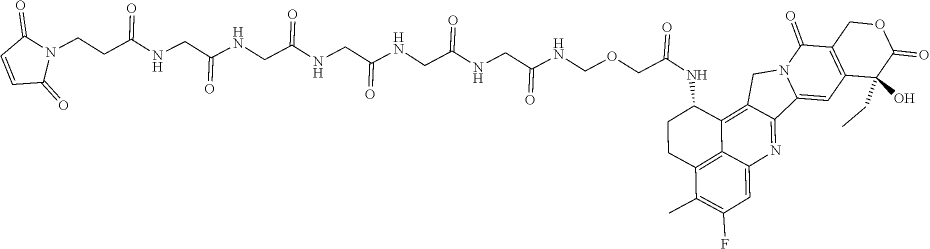 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)CNC(=O)CNC(=O)CNC(=O)CNC(=O)CCN1C(=O)C=CC1=O)CC3] |
| 96 | 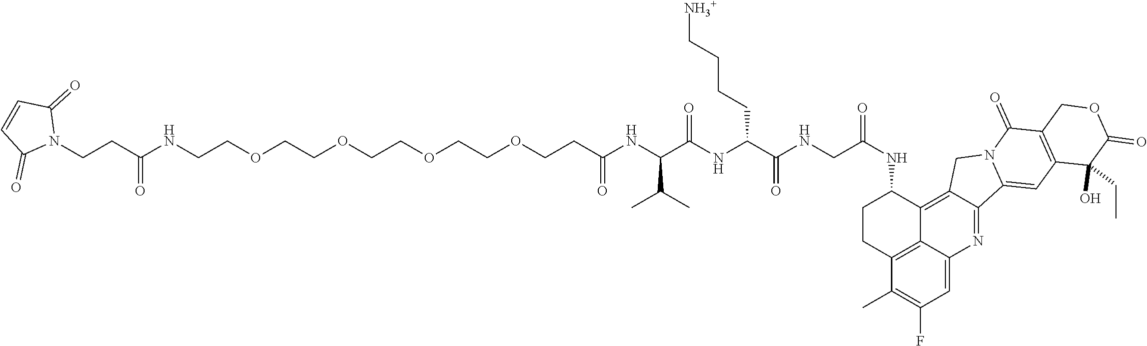 [SMILES: CC[C@@]1(O)C(=O)OCc2c1cc1n(c2=O)Cc2c-1nc1cc(F)c(C)c3c1c2[C@@H](NC(=O)CNC(=O)[C@@H](CCCC[NH3+])NC(=O)[C@H](NC(=O)CCOCCOCCOCCOCCNC(=O)CCN1C(=O)C=CC1=O)C(C)C)CC3] |
| 97 | 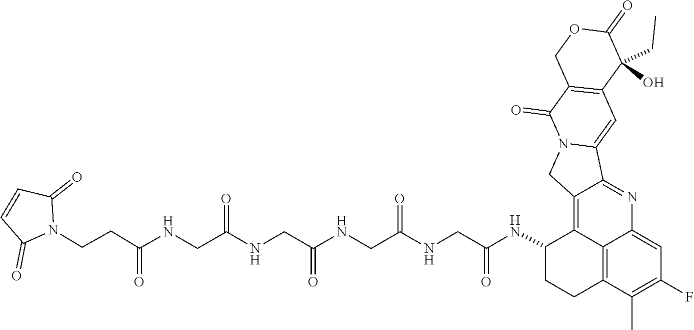 |
| 98 | 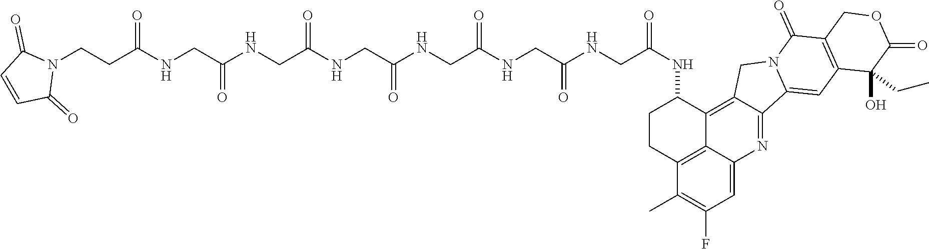 |
| 99 | 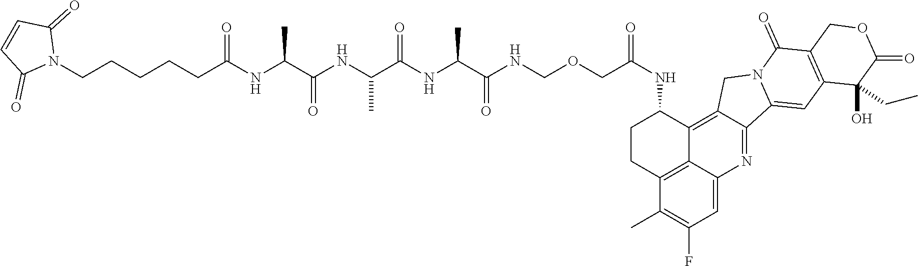 [SMILES: CC[C@@]1(O)C(=O)OCc2c1cc1n(c2=O)Cc2c-1nc1cc(F)c(C)c3c1c2[C@@H](NC(=O)COCNC(=O)[C@H](C)NC(=O)[C@H](C)NC(=O)[C@H](C)NC(=O)CCCCCN1C(=O)C=CC1=O)CC3] |
| 100 | 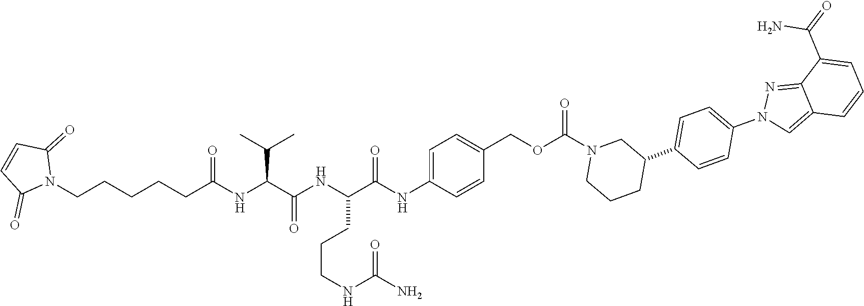 [SMILES: CC(C)[C@H](NC(=O)CCCCCN1C(=O)C=CC1=O)C(=O)N[C@@H](CCCNC(N)=O)C(=O)Nc1ccc(COC(=O)N2CCC[C@@H](c3ccc(-n4cc5cccc(C(N)=O)c5n4)cc3)C2)cc1] |
| 101 | 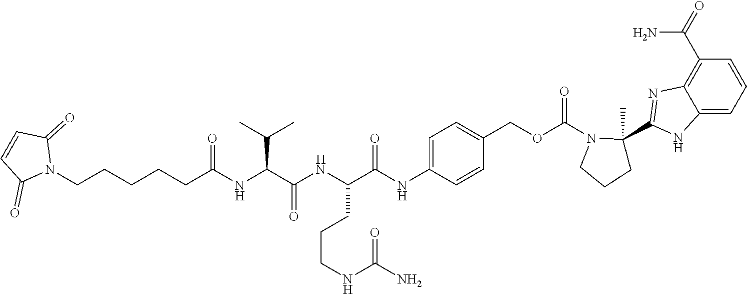 [SMILES: CC(C)[C@H](NC(=O)CCCCCN1C(=O)C=CC1=O)C(=O)N[C@@H](CCCNC(N)=O)C(=O)Nc1ccc(COC(=O)N2CCC[C@]2(C)c2nc3c(C(N)=O)cccc3[nH]2)cc1] |
| 102 | 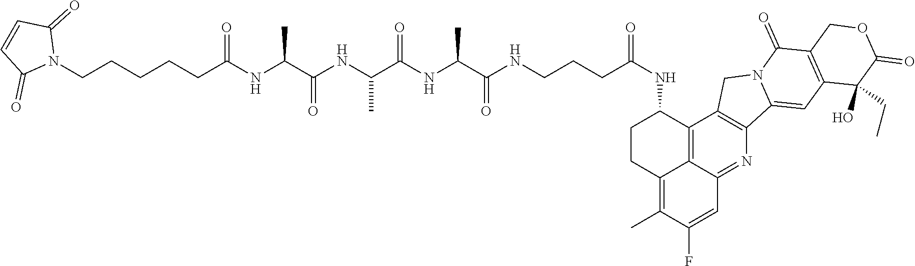 [SMILES: CC[C@@]1(O)C(=O)OCc2c1cc1n(c2=O)Cc2c-1nc1cc(F)c(C)c3c1c2[C@@H](NC(=O)CCCNC(=O)[C@H](C)NC(=O)[C@H](C)NC(=O)[C@H](C)NC(=O)CCCCCN1C(=O)C=CC1=O)CC3] |
| 103 | 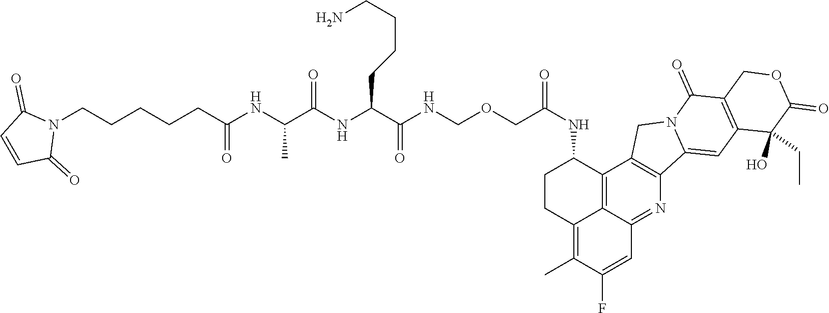 [SMILES: CC[C@@]1(O)C(=O)OCc2c1cc1n(c2=O)Cc2c-1nc1cc(F)c(C)c3c1c2[C@@H](NC(=O)COCNC(=O)[C@H](CCCCN)NC(=O)[C@H](C)NC(=O)CCCCCN1C(=O)C=CC1=O)CC3] |
| 104 | 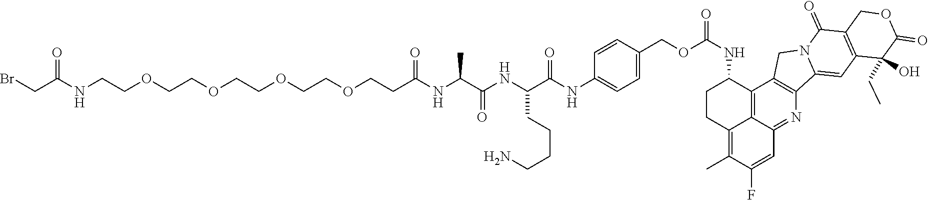 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCCN)NC(=O)[C@H](C)NC(=O)CCOCCOCCOCCOCCNC(=O)CBr)cc1)CC3] |
| 105 | 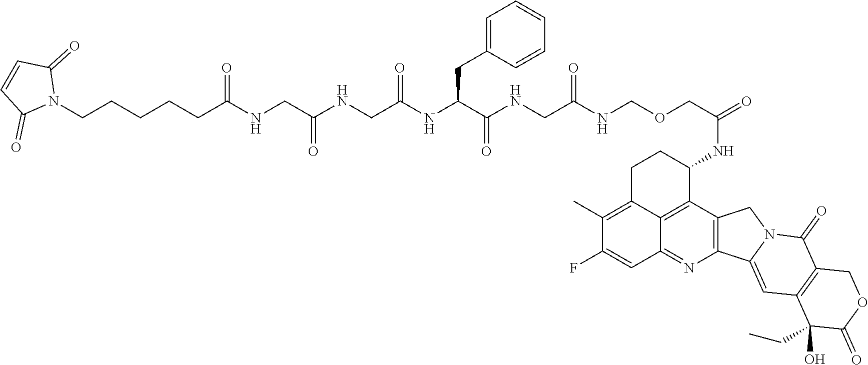 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3] |
| 106 | 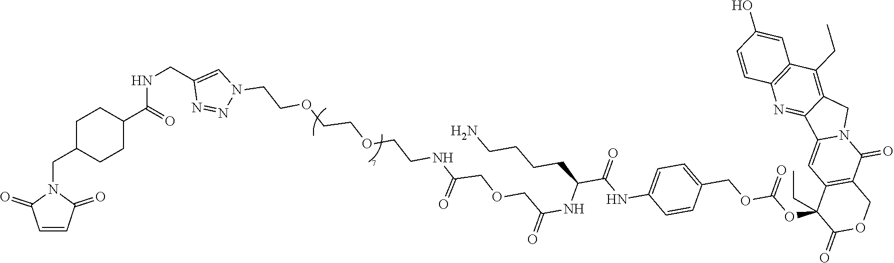 |

In some embodiments, the compounds described herein have a structure provided in Table 2.

TABLE 2

| Compound # | Chemical Structure |
|---|---|
| 23 | 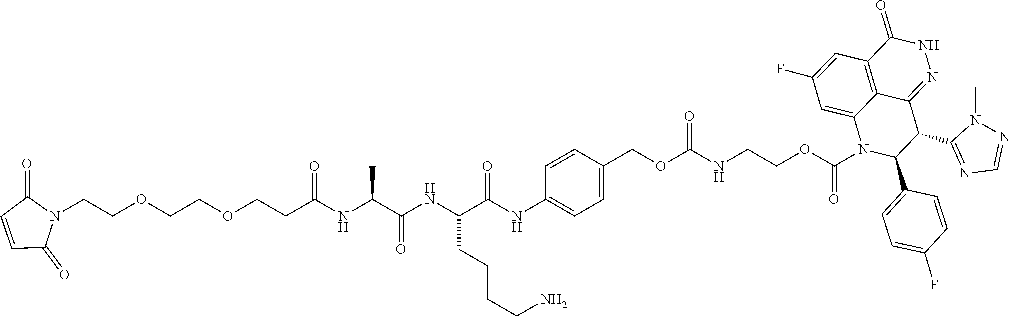 [SMILES: C[C@H](NC(=O)CCOCCOCCN1C(=O)C=CC1=O)C(=O)N[C@@H](CCCCN)C(=O)Nc1ccc(COC(=O)NCCOC(=O)N2c3cc(F)cc4c(=O)[nH]nc(c34)[C@H](c3ncnn3C)[C@H]2c2ccc(F)cc2)cc1] |
| 24 | 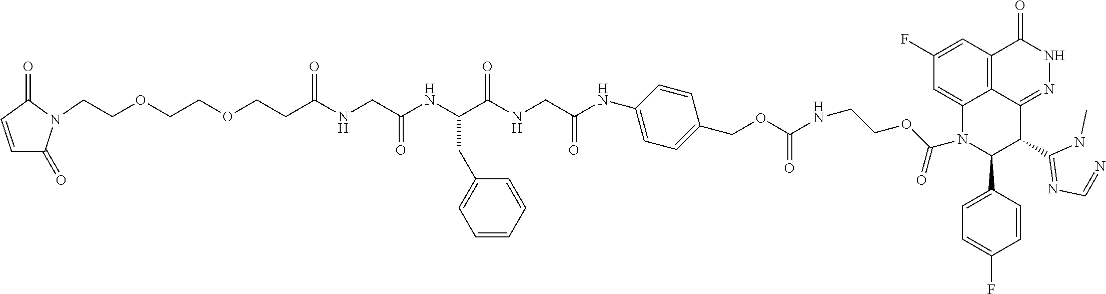 [SMILES: CN/C(=N\C=N)[C@H]1c2n[nH]c(=O)c3cc(F)cc(c23)N(C(=O)OCCNC(=O)OCc2ccc(NC(=O)CNC(=O)[C@H](Cc3ccccc3)NC(=O)CNC(=O)CCOCCOCCN3C(=O)C=CC3=O)cc2)[C@@H]1c1ccc(F)cc1] |
| 25 | 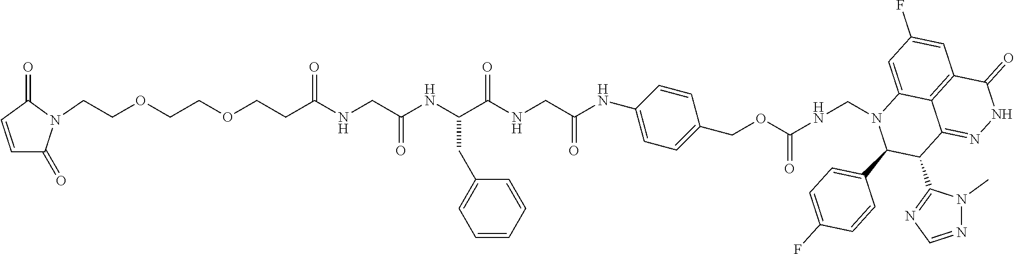 [SMILES: Cn1ncnc1[C@H]1c2n[nH]c(=O)c3cc(F)cc(c23)N(CNC(=O)OCc2ccc(NC(=O)CNC(=O)[C@H](Cc3ccccc3)NC(=O)CNC(=O)CCOCCOCCN3C(=O)C=CC3=O)cc2)[C@@H]1c1ccc(F)cc1] |
| 26 | 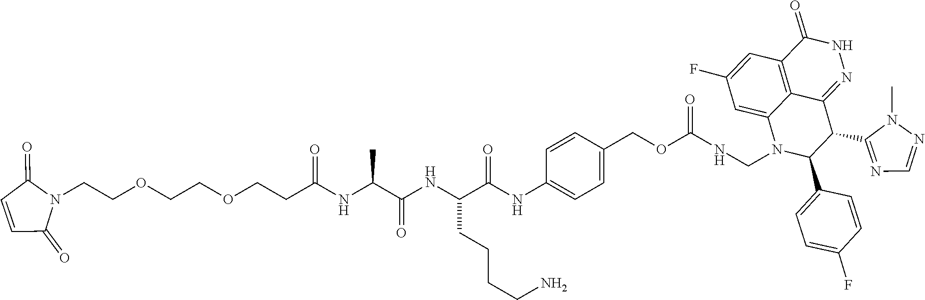 [SMILES: C[C@H](NC(=O)CCOCCOCCN1C(=O)C=CC1=O)C(=O)N[C@@H](CCCCN)C(=O)Nc1ccc(COC(=O)NCN2c3cc(F)cc4c(=O)[nH]nc(c34)[C@H](c3ncnn3C)[C@H]2c2ccc(F)cc2)cc1] |
| 27 | 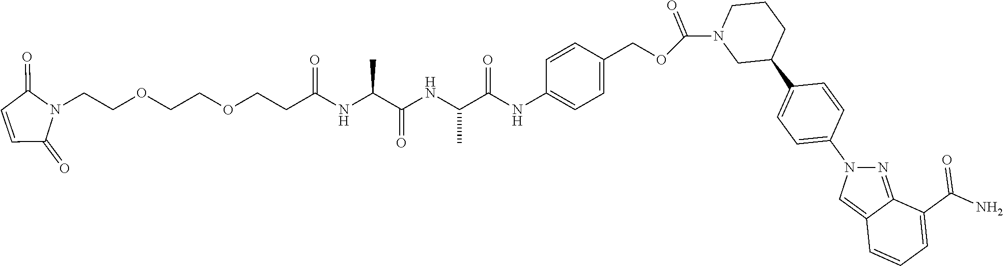 [SMILES: C[C@H](NC(=O)CCOCCOCCN1C(=O)C=CC1=O)C(=O)N[C@@H](C)C(=O)Nc1ccc(COC(=O)N2CCC[C@@H](c3ccc(-n4cc5cccc(C(N)=O)c5n4)cc3)C2)cc1] |
| 28 | 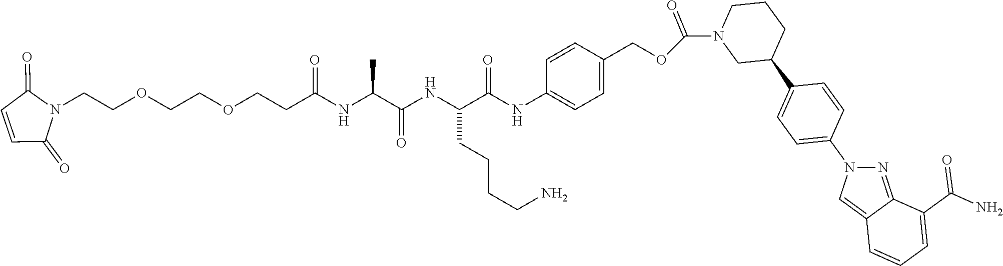 [SMILES: C[C@H](NC(=O)CCOCCOCCN1C(=O)C=CC1=O)C(=O)N[C@@H](CCCCN)C(=O)Nc1ccc(COC(=O)N2CCC[C@@H](c3ccc(-n4cc5cccc(C(N)=O)c5n4)cc3)C2)cc1] |
| 29 | 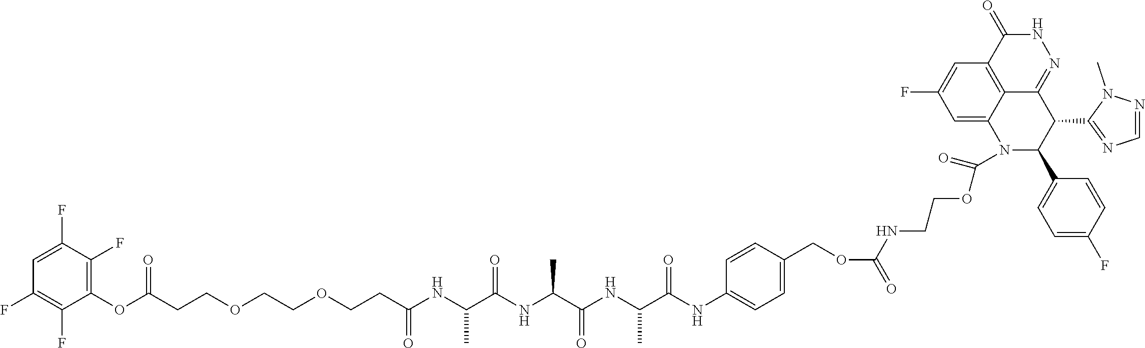 [SMILES: C[C@H](NC(=O)CCOCCOCCC(=O)Oc1c(F)c(F)cc(F)c1F)C(=O)N[C@@H](C)C(=O)N[C@@H](C)C(=O)Nc1ccc(COC(=O)NCCOC(=O)N2c3cc(F)cc4c(=O)[nH]nc(c34)[C@H](c3ncnn3C)[C@H]2c2ccc(F)cc2)cc1] |
| 30 | 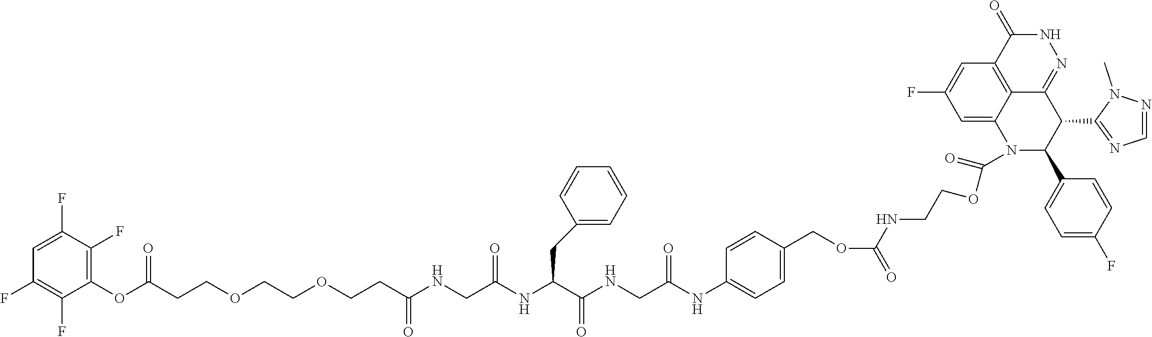 [SMILES: Cn1ncnc1[C@H]1c2n[nH]c(=O)c3cc(F)cc(c23)N(C(=O)OCCNC(=O)OCc2ccc(NC(=O)CNC(=O)[C@H](Cc3ccccc3)NC(=O)CNC(=O)CCOCCOCCC(=O)Oc3c(F)c(F)cc(F)c3F)cc2)[C@@H]1c1ccc(F)cc1] |
| 31 | 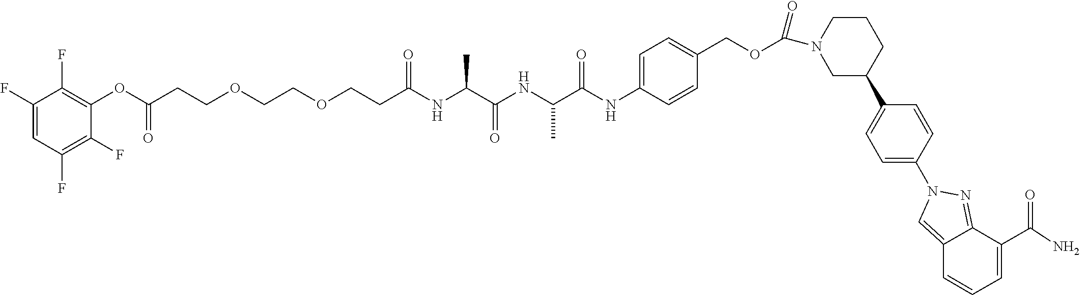 [SMILES: C[C@H](NC(=O)CCOCCOCCC(=O)Oc1c(F)c(F)cc(F)c1F)C(=O)N[C@@H](C)C(=O)Nc1ccc(COC(=O)N2CCC[C@@H](c3ccc(-n4cc5cccc(C(N)=O)c5n4)cc3)C2)cc1] |
| 32 | 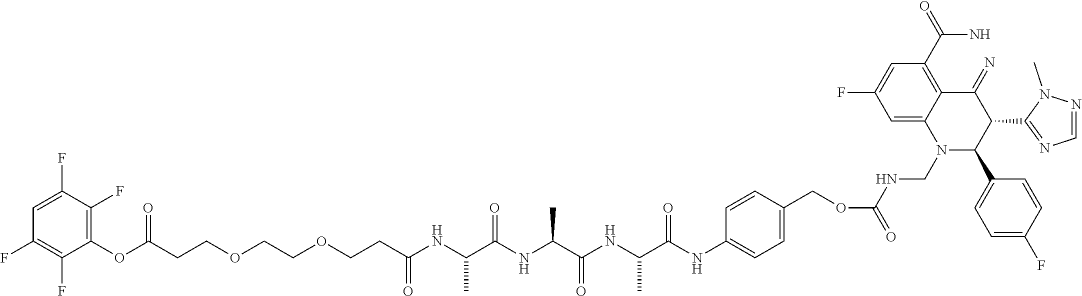 [SMILES: C[C@H](NC(=O)CCOCCOCCC(=O)Oc1c(F)c(F)cc(F)c1F)C(=O)N[C@@H](C)C(=O)N[C@@H](C)C(=O)Nc1ccc(COC(=O)NCN2c3cc(F)cc(C(N)=O)c3C(=N)[C@H](c3ncnn3C)[C@H]2c2ccc(F)cc2)cc1] |
| 35 | 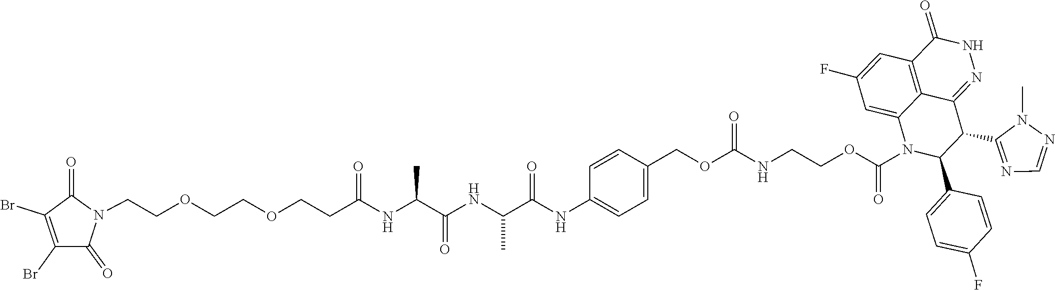 [SMILES: C[C@H](NC(=O)CCOCCOCCN1C(=O)C(Br)=C(Br)C1=O)C(=O)N[C@@H](C)C(=O)Nc1ccc(COC(=O)NCCOC(=O)N2c3cc(F)cc4c(=O)[nH]nc(c34)[C@H](c3ncnn3C)[C@H]2c2ccc(F)cc2)cc1] |
| 36 | 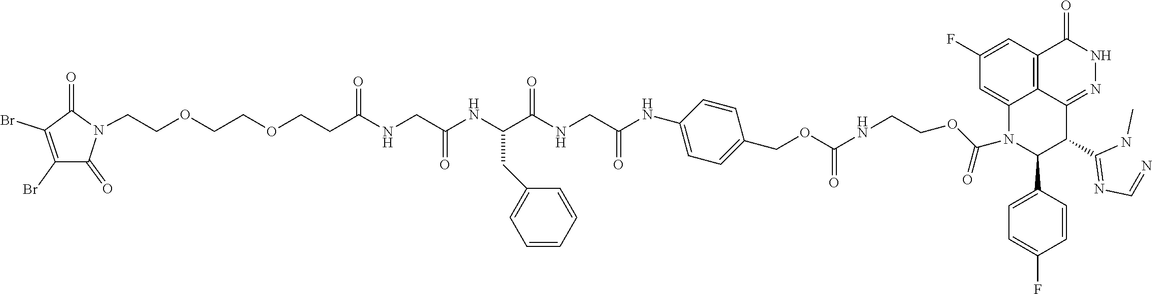 [SMILES: CN/C(=N\C=N)[C@H]1c2n[nH]c(=O)c3cc(F)cc(c23)N(C(=O)OCCNC(=O)OCc2ccc(NC(=O)CNC(=O)[C@H](Cc3ccccc3)NC(=O)CNC(=O)CCOCCOCCN3C(=O)C(Br)=C(Br)C3=O)cc2)[C@@H]1c1ccc(F)cc1] |
| 65 | 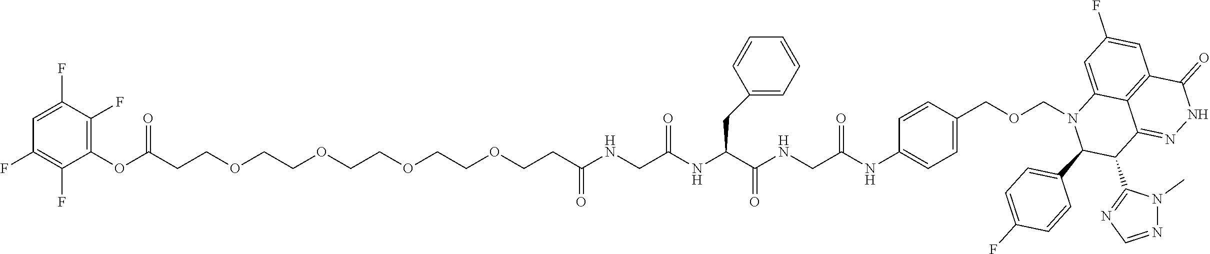 [SMILES: Cn1ncnc1[C@H]1c2n[nH]c(=O)c3cc(F)cc(c23)N(COCc2ccc(NC(=O)CNC(=O)[C@H](Cc3ccccc3)NC(=O)CNC(=O)CCOCCOCCOCCOCCC(=O)Oc3c(F)c(F)cc(F)c3F)cc2)[C@@H]1c1ccc(F)cc1] |
| 66 | 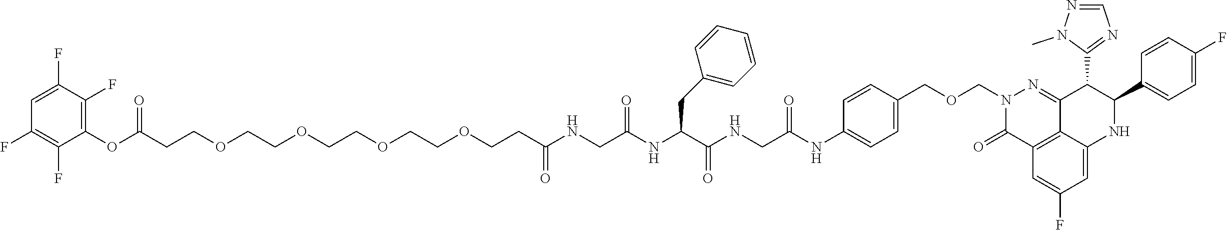 [SMILES: Cn1ncnc1[C@H]1c2nn(COCc3ccc(NC(=O)CNC(=O)[C@H](Cc4ccccc4)NC(=O)CNC(=O)CCOCCOCCOCCOCCC(=O)Oc4c(F)c(F)cc(F)c4F)cc3)c(=O)c3cc(F)cc(c23)N[C@@H]1c1ccc(F)cc1] |
| 67 | 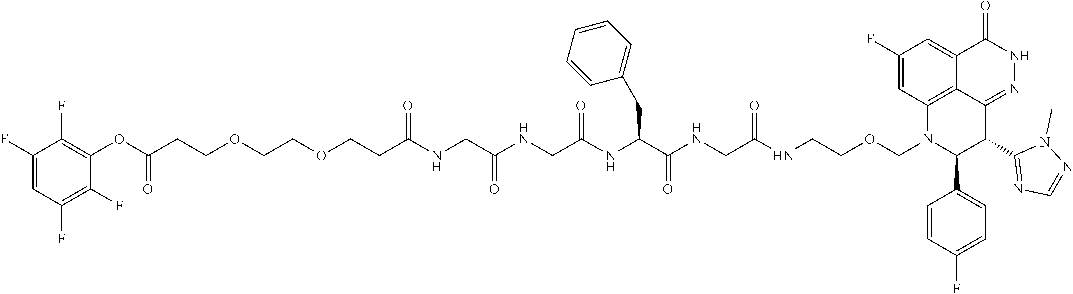 [SMILES: Cn1ncnc1[C@H]1c2n[nH]c(=O)c3cc(F)cc(c23)N(COCCNC(=O)CNC(=O)[C@H](Cc2ccccc2)NC(=O)CNC(=O)CNC(=O)CCOCCOCCC(=O)Oc2c(F)c(F)cc(F)c2F)[C@@H]1c1ccc(F)cc1] |
| 68 | 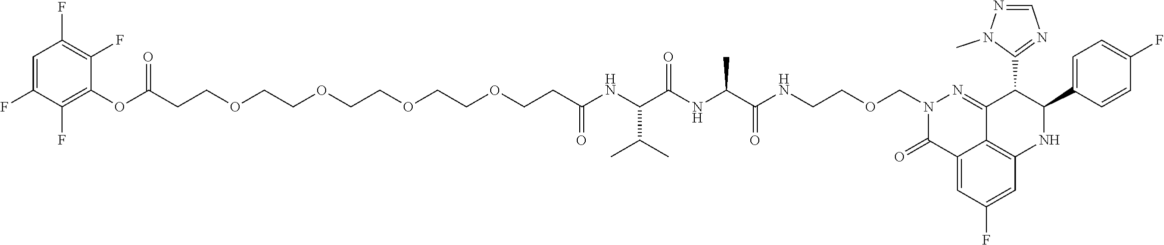 [SMILES: CC(C)[C@H](NC(=O)CCOCCOCCOCCOCCC(=O)Oc1c(F)c(F)cc(F)c1F)C(=O)N[C@@H](C)C(=O)NCCOCn1nc2c3c(cc(F)cc3c1=O)N[C@H](c1ccc(F)cc1)[C@H]2c1ncnn1C] |
| 45 | 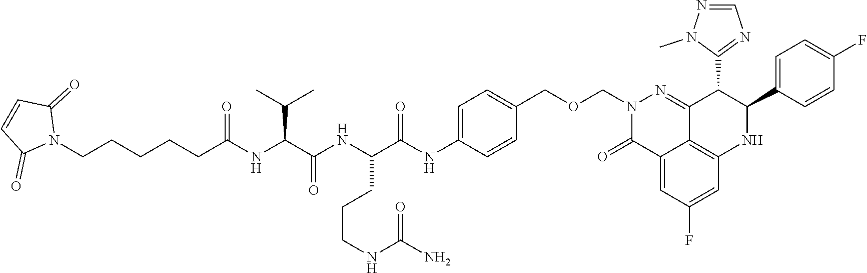 [SMILES: CC(C)[C@H](NC(=O)CCCCCN1C(=O)C=CC1=O)C(=O)N[C@@H](CCCNC(N)=O)C(=O)Nc1ccc(COCn2nc3c4c(cc(F)cc4c2=O)N[C@H](c2ccc(F)cc2)[C@H]3c2ncnn2C)cc1] |
| 46 | 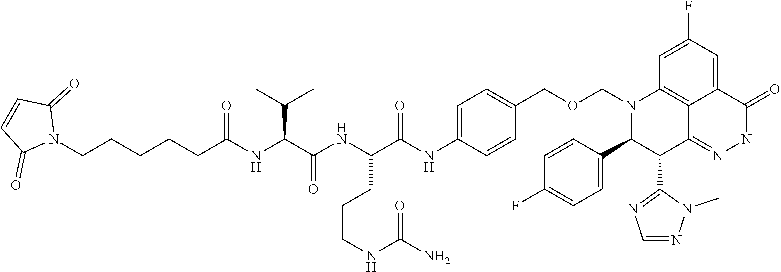 |
| 47 | 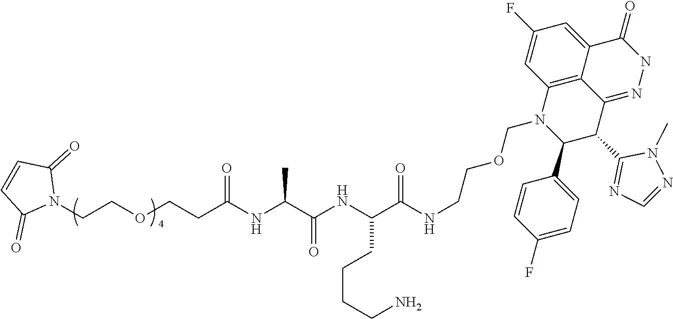 |
| 48 | 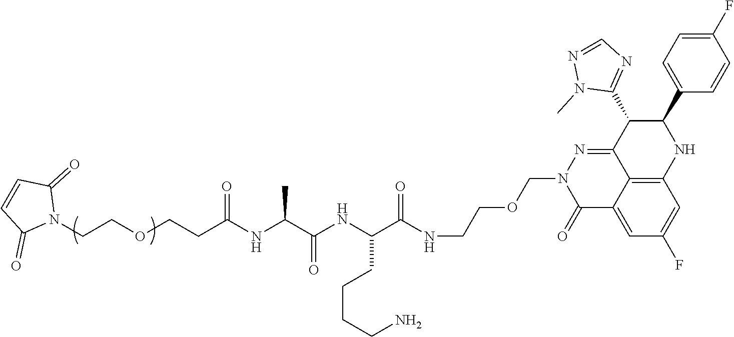 |
| 49 | 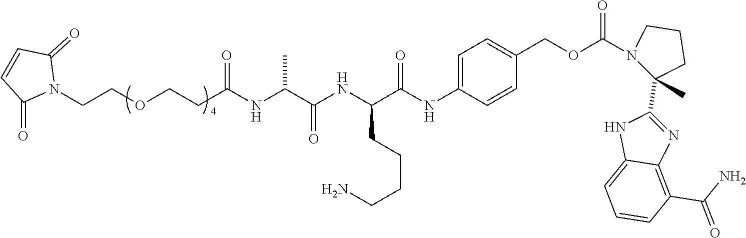 |
| 50 | 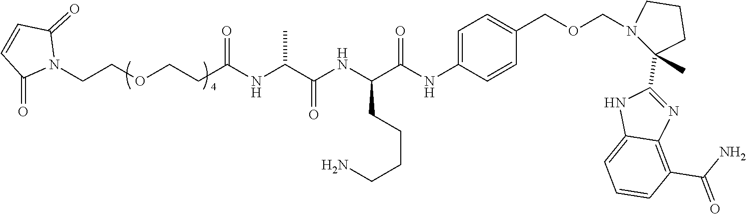 |
| 51 | 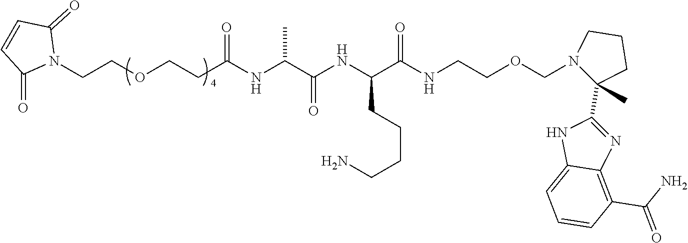 |
| 52 | 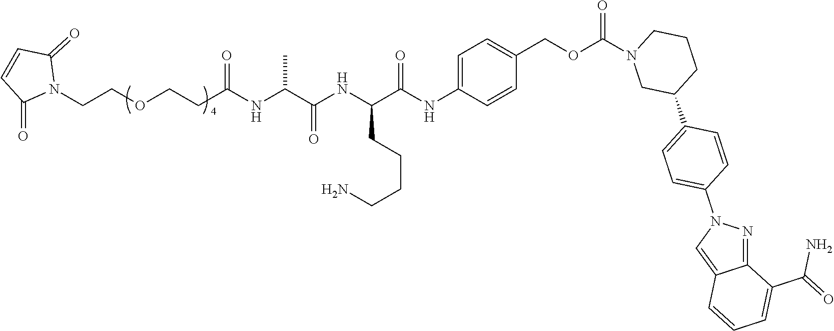 |
| 53 | 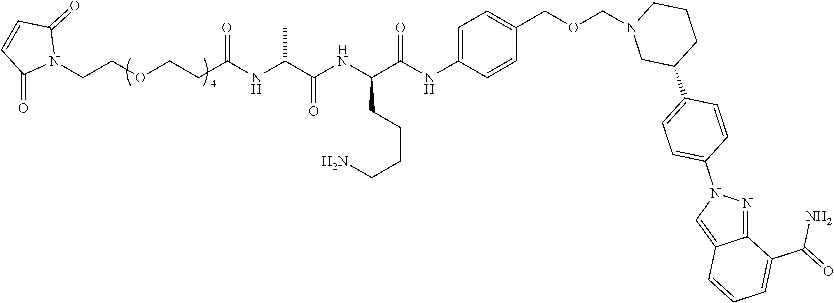 |
| 54 | 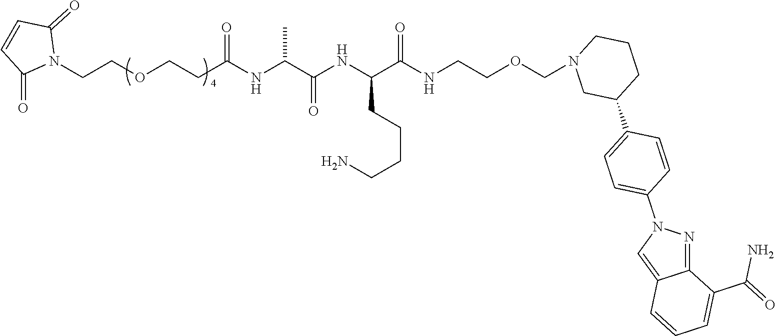 |
| 55 | 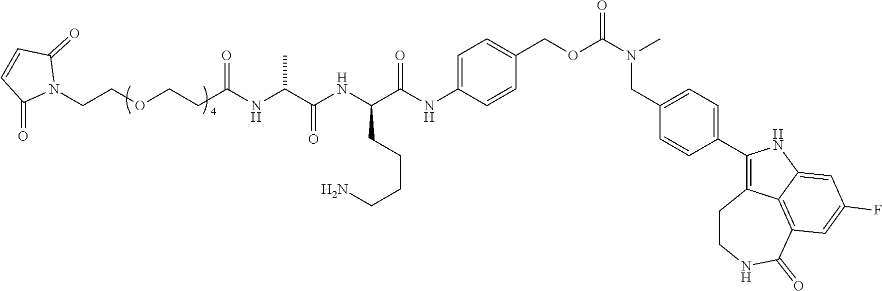 |
| 56 | 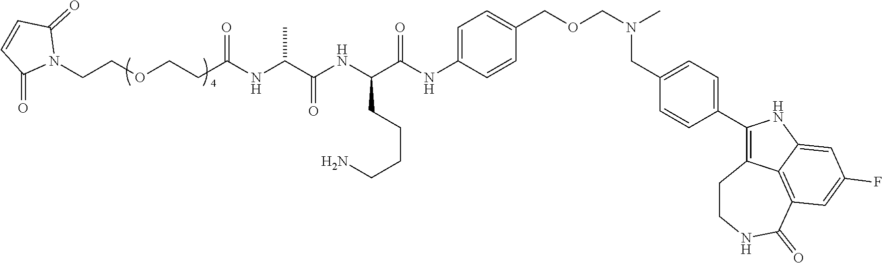 |
| 57 | 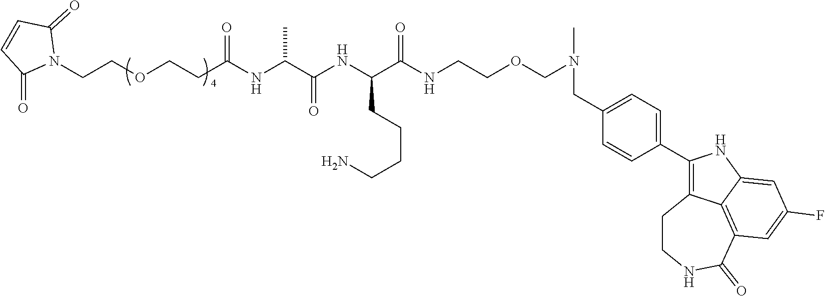 |
| 75 | 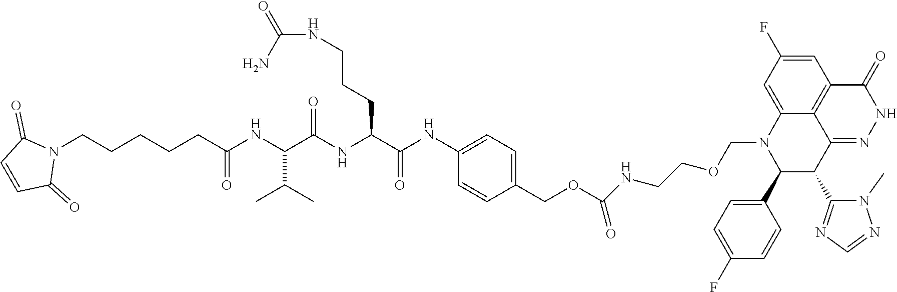 [SMILES: CC(C)[C@H](NC(=O)CCCCCN1C(=O)C=CC1=O)C(=O)N[C@@H](CCCNC(N)=O)C(=O)Nc1ccc(COC(=O)NCCOCN2c3cc(F)cc4c(=O)[nH]nc(c34)[C@H](c3ncnn3C)[C@H]2c2ccc(F)cc2)cc1] |
| 76 | 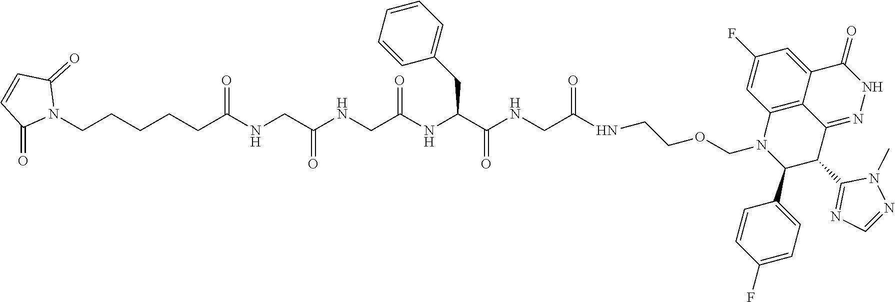 [SMILES: Cn1ncnc1[C@H]1c2n[nH]c(=O)c3cc(F)cc(c23)N(COCCNC(=O)CNC(=O)[C@H](Cc2ccccc2)NC(=O)CNC(=O)CNC(=O)CCCCCN2C(=O)C=CC2=O)[C@@H]1c1ccc(F)cc1] |
| 77 | 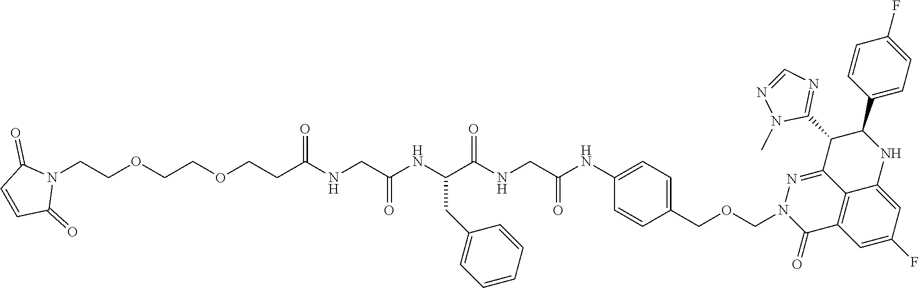 [SMILES: Cn1ncnc1[C@H]1c2nn(COCc3ccc(NC(=O)CNC(=O)[C@H](Cc4ccccc4)NC(=O)CNC(=O)CCOCCOCCN4C(=O)C=CC4=O)cc3)c(=O)c3cc(F)cc(c23)N[C@@H]1c1ccc(F)cc1] |
| 78 | 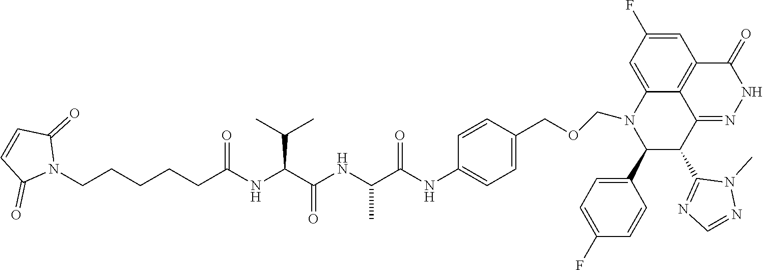 [SMILES: CC(C)[C@H](NC(=O)CCCCCN1C(=O)C=CC1=O)C(=O)N[C@@H](C)C(=O)Nc1ccc(COCN2c3cc(F)cc4c(=O)[nH]nc(c34)[C@H](c3ncnn3C)[C@H]2c2ccc(F)cc2)cc1] |
| 79 | 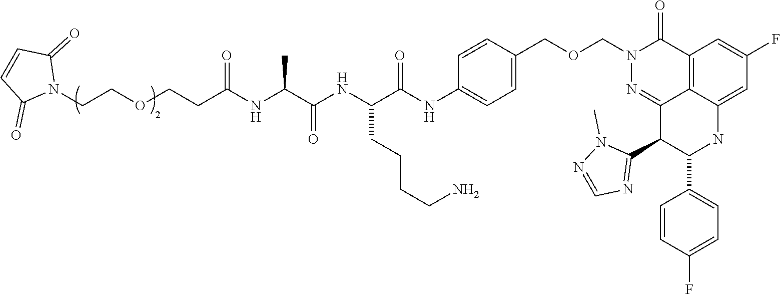 |
| 80 | 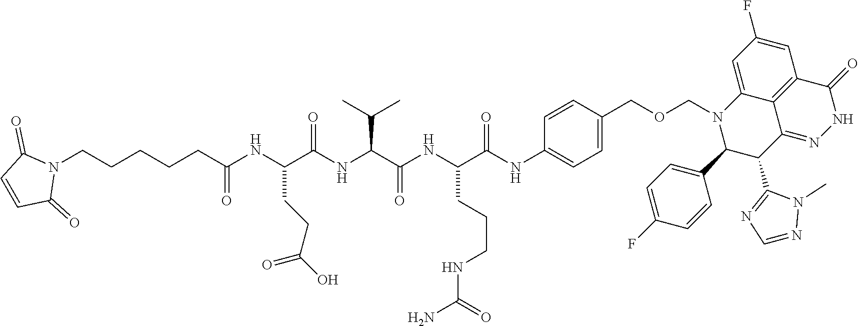 [SMILES: CC(C)[C@H](NC(=O)[C@H](CCC(=O)O)NC(=O)CCCCCN1C(=O)C=CC1=O)C(=O)N[C@@H](CCCNC(N)=O)C(=O)Nc1ccc(COCN2c3cc(F)cc4c(=O)[nH]nc(c34)[C@H](c3ncnn3C)[C@H]2c2ccc(F)cc2)cc1] |
| 81 | 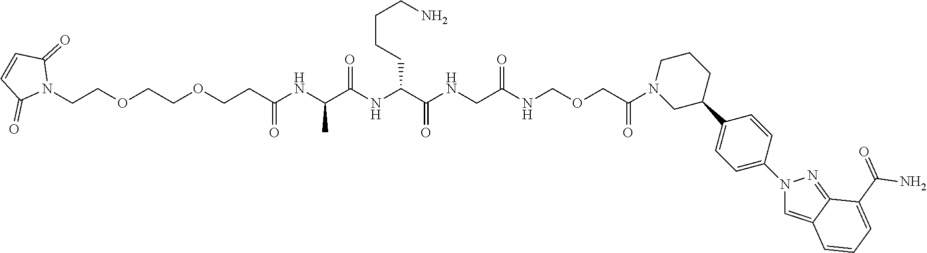 [SMILES: C[C@@H](NC(=O)CCOCCOCCN1C(=O)C=CC1=O)C(=O)N[C@H](CCCCN)C(=O)NCC(=O)NCOCC(=O)N1CCC[C@@H](c2ccc(-n3cc4cccc(C(N)=O)c4n3)cc2)C1] |
| 82 | 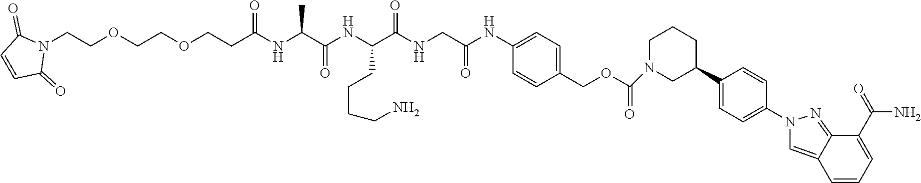 [SMILES: C[C@H](NC(=O)CCOCCOCCN1C(=O)C=CC1=O)C(=O)N[C@@H](CCCCN)C(=O)NCC(=O)Nc1ccc(COC(=O)N2CCC[C@@H](c3ccc(-n4cc5cccc(C(N)=O)c5n4)cc3)C2)cc1] |
| 83 | 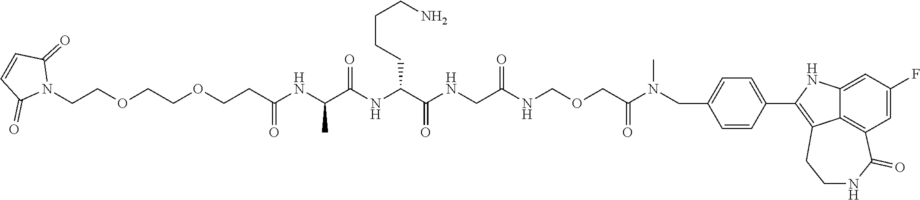 [SMILES: C[C@@H](NC(=O)CCOCCOCCN1C(=O)C=CC1=O)C(=O)N[C@H](CCCCN)C(=O)NCC(=O)NCOCC(=O)N(C)Cc1ccc(-c2[nH]c3cc(F)cc4c3c2CCNC4=O)cc1] |
| 84 | 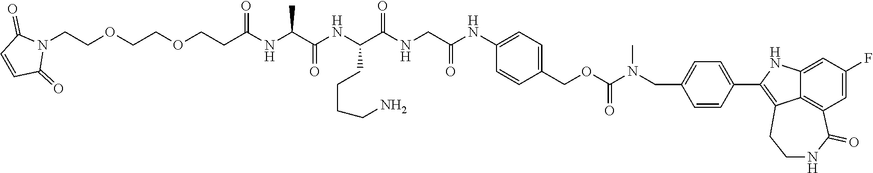 [SMILES: C[C@H](NC(=O)CCOCCOCCN1C(=O)C=CC1=O)C(=O)N[C@@H](CCCCN)C(=O)NCC(=O)Nc1ccc(COC(=O)N(C)Cc2ccc(-c3[nH]c4cc(F)cc5c4c3CCNC5=O)cc2)cc1] |

In some embodiments, the compounds described herein has a structure provided in Table 3.

TABLE 3

| Compound # | Chemical Structure |
|---|---|
| 85 | 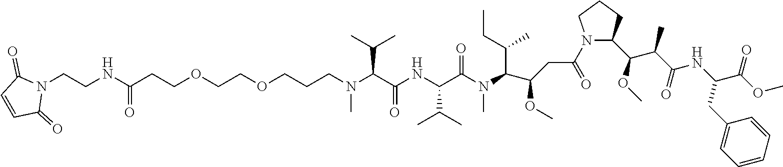 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)CCCOCCOCCC(=O)NCCN1C(=O)C=CC1=O)C(C)C] |
| 86 | 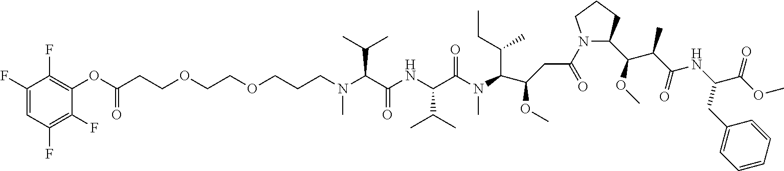 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)CCCOCCOCCC(=O)Oc1c(F)c(F)cc(F)c1F)C(C)C] |
| 87 | 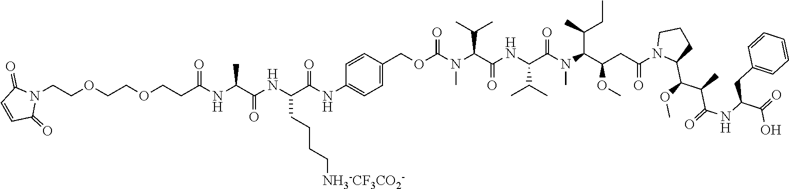 |

In some embodiments, the compounds described herein has a structure provided in Table 4.

TABLE 4

| Compound # | Chemical Structure |
|---|---|
| 88 | 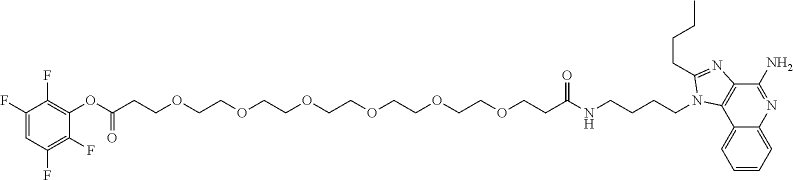 [SMILES: CCCCc1nc2c(N)nc3ccccc3c2n1CCCCNC(=O)CCOCCOCCOCCOCCOCCOCCC(=O)Oc1c(F)c(F)cc(F)c1F] |
| 89 | 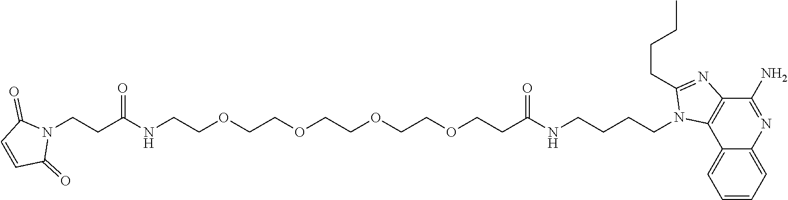 [SMILES: CCCCc1nc2c(N)nc3ccccc3c2n1CCCCNC(=O)CCOCCOCCOCCOCCNC(=O)CCN1C(=O)C=CC1=O] |
| 90 | 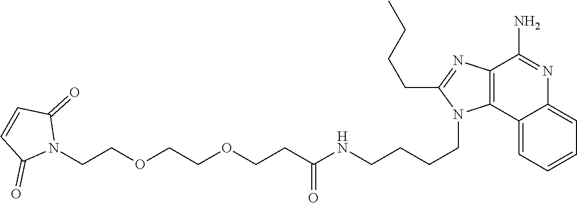 [SMILES: CCCCc1nc2c(N)nc3ccccc3c2n1CCCCNC(=O)CCOCCOCCN1C(=O)C=CC1=O] |
| 91 | 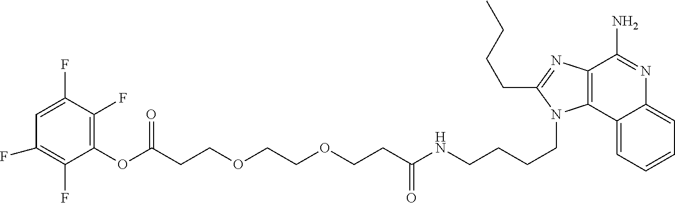 [SMILES: CCCCc1nc2c(N)nc3ccccc3c2n1CCCCNC(=O)CCOCCOCCC(=O)Oc1c(F)c(F)cc(F)c1F] |
| 93 | 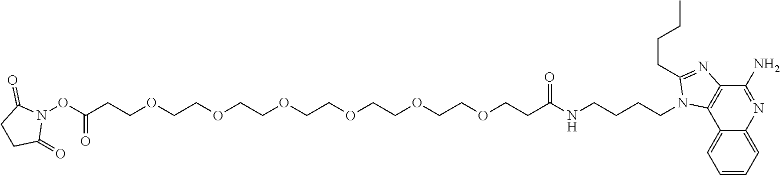 [SMILES: CCCCc1nc2c(N)nc3ccccc3c2n1CCCCNC(=O)CCOCCOCCOCCOCCOCCOCCC(=O)ON1C(=O)CCC1=O] |

In certain embodiments, the compound of Formula (I) is a compound listed in Table 1, Table 2, Table 3, or Table 4. In certain embodiments, the compound of Formula (I) is a compound listed in Table 1 or Table 2. In certain embodiments, the compound of Formula (I) is a compound listed in Table 3 or Table 4. In certain embodiments, the compound of Formula (I) is a compound listed in Table 3. In certain embodiments, the compound of Formula (I) is a compound listed in Table 4. In certain embodiment, the compound of Formula (I) is a compound selected from Compounds 1-84, 92, and 04-104. In certain embodiment, the compound is selected form Compounds 85-87, 88-91, and 93. In one embodiment, the compound is selected from Compounds 85-87. In another embodiment, the compound is selected from Compounds 88-91, and 93.

In certain embodiments, the compound of Formula (I) is selected from the group consisting of:

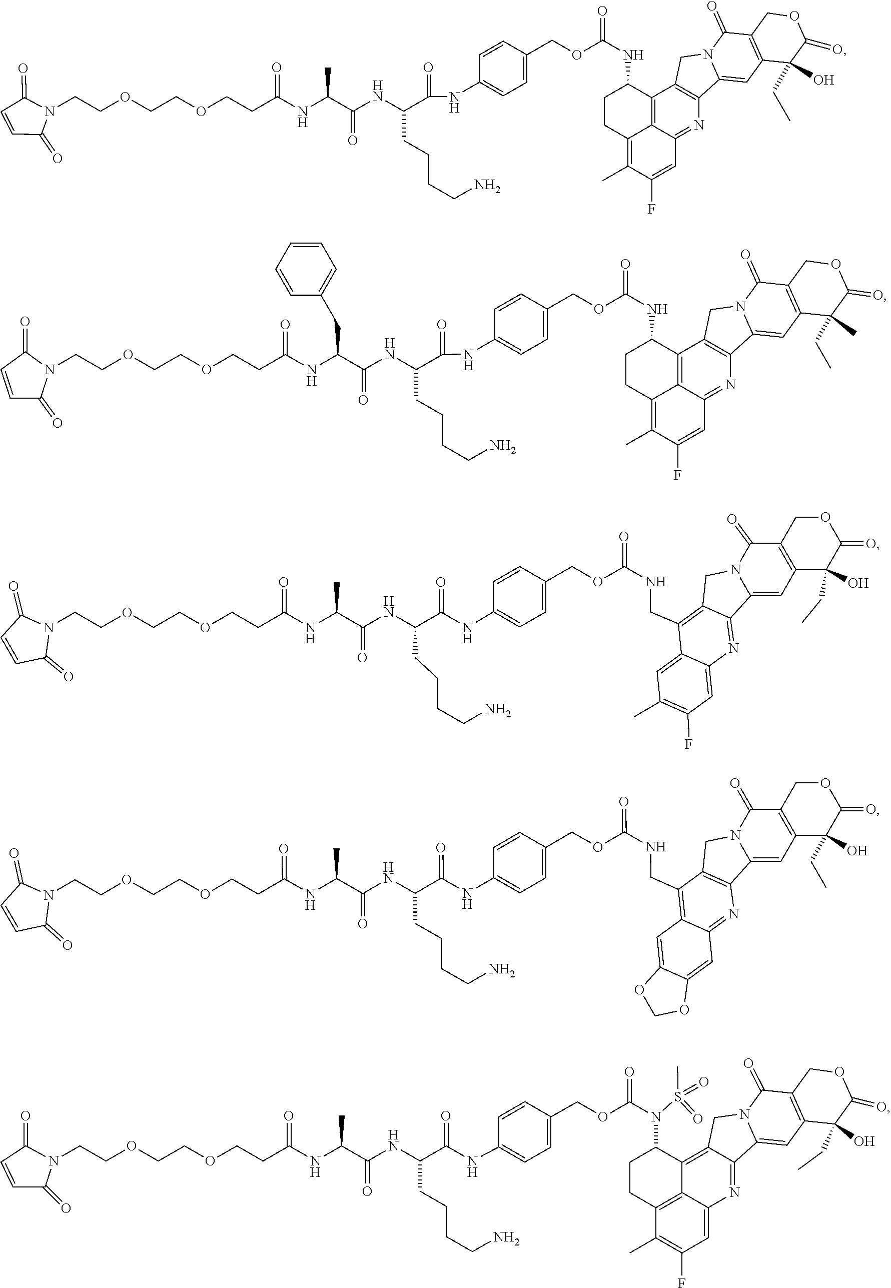

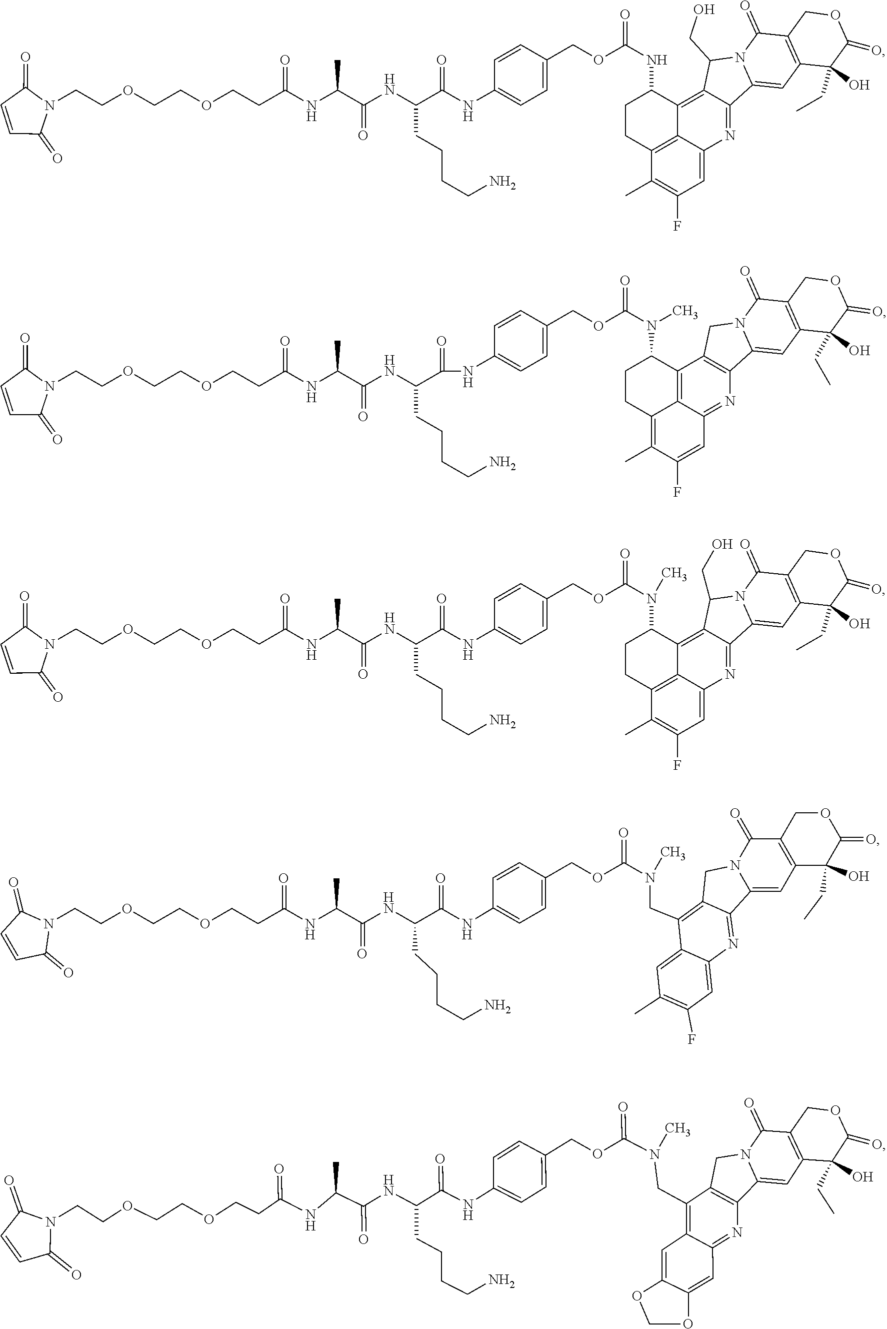

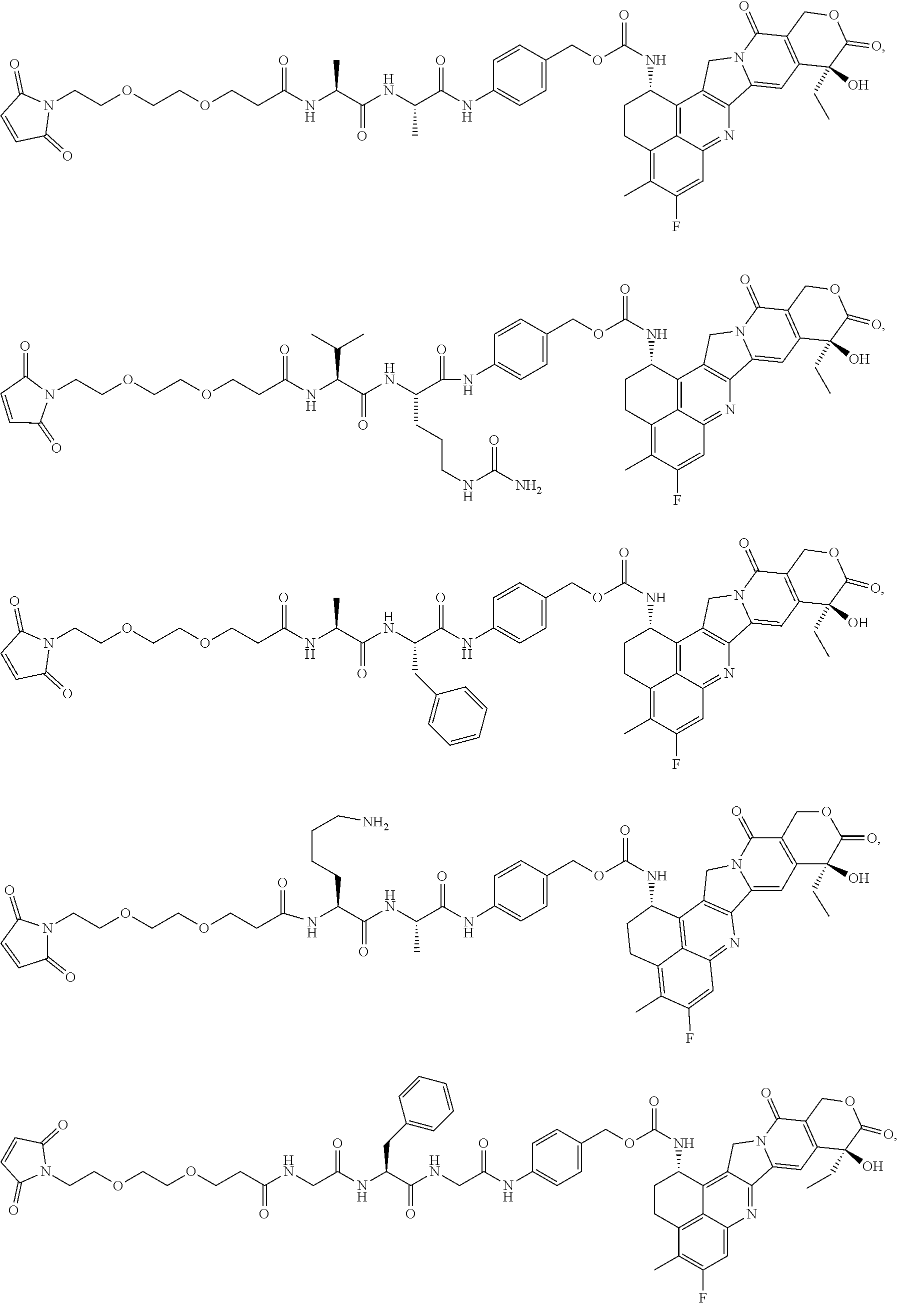

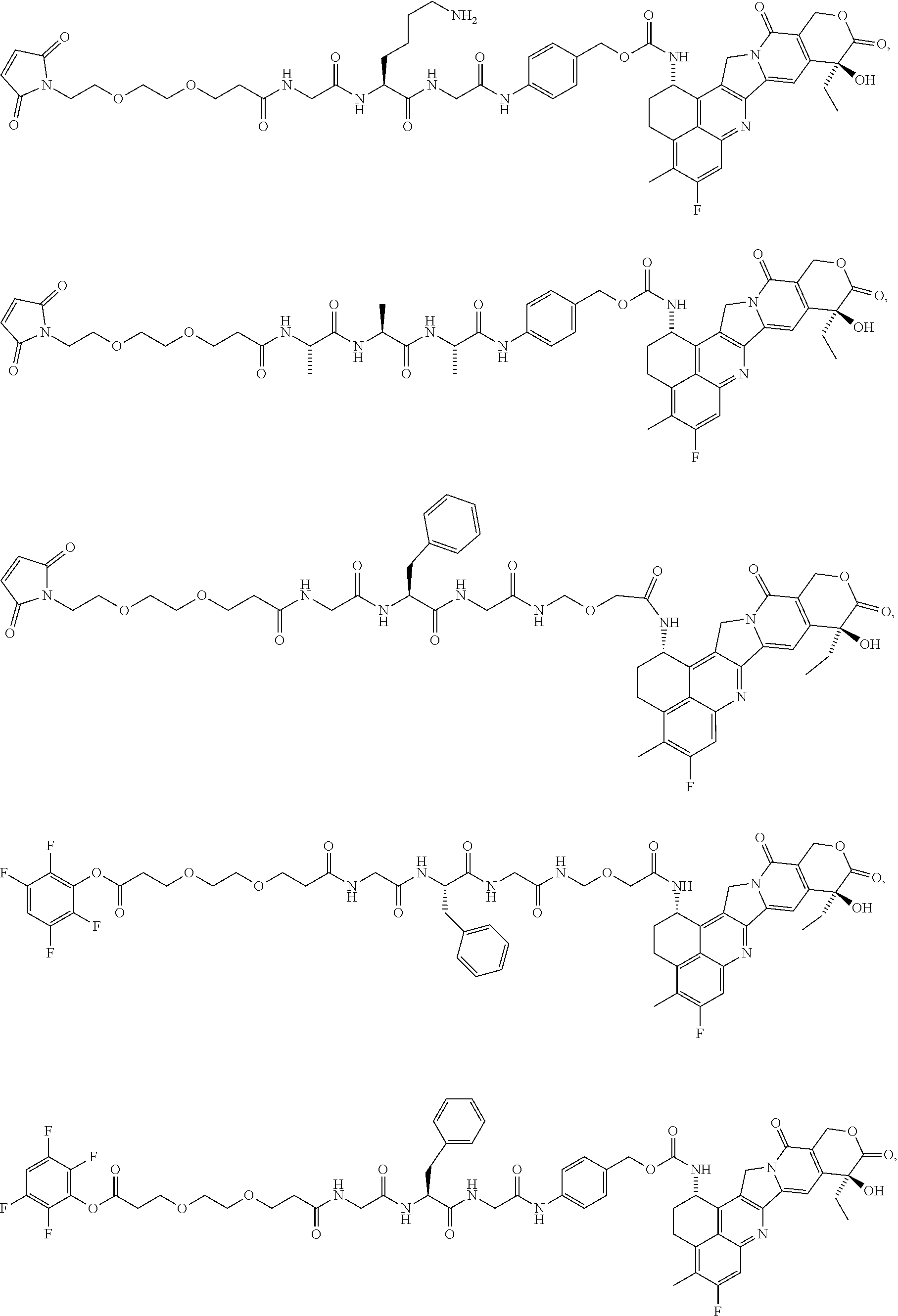

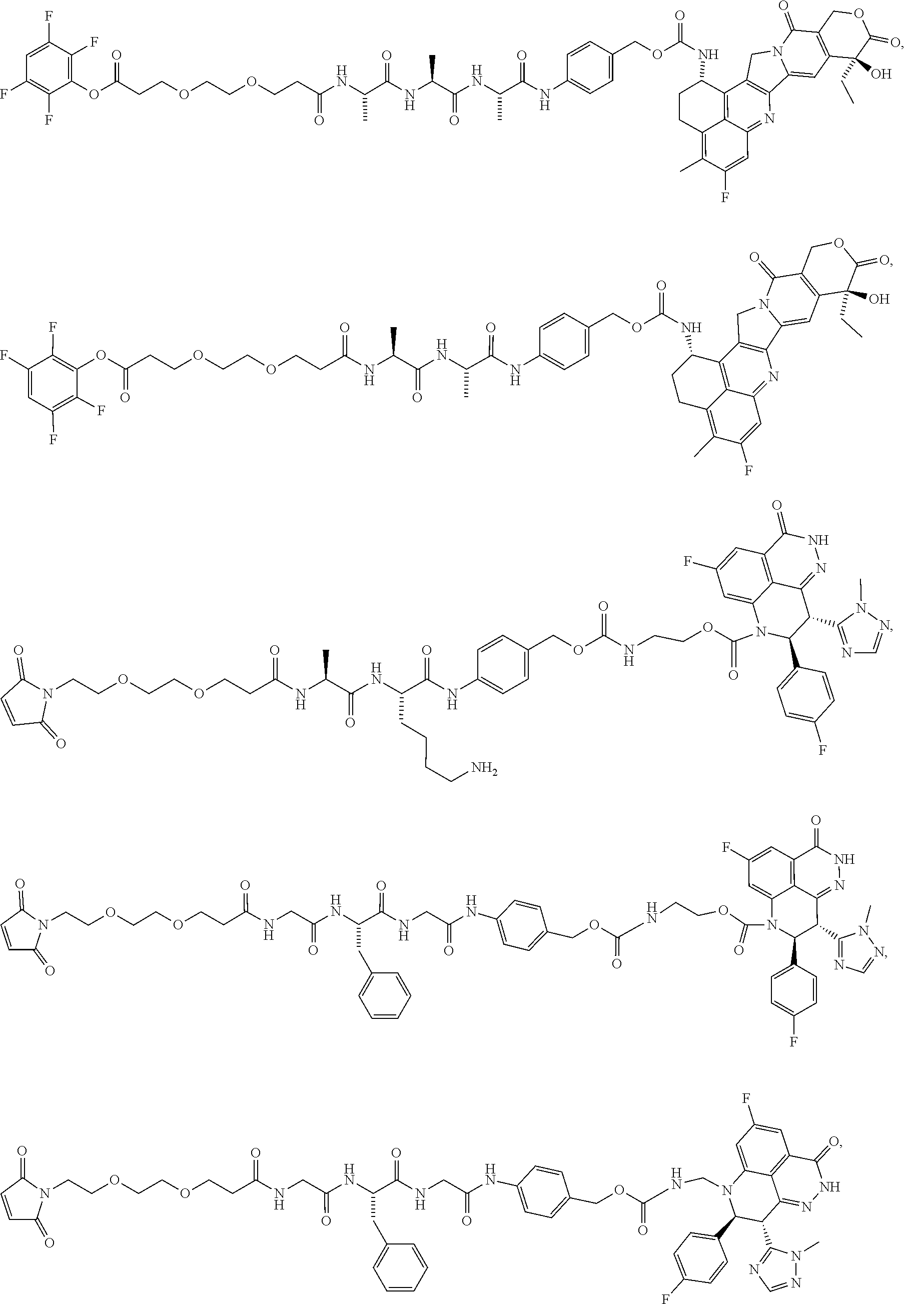

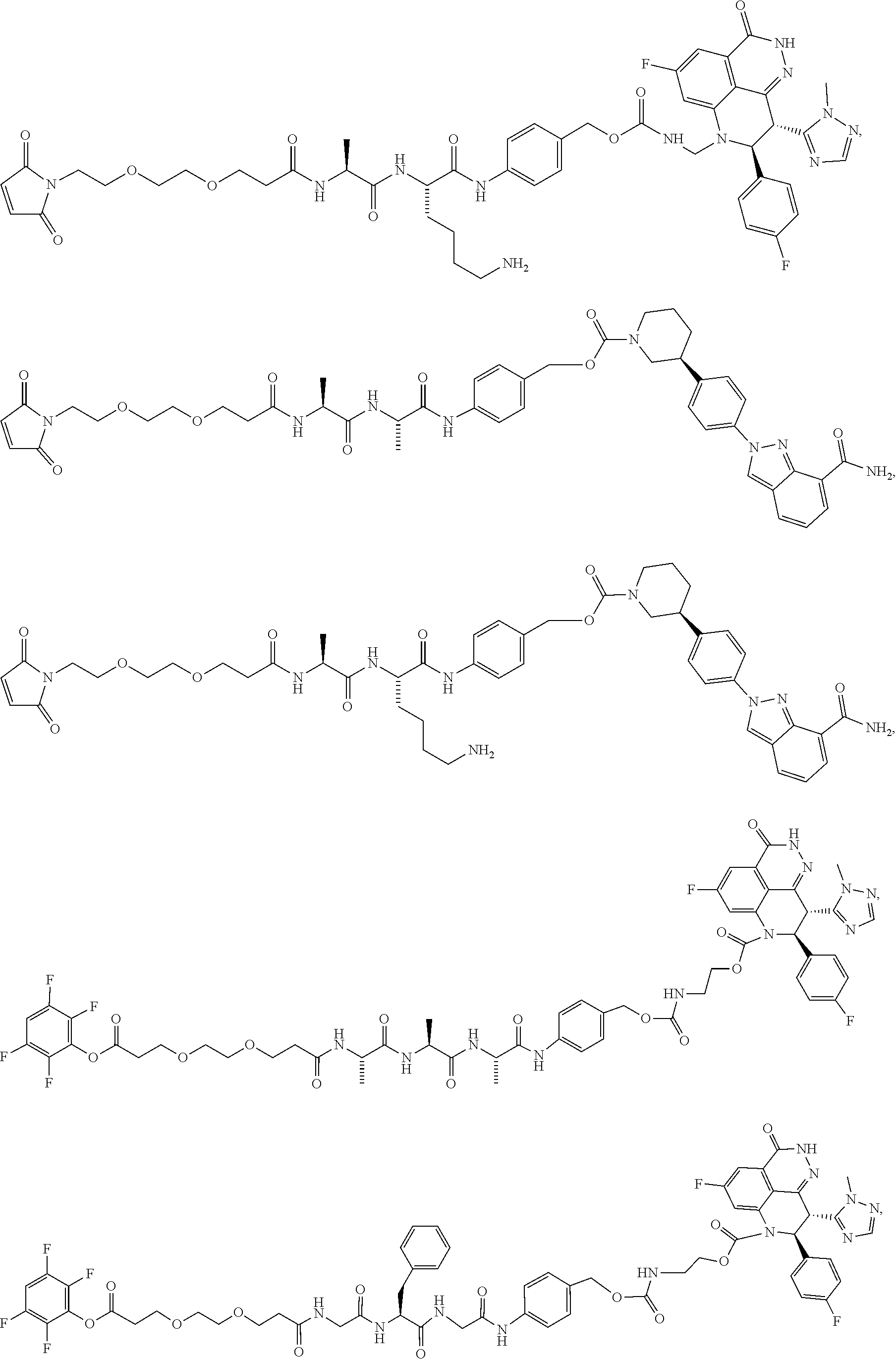

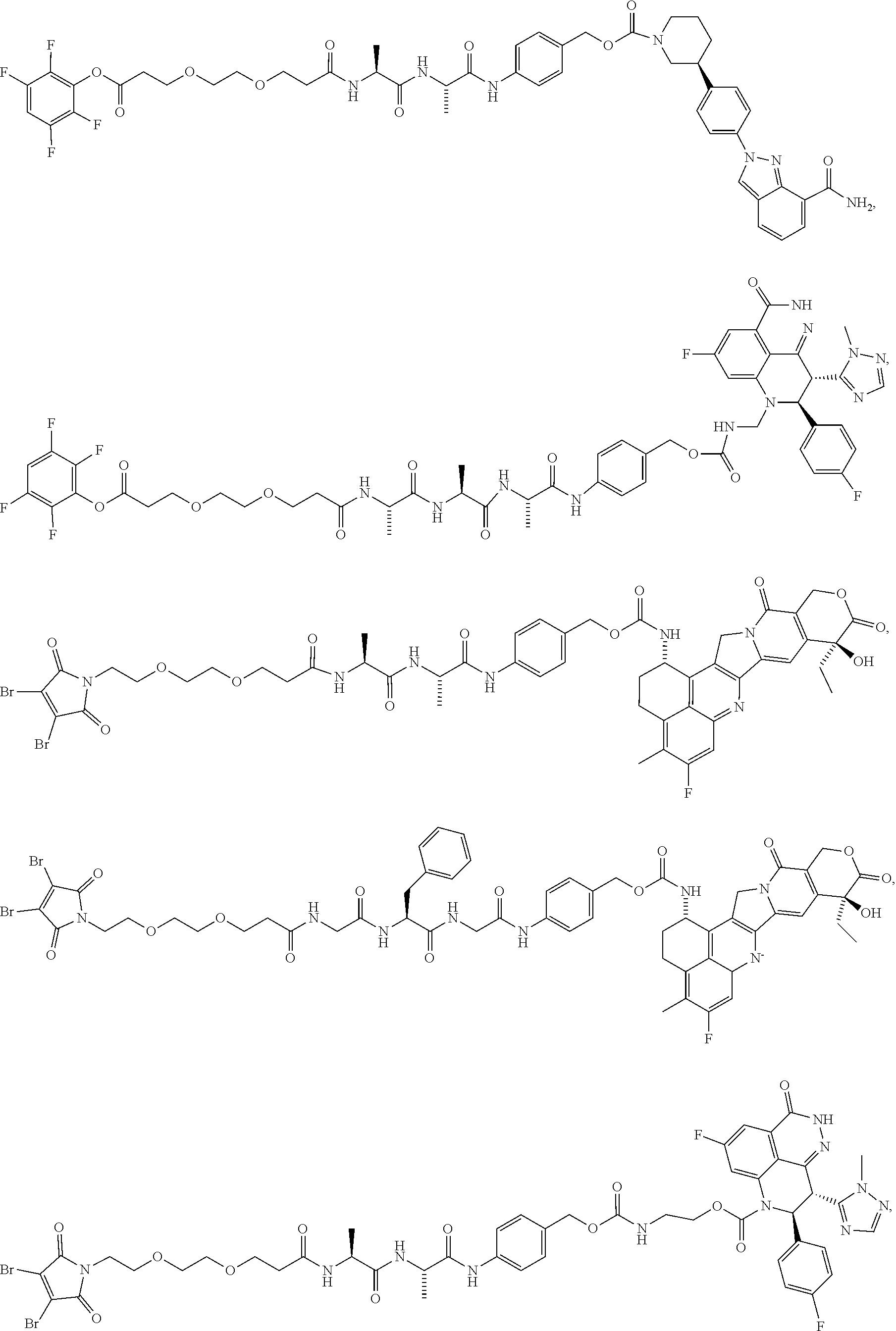

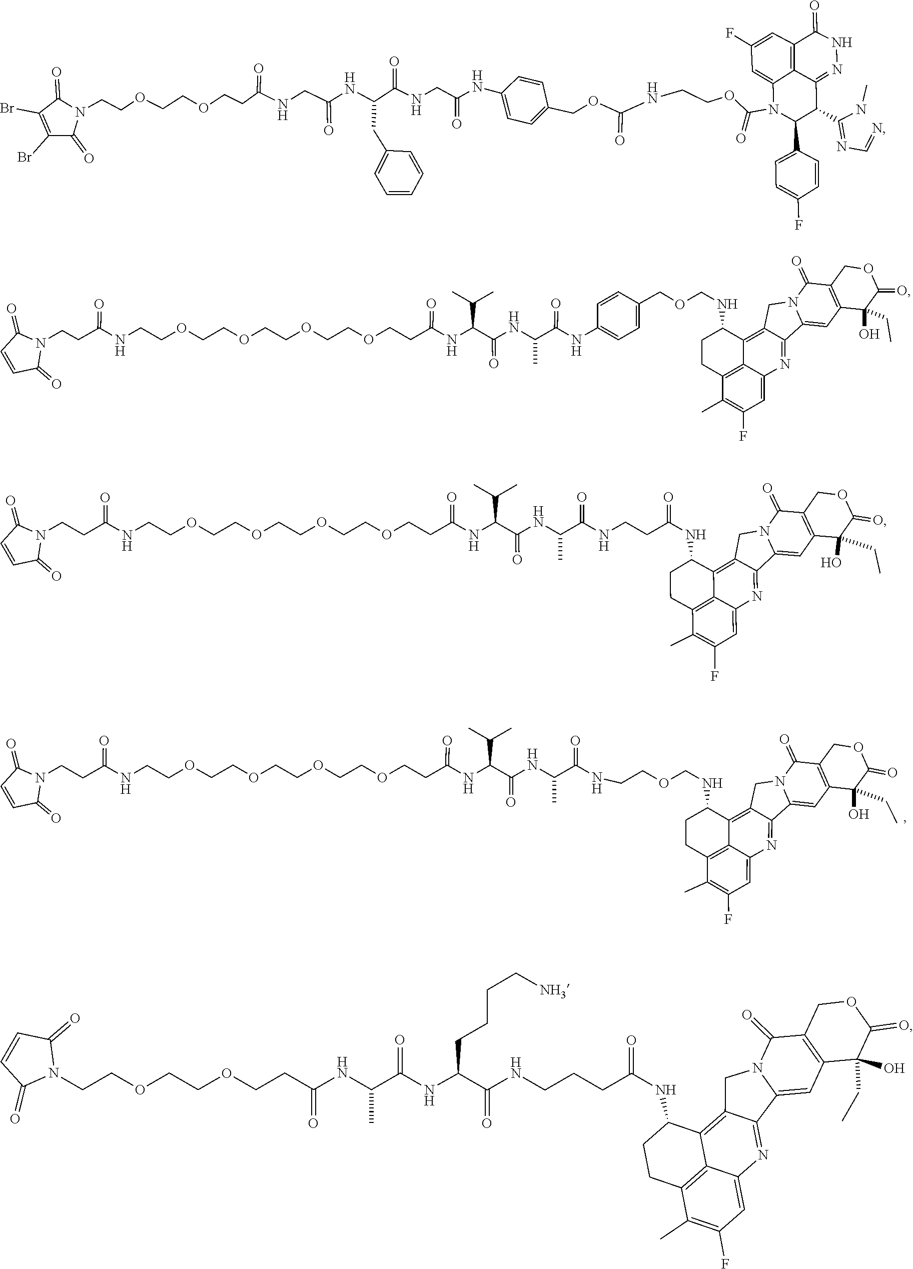

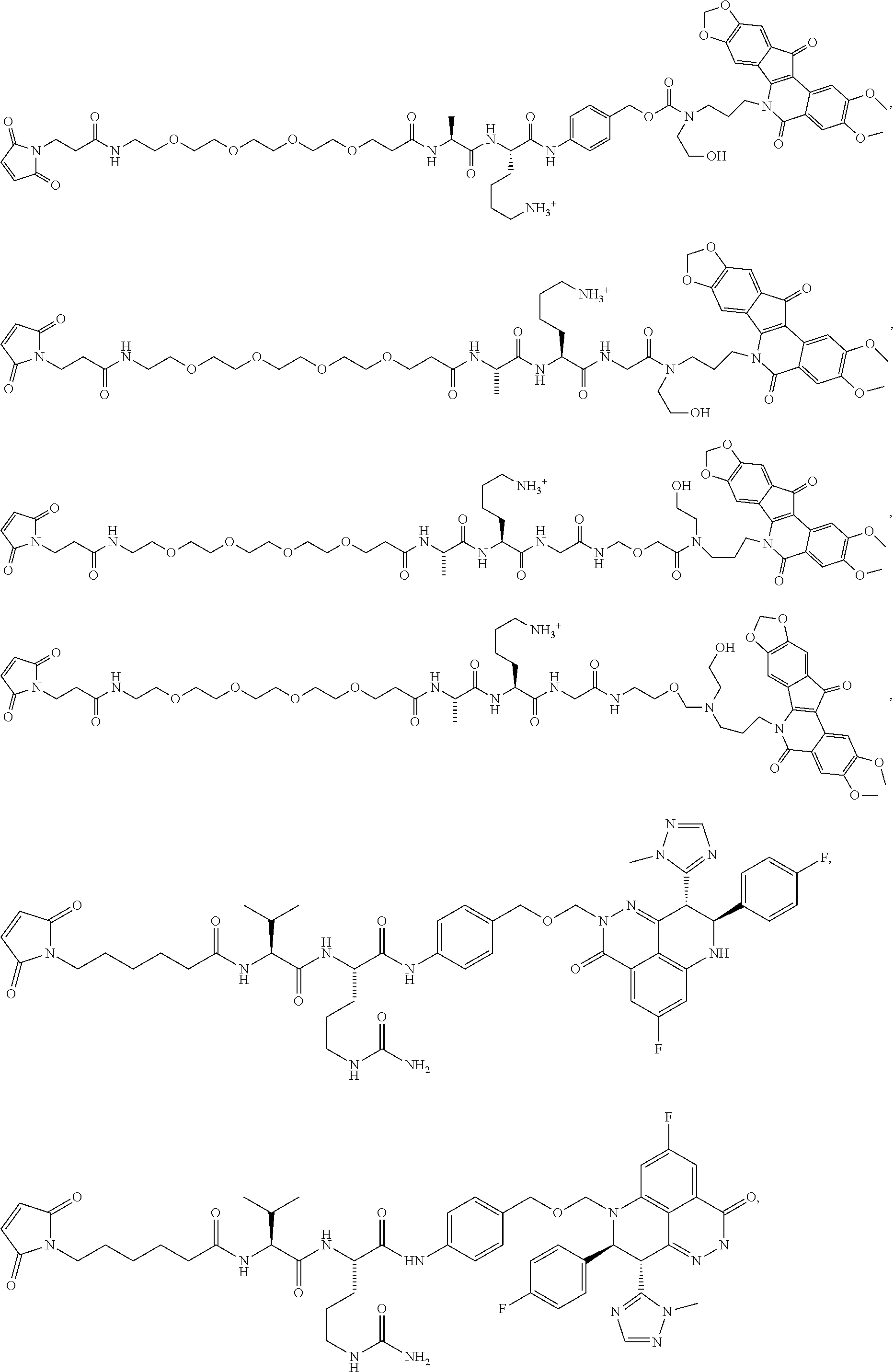

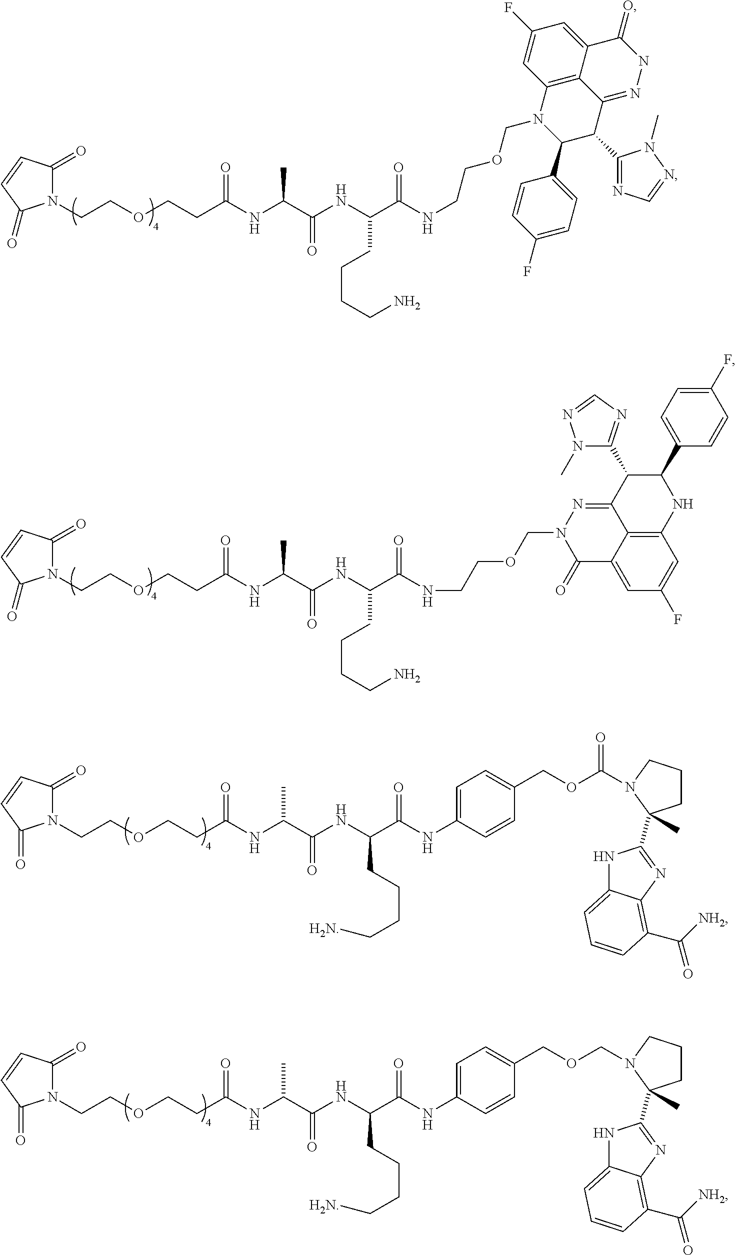

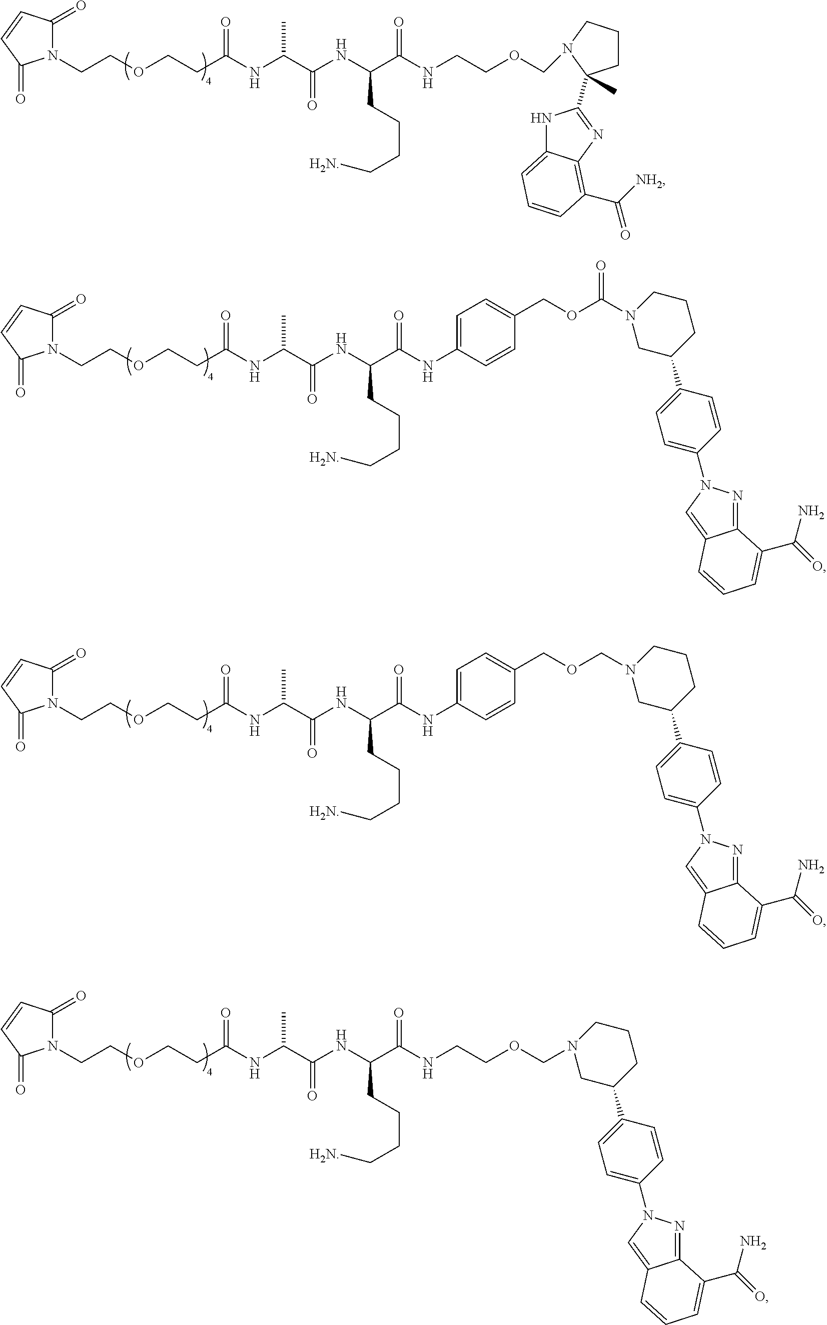

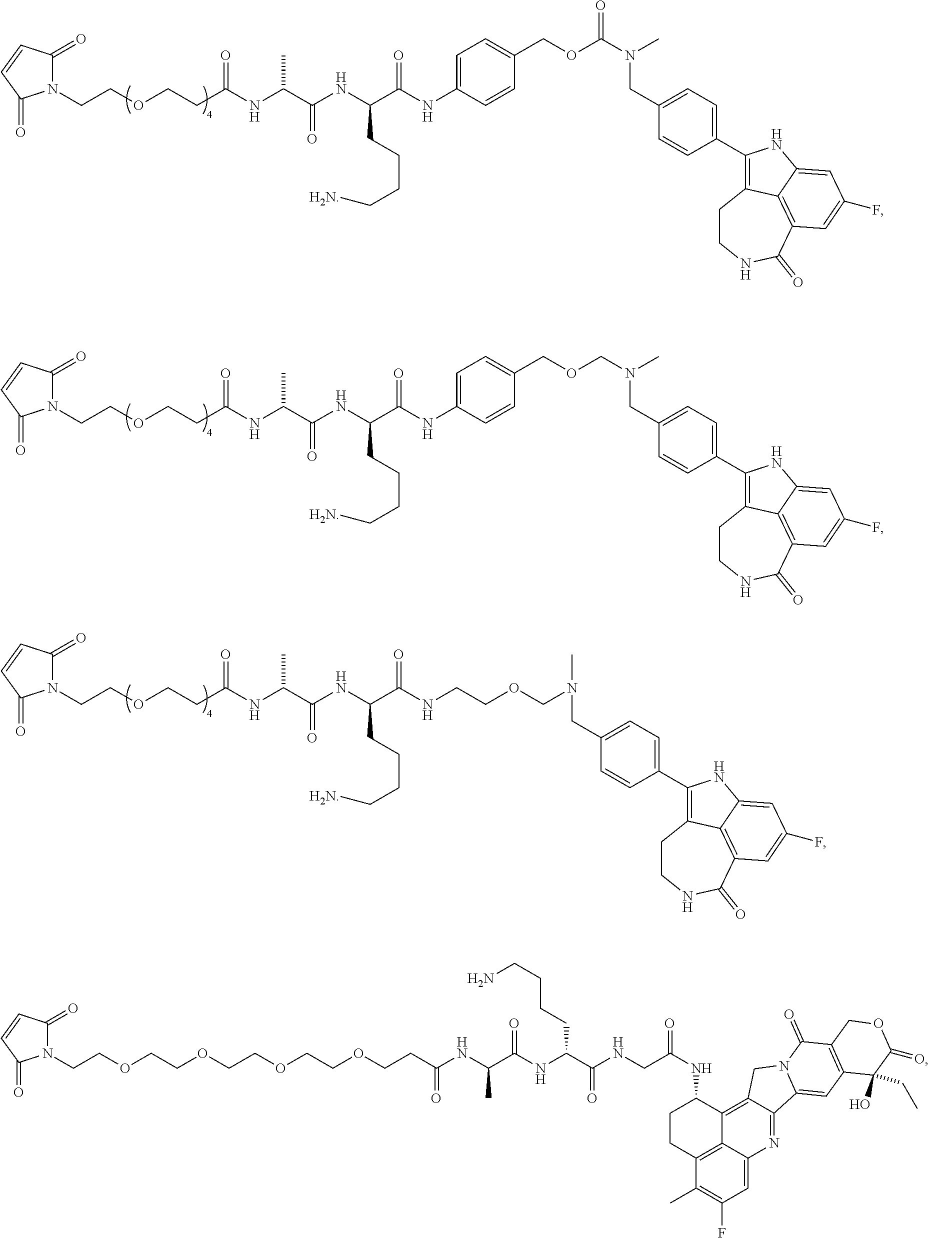

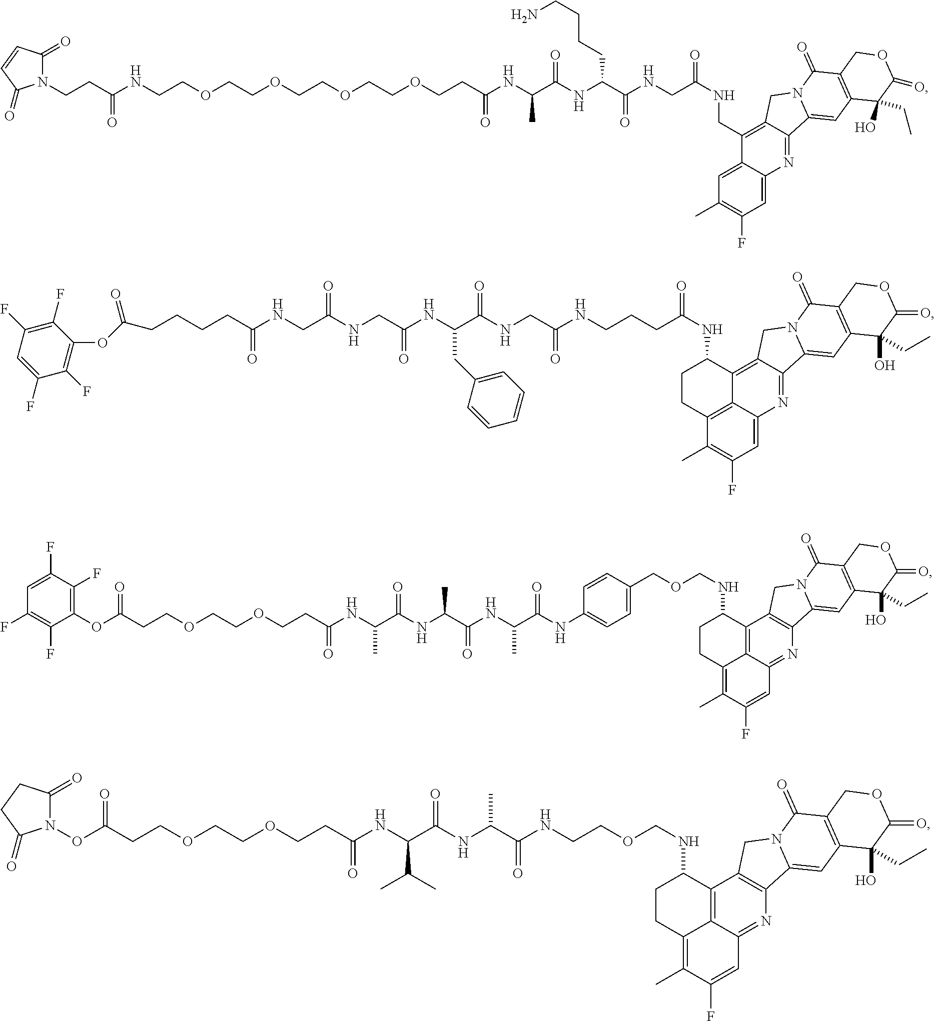

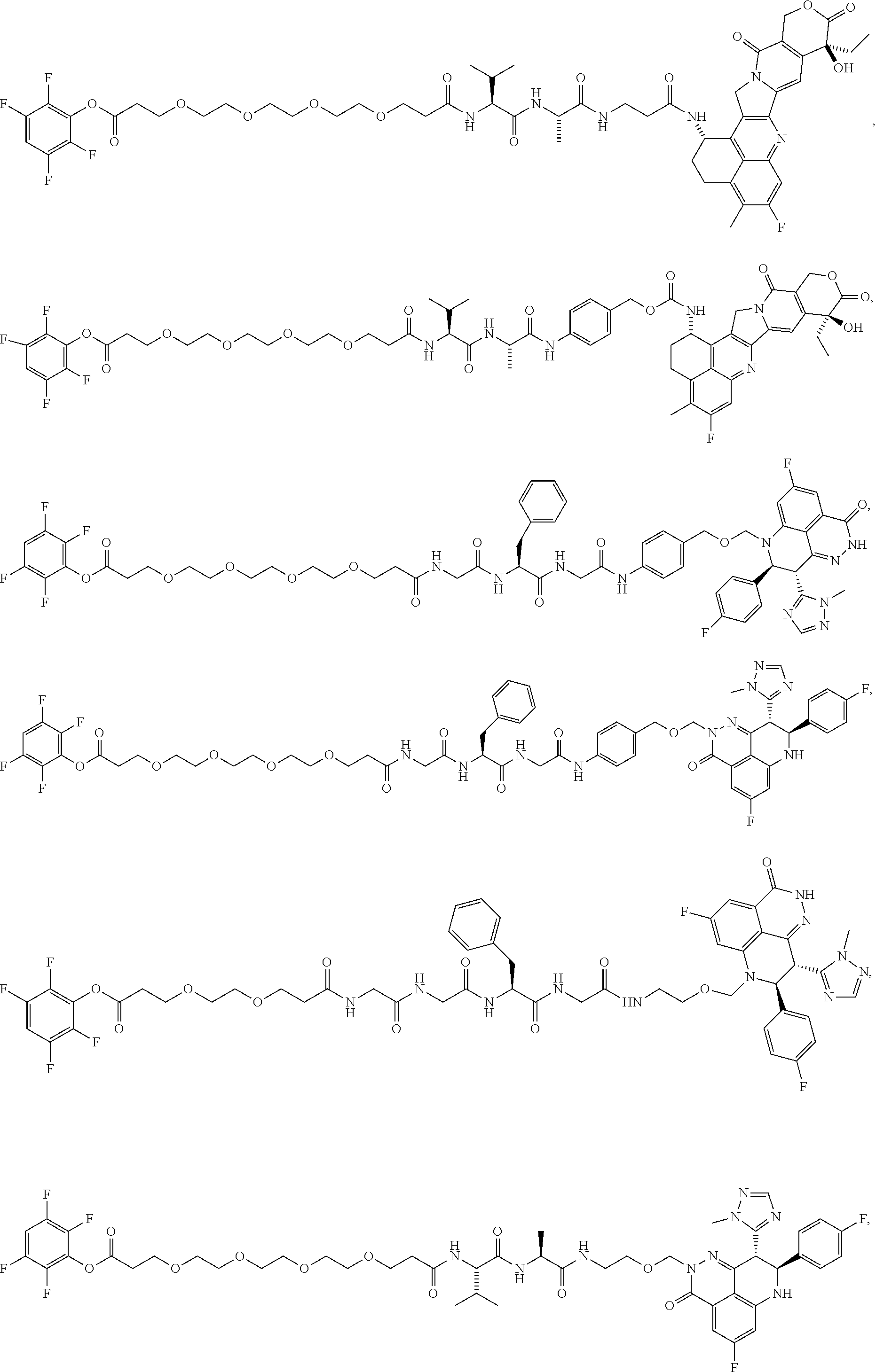

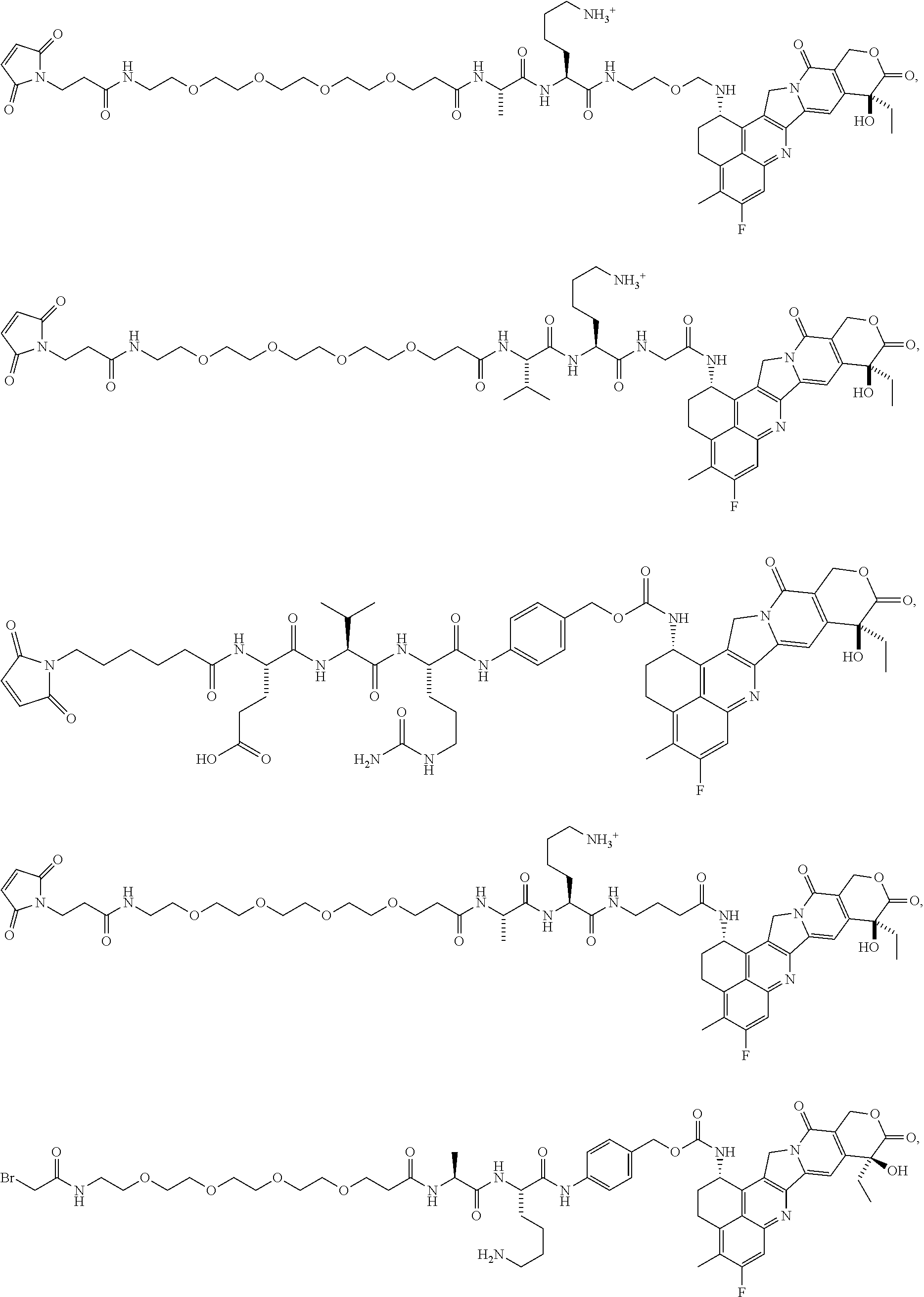

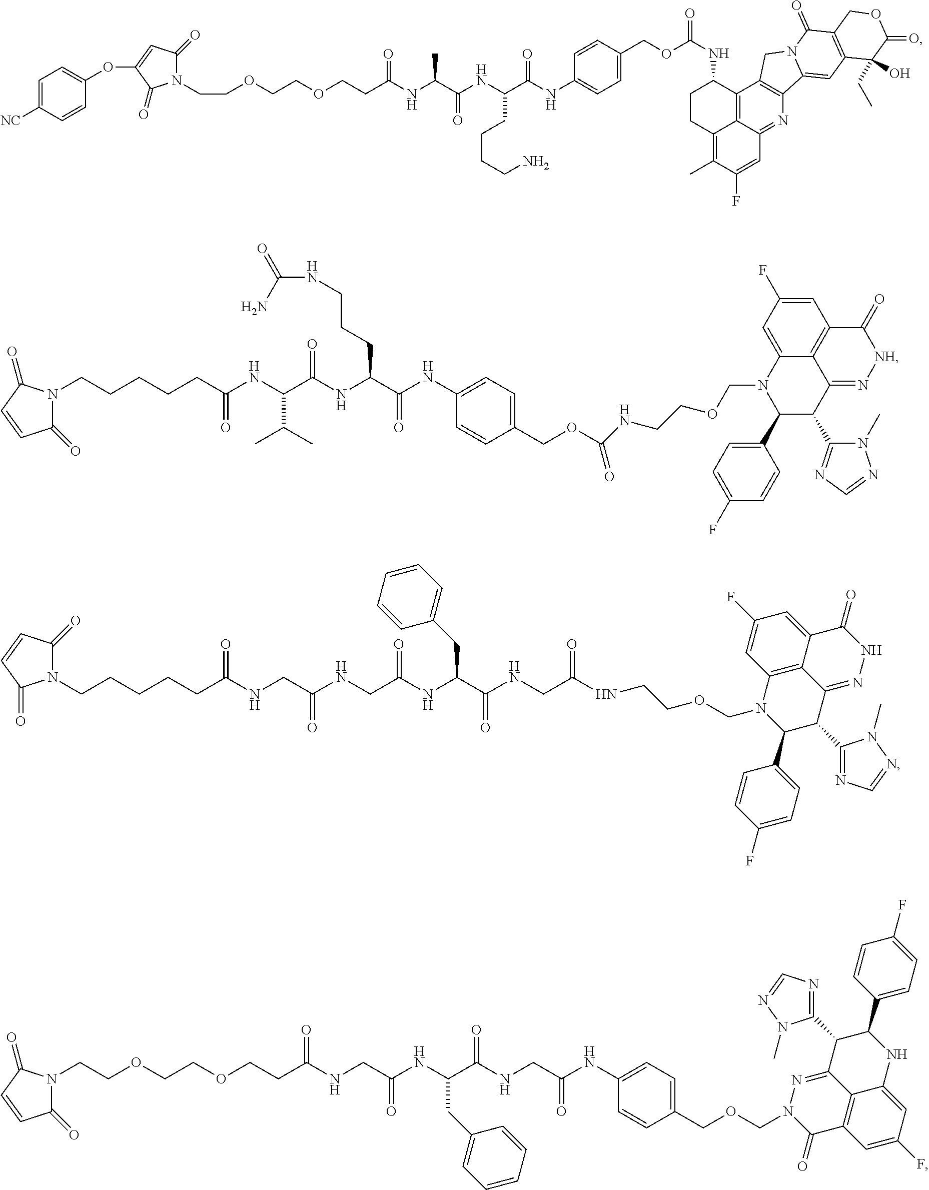

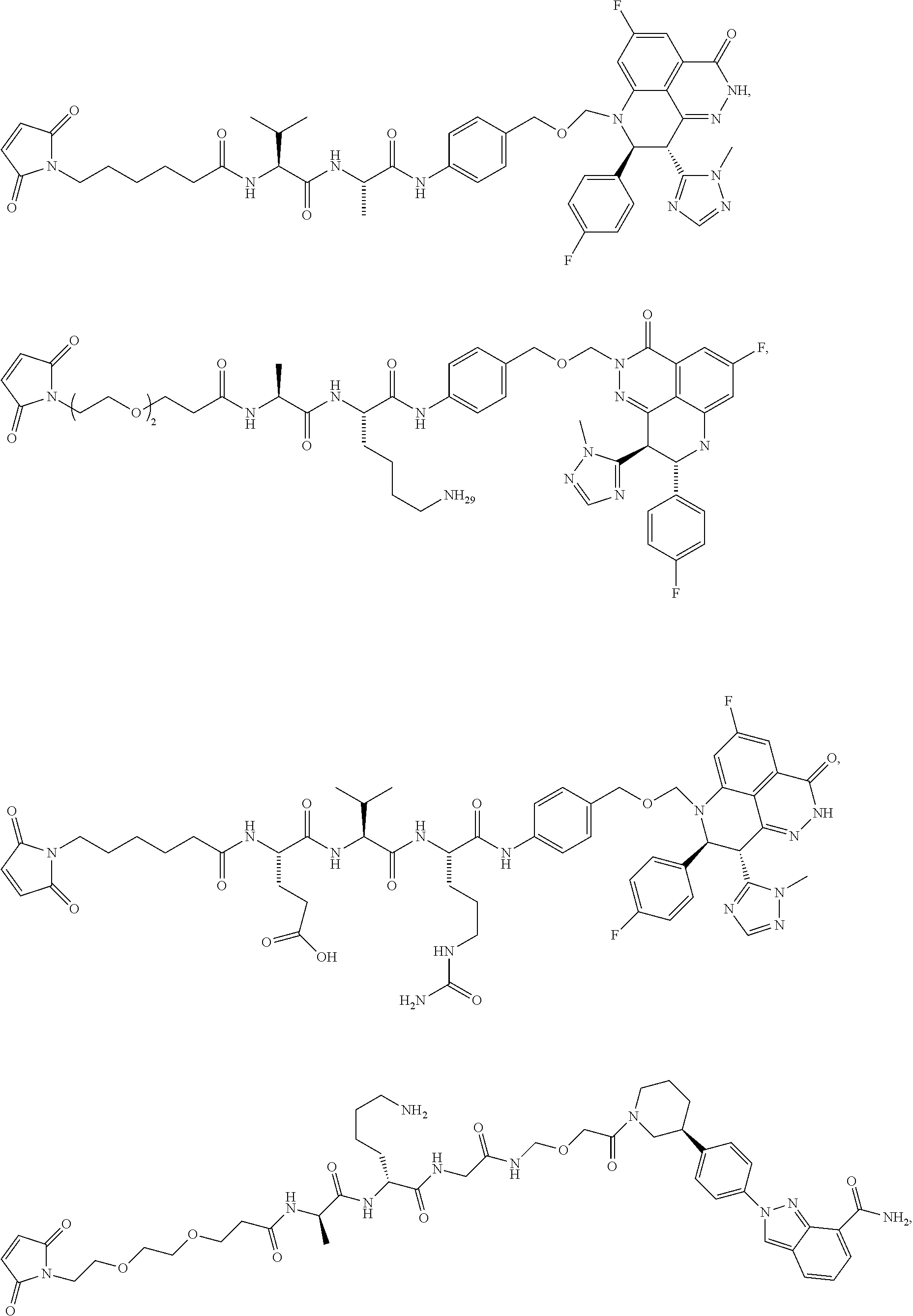

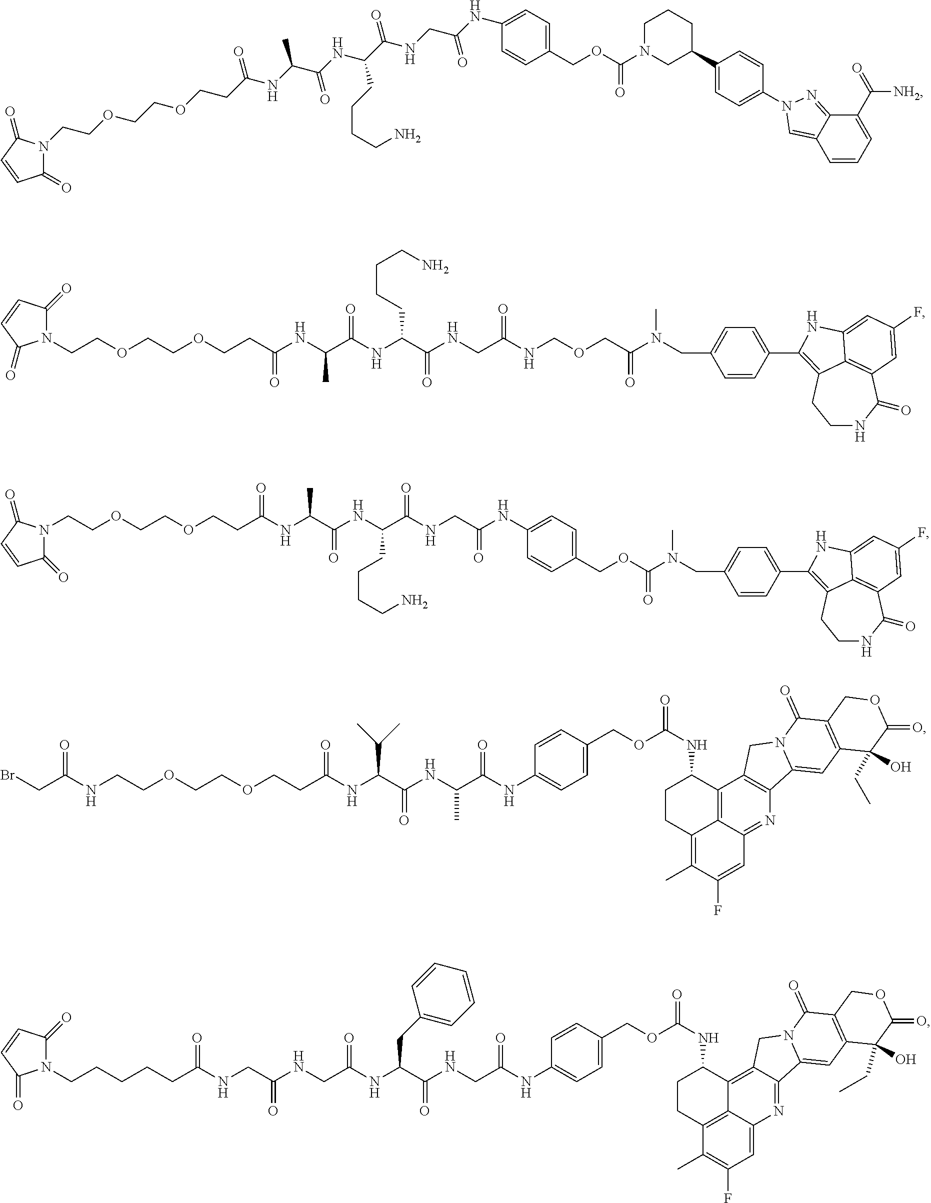

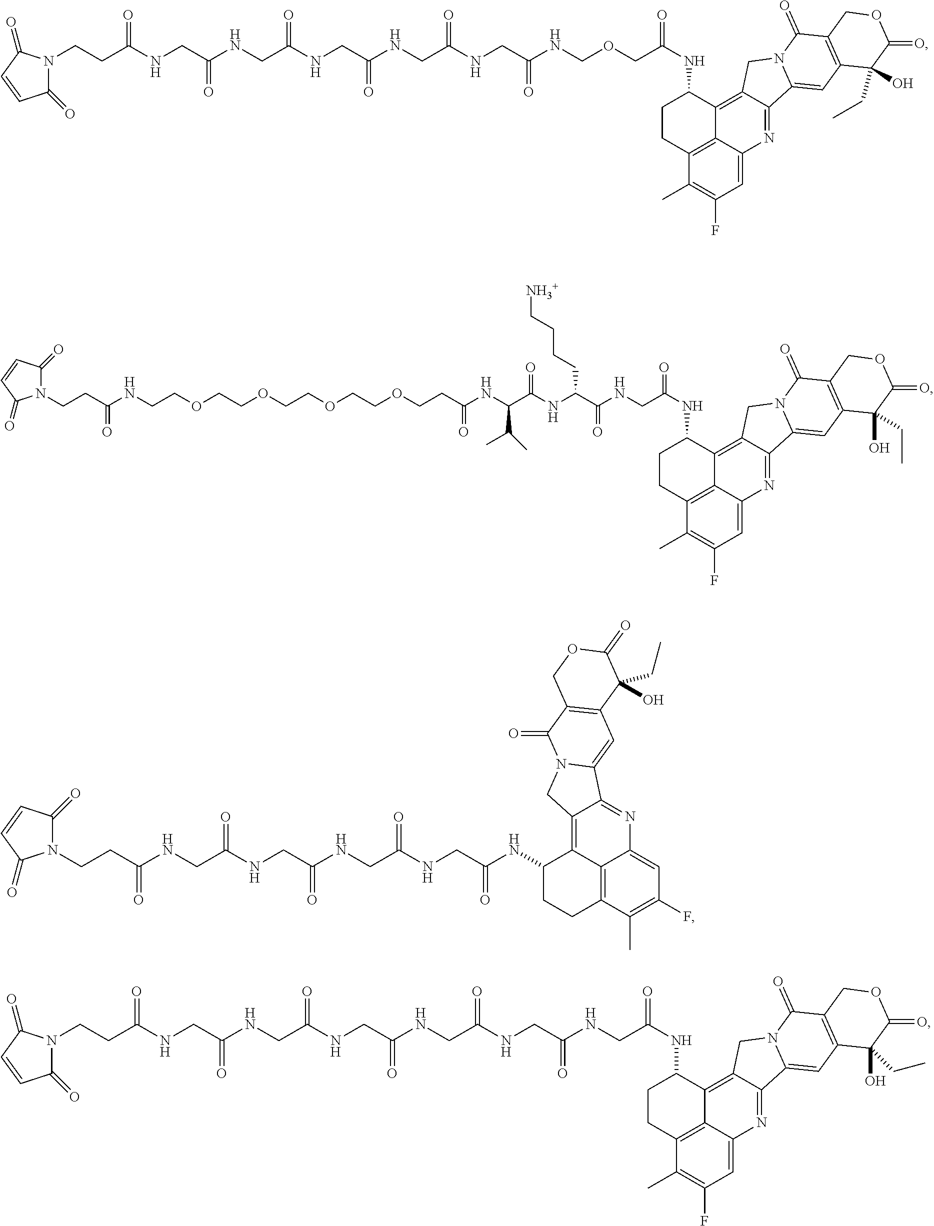

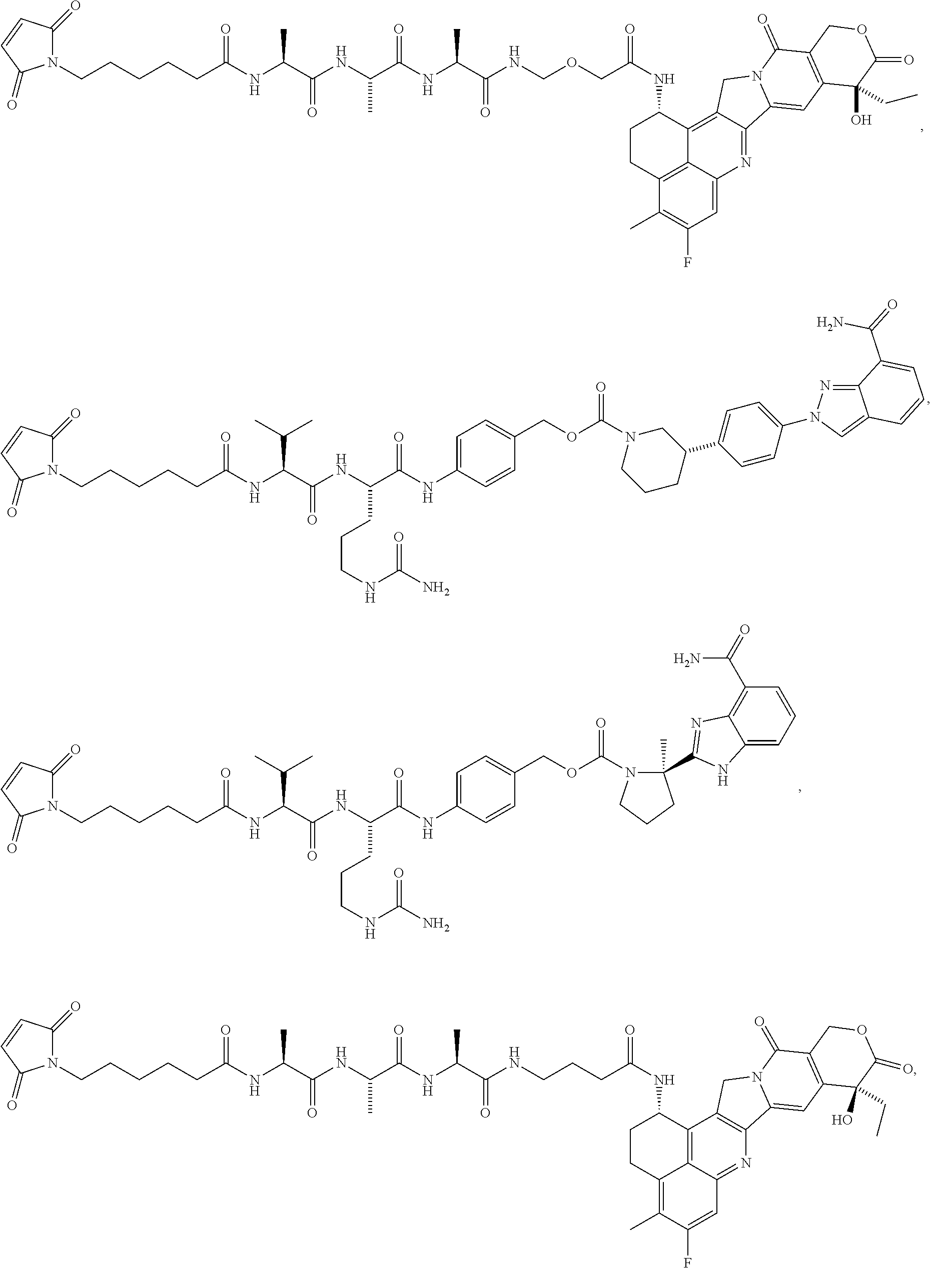

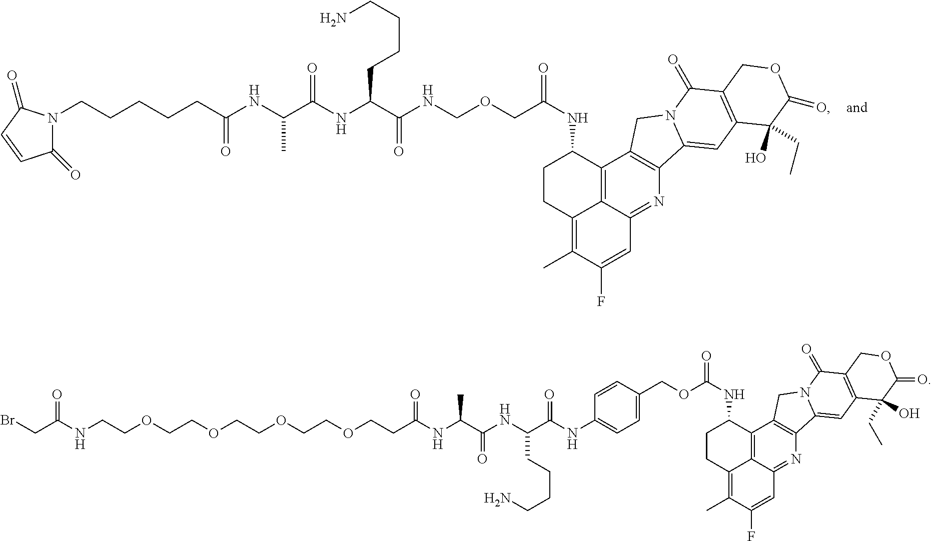 [SMILES: CC[C@@]1(O)C(=O)OCc2c1cc1n(c2=O)Cc2c-1nc1cc(F)c(C)c3c1c2[C@@H](NC(=O)COCNC(=O)[C@H](CCCCN)NC(=O)[C@H](C)NC(=O)CCCCCN1C(=O)C=CC1=O)CC3 ; CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCCN)NC(=O)[C@H](C)NC(=O)CCOCCOCCOCCOCCNC(=O)CBr)cc1)CC3]

or a salt or a hydrate thereof.

In certain embodiments, the compound is selected from the group consisting of:

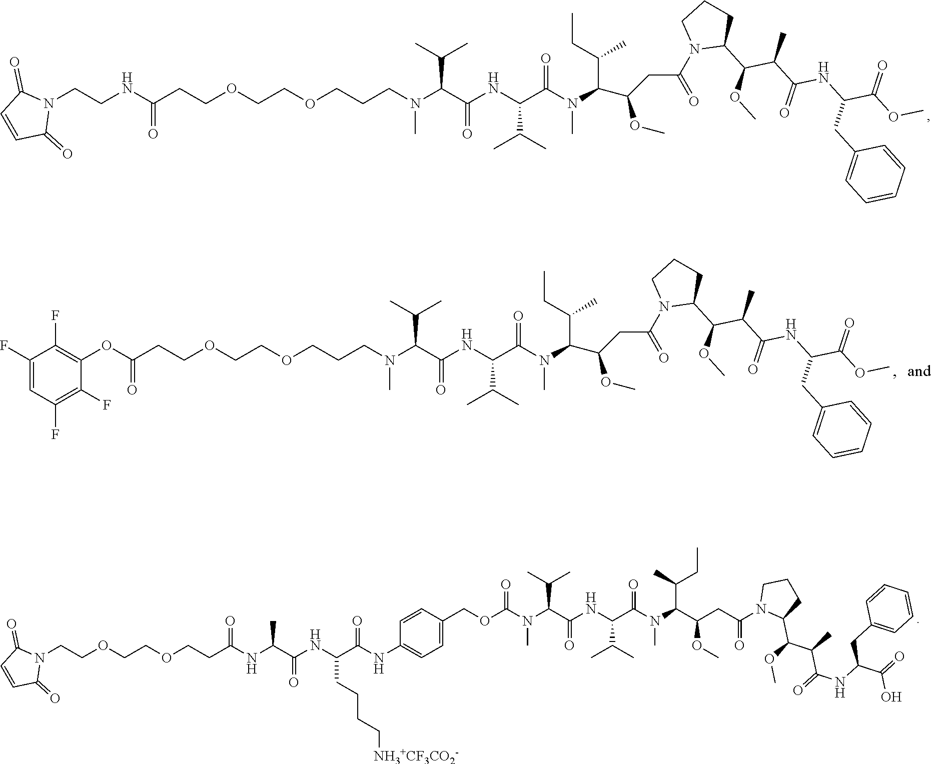

In certain embodiments, the compound is selected from the group consisting of:

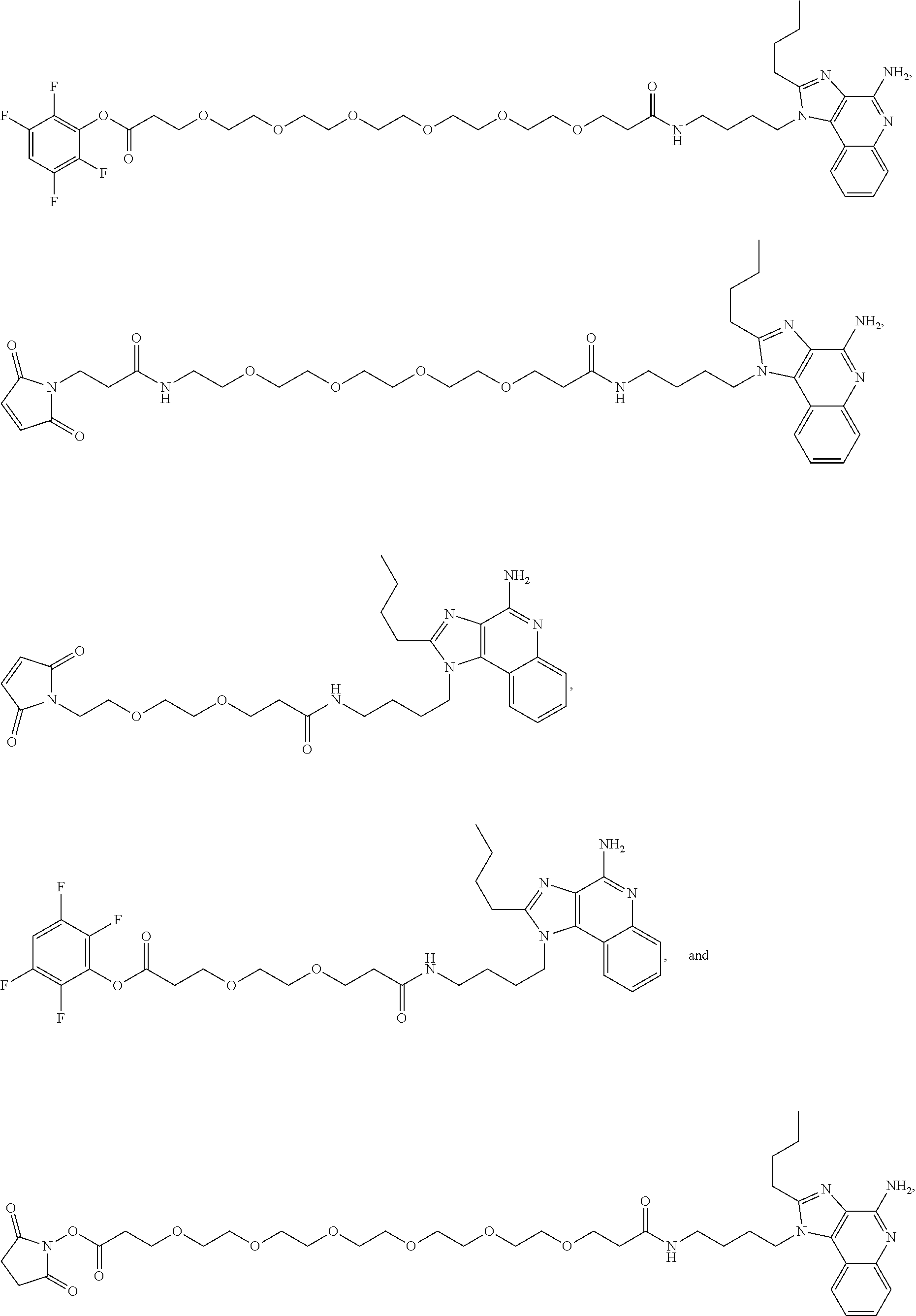 [SMILES: CCCCc1nc2c(N)nc3ccccc3c2n1CCCCNC(=O)CCOCCOCCOCCOCCOCCOCCC(=O)Oc1c(F)c(F)cc(F)c1F ; CCCCc1nc2c(N)nc3ccccc3c2n1CCCCNC(=O)CCOCCOCCOCCOCCNC(=O)CCN1C(=O)C=CC1=O ; CCCCc1nc2c(N)nc3ccccc3c2n1CCCCNC(=O)CCOCCOCCN1C(=O)C=CC1=O ; CCCCc1nc2c(N)nc3ccccc3c2n1CCCCNC(=O)CCOCCOCCC(=O)Oc1c(F)c(F)cc(F)c1F ; CCCCc1nc2c(N)nc3ccccc3c2n1CCCCNC(=O)CCOCCOCCOCCOCCOCCOCCC(=O)ON1C(=O)CCC1=O]

### Antibody-Drug Conjugates

An antibody-drug conjugate (ADC) allows the targeted delivery of therapeutic agent(s) to specific cells and/or tissues. In certain embodiments, the ADC comprises an agent (e.g., a therapeutic agent) capable of inhibiting topoisomerase (e.g., topoisomerase I (TopoI)). In certain embodiments, the ADC comprises an agent (e.g., a therapeutic agent) capable of inhibiting poly (ADP-ribose) polymerase (PARP). In certain embodiments, the ADC provided herein comprises an agent (e.g., a therapeutic agent) capable of inhibiting tubulin. In certain embodiments, the ADC provided herein comprises an agent (e.g., a therapeutic agent) capable of activating toll-like receptor 7 or 8 (TLR7 or TLR8).

In another aspect, provided herein are conjugates, e.g., antibody-drug conjugates. In certain embodiments, the ADC comprises. a therapeutic agent (indicated as T); an optional spacer (indicated as SP); a linking moiety (indicated as L); and a residual moiety (indicated as Z) resulting from the covalent linkage of a conjugation moiety Y to an antibody (indicated as [symbol] ) by nucleophilic attack by a thiol group of a cystine or by an amino group of a lysine of an antibody. In some embodiments, intracellular cleavage of the L linker or the SP spacer allows the separation of the therapeutic agent T from the [symbol] , thereby promoting the uptake or retention therapeutic agent T into cells or tissue retention.

In certain embodiments, T is a moiety derived from a compound capable of binding to topoisomerase (e.g., topoisomerase I), poly (ADP-ribose) polymerase (PARP), tubulin, or toll-like receptor 7 or 8 (TLR7 or TLR8). In certain embodiments, T is a moiety derived from a compound capable of binding to topoisomerase (e.g., topoisomerase I) or poly (ADP-ribose) polymerase (PARP). In other embodiments, T is a moiety derived from a compound capable of binding to tubulin, or toll-like receptor 7 or 8 (TLR7 or TLR8). In one embodiment, T is a moiety derived from a compound capable of binding to (e.g., inhibiting) tubulin. In one embodiment, T is a moiety derived from tubulin inhibitor. In another embodiment, T is a moiety derived from a compound capable of binding to (e.g., activating) TLR7 or TLR8. In another embodiment, T is a moiety derived from a TLR7 or TLR8 agonist.

In another aspect, provided herein are conjugates of Formula (II):

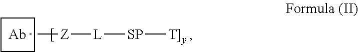

wherein:
T is a moiety derived from a compound capable of inhibiting topoisomerase enzyme or poly (ADP-ribose) polymerase (PARP);
SP is absent, a di- or tri-peptide linking moiety having L bonded to the N-terminus and T bonded to the C-terminus, or a linking moiety capable of self-immolation at pH less than 8;
L is a di- or tri-peptide linking moiety having Y bonded to the N-terminus and SP bonded to the C-terminus;
[symbol] is an antibody;
Z is a residual moiety resulting from the covalent linkage of Y to [symbol] ; and Y is a conjugation moiety capable of forming a covalent bond with a nitrogen atom of a lysine residue or a sulfur atom of a cysteine residue; and
wherein y is an integer from 1 to 20.

In certain embodiments, Z is selected from:

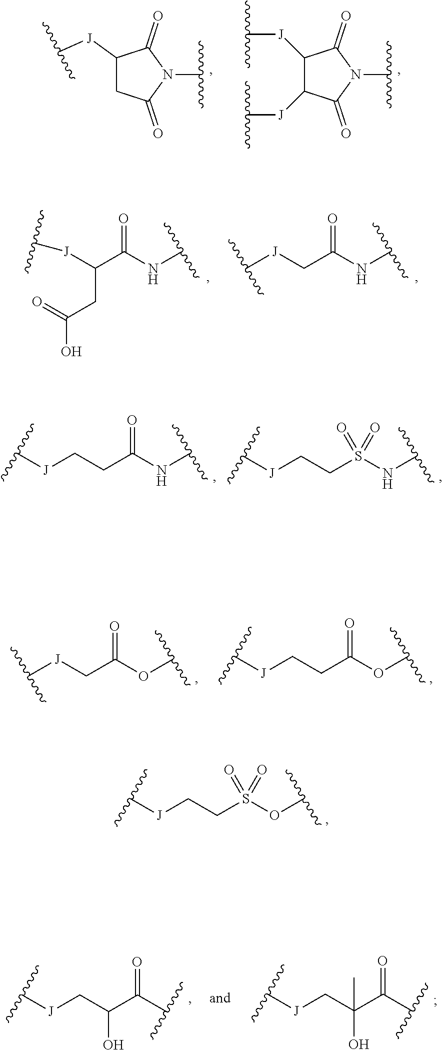

wherein J is —S— or —NH—.

In some embodiments, Z is

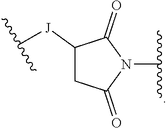

In some embodiments, Z is

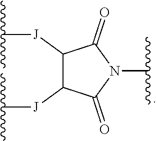

In some embodiments, Z is

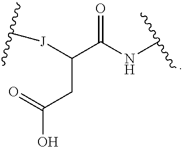

In some embodiments, Z is

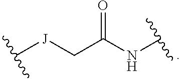

In some embodiments, Z is

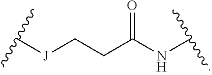

In some embodiments, Z is

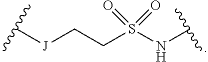

In some embodiments, Z is

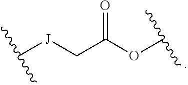

In some embodiments, Z is

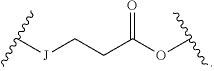

In some embodiments, Z is

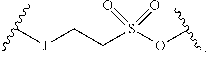

In some embodiments, Z is

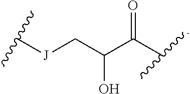

In some embodiments, Z is

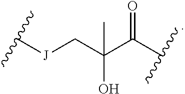

In certain embodiments, Z is selected from

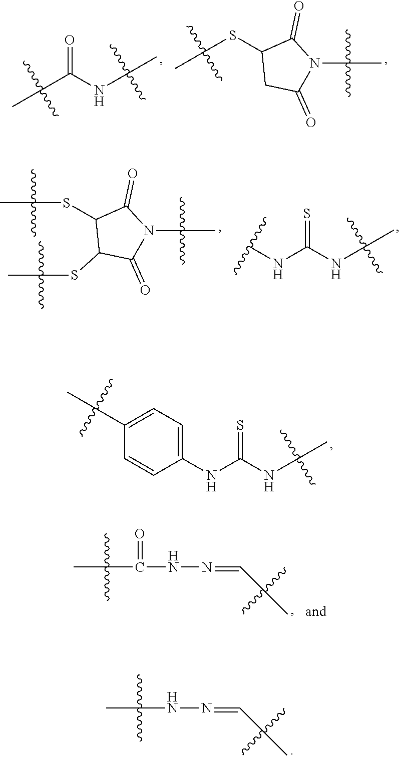

In certain embodiments, Z is

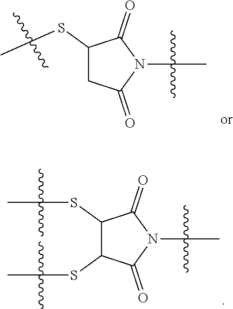 [SMILES: *SC1CC(=O)N(*)C1=O ; *SC1C(=O)N(*)C(=O)C1S*]

In certain embodiments, Z is

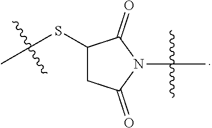 [SMILES: *SC1CC(=O)N(*)C1=O]

In certain embodiments, Z is

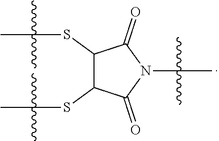 [SMILES: *SC1C(=O)N(*)C(=O)C1S*]

In certain embodiments, Z is

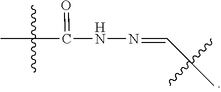 [SMILES: *C=NNC(*)=O]

In certain embodiments, Z is

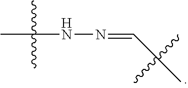 [SMILES: *C=NN*]

In certain embodiments, Z is

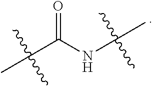 [SMILES: *NC(*)=O]

In certain embodiments, Z is

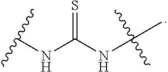 [SMILES: *NC(=S)N*]

### Z is

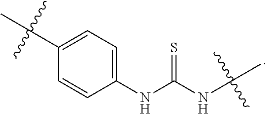 [SMILES: *NC(=S)Nc1ccc(*)cc1]

In some embodiments, the conjugate is a conjugate of Formula (IIa),

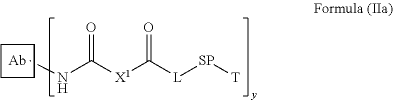

wherein,
   X¹ is selected from bond, —(CH₂)ₙ—, —(CH₂)ₙ—(O—CH₂CH₂)ₘ—, —(CH₂)ₘ—O—(CH₂)ₘ—, and —(CH₂)ₙ—NH—(CH₂)ₘ—, wherein n is an integer from 1 to 5, and m is an integer from 1 to 8;
   T is a moiety derived from a compound capable of inhibiting topoisomerase or poly (ADP-ribose) polymerase (PARP);
   SP is absent, a di- or tri-peptide linking moiety having L bonded to the N-terminus and T bonded to the C-terminus, or a linking moiety capable of self-immolation at pH less than 8;
   L is a di- or tri-peptide linking moiety having SP bonded to the C-terminus;
   [symbol] is an anti-ICAM1 or anti-EphA2 antibody; and
   wherein y is an integer from 1 to 20.

In some embodiments, the conjugate is a conjugate of Formula (IIb):

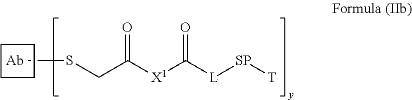

wherein,
   X¹ is selected from bond, —(CH₂)ₙ—, —(CH₂)ₙ—(OCH₂CH₂)ₘ—, —(CH₂)ₙ—O—(CH₂)ₘ—, —(CH₂)ₙ—NH—(CH₂)ₘ—, wherein n is 1 to 5, and m is 1 to 8;
   T is a moiety derived from a compound capable of inhibiting topoisomerase or poly (ADP-ribose) polymerase (PARP);
   SP is absent, a di- or tri-peptide linking moiety having L bonded to the N-terminus and T bonded to the C-terminus, or a linking moiety capable of self-immolation at pH less than 8;
   L is a di- or tripeptide linking moiety having SP bonded to the C-terminus;
   [symbol] is an anti-ICAM1 or anti-EphA2 antibody;
wherein y is an integer from 1 to 8.

In some embodiments, the conjugate is a conjugate of Formula (IIc):

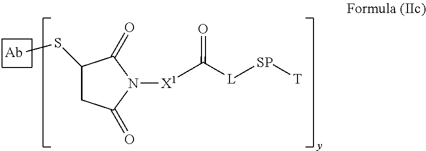

wherein,
   X¹ is selected from bond, —(CH₂)ₙ—, —(CH₂)ₙ—(OCH₂CH₂)ₘ—, —(CH₂)ₙ—O—(CH₂)ₘ—, —(CH₂)ₙ—NH—(CH₂)ₘ—, wherein n is 1 to 5, and m is 1 to 8;
   T is a moiety derived from a compound capable of inhibiting topoisomerase or poly (ADP-ribose) polymerase (PARP);
   SP is absent, a di- or tri-peptide linking moiety having L bonded to the N-terminus and T bonded to the C-terminus, or a linking moiety capable of self-immolation at pH less than 8;
   L is a di- or tripeptide linking moiety SP bonded to the C-terminus;
   [symbol] is an anti-ICAM1 or anti-EphA2 antibody; and
wherein y is an integer from 1 to 8.

In some embodiments, the conjugate is a conjugate of Formula (IId):

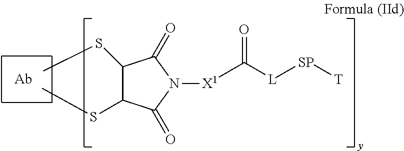

wherein,
   X¹ is selected from bond, —(CH₂)ₙ—, —(CH₂)ₙ—(OCH₂CH₂)ₘ—, —(CH₂)ₙ—O—(CH₂)ₘ—, —(CH₂)ₙ—NH—(CH₂)ₘ—, wherein n is 1 to 5, and m is 1 to 8;
   T is a moiety derived from a compound capable of inhibiting topoisomerase or poly (ADP-ribose) polymerase (PARP);
   SP is absent, a di- or tr-peptide linking moiety having L bonded to the N-terminus and T bonded to the C-terminus, or a linking moiety capable of self-immolation at pH less than 8;
   L is a di- or tri-peptide linking moiety having SP bonded to the C-terminus;
   [symbol] is an anti-ICAM1 or anti-EphA2 antibody; and
wherein y is an integer from 1 to 4.

In certain embodiments, Z is:

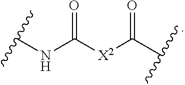

In certain embodiments, wherein X² is selected from bond, —(CH₂)ₙ—, —(CH₂)ₙ—(OCH₂CH₂)ₚ—, —(CH₂)ₙ—O—(CH₂)ₚ—, and —(CH₂)ₙ—NH—(CH₂)ₚ—, wherein n is an integer from 1 to 5, and p is an integer from 1 to 8. In some embodiments, X² is selected from —(CH₂)ₙ—(OCH₂CH₂)ₚ—, —(CH₂)ₙ—O—(CH₂)ₚ—, and —(CH₂)ₙ—NH—(CH₂)ₚ—. In some embodiments, X² is selected from —(CH₂)ₙ—, —(CH₂)ₙ—O—(CH₂)ₚ—, and —(CH₂)ₙ—NH—(CH₂)ₚ—. In some embodiments, X² is selected from —(CH₂)ₙ—, —(CH₂)ₙ—(OCH₂CH₂)ₚ—, and —(CH₂)ₙ—NH—(CH₂)ₚ—. In some embodiments, X² is selected from —(CH₂)ₙ—, —(CH₂)ₙ—(OCH₂CH₂)ₚ—, and —(CH₂)ₙ—O—(CH₂)ₚ—. In one embodiment, X² is —(CH₂)ₙ—. In another embodiment, X² is —(CH₂)ₙ—(OCH₂CH₂)ₚ—. In yet another embodiment, X² is —(CH₂)ₙ—O—(CH₂)ₚ—. In yet another embodiment, X² is —(CH₂)ₙ—NH—(CH₂)ₚ—. In yet another embodiment, X² is bond.

In certain embodiments, Z is:

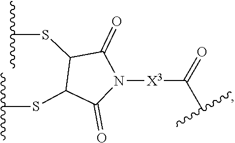

wherein X³ is selected from bond, —(CH₂)ₙ—, —(CH₂)ₙ—(OCH₂CH₂)ₘ—, —(CH₂)ₙ—O—(CH₂)ₚ—, —(CH₂)ₙ—NH—(CH₂)ₚ—, wherein n is an integer from 1 to 5, and p is an integer from 1 to 8; wherein x′ is H, Cl, Br, I, 2-thiopyridyl, or 4-cyanophenoxy; and x″ is H, Cl, Br, I, 2-thiopyridyl, or 4-cyanphenoxy.

In certain embodiments, wherein X³ is selected from bond, —(CH₂)ₙ—, —(CH₂)ₙ—(OCH₂CH₂)ₚ—, —(CH₂)ₙ—O—(CH₂)ₚ—, and —(CH₂)ₙ—NH—(CH₂)ₚ—, wherein n is an integer from 1 to 5, and p is an integer from 1 to 8. In some embodiments, X³ is selected from —(CH₂)ₙ—(OCH₂CH₂)ₚ—, —(CH₂)ₙ—O—(CH₂)ₚ—, and —(CH₂)ₙ—NH—(CH₂)ₚ—. In some embodiments, X³ is selected from —(CH₂)ₙ—, —(CH₂)ₙ—O—(CH₂)ₚ—, and —(CH₂)ₙ—NH—(CH₂)ₚ—. In some embodiments, X³ is selected from —(CH₂)ₙ—, —(CH₂)ₙ—(OCH₂CH₂)ₚ—, and —(CH₂)ₙ—NH—(CH₂)ₚ—. In some embodiments, X³ is selected from —(CH₂)ₙ—, —(CH₂)ₙ—(OCH₂CH₂)ₚ—, and —(CH₂)ₙ—O—(CH₂)ₚ—. In one embodiment, X³ is —(CH₂)ₙ—. In another embodiment, X³ is —(CH₂)ₙ—(OCH₂CH₂)ₚ—. In yet another embodiment, X³ is —(CH₂)ₙ—O—(CH₂)ₚ—. In yet another embodiment, X³ is —(CH₂)ₙ—NH—(CH₂)ₚ—. In yet another embodiment, X³ is bond.

In certain embodiments, L is a di-peptide linking moiety having the structure of:

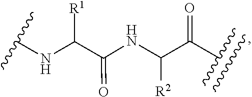

wherein R¹ and R² are independently selected from H, —CH₂CH₂CH₂NHCONH₂, or the side chain of a naturally occurring amino acid, wherein [symbol] represents the point of attachment to Z and [symbol] represents the point of attachment to SP.

In certain embodiments, R¹ and R² are independently selected from the group consisting of hydrogen, —CH₃, —CH(CH₃)₂, —CH₂CH₂CH₂CH₂NH₂, —CH₂C₆H₅, —CH₂C₆H₄OH, —CH₂CH₂CH₂NH(NH)NH₂, —CH₂CH₂CH₂NHCONH₂, and —CH₂CH₂CO₂H. In certain embodiments, R¹ is selected from the group consisting of —CH₃, —CH(CH₃)₂, —CH₂CH₂CH₂CH₂NH₂, and —CH₂C₆H₅. In certain embodiments, R² is selected from the group consisting of —CH₃, —CH₂CH₂CH₂CH₂NH₂, —CH₂C₆H₅, and —CH₂CH₂CH₂NHCONH₂. In certain embodiments, R¹ is —CH₃ and R² is —CH₂CH₂CH₂CH₂NH₂. In certain embodiments, R¹ is —CH₃ and R² is —CH₃. In certain embodiments, R¹ is —CH(CH₃)₂ and R² is —CH₂CH₂CH₂NHCONH₂. In certain embodiments, R¹ is —CH₂C₆H₅ and R² is —CH₂CH₂CH₂CH₂NH₂. In certain embodiments, R¹ is —CH₃ and R² is —CH₂C₆H₅. In certain embodiments, R¹ is —CH₂CH₂CH₂CH₂NH₂ and R² is —CH₃.

In certain embodiments, L is a tri-peptide linking moiety having the structure of:

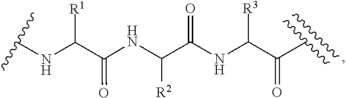

wherein R¹, R² and R³ are independently H, —CH₂CH₂CH₂NHCONH₂, or the side chain of a naturally occurring amino acid, and wherein [symbol] represents the point of attachment to Z and [symbol] represents the point of attachment to SP. In certain embodiments, R¹, R², and R³ are independently selected from the group consisting of hydrogen, CH₃, —CH(CH₃)₂, —CH₂CH₂CH₂CH₂NH₂, —CH₂C₆H₅, CH₂C₆H₄OH, —CH₂CH₂CH₂NH(NH)NH₂, —CH₂CH₂CH₂NHCONH₂, and —CH₂CH₂CO₂H. In certain embodiments, R¹ is H or CH₃. In certain embodiments, R² is —CH₂CH₂CH₂CH₂NH₂, —CH₂C₆H₅, or CH₃. In certain embodiments, R³ is H or CH₃. In certain embodiments, R¹ is H, R² is —CH₂C₆H₅, and R³ is H. In certain embodiments, R¹ is H, R² is —CH₂CH₂CH₂CH₂NH₂, and R³ is H. In certain embodiments, R¹ is CH₃, R² is CH₃, and R³ is CH₃. In certain embodiments, R² is —CH₂CH₂CH₂CH₂NH₂. In certain embodiments, R¹ is CH₃, R² is —CH₂CH₂CH₂CH₂NH₂, and R³ is H. In certain embodiments, R² is —CH₂CH₂CH₂CH₂NH₂, and R³ is H or CH₃.

L is a peptide linking moiety having the structure of: -(AA¹)ₐ(AA²)_{b}(AA³)_{c}(AA⁴)_{d}-, wherein AA¹, AA², AA³, and AA⁴ are each independently a natural or unnatural amino acid, and wherein a, b, c, and d are each independently 0 or 1. L is a peptide linking moiety having the structure of: -(AA¹)ₐ(AA²)_{b}(AA³)_{c}-, wherein AA¹, AA², and AA³ are each independently a natural or unnatural amino acid, and wherein a, b, c, and d are each independently 0 or 1.

In certain embodiments, SP is absent. In certain embodiments, SP is a di- or tri-peptide linking moiety having L bonded to the N-terminus, and T bonded to the C-terminus. In certain embodiments, SP is a di-peptide linking moiety. In certain embodiments, the di-peptide linking moiety of SP is

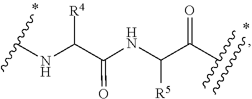

wherein R⁴ and R⁵ are each independently H or optionally substituted C₁₋₆ alkyl, wherein [symbol] represents the point of attachment to L and [symbol] represents the point of attachment to T. In certain embodiments, R⁴ and R⁵ are each independently H or —CH₃. In certain embodiments, R⁴ is H, and R⁵ is H. In certain embodiments, SP is a tri-peptide linking moiety. In certain embodiments, the tri-peptide linking moiety of SP is

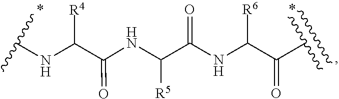

wherein R⁴, R⁵ and R⁶ are each independently H or optionally substituted C₁₋₆ alkyl, wherein [symbol] represents the point of attachment to L and [symbol] represents the point of attachment to T. In certain embodiments, R⁴, R⁵ and R⁶ are each independently H or —CH₃. In certain embodiments, R⁴ is H; R⁵ is H; and R⁶ is H.

In certain embodiments, SP is a linking moiety capable of self-immolation at pH less than 8. In certain embodiments, SP is

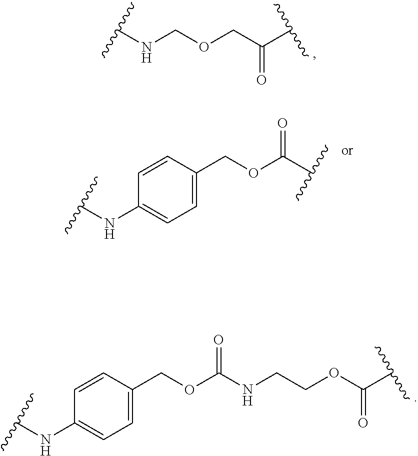 [SMILES: *NCOCC(*)=O ; *Nc1ccc(COC(*)=O)cc1 ; *Nc1ccc(COC(=O)NCCOC(*)=O)cc1]

In certain embodiments of the conjugates described herein, —Z-L-SP-T is bonded through an amide bond to a lysine residue of a polypeptide, e.g., an antibody. In certain embodiments of the conjugates described herein, —Z-L-SP-T is bonded through a thioether bond to a cysteine residue of a polypeptide, e.g., an antibody. In certain embodiments of the conjugates described herein, —Z-L-SP-T is bonded through an amide bond to a lysine residue of [symbol] , as depicted above. In certain embodiments of the conjugates described herein, —Z-L-SP-T is bonded through a thioether bond to a cysteine residue of [symbol] , as depicted above. In certain embodiments of the conjugates described herein, —Z-L-SP-T is bonded through two thioether bonds to two cysteine residues of [symbol] , wherein the two cysteine residues are from an opened cysteine-cysteine disulfide bond in [symbol] , as depicted above. In certain embodiments, the opened cysteine-cysteine disulfide bond is an interchain disulfide bond.

In certain embodiments of the conjugates described herein, when —Z-L-SP-T is bonded through an amide bond to a lysine residue of a polypeptide, e.g., an antibody, y is an integer from 1 to 80. In certain embodiments of the conjugates described herein, when —Z-L-SP-T is bonded through a thioether bond to a cysteine residue of a polypeptide, e.g., an antibody, y is an integer from 1 to 8. In certain embodiments of the conjugates described herein, when —Z-L-SP-T is bonded through two thioether bonds to two cysteine residues of a polypeptide, e.g., an antibody, y is an integer from 1 to 4.

In certain embodiments, conjugation to the polypeptide or the antibody Ab may be via site-specific conjugation. Site-specific conjugation may, for example, result in homogeneous loading and minimization of conjugate subpopulations with potentially altered antigen-binding or pharmacokinetics. In certain embodiments, for example, conjugation may comprise engineering of cysteine substitutions at positions on the polypeptide or antibody, e.g., on the heavy and/or light chains of an antibody that provide reactive thiol groups and do not disrupt polypeptide or antibody folding and assembly or alter polypeptide or antigen binding (see, e.g., Junutula et al., J. Immunol. Meth. 2008; 332: 41-52; and Junutula et al., Nature Biotechnol. 2008; 26: 925-32; see also WO2006/034488 (herein incorporated by reference in its entirety)). In another non-limiting approach, selenocysteine is cotranslationally inserted into a polypeptide or antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., Proc. Natl. Acad. Sci. USA 2008; 105: 12451-56; and Hofer et al., Biochemistry 2009; 48(50): 12047-57). All such methodologies are contemplated for use in connection with making the conjugates described herein.

Loading of the compounds of Formula (I) to the antibodies described herein is represented by “y” in Formulas (II), (IIa), (IIb), (IIc) and/or (IId), and is the average number of units of “—Z-L-SP-T” per conjugate molecule. As used herein, the term “DAR” refers to the average value of “y” or the loading of the conjugate. The number of “T” moieties (e.g., a Topo1 or PARP inhibitor) per each unit of “—Z-L-SP-T” is represented by “y” in Formulas (II), (IIa), (IIb), (IIc) and/or (IId).

DAR (loading of “—Z-L-SP-T” units) may range from 1 to 80 units per conjugate. The average number of units per polypeptide or antibody in preparations of the conjugate from conjugation reactions may be characterized by conventional means such as mass spectroscopy. The quantitative distribution of DAR (loading of “—Z-L-SP-T” units) in terms of y may also be determined. In some instances, separation, purification, and characterization of homogeneous conjugate where y is a certain value may be achieved by means such as electrophoresis.

In certain embodiments, the DAR for a conjugate provided herein ranges from 1 to 80. In certain embodiments, the DAR for a conjugate provided herein ranges from 1 to 60. In certain embodiments, the DAR for a conjugate provided herein ranges from 1 to 40. In certain embodiments, the DAR for a conjugate provided herein ranges from 1 to 20. In certain embodiments, the DAR for a conjugate provided herein ranges from 1 to 15. In certain embodiments, the DAR for a conjugate provided herein ranges from 1 to 10. In certain embodiments, the DAR for a conjugate provided herein ranges from 1 to 8. In certain embodiments, the DAR for a conjugate provided herein ranges from 1 to 7. In certain embodiments, the DAR for a conjugate provided herein ranges from 1 to 6. In certain embodiments, the DAR for a conjugate provided herein ranges from 1 to 5. In certain embodiments, the DAR for a conjugate provided herein ranges from 1 to 4.

In certain embodiments, the DAR for a conjugate provided herein is about 1, about 2, about 3, about 4, about 5, about 6, about 7, about 8, about 9, about 10, about 11, or about 12. In some embodiments, the DAR for a conjugate provided herein is about 3.1, about 3.2, about 3.3, about 3.4, about 3.5, about 3.6, about 3.7, about 3.8, or about 3.9. In some embodiments, the DAR for a conjugate provided herein is about 7.1, about 7.2, about 7.3, about 7.4, about 7.5, about 7.6, about 7.7, about 7.8, about 7.9, or about 8.0.

In some embodiments, the DAR for a conjugate provided herein is about 1. In some embodiments, the DAR for a conjugate provided herein is about 2. In some embodiments, the DAR for a conjugate provided herein is about 3. In some embodiments, the DAR for a conjugate provided herein is about 4. In some embodiments, the DAR for a conjugate provided herein is about 3.8. In some embodiments, the DAR for a conjugate provided herein is about 5. In some embodiments, the DAR for a conjugate provided herein is about 6. In some embodiments, the DAR for a conjugate provided herein is about 7. In some embodiments, the DAR for a conjugate provided herein is about 8.

In certain embodiments, fewer than the theoretical maximum of units are conjugated to the polypeptide, e.g., antibody, during a conjugation reaction.

In certain embodiments, the amino acid that attaches to a unit is in the heavy chain of an antibody. In certain embodiments, the amino acid that attaches to a unit is in the light chain of an antibody. In certain embodiments, the amino acid that attaches to a unit is in the hinge region of an antibody. In certain embodiments, the amino acid that attaches to a unit is in the Fc region of an antibody. In certain embodiments, the amino acid that attaches to a unit is in the constant region (e.g., CH1, CH2, or CH3 of a heavy chain, or CH1 of a light chain) of an antibody. In yet other embodiments, the amino acid that attaches to a unit or a drug unit is in the VH framework regions of an antibody. In yet other embodiments, the amino acid that attaches to unit is in the VL framework regions of an antibody.

It is to be understood that the preparation of the conjugates described herein may result in a mixture of conjugates with a distribution of one or more units attached to a polypeptide (i.e., heterogenous), for example, an antibody. Individual conjugate molecules may be identified in the mixture by mass spectroscopy and separated by HPLC, e.g. hydrophobic interaction chromatography, including such methods known in the art. In certain embodiments, a homogeneous conjugate with a single DAR (loading) value may be isolated from the conjugation mixture by electrophoresis or chromatography.

### Antibodies

An antibody (Ab) that binds to a polypeptide of interest binds as “binding” in this context is understood by one skilled in the art. For example, an antibody, or a conjugate as described herein comprising such Ab, may bind to other polypeptides or proteins, generally with lower affinity as determined by, e.g., immunoassays or other assays known in the art. In a specific embodiment, Ab, or a conjugate as described herein comprising such Ab that specifically bind to a polypeptide of interest binds to the polypeptide of interest with an affinity that is at least 2 logs, 2.5 logs, 3 logs, 4 logs or greater than the affinity when Ab or the conjugate bind to another polypeptide. In another specific embodiment, Ab, or a conjugate as described herein comprising such Ab, does not specifically bind a polypeptide other than the polypeptide of interest. In a specific embodiment, Ab, or a conjugate as described herein comprising Ab, specifically binds to a polypeptide of interest with an affinity (Kd) less than or equal to 20 mM. In particular embodiments, such binding is with an affinity (Kd) less than or equal to about 20 mM, about 10 mM, about 1 mM, about 100 μM, about 10 μM, about 1 μM, about 100 nM, about 10 nM, or about 1 nM. Unless otherwise noted, “binds,” “binds to,” “specifically binds” or “specifically binds to” in this context are used interchangeably.

In certain embodiments, the antibody comprises about 10, about 20, about 30, about 40, about 50, about 100, about 150, about 200, about 250, about 300, about 350, about 400, about 450, about 500, about 550, about 600, about 650, about 700, about 750, about 800, about 850, about 900, or about 950 amino acids.

In certain embodiments, the antibody comprises about 10-50, about 50-100, about 100-150, about 150-200, about 200-250, about 250-300, about 300-350, about 350-400, about 400-450, about 450-500, about 500-600, about 600-700, about 700-800, about 800-900, or about 900-1000 amino acids.

In certain embodiments, the conjugate comprises an antibody, Ab. In certain embodiments, the Ab is a monoclonal antibody. In certain embodiments, the Ab is a human antibody. In certain embodiments, the Ab is a humanized antibody. In certain embodiments, the Ab is a chimeric antibody. In certain embodiments, the Ab is a full-length antibody that comprises two heavy chains and two light chains. In particular embodiments, the Ab is an IgG antibody, e.g., is an IgG1, IgG2, IgG3 or IgG4 antibody. In certain embodiments, the Ab is a single chain antibody. In yet other embodiments, the Ab is an antigen-binding fragment of an antibody, e.g., a Fab fragment.

In particular embodiments, the Ab is an IgG1 antibody.

In certain embodiments, the antibody specifically binds to a cell surface protein. In certain embodiments, the antibody specifically binds to a cell surface receptor. In certain embodiments, the antibody specifically binds to a cell surface receptor ligand.

In certain embodiments, the antibody specifically binds to a cancer antigen.

In certain embodiments, the antibody specifically binds to a hepatocyte antigen.

In certain embodiments, the antibody specifically binds to an antigen presented on a macrophage.

In certain embodiments, the antibody specifically binds to an intact complement or a fragment thereof. In certain embodiments, the antibody specifically binds to one or more immunodominant epitope(s) within intact complement or a fragment thereof.

In certain embodiments, the antibody specifically binds to a CD54 protein, e.g., a human CD54 protein. In certain embodiments, the antibody specifically binds to one or more immunodominant epitope(s) within a CD54 protein. In certain embodiments, the antibody is an anti-ICAM1 antibody. In certain embodiments, the antibody is an anti-ICAM1 IgG1. In one embodiment, the anti-ICAM1 antibody is VP0270, comprising a heavy chain of an amino acid sequence of SEQ ID NO:2, and a light chain of an amino acid sequence of SEQ ID NO:4.

In another embodiment, the anti-ICAM1 antibody is VP1157, comprising a heavy chain of an amino acid sequence of SEQ ID NO:14, and a light chain of an amino acid sequence of SEQ ID NO:16. In certain embodiments, the antibody specifically binds to EphA2. In a certain embodiment, the antibody is an anti-EphA2 antibody. In certain embodiments, the antibody is an anti-EphA2 IgG1. In one embodiment, the anti-EphA2 IgG1 is VP0633, comprising a heavy chain of an amino acid sequence of SEQ ID NO:6, and a light chain of an amino acid sequence of SEQ ID NO:8. In another embodiment, the anti-EphA2 IgG1 is VP0253, comprising a heavy chain of an amino acid sequence of SEQ ID NO:10, and a light chain of an amino acid sequence of SEQ ID NO:12.

In another embodiment, the anti-EphA2 IgG1 is VP1127, comprising a heavy chain of an amino acid sequence of SEQ ID NO:18, and a light chain of an amino acid sequence of SEQ ID NO:20.

In another embodiment, the anti-EphA2 IgG1 is VP1342, comprising a heavy chain of an amino acid sequence of SEQ ID NO:22, and a light chain of an amino acid sequence of SEQ ID NO:24.

In certain embodiments, the antibody specifically binds to B7-H3. In a certain embodiment, the antibody is an anti-B7-H3 antibody.

In certain embodiments, the antibody specifically binds to an immune checkpoint inhibitor. In certain embodiments, the antibody binds to one or more immunodominant epitope(s) within an immune checkpoint inhibitor.

In certain embodiments, the antibody specifically binds to a programmed death protein, e.g., a human PD-1. In certain embodiments, the antibody specifically binds to one or more immunodominant epitope(s) within PD-1 protein. In a certain embodiment, the antibody comprises the CDRs present in nivolumab. In another certain embodiment, the antibody comprises the variable light chain and variable heavy chain present in nivolumab. In a particular embodiment, the antibody is nivolumab. In a certain embodiment, the antibody comprises the CDRs present in pembrolizumab. In another certain embodiment, the antibody comprises the variable light chain and variable heavy chain present in pembrolizumab. In a particular embodiment, the antibody is pembrolizumab.

In certain embodiments, the antibody specifically binds to a programmed death ligand-1 (PD-L1) protein, e.g., a human PD-L1. In certain embodiments, the antibody specifically binds to one or more immunodominant epitope(s) within PD-L1 protein. In a certain embodiment, the antibody comprises the CDRs present in atezolizumab. In another certain embodiment, the antibody comprises the variable light chain and variable heavy chain present in atezolizumab. In a particular embodiment, the antibody is atezolizumab.

In certain embodiments, the antibody specifically binds to an epidermal growth factor (EGF) protein, e.g., a human EGF. In certain embodiments, the antibody specifically binds to one or more immunodominant epitope(s) within an EGF protein.

In certain embodiments, the antibody specifically binds to an epidermal growth factor receptor (EGFR) protein, e.g., a human EGFR. In certain embodiments, the antibody specifically binds to one or more immunodominant epitope(s) within an EGFR protein.

In certain embodiments, the antibody specifically binds to vascular endothelial growth factor (VEGF) protein, e.g., human VEGF protein. In certain embodiments, the antibody specifically binds to one or more immunodominant epitope(s) within a VEGF protein.

In certain embodiments, the antibody specifically binds to a vascular endothelial growth factor receptor (VEGFR) protein, e.g., human VEGFR protein.

In certain embodiments, the antibody specifically binds to a fibroblast growth factor (FGF), e.g., a human FGF. In certain embodiments, the antibody specifically binds to one or more immunodominant epitope(s) within an FGF protein.

In certain embodiments, the antibody specifically binds to a fibroblast growth factor receptor (FGFR), e.g., a human FGFR.

Disclosed herein, in some embodiments, are conjugates comprising an antibody, a linking moiety, and a payload, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise HC-CDR1: SEQ ID NO: 25, HC-CDR2: SEQ ID NO: 26, HC-CDR3: SEQ ID NO: 27, LC-CDR1: SEQ ID NO: 43, LC-CDR2: SEQ ID NO: 44, LC-CDR3: SEQ ID NO: 45.

Disclosed herein, in some embodiments, are conjugates comprising an antibody, a linking moiety, and a payload, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise HC-CDR1: SEQ ID NO: 28, HC-CDR2: SEQ ID NO: 29, HC-CDR3: SEQ ID NO: 30, LC-CDR1: SEQ ID NO: 46, LC-CDR2: SEQ ID NO: 47, LC-CDR3: SEQ ID NO: 48.

Disclosed herein, in some embodiments, are conjugates comprising an antibody, a linking moiety, and a payload, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise HC-CDR1: SEQ ID NO: 31, HC-CDR2: SEQ ID NO: 32, HC-CDR3: SEQ ID NO: 33, LC-CDR1: SEQ ID NO: 49, LC-CDR2: SEQ ID NO: 50, LC-CDR3: SEQ ID NO: 51.

Disclosed herein, in some embodiments, are conjugates comprising an antibody, a linking moiety, and a payload, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise HC-CDR1: SEQ ID NO: 34, HC-CDR2: SEQ ID NO: 35, HC-CDR3: SEQ ID NO: 36, LC-CDR1: SEQ ID NO: 52, LC-CDR2: SEQ ID NO: 53, LC-CDR3: SEQ ID NO: 54.

Disclosed herein, in some embodiments, are conjugates comprising an antibody, a linking moiety, and a payload, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise HC-CDR1: SEQ ID NO: 37, HC-CDR2: SEQ ID NO: 38, HC-CDR3: SEQ ID NO: 39, LC-CDR1: SEQ ID NO: 55, LC-CDR2: SEQ ID NO: 56, LC-CDR3: SEQ ID NO: 57.

Disclosed herein, in some embodiments, are conjugates comprising an antibody, a linking moiety, and a payload, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise HC-CDR1: SEQ ID NO: 40, HC-CDR2: SEQ ID NO: 41, HC-CDR3: SEQ ID NO: 42, LC-CDR1: SEQ ID NO: 58, LC-CDR2: SEQ ID NO: 59, LC-CDR3: SEQ ID NO: 60.

The heavy chain and light chain sequences of an exemplary anti-ICAM1 antibody (VP0270 anti-ICAM1 IgG1) are as follows:

VP0270 anti-ICAM1 IgG1
H-Chain: DNA sequence - P00334 - 1350 bases
(SEQ ID NO: 1)
GAGGTGCAGGTGCTGGAGAGCGGAGGAGGACTGGTGCAGCCCGGAGGGTCACTGA
GACTGTCATGTGCCGTGTCAGGATTTTCACTGAGCTCCCACGCTATGGGCTGGGTGC
GGCAGGCACCAGGCAAGGGCCTGGAGTGGATCGGCATCATCGGCTCTAGCGACCGG
ACCTACTATGCCAGCTGGGCCAAGGGCAGATTCACCATCTCCAAGGATAACTCTAA
GAATACAGTGTACCTGCAGATCAACTCCCCCAGGGCCGAGGACACAGCCGTGTACT
ATTGCGTGCGCGATCCTTATGATTCTTATGATGAGGGCTACCGACTGTGGGGGCAGG
GGACACTGGTGACCGTCTCCTCAGCTAGCACCAAAGGTCCCAGCGTGTTCCCACTCG
CCCCGAGTTCAAAATCAACTTCTGGAGGCACCGCCGCCCTGGGTTGCCTGGTAAAG
GACTACTTCCCAGAGCCCGTGACCGTGAGCTGGAACTCCGGGGCACTGACATCTGG
CGTTCATACTTTCCCGGCCGTGCTCCAGTCTTCAGGTCTGTATAGTCTCTCCTCTGTG
GTCACTGTCCCATCTAGCTCTCTGGGCACCCAAACCTACATATGCAACGTTAATCAC
AAGCCGAGCAATACTAAAGTTGACAAAAAGGTGGAACCCAAGTCTTGTGACAAGA
CCCACACGTGTCCCCCCTGCCCGGCTCCTGAGCTGCTTGGCGGCCCCAGCGTCTTTC
TCTTTCCCCCAAAGCCAAAAGATACCTTGATGATCAGCAGAACTCCCGAGGTGACA
TGCGTCGTCGTGGACGTAAGCCATGAAGATCCCGAGGTTAAGTTCAACTGGTATGT
CGATGGCGTGGAAGTCCATAATGCTAAGACTAAACCTCGCGAAGAGCAGTACAATT
CAACTTACCGGGTCGTTTCCGTTCTGACCGTGCTGCATCAGGACTGGCTGAATGGTA
AAGAGTACAAGTGCAAAGTGTCTAACAAGGCACTCCCCGCCCCAATTGAGAAGACT
ATCTCCAAAGCTAAAGGGCAACCAAGAGAGCCCCAGGTCTACACCCTGCCCCCCTC
AAGGGATGAGCTTACTAAGAACCAGGTTAGTCTCACCTGCTTGGTTAAAGGATTTTA
TCCAAGCGATATTGCTGTGGAGTGGGAGTCCAACGGCCAGCCTGAGAACAATTATA
AAACCACCCCCCCTGTTCTTGACAGTGACGGTAGTTTCTTCCTGTATTCCAAACTGA
CCGTCGATAAGAGCAGATGGCAACAGGGAAATGTGTTCAGCTGCTCCGTGATGCAT
GAGGCGCTCCATAATCATTACACACAAAAAAGTTTGTCCCTGAGCCCAGGCAAG
H-Chain: AA sequence - CDS00429 - 450 AAs
(SEQ ID NO: 2)
EVQVLESGGGLVQPGGSLRLSCAVSGFSLSSHAMGWVRQAPGKGLEWIGIIGSSDRTYY
ASWAKGRFTISKDNSKNTVYLQINSPRAEDTAVYYCVRDPYDSYDEGYRLWGQGTLV
TVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
VP0270: L-Chain - DNA sequence - P00283 - 651 bases
(SEQ ID NO: 3)
GATATTCAGATGACCCAGAGCCCCAGCACACTGAGCGCAAGCGTCGGAGATAGAGT
GACCATTACCTGCCAGGCCAGCCAGAGCATTTACAGATACCTGTCTTGGTATCAGCA
GAAGCCCGGCAAGGCCCCTAAGTTCCTGATCTATGACGCCTCTAAGCTGGCCAGCG
GCGTGCCATCCAGATTCTCTGGCAGCGGCTCCGGCACCGAGTTTACCCTGACAATCA
GCTCCGTGCAGCCAGACGATTTCGCAACATACTATTGCCAGCAGGCTTACAGCAGC
GGCAGCATTGACAACGCATTCGGAGGAGGGACTAAAGTGGAGATCAAGCGTACGG
TGGCAGCTCCATCAGTTTTTATCTTCCCACCAAGCGACGAGCAATTGAAGTCCGGCA
CTGCCTCTGTGGTGTGCCTTCTGAACAACTTCTATCCAAGGGAGGCCAAGGTCCAGT
GGAAGGTCGATAATGCGCTGCAGAGCGGGAACAGCCAAGAGTCAGTGACCGAGCA
GGACTCAAAAGATAGCACATACTCTCTGAGTTCCACCCTGACCCTGTCAAAGGCTG
ACTACGAAAAGCATAAGGTATACGCATGCGAAGTGACCCATCAGGGTCTCTCATCT
CCCGTAACCAAATCTTTTAATAGAGGAGAATGC
VP0270: L-Chain - AA sequence - CDS00382 - 217 AAs
(SEQ ID NO: 4)
DIQMTQSPSTLSASVGDRVTITCQASQSIYRYLSWYQQKPGKAPKFLIYDASKLASGVPS
RFSGSGSGTEFTLTISSVQPDDFATYYCQQAYSSGSIDNAFGGGTKVEIKRTVAAPSVFIF
PPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

The heavy chain and light chain sequences of an exemplary anti-ICAM1 antibody (VP1157: anti-ICAM1 IgG1) are as follows:

VP1157: anti-ICAM1 IgG1
VP1157: H-Chain - DNA sequence
(SEQ ID NO: 13)
GAGGTGCAGCTGCTGGAGAGCGGGGGGGGACTGGTGCAGCCAGGAGGAAGCCTGA
GACTGAGCTGTGCCGCCAGCGGGTTTAGCCTGAGCAGCCACGCCATGGGATGGGTG
AGGCAGGCCCCTGGCAAAGGCCTGGAGTGGGTGAGCATTATTGGCAGCAGTGACAG
AACCTACTACGCCGACAGCGTGAAAGGGCGGTTCACCATCAGCAGAGATAACAGCA
AGAACACACTGTACCTGCAGATGAACAGCCTGAGAGCCGAGGACACCGCTGTGTAC
TACTGCGCCAAGGACCCATACGATAGTTACGACGAAGGATACAGACTGTGGGGGCA
GGGAACCCTGGTGACCGTGAGCAGCGCTAGCACCAAAGGTCCCAGCGTGTTCCCAC
TCGCCCCGAGTTCAAAATCAACTTCTGGAGGCACCGCCGCCCTGGGTTGCCTGGTAA
AGGACTACTTCCCAGAGCCCGTGACCGTGAGCTGGAACTCCGGGGCACTGACATCT
GGCGTTCATACTTTCCCGGCCGTGCTCCAGTCTTCAGGTCTGTATAGTCTCTCCTCTG
TGGTCACTGTCCCATCTAGCTCTCTGGGCACCCAAACCTACATATGCAACGTTAATC
ACAAGCCGAGCAATACTAAAGTTGACAAAAAGGTGGAACCCAAGTCTTGTGACAA
GACCCACACGTGTCCCCCCTGCCCGGCTCCTGAGCTGCTTGGCGGCCCCAGCGTCTT
TCTCTTTCCCCCAAAGCCAAAAGATACCTTGATGATCAGCAGAACTCCCGAGGTGA
CATGCGTCGTCGTGGACGTAAGCCATGAAGATCCCGAGGTTAAGTTCAACTGGTAT
GTCGATGGCGTGGAAGTCCATAATGCTAAGACTAAACCTCGCGAAGAGCAGTACAA
TTCAACTTACCGGGTCGTTTCCGTTCTGACCGTGCTGCATCAGGACTGGCTGAATGG
TAAAGAGTACAAGTGCAAAGTGTCTAACAAGGCACTCCCCGCCCCAATTGAGAAGA
CTATCTCCAAAGCTAAAGGGCAACCAAGAGAGCCCCAGGTCTACACCCTGCCCCCC
TCAAGGGATGAGCTTACTAAGAACCAGGTTAGTCTCACCTGCTTGGTTAAAGGATTT
TATCCAAGCGATATTGCTGTGGAGTGGGAGTCCAACGGCCAGCCTGAGAACAATTA
TAAAACCACCCCCCCTGTTCTTGACAGTGACGGTAGTTTCTTCCTGTATTCCAAACT
GACCGTCGATAAGAGCAGATGGCAACAGGGAAATGTGTTCAGCTGCTCCGTGATGC
ATGAGGCGCTCCATAATCATTACACACAAAAAAGTTTGTCCCTGAGCCCAGGCAAG
VP1157: H-Chain - AA sequence
(SEQ ID NO: 14)
EVQLLESGGGLVQPGGSLRLSCAASGFSLSSHAMGWVRQAPGKGLEWVSIIGSSDRTY
YADSVKGRFTISRDNSKNTLYLQMNSLRAEDTAVYYCAKDPYDSYDEGYRLWGQGTL
VTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK
VP1157: L-Chain DNA sequence
(SEQ ID NO: 15)
GACATTCAGATGACCCAGAGCCCCAGCACCCTGAGCGCCAGCGTGGGAGACAGAGT
GACCATCACATGCCAGGCTAGCCAGAGCATCTACAGATATTTGAGCTGGTACCAGC
AGAAACCCGGCAAAGCCCCCAAACTGCTGATCTACGACGCCAGTAAGCTGGCCAGC
GGAGTGCCCAGCAGATTCAGCGGCAGCGGCAGCGGAACCGAGTTCACCCTGACCAT
CAGTAGCCTGCAGCCCGACGACTTCGCCACCTACTACTGCCAGCAGGCCTACTCATC
CGGAAGCATTGACAACGCCTTCGGCGGCGGGACAAAGGTGGAGATTAAGCGTACG
GTGGCAGCTCCATCAGTTTTTATCTTCCCACCAAGCGACGAGCAATTGAAGTCCGGC
ACTGCCTCTGTGGTGTGCCTTCTGAACAACTTCTATCCAAGGGAGGCCAAGGTCCAG
TGGAAGGTCGATAATGCGCTGCAGAGCGGGAACAGCCAAGAGTCAGTGACCGAGC
AGGACTCAAAAGATAGCACATACTCTCTGAGTTCCACCCTGACCCTGTCAAAGGCT
GACTACGAAAAGCATAAGGTATACGCATGCGAAGTGACCCATCAGGGTCTCTCATC
TCCCGTAACCAAATCTTTTAATAGAGGAGAATGC
VP1157: L-Chain - AA sequence
(SEQ ID NO: 16)
DIQMTQSPSTLSASVGDRVTITCQASQSIYRYLSWYQQKPGKAPKLLIYDASKLASGVPS
RFSGSGSGTEFTLTISSLQPDDFATYYCQQAYSSGSIDNAFGGGTKVEIKRTVAAPSVFIF
PPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

The heavy chain and light chain sequences of an exemplary anti-EphA2 antibody (VP0633: anti-EphA2 IgG1) are as follows:

VP0633: anti-EphA2 IgG1
VP0633: H-Chain - DNA sequence - P00685 - 1368 bases
(SEQ ID NO: 5)
GAGGTGCAGCTGGTGGAGAGCGGCGGCGGCCTGGTGCAGCCCGGCGGCAGCCTGA
GACTGAGCTGCGCCGCCAGCGGCTTCACCTTCAGCAGCTACAGCATGAACTGGGTG
AGACAGGCCCCCGGCAAGGGCCTGGAGTGGGTGAGCTACATCAGCAGCAGCAGCA
GCACCATCTACTACGCCGACAGCGTGAAGGGCAGATTCACCATCAGCAGAGACAAC
GCCAAGAACAGCCTGTACCTGCAGATGAACAGCCTGAGAGCCGAGGACACCGCCGT
GTACTACTGCGCCAGATACAGACTGCCCAACTTCTGGAGCGGCTACCCCAACTACG
GCACCGACGTGTGGGGCCAGGGCACCACCGTGACCGTGAGCAGCGCTAGCACCAA
GGGCCCATCGGTCTTCCCCCTGGCACCCTCCTCCAAGAGCACCTCTGGGGGCACAGC
GGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAAAGT
TGAGCCCAAATCTTGTGACAAAACTCACACATGCCCACCGTGCCCAGCACCTGAAC
TCCTGGGGGGACCGTCAGTCTTCCTCTTCCCCCCAAAACCCAAGGACACCCTCATGA
TCTCCCGGACCCCCGAGGTCACATGCGTGGTGGTGGACGTGAGCCACGAAGACCCT
GAGGTCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACCGTCC
TGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGG
CTCCTTCTTCCTCTACAGCAAGCTCACCGTGGACAAGAGCAGGTGGCAGCAGGGGA
ACGTCTTCTCATGCTCCGTGATGCATGAGGCTCTGCACAACCACTACACGCAGAAG
AGCCTCTCCCTGTCTCCGGGTAAA
VP0633: H-Chain - AA sequence - CDS00827 - 456 AAs
(SEQ ID NO: 6)
EVQLVESGGGLVQPGGSLRLSCAASGFTFSSYSMNWVRQAPGKGLEWVSYISSSSSTIY
YADSVKGRFTISRDNAKNSLYLQMNSLRAEDTAVYYCARYRLPNFWSGYPNYGTDVW
GQGTTVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
VP0633: L-Chain - DNA sequence - P00349 - 654 bases
(SEQ ID NO: 7)
CAGTCTGTGCTGACACAGCCACCCAGCGTGAGCGGAGCCCCTGGACAGAGAGTGAC
CATCAGCTGCACCGGAAGCAGCAGCAATATCGGAGCCGGATATGACGTGCATTGGT
ACCAGCAGCTGCCCGGCACCGCCCCAAAACTGCTGATCTACGGAAACAGCAACAGA
CCCAGCGGCGTGCCCGACAGATTCAGCGGCAGCAAGAGCGGCACAAGCGCCAGTCT
GGCTATCACCGGCCTGCAGGCCGAGGACGAAGCCGACTACTACTGCCAGAGCTACG
ACAGCAGCCTGAGCGGCCACGTGGTGTTTGGAGGAGGAACCAAGCTGACCGTGCTG
GGTCAGCCCAAGGCCAACCCCACTGTCACTCTGTTCCCGCCCTCCTCTGAGGAGCTC
CAAGCCAACAAGGCCACACTAGTGTGTCTGATCAGTGACTTCTACCCGGGAGCTGT
GACAGTGGCCTGGAAGGCAGATGGCAGCCCCGTCAAGGCGGGAGTGGAGACCACC
AAACCCTCCAAACAGAGCAACAACAAGTACGCGGCCAGCAGCTACCTGAGCCTGAC
GCCCGAGCAGTGGAAGTCCCACAGAAGCTACAGCTGCCAGGTCACGCATGAAGGG
AGCACCGTGGAGAAGACAGTGGCCCCTACAGAATGTTCA
VP0633: L-Chain - AA sequence - CDS00444 - 218 AAs
(SEQ ID NO: 8)
QSVLTQPPSVSGAPGQRVTISCTGSSSNIGAGYDVHWYQQLPGTAPKLLIYGNSNRPSG
VPDRFSGSKSGTSASLAITGLQAEDEADYYCQSYDSSLSGHVVFGGGTKLTVLGQPKAN
PTVTLFPPSSEELQANKATLVCLISDFYPGAVTVAWKADGSPVKAGVETTKPSKQSNNK
YAASSYLSLTPEQWKSHRSYSCQVTHEGSTVEKTVAPTECS

The heavy chain and light chain sequences of an exemplary anti-EphA2 antibody (VP0253: anti-EphA2 IgG1) are as follows:

VP0253: anti-EphA2 IgG1
VP0253: H-Chain - DNA sequence - P00311
(SEQ ID NO: 9)
CAGGTGCAGCTGCAGGAGAGCGGCGGCGGACTGGTGCAGCCAGGAGGAAGCCTGA
GACTGTCTTGCGCTGCTTCCGGCTTCACATTCAGCAGCTACAGCATGAACTGGGTGA
GACAGGCCCCCGGAAAAGGACTGGAGTGGGTGAGCTACATCAGCAGCAGTAGTAG
CACCATCTATTACGCTGATTCCGTGAAAGGCAGATTCACAATCTCCCGGGACAACG
CCAAGAACAGCCTGTACCTGCAGATGAACAGCCTGAGAGCCGAGGACACCGCCGTG
TACTACTGCGCCAGGTACCGGCTGCCCGATTTCTGGTCCGGCTACCCCAACTACGGC
ATGGATGTGTGGGGCCAGGGCACCACCGTGACAGTGAGCAGCGCTAGCACCAAGG
GCCCATCGGTCTTCCCCCTGGCACCCTCCTCCAAGAGCACCTCTGGGGGCACAGCGG
CCCTGGGCTGCCTGGTCAAGGACTACTTCCCCGAACCGGTGACGGTGTCGTGGAAC
TCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACC
TACATCTGCAACGTGAATCACAAGCCCAGCAACACCAAGGTGGACAAGAAAGTTGA
GCCCAAATCTTGTGACAAAACTCACACATGCCCACCGTGCCCAGCACCTGAACTCCT
GGGGGGACCGTCAGTCTTCCTCTTCCCCCCAAAACCCAAGGACACCCTCATGATCTC
CCGGACCCCCGAGGTCACATGCGTGGTGGTGGACGTGAGCCACGAAGACCCTGAGG
TCAAGTTCAACTGGTACGTGGACGGCGTGGAGGTGCATAATGCCAAGACAAAGCCG
CGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACCACA
GGTGTACACCCTGCCCCCATCCCGGGAGGAGATGACCAAGAACCAGGTCAGCCTGA
CCTGCCTGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGACGGCTC
CTTCTTCCTCTACAGCAAGCTCACCGTGGACAAGAGCAGGTGGCAGCAGGGGAACG
TCTTCTCATGCTCCGTGATGCATGAGGCTCTGCACAACCACTACACGCAGAAGAGCC
TCTCCCTGTCTCCGGGTAAA
VP0253: H-Chain - AA sequence - CDS00412
(SEQ ID NO: 10)
QVQLQESGGGLVQPGGSLRLSCAASGFTFSSYSMNWVRQAPGKGLEWVSYISSSSSTIY
YADSVKGRFTISRDNAKNSLYLQMNSLRAEDTAVYYCARYRLPDFWSGYPNYGMDV
WGQGTTVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
VP0253: L-Chain DNA sequence - P00349
(SEQ ID NO: 11)
CAGTCTGTGCTGACACAGCCACCCAGCGTGAGCGGAGCCCCTGGACAGAGAGTGAC
CATCAGCTGCACCGGAAGCAGCAGCAATATCGGAGCCGGATATGACGTGCATTGGT
ACCAGCAGCTGCCCGGCACCGCCCCAAAACTGCTGATCTACGGAAACAGCAACAGA
CCCAGCGGCGTGCCCGACAGATTCAGCGGCAGCAAGAGCGGCACAAGCGCCAGTCT
GGCTATCACCGGCCTGCAGGCCGAGGACGAAGCCGACTACTACTGCCAGAGCTACG
ACAGCAGCCTGAGCGGCCACGTGGTGTTTGGAGGAGGAACCAAGCTGACCGTGCTG
GGTCAGCCCAAGGCCAACCCCACTGTCACTCTGTTCCCGCCCTCCTCTGAGGAGCTC
CAAGCCAACAAGGCCACACTAGTGTGTCTGATCAGTGACTTCTACCCGGGAGCTGT
GACAGTGGCCTGGAAGGCAGATGGCAGCCCCGTCAAGGCGGGAGTGGAGACCACC
AAACCCTCCAAACAGAGCAACAACAAGTACGCGGCCAGCAGCTACCTGAGCCTGAC
GCCCGAGCAGTGGAAGTCCCACAGAAGCTACAGCTGCCAGGTCACGCATGAAGGG
AGCACCGTGGAGAAGACAGTGGCCCCTACAGAATGTTCA
VP0253: L-Chain - AA sequence - CDS00444
(SEQ ID NO: 12)
QSVLTQPPSVSGAPGQRVTISCTGSSSNIGAGYDVHWYQQLPGTAPKLLIYGNSNRPSG
VPDRFSGSKSGTSASLAITGLQAEDEADYYCQSYDSSLSGHVVFGGGTKLTVLGQPKAN
PTVTLFPPSSEELQANKATLVCLISDFYPGAVTVAWKADGSPVKAGVETTKPSKQSNNK
YAASSYLSLTPEQWKSHRSYSCQVTHEGSTVEKTVAPTECS

The heavy chain and light chain sequences of an exemplary anti-EphA2 antibody (VP1127: anti-EphA2 IgG1) are as follows:

VP1127: anti-EphA2 IgG1
VP1127: H-Chain - DNA sequence
(SEQ ID NO: 17)
GAGGTGCAGCTGCAGGAGAGCGGGGGGGGACTGGTGCAGCCAGGAGGAAGCCTGA
GACTGAGCTGTGCCGCCAGCGGATTTACATTTAGTAGTTATAGTATGAACTGGGTGA
GGCAGGCCCCCGGAAAAGGCCTGGAGTGGGTGAGCTACATTAGCAGCAGTAGCAG
CACCATCTATTACGCTGACTCCGTGAAGGGCAGATTCACCATCTCCAGAGACAACG
CCAAAAACAGCCTGTACCTGCAGATGAACAGCCTGAGAGCCGAGGACACAGCCGT
GTACTACTGCGCCAGATACAGACTGCCCGATTTCTGGAGCGGCTACCCCAACTACG
GAATGGATGTGTGGGGCCAGGGCACCACCGTGACAGTGAGCAGTGCTAGCACCAA
GGGCCCATCGGTCTTCCCCCTGGCACCCTCCTCCAAGAGCACCTCTGGGGGCACAGC
GGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAAAGT
TGAGCCCAAATCTTGTGACAAAACTCACACATGCCCACCGTGCCCAGCACCTGAAC
TCCTGGGGGGACCGTCAGTCTTCCTCTTCCCCCCAAAACCCAAGGACACCCTCATGA
TCTCCCGGACCCCCGAGGTCACATGCGTGGTGGTGGACGTGAGCCACGAAGACCCT
GAGGTCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACCGTCC
TGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGG
CTCCTTCTTCCTCTACAGCAAGCTCACCGTGGACAAGAGCAGGTGGCAGCAGGGGA
ACGTCTTCTCATGCTCCGTGATGCATGAGGCTCTGCACAACCACTACACGCAGAAG
AGCCTCTCCCTGTCTCCGGGTAAA
VP1127: H-Chain - AA sequence
(SEQ ID NO: 18)
EVQLQESGGGLVQPGGSLRLSCAASGFTFSSYSMNWVRQAPGKGLEWVSYISSSSSTIY
YADSVKGRFTISRDNAKNSLYLQMNSLRAEDTAVYYCARYRLPDFWSGYPNYGMDV
WGQGTTVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
VP1127: L-Chain DNA sequence
(SEQ ID NO: 19)
CAGAGCGTGCTGACCCAGCCCCCCAGCGTGAGCGGAGCTCCAGGACAGAGAGTGA
CAATCAGCTGCACCGGCAGCAGCAGCAACATCGGCGCCGGCTACGACGTGCACTGG
TACCAGCAGCTGCCTGGCACAGCCCCCAAACTGCTGATCTACGGCCAGAGCAACAG
ACCCAGCGGCGTGCCCGACAGGTTCAGCGGAAGCAAGTCAGGAACCAGCGCCTCCC
TGGCCATCACCGGACTGCAGGCCGAAGATGAAGCTGACTACTACTGCCAGAGCTAC
GACAGCAGCCTGAGTGGCCACGTGGTGTTTGGCGGAGGAACCAAGCTGACCGTGCT
GGGTCAGCCCAAGGCCAACCCCACTGTCACTCTGTTCCCGCCCTCCTCTGAGGAGCT
CCAAGCCAACAAGGCCACACTAGTGTGTCTGATCAGTGACTTCTACCCGGGAGCTG
TGACAGTGGCCTGGAAGGCAGATGGCAGCCCCGTCAAGGCGGGAGTGGAGACCAC
CAAACCCTCCAAACAGAGCAACAACAAGTACGCGGCCAGCAGCTACCTGAGCCTGA
CGCCCGAGCAGTGGAAGTCCCACAGAAGCTACAGCTGCCAGGTCACGCATGAAGG
GAGCACCGTGGAGAAGACAGTGGCCCCTACAGAATGTTCA
VP1127: L-Chain - AA sequence
(SEQ ID NO: 20)
QSVLTQPPSVSGAPGQRVTISCTGSSSNIGAGYDVHWYQQLPGTAPKLLIYGQSNRPSG
VPDRFSGSKSGTSASLAITGLQAEDEADYYCQSYDSSLSGHVVFGGGTKLTVLGQPKAN
PTVTLFPPSSEELQANKATLVCLISDFYPGAVTVAWKADGSPVKAGVETTKPSKQSNNK
YAASSYLSLTPEQWKSHRSYSCQVTHEGSTVEKTVAPTECS

The heavy chain and light chain sequences of an exemplary anti-EphA2 antibody (VP1342: anti-EphA2 IgG1) are as follows:

VP1342: anti-EphA2 IgG1
VP1342: H-Chain - DNA sequence
(SEQ ID NO: 21)
GAGGTGCAGCTGCAGGAGAGCGGGGGGGGACTGGTGCAGCCAGGAGGAAGCCTGA
GACTGAGCTGTGCCGCCAGCGGATTTACATTTAGTAGTTATAGTATGAACTGGGTGA
GGCAGGCCCCCGGAAAAGGCCTGGAGTGGGTGAGCTACATTAGCAGCAGTAGCAG
CACCATCTATTACGCTGACTCCGTGAAGGGCAGATTCACCATCTCCAGAGACAACG
CCAAAAACAGCCTGTACCTGCAGATGAACAGCCTGAGAGCCGAGGACACAGCCGT
GTACTACTGCGCCAGATACAGACTGCCCGATTTCTGGAGCGGCTACCCCAACTACG
GAATGGATGTGTGGGGCCAGGGCACCACCGTGACAGTGAGCAGTGCTAGCACCAA
GGGCCCATCGGTCTTCCCCCTGGCACCCTCCTCCAAGAGCACCTCTGGGGGCACAGC
GGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAG
GACTCTACTCCCTCAGCAGCGTGGTGACCGTGCCCTCCAGCAGCTTGGGCACCCAG
ACCTACATCTGCAACGTGAATCACAAGCCCAGCAACACCAAGGTGGACAAGAAAGT
TGAGCCCAAATCTTGTGACAAAACTCACACATGCCCACCGTGCCCAGCACCTGAAC
TCCTGGGGGGACCGTCAGTCTTCCTCTTCCCCCCAAAACCCAAGGACACCCTCATGA
TCTCCCGGACCCCCGAGGTCACATGCGTGGTGGTGGACGTGAGCCACGAAGACCCT
GAGGTCAAGTTCAACTGGTACGTGGACGGCGTGGAGGTGCATAATGCCAAGACAAA
GCCGCGGGAGGAGCAGTACAACAGCACGTACCGTGTGGTCAGCGTCCTCACCGTCC
TGCACCAGGACTGGCTGAATGGCAAGGAGTACAAGTGCAAGGTCTCCAACAAAGCC
CTCCCAGCCCCCATCGAGAAAACCATCTCCAAAGCCAAAGGGCAGCCCCGAGAACC
ACAGGTGTACACCCTGCCCCCATCCCGGGAGGAGATGACCAAGAACCAGGTCAGCC
TGACCTGCCTGGTCAAAGGCTTCTATCCCAGCGACATCGCCGTGGAGTGGGAGAGC
AATGGGCAGCCGGAGAACAACTACAAGACCACGCCTCCCGTGCTGGACTCCGACGG
CTCCTTCTTCCTCTACAGCAAGCTCACCGTGGACAAGAGCAGGTGGCAGCAGGGGA
ACGTCTTCTCATGCTCCGTGATGCATGAGGCTCTGCACAACCACTACACGCAGAAG
AGCCTCTCCCTGTCTCCGGGTAAA
VP1342: H-Chain - AA sequence
(SEQ ID NO: 22)
EVQLQESGGGLVQPGGSLRLSCAASGFTFSSYSMNWVRQAPGKGLEWVSYISSSSSTIY
YADSVKGRFTISRDNAKNSLYLQMNSLRAEDTAVYYCARYRLPDFWSGYPNYGMDV
WGQGTTVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
VP1342: L-Chain DNA sequence
(SEQ ID NO: 23)
CAGTCCGTCCTCACCCAGCCCCCATCCGTCTCCGGCGCACCAGGACAGCGGGTCAC
CATCTCCTGCACCGGCTCCTCCTCCAACATCGGCGCCGGCTACGACGTCCACTGGTA
CCAGCAGCTCCCAGGCACCGCCCCCAAACTCCTGATCTACGGCCAGAGCAACAGAC
CCTCCGGCGTCCCCGACAGATTCTCCGGCTCCAAGTCTGGAACATCCGCCTCCCTCG
CTATCACCGGCCTCCAGGCAGAAGATGAAGCTGACTACTACTGCCAGTCCTACTCCT
CCTCCCTCAGCGGTCACGTGGTGTTCGGCGGGGGCACCAAGCTGACAGTGCTGGGT
CAGCCCAAGGCCAACCCCACTGTCACTCTGTTCCCGCCCTCCTCTGAGGAGCTCCAA
GCCAACAAGGCCACACTAGTGTGTCTGATCAGTGACTTCTACCCGGGAGCTGTGAC
AGTGGCCTGGAAGGCAGATGGCAGCCCCGTCAAGGCGGGAGTGGAGACCACCAAA
CCCTCCAAACAGAGCAACAACAAGTACGCGGCCAGCAGCTACCTGAGCCTGACGCC
CGAGCAGTGGAAGTCCCACAGAAGCTACAGCTGCCAGGTCACGCATGAAGGGAGC
ACCGTGGAGAAGACAGTGGCCCCTACAGAATGTTCA
VP1342: L-Chain - AA sequence
(SEQ ID NO: 24)
QSVLTQPPSVSGAPGQRVTISCTGSSSNIGAGYDVHWYQQLPGTAPKLLIYGQSNRPSG
VPDRFSGSKSGTSASLAITGLQAEDEADYYCQSYSSSLSGHVVFGGGTKLTVLGQPKAN
PTVTLFPPSSEELQANKATLVCLISDFYPGAVTVAWKADGSPVKAGVETTKPSKQSNNK
YAASSYLSLTPEQWKSHRSYSCQVTHEGSTVEKTVAPTECS

In some embodiments, the conjugates described herein has a structure provided in Table 5.

TABLE 5

| Conjugate | Structure |
|---|---|
| IC-1 | 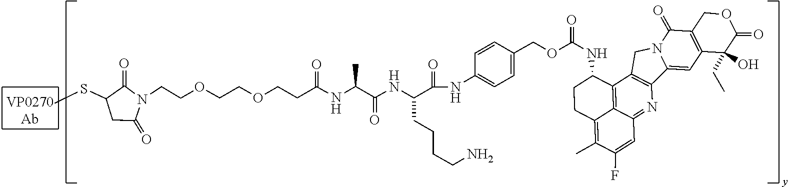 |
| IC-3 | 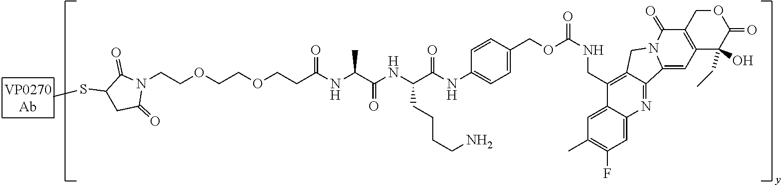 |
| IC-4 | 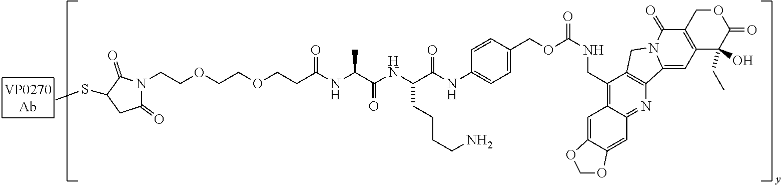 |
| IC-7 | 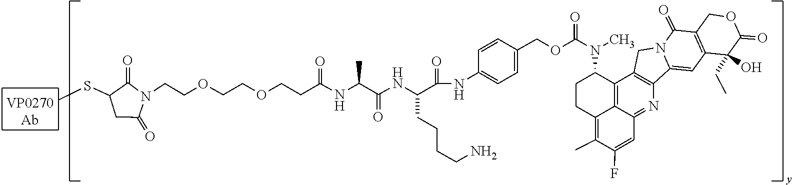 |
| IC-9 | 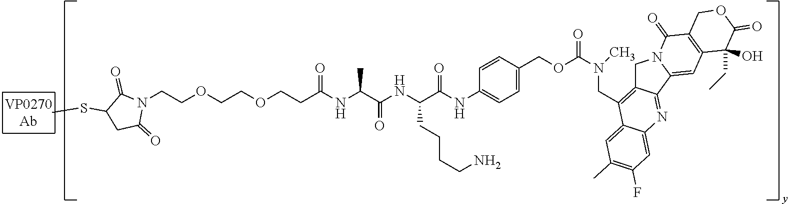 |
| IC-12 | 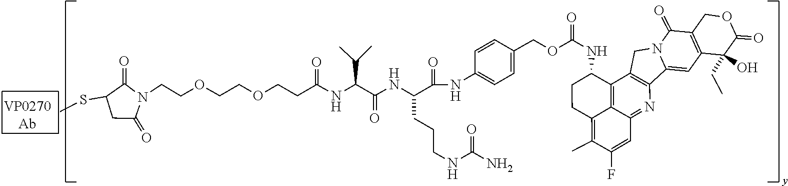 |
| IC-40 | 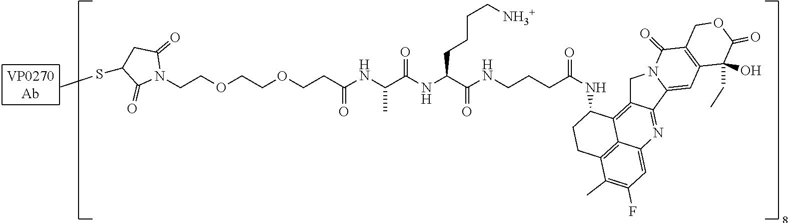 |
| | IC-40 (DAR8) |
| IC-105 | 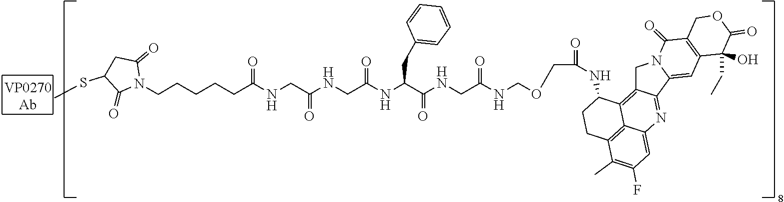 |
| | IC-105 (DAR8) |
| IC-106 | 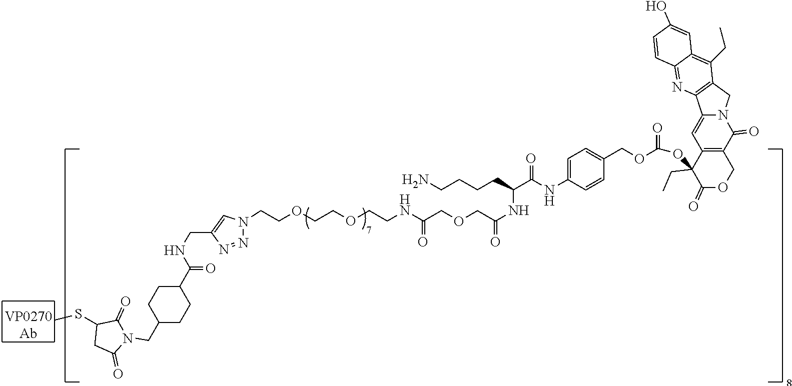 |
| | IC-106 (DAR8) |
| IC-A1 | 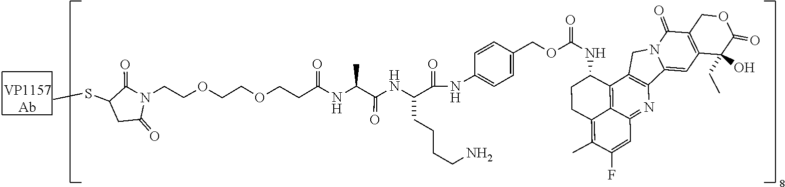 |
| | IC-A1 (DAR8) |
| IC-A40 | 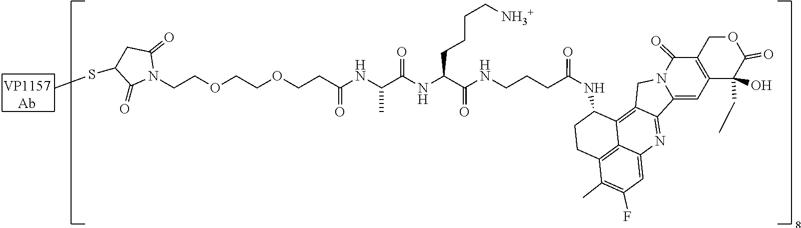 |
| | IC-A-40 (DAR8) |
| IC-A105 | 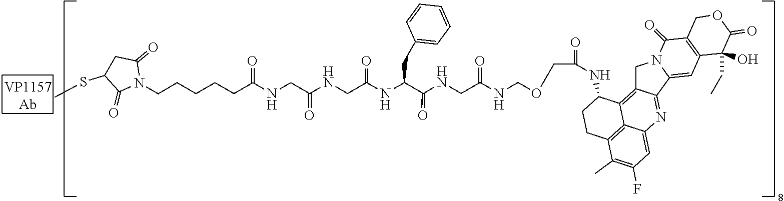 |
| | IC-A-105 (DAR8) |

In some embodiments, the conjugates described herein has a structure provided in Table 6.

TABLE 6

| Conjugate | Structure |
|---|---|
| AC-1 | 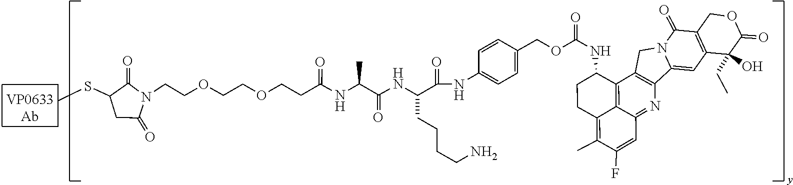 |
| AC-3 | 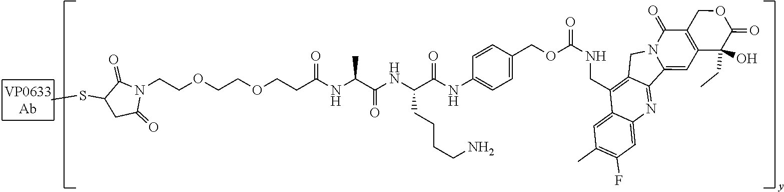 |
| AC-4 | 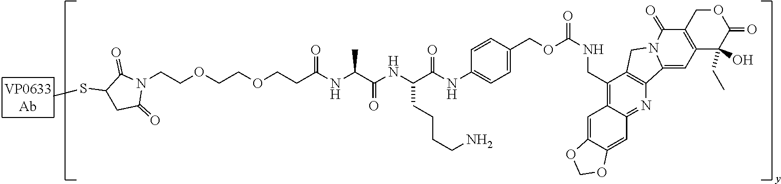 |
| AC-7 | 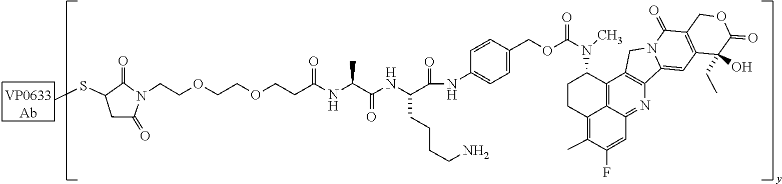 |
| AC-9 | 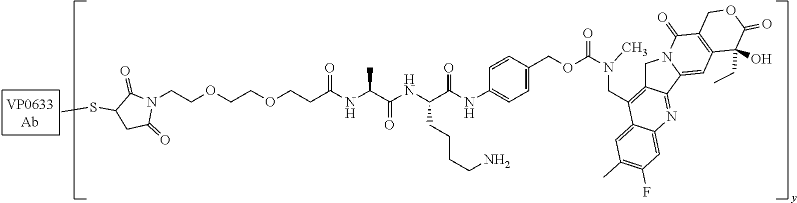 |
| AC-12 | 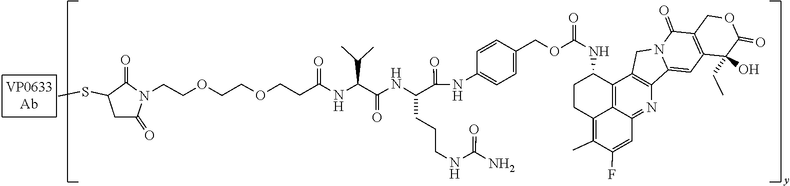 |
| AC-40 | 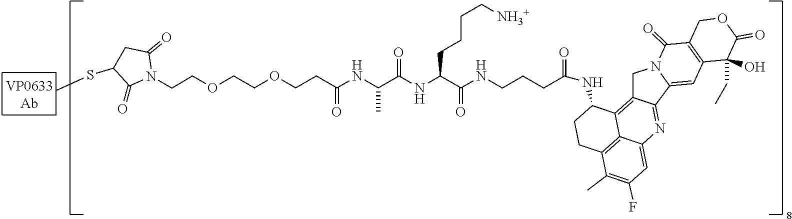 |
| | AC-40 (DAR8) |
| AC-105 | 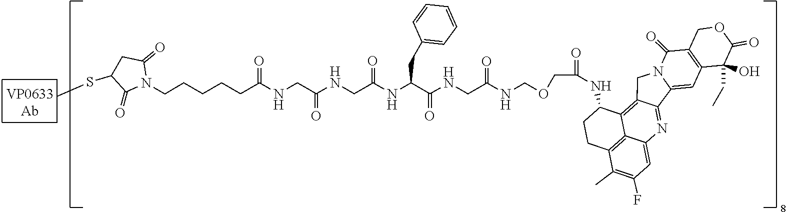 |
| | AC-105 (DAR8) |
| AC-A1 | 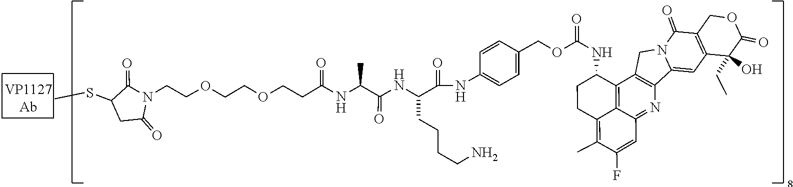 |
| | AC-A1 (DAR8) |
| AC-B1 | 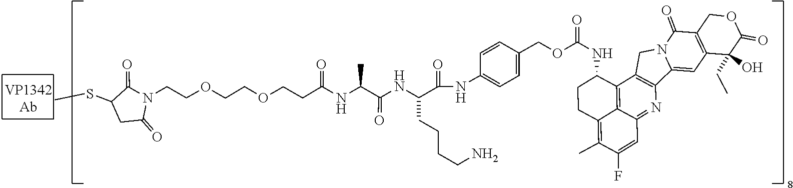 |
| | AC-B1 (DAR8) |
| AC-C1 | 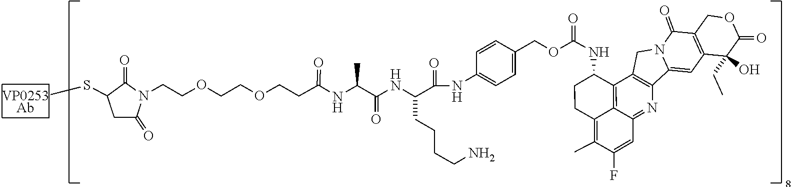 |
| | AC-C1 (DAR8) |
| AC-A40 | 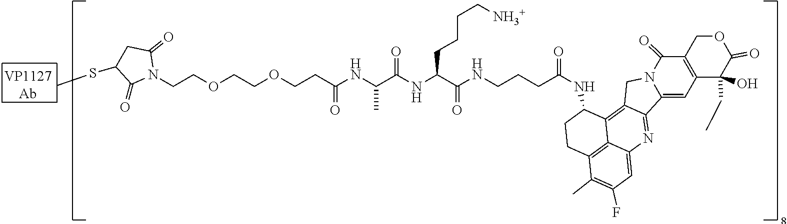 |
| | AC-A40 (DAR8) |
| AC-A105 | 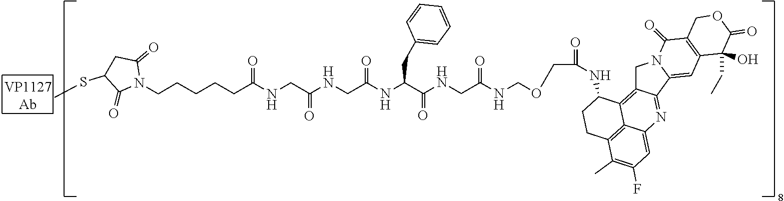 |
| | AC-A105 (DAR8) |
| AC-B40 | 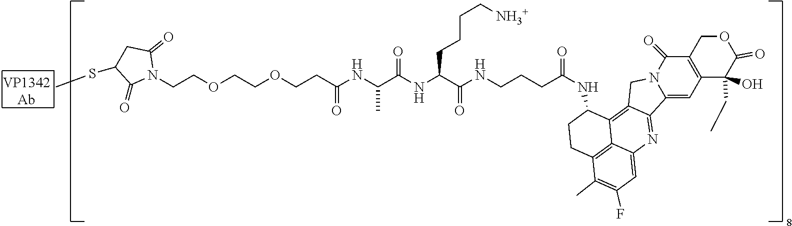 |
| | AC-B40 (DAR8) |
| AC-B105 | 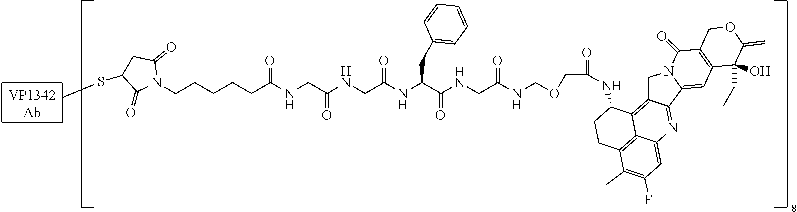 |
| | AC-B105 (DAR8) |
| AC-C40 | 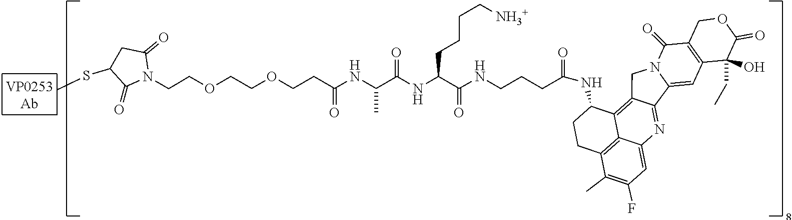 |
| | AC-C40 (DAR8) |
| AC-C105 | 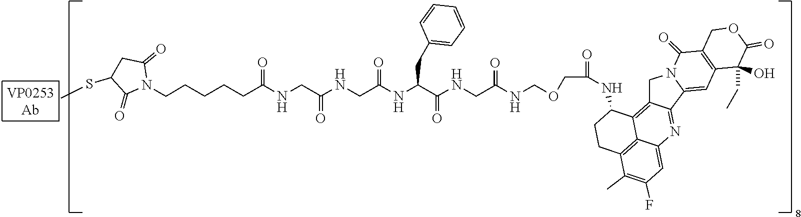 |
| | AC-C105 (DAR8) |

### Pharmaceutical Compositions

In another aspect, provided herein are pharmaceutical compositions comprising the conjugates (e.g., ADCs) as disclosed herein. In some embodiments, the pharmaceutical composition comprises the conjugate of Formula (II), (IIa), (IIb), (IIc) or (IId), and a pharmaceutically acceptable carrier.

Pharmaceutical compositions herein are formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, a pharmaceutical composition disclosed herein further comprises a pharmaceutically acceptable diluent(s), excipient(s), or carrier(s). In some embodiments, the pharmaceutical compositions include other medicinal or pharmaceutical agents, carriers, adjuvants, such as preserving, stabilizing, wetting or emulsifying agents, solution promoters, salts for regulating the osmotic pressure, and/or buffers.

In certain embodiments, a pharmaceutical composition disclosed herein is administered to a subject by any suitable administration route, including but not limited to, parenteral (intravenous, subcutaneous, intraperitoneal, intramuscular, intravascular, intrathecal, intravitreal, infusion, or local) administration.

Formulations suitable for intramuscular, subcutaneous, peritumoral, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent is optionally formulated in aqueous solutions, preferably in physiologically compatible buffers such as Hank's solution, Ringer's solution, or physiological saline buffer.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

In some embodiments, the pharmaceutical composition described herein is in unit dosage forms suitable for single administration of precise dosages. In unit dosage form, the formulation is divided into unit doses containing appropriate quantities of an active agent disclosed herein. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

### Methods of Use

The conjugates of Formula (II), (IIa), (IIb), (IIc) or (IId) as described herein allow the delivery of a therapeutic agent to specific cells and/or tissues. In some embodiments, a conjugate of Formula (II), (IIa), (IIb), (IIc) or (IId) enables targeted delivery of a therapeutic agent to a cell tissue. In some embodiments, targeted delivery of a therapeutic agent to a cell or tissue decreases contact of the therapeutic agent with healthy tissue. In some embodiments, targeted delivery of a therapeutic agent to a cell or tissue decreases unwanted side-effects arising from use of high concentrations of a therapeutic agent.

Disclosed herein, in certain embodiments, are methods of delivering a conjugate of Formula (II), (IIa), (IIb), (IIc) or (IId) to a tissue of interest, comprising contacting the tissue of interest with a conjugate of Formula (II), (IIa), (IIb), (IIc) or (IId).

In some embodiments, the conjugates of Formula (II), (IIa), (IIb), (IIc) or (IId) allow the delivery of a therapeutic agent to a tissue of interest. In some embodiments, the tissue of interest is cancerous tissue (or, cancer). In some embodiments, the cancerous tissue is a cancerous tissue that has overexpression of ICAM1 and/or EphA2. In some embodiments, the cancerous tissue comprises colon cancer, lung cancer (e.g., non-small cell lung cancer) tissue, prostate, or pancreatic cancer tissue. In some embodiments, the cancerous tissue is colon cancer tissue. In some embodiments, the cancerous tissue is lung cancer (e.g., non-small cell lung cancer) tissue. In some embodiments, the cancerous tissue is prostate cancer tissue. In some embodiments, the cancerous tissue is pancreatic cancer tissue or multiple myeloma.

Provided herein are methods of treating a cancer in a subject in need thereof comprising administering to the subject in need thereof a therapeutically effective amount of a conjugate of Formula (II), (IIa), (IIb), (IIc) or (IId) disclosed herein, or a pharmaceutically acceptable salt or solvate thereof.

In some embodiments, the cancer is a cancer that has overexpression of ICAM1 and/or EphA2. In some embodiments, the cancer is a solid tumor. In some embodiments, the cancer is selected from head and neck cancer, mesothelioma, hepatocellular carcinoma, meningioma, malignant peripheral nerve sheath tumor, Schwannoma, lung cancer, colorectal cancer, bladder carcinoma, cutaneous neurofibromas, prostate cancer, pancreatic cancer, glioblastoma, endometrial adenosquamous carcinoma, anaplastic thyroid carcinoma, gastric adenocarcinoma, esophageal adenocarcinoma, ovarian cancer, ovarian serous adenocarcinoma, melanoma, and breast cancer. In some embodiments, the cancer is breast cancer. In some embodiments, the cancer is lung cancer (e.g., non-small cell lung cancer). In some embodiments, the cancer is colorectal cancer. In some embodiments, the cancer is prostate cancer. In some embodiments, the cancer is pancreatic cancer. In some embodiments, the cancer is hepatocellular carcinoma.

In certain embodiments, the cancer is a hematological malignancy. In certain embodiments, the hematological malignancy is leukemia, lymphoma, or myeloma. In certain embodiments, the hematological malignancy is multiple myeloma (MM).

In certain embodiments, the hematological malignancy is selected from the group consisting of: Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, mantle zone lymphoma, low grade follicular lymphoma, multiple myeloma (MM), chronic lymphocytic leukemia (CLL), myelodysplastic syndrome (MDS), acute T cell leukemia, acute myeloid leukemia (AMIL), and chronic myelogenous leukemia (CML).

Also provided herein are methods of treating or preventing a disease or disorder amenable to treatment with a compound that inhibits topoisomerase (e.g., topoisomerase I) or PARP in a subject comprising administering to a subject in need thereof a therapeutically acceptable amount of a conjugate of Formula (II), (IIa), (IIb), (IIc) or (IId), or a pharmaceutically acceptable salt or solvate thereof.

In yet another aspect, provided herein are uses of a conjugate of Formula (II), (IIa), (IIb), (IIc) or (IId) as disclosed herein or a pharmaceutically acceptable salt thereof, or the pharmaceutical composition thereof as described herein, in the preparation of a medicament for treating a disease or disorder (e.g., a disease or disorder conducive to treatment to prevention by inhibiting topoisomerase I or PARP, such as a cancer) in a subject in need thereof.

In yet another aspect, a conjugate of Formula (II), (IIa), (IIb), (IIc) or (IId) disclosed herein is for use in a method of treating a disease or disorder (e.g., a disease or disorder conducive to treatment to prevention by inhibiting topoisomerase I or PARP, such as a cancer) in a subject in need thereof, such cancer. Such a compound is, for example, a conjugate of Formula (II), (IIa), (IIb), (IIc) or (IId) as disclosed herein, or a pharmaceutical composition comprising the compound disclosed herein, and a pharmaceutically acceptable excipient, as disclosed herein.

In yet another aspect, provided herein are pharmaceutical compositions comprising a conjugate of Formula (II), (IIa), (IIb), (IIc) or (IId) as disclosed herein or a pharmaceutically acceptable salt thereof, for use in treating a disease or disorder (e.g., a disease or disorder conducive to treatment to prevention by inhibiting topoisomerase I or PARP, such as a cancer) in a subject in need thereof.

### EXAMPLES

The following schemes and examples are illustrative of how the compounds described herein can be prepared and tested. Although the examples can represent only some embodiments, it should be understood that the following examples are illustrative and not limiting. The specific synthetic steps for each of the routes described may be combined in different ways, or in conjunction with steps from different schemes, to prepare the compounds described herein.

### I. Chemical Synthesis

### Abbreviations

| Acronym/ | |
|---|---|
| Abbreviation | Meaning |
| DAR | Drug-to-antibody ratio |
| DCC | N,N′-Dicyclohexylcarbodiimide |
| DCM | Dichloromethane |
| DMA | N,N-dimethyl acetamide |
| DMF | N,N-dimethyl formamide |
| DIPEA | N,N-Diisopropylethylamine |
| EEDQ | 2-Ethoxy-1-ethoxycarbonyl-1,2-dihydroquinoline |
| FMOC | Fluorenylmethyloxycarbonyl |
| HBTU | (2-(1H-benzotriazol-1-yl)-1,1,3,3-tetramethyluronium |
| | hexafluorophosphate |
| HIC | Hydrophobic interaction chromatography |
| HOBT | Hydroxybenzotriazole |
| HPLC | High performance liquid chromatography |
| PBS | Phosphate-buffered saline |
| TFA | Trifluoroacetic acid |
| THF | Tetrahydrofuran |
| SEC | Size exclusion chromatography |
| SPPS | Solid phase peptide synthesis |
| TEA | Triethylamine |

Solvents, reagents and starting materials were purchased from commercial vendors and used without further purification unless otherwise described. All reactions were performed at room temperature unless otherwise stated. Starting materials were purchased from commercial sources or synthesized according to the methods described herein or using literature procedures or the present disclosure.

### Example I-1: Synthesis of Compounds 1-14

Compounds 1-14 were synthesized using the synthetic route as shown in Scheme 1 using a combination of solid phase peptide synthesis (SPPS) with FMOC protected amino acids followed by solution based methods.

As shown in Scheme 1, 2 g of chlorotrityl resin (1 mmol/g) containing FMOC protected first amino acid AA₁ (I) was placed in a glass funnel and 50 mL of a 20% solution of piperidine in dimethylacetamide (DMA) was added. The suspension was percolated with nitrogen and mixing continued for 15 minutes. The liquid phase was removed under vacuum and the resin washed with 50 mL DMA followed by 50 mL DCM. In a separate flask the second FMOC-protected amino acid AA₂ (4 mmol) was dissolved in 20 mL DMA and HBTU (4 mmol) was added followed by 8 mmol of DIPEA. The mixture was then added to the resin and gently mixed via nitrogen percolation for 30 minutes. The resin was filtered to remove excess reagents and washed with DMA and DCM as before. The FMOC group was removed via treatment with 20% piperidine and the resin washed with DMA and DCM. In a separate vessel, 4 mmol of the maleimide connector II (3-(2-(2-(2,5-dioxo-2,5-dihydro-1H-pyrrol-1-yl)ethoxy)ethoxy)propanoic acid) was mixed with HBTU (4 mmol) in 20 mL of DMA and 8 mmol of DIPEA was added. The mixture was then added to the deprotected resin and perfused with nitrogen for 30 minutes. The resin was filtered and washed 2×50 mL DMA, 2×50 mL DCM, and once with methanol. The washed resin was air dried and intermediate III was cleaved from the resin by mixing with 100 mL of DCM containing 10% acetic acid. The mixture was filtered to remove the resin and the filtrate was evaporated to dryness to yield crude intermediate III. Purification by preparative HPLC using CH₃CN/H₂O afforded dipeptide intermediates III after lyophilization.

Intermediate III was dissolved in a minimal amount of DMF and 1.1 eq. of p-amino benzyl alcohol was added followed by 1.1 eq of EEDQ. The reaction mixture was stirred overnight at room temp and diluted with 5 volumes of DCM. The mixture was transferred to a sep funnel and washed 2× with water to remove DMF. The organic phase was evaporated to yield crude intermediate IV. After purification by preparative HPLC, intermediate VI was dissolved in dichloromethane and treated with 1.5 eq. of p-nitrophenyl chloroformate with excess DIPEA and stirred overnight at room temperature to yield crude intermediate V. The reaction mixture was concentrated to dryness and the crude residue was purified via flash chromatography eluted with a gradient of DCM and acetone to afford intermediate V.

The final products of Compounds 1-14 were obtained by mixing equal molar amounts of the desired topoisomerase 1 inhibitor (e.g., Exatecan) with the 4-nitrophenylcarbonate intermediate V in DMF followed by addition of 1 eq. HOBT and 2 eq. DIPEA. For Compounds 1-10 containing a lysine residue, the BOC protecting group of the lysine sidechain was removed via treatment with TFA in DCM. The reaction mixture was purified via preparative HPLC eluted with CH₃CN/H₂O gradient, and lyophilized to afford the desired Compounds 1-14.

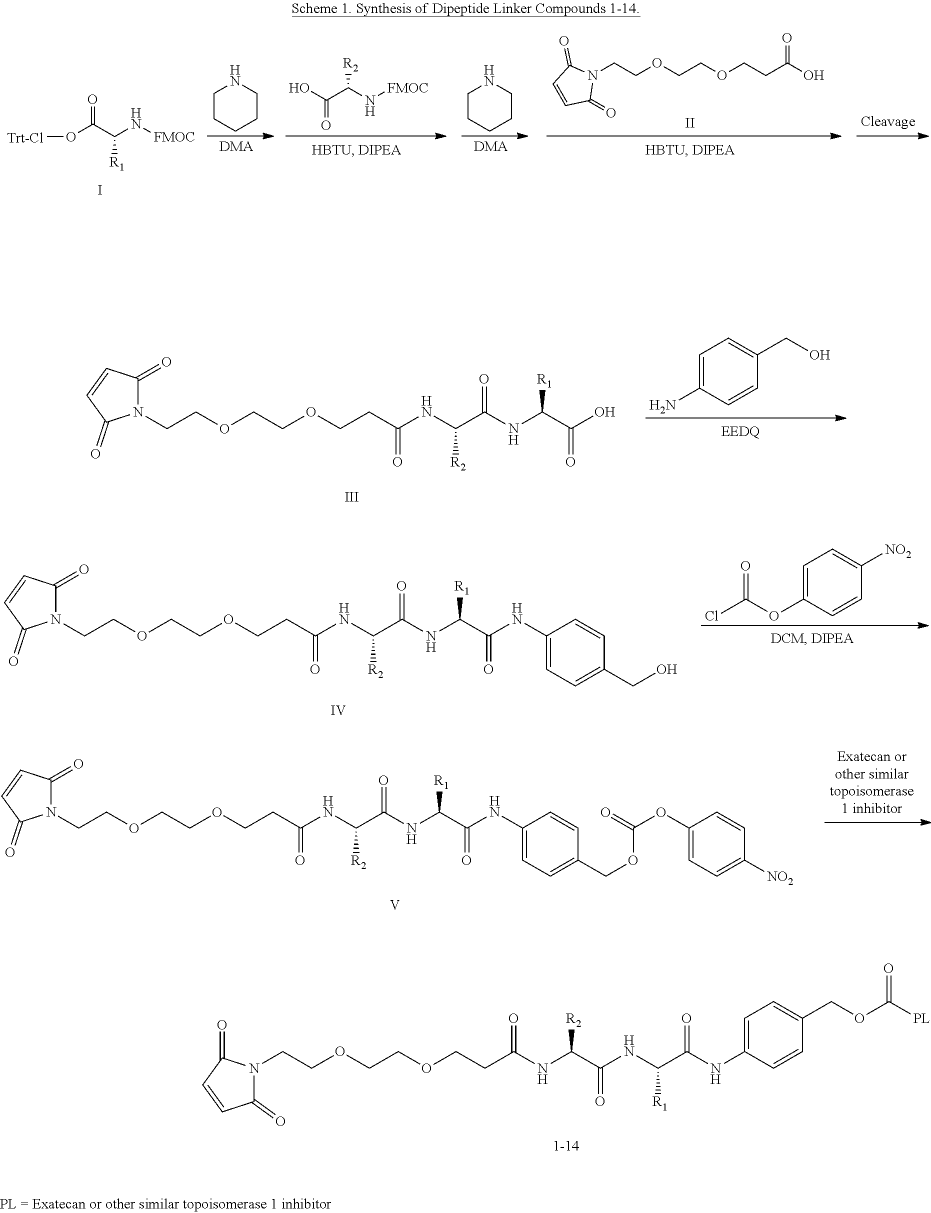

### Example I-2: Synthesis of Tripeptide Linker Compounds 15-18

Compounds 15-18 with a tripeptide linker were synthesized using a similar procedure for that was used for Compounds 1-14 except that an additional coupling step with a third amino acid AA₃ and a deprotection step were performed to afford a tripeptide linker prior to reacting with p-amino benzyl alcohol in the presence of EEDQ. The synthesis of Compounds 15-18 is shown in Scheme 2.

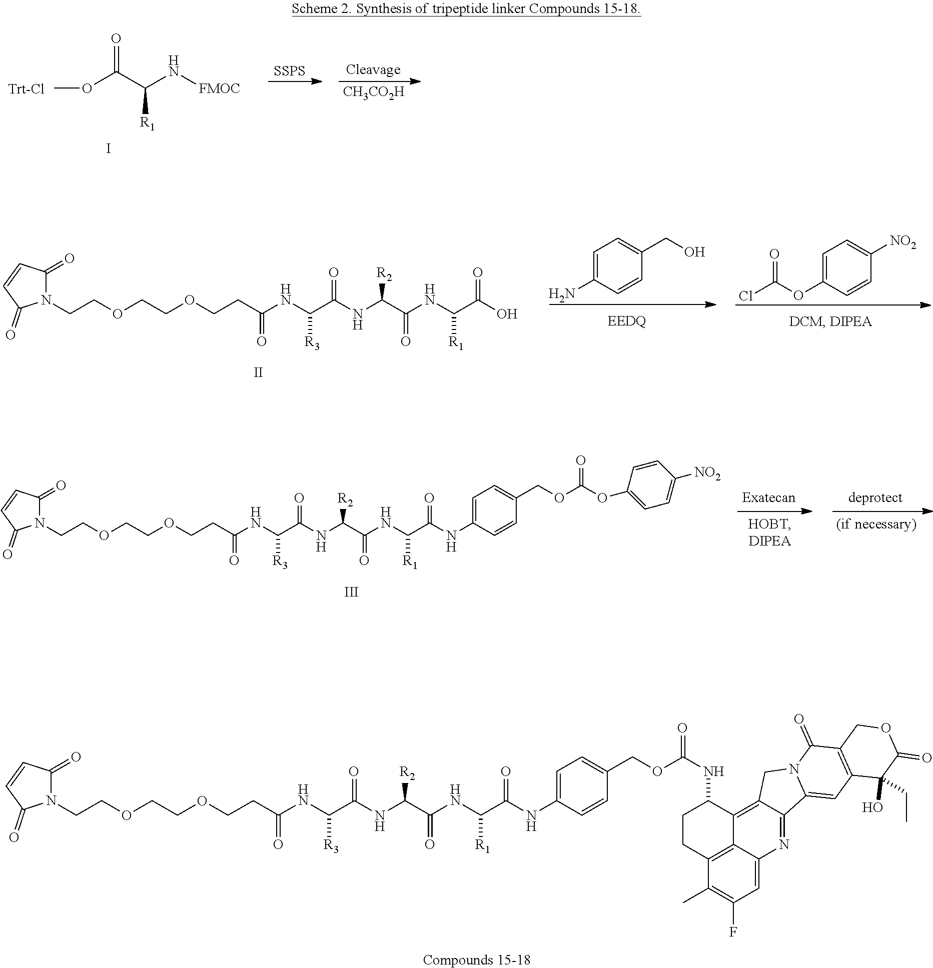

### Example I-3: Synthesis of Lysine Specific Compounds 19-22

Compounds 19-22 were synthesized using the synthetic route shown in Scheme 3. Specifically, a tripeptide linker (I) was synthesized on a chlorotrityl resin using standard SPPS protocols as previously described and coupled to a t-butyl protected diacid intermediate II. The resulting intermediate III was then cleaved from the resin with 10% acetic acid and the crude product was purified via preparative HPLC eluted with a CH₃CN/H₂O gradient. Fractions containing intermediate III were combined and concentrated to near dryness. The remaining aqueous solution was then lyophilized to yield intermediate III as a white powder.

Coupling of intermediate III top-aminobenzyl alcohol (1.1 eq.) using a slight excess of EEDQ (1.2 eq) followed by preparative HPLC purification afforded intermediate IV.

Treatment of intermediate IV with p-nitrophenyl chloroformate (1.2 eq.) in THF afforded the nitrophenyl carbonate ester V after flash chromatography on silica gel. Treatment of intermediate V with 1.1 molar equivalent of exatecan, HOBT, and DIPEA in DMF followed by removal of the t-butyl ester with TFA yielded intermediate VI.

Esterification of intermediates VI with tetrafluorophenol afforded the desired Compound 19-22, respectively. HPLC purification of the resulting tetrafluorophenyl (TFP) esters followed by lyophilization afforded the purified Compounds 19-22, which were stored at −20° C. or cooler until used for conjugation with an antibody.

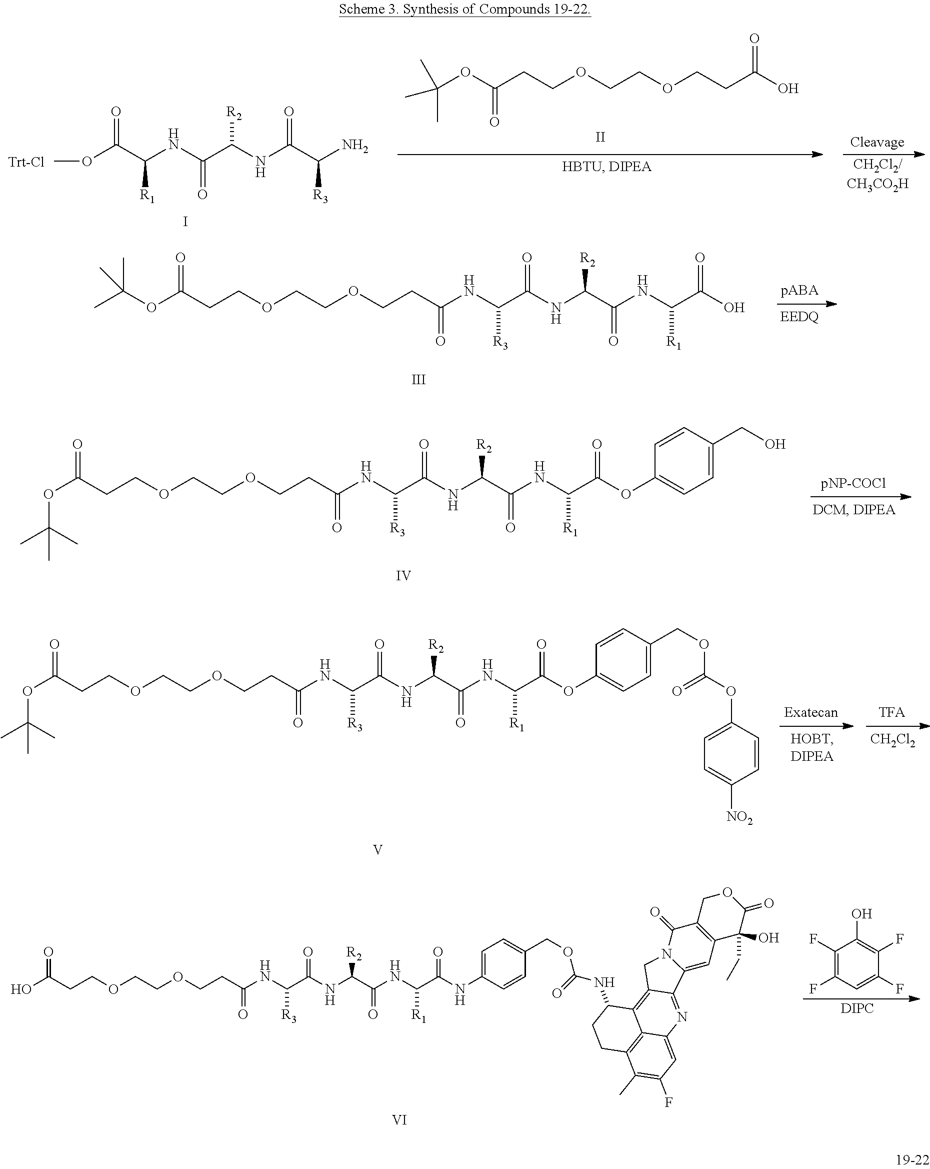

### Example I-4: Synthesis of Compounds 23-28

Compounds 23 synthesized using similar synthetic route for compounds 19-22 as shown in Scheme 3 above, except that Compound 23 has a dipeptide linker.

Compound 24 was synthesized using the synthetic route as shown in Scheme 4. Specifically, a maleimide dipeptide II was synthesized via SPPS as previously described. Coupling top-aminobenzoic alcohol (PABA) followed by p-nitrophenylcarbonate activation afforded intermediate III in a 40% overall yield. A PARP inhibitor, Talazoparib (IV) was coupled to N-Boc protected hydroxyethyl amine with phosgene and 2 eq. of TEA in DCM to yield intermediate V after aqueous workup, drying over MgSO₄, and evaporation of solvent. Treatment of intermediate V with TFA in DCM afforded intermediate VI which was then coupled to intermediate III in the presence of HOBT to yield Compound 24 in 20% overall yield.

Compound 26 was synthesized using the synthetic route as shown in Scheme 5. Specifically, the dipeptide linker (Ala-Lys) was synthesized on a chlorotrityl resin as previously described herein using standard protocols.

The PEG2-maleimide group was coupled to the dipeptide which was cleaved from the resin as described previously. EEDQ mediated coupling top-aminobenzyl alcohol afforded intermediate II which was purified by preparative HPLC as previously described.

A PARP inhibitor, Talazoparib (III) was dissolved in THF, cooled to −78° C., and treated with 1 eq. of n-BuLi. The reaction mixture was stirred for 15 minutes and 1.5 eq. of iodoethyl acetate was added. The reaction mixture was warmed to room temperature and treated with 2 eq. of LiOH and stirred for 2 hours to hydrolyze the ethyl ester. The solvent was removed under vacuum and the crude product IV was purified via preparative HPLC eluted with a 10-40% CH₃CN/H₂O gradient. The purified intermediate IV was treated with DPPA at room temperature to afford acyl azide intermediate V. Addition of benzyl alcohol intermediate II to azide intermediate V resulted in a Curtius rearrangement to yield the dipeptide carbamate intermediate VI. Removal of the Boc protecting group with TFA afforded desired Compound 26.

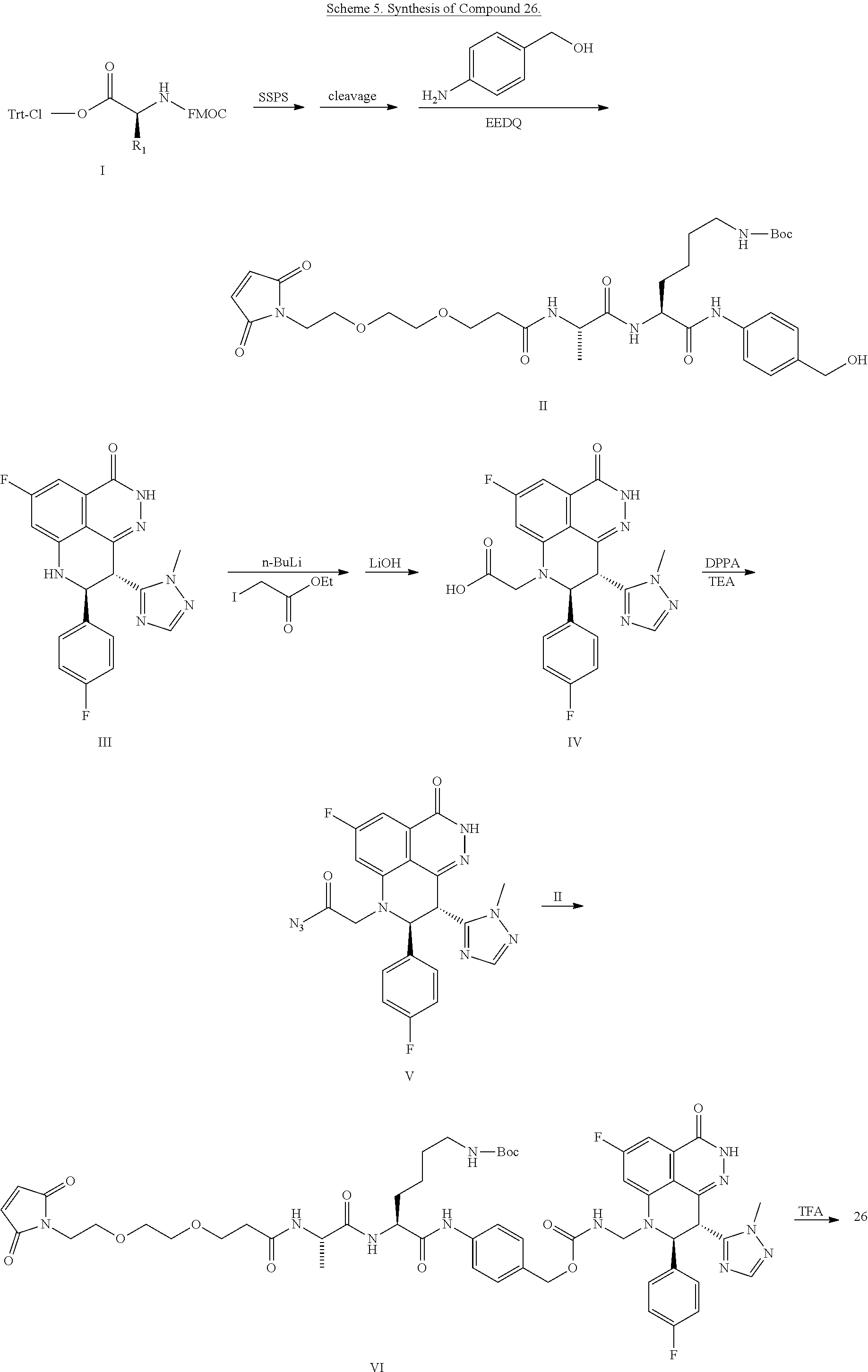

Compound 25 was synthesized using a similar procedure for that was used for Compound 26 except that Compound 25 was synthesized starting with a tripeptide linker (Gly-Phe-Gly) instead of dipeptide linker (Ala-Lys) on a chlorotrityl resin.

Compounds 27 and 28, which were synthesized using another PARP inhibitor Niraparib, were synthesized using the synthetic route as shown in Scheme 6. Compounds 27 and 28 differ in the dipeptide linker, but are attached to the same moiety from Niraparib. Compounds 27 has an Ala-Ala linker and Compound 28 has an Ala-Lys linker.

Specifically, the dipeptide linker intermediate II was synthesized as previously described using SPPS with FMOC protocols. After cleavage from the resin, intermediate II was coupled with p-aminobenzoic alcohol (PABA) in the presence of EEDQ, and then activated with p-nitrophenyl chloroformate to afford crude intermediate III. Purification via silica gel chromatography eluted with 5% MeOH in CH₂Cl₂ afforded pure carbonate ester III as a pale yellow solid. HOBT mediated coupling of intermediate III with commercially available Niraparib (IV) yielded Compounds 27 and 28, respectively, after HPLC purification followed by lyophilization.

### Example I-5: Synthesis of Compounds 29-32

Compounds 29 and 30 were synthesized using the synthetic route as shown in Scheme 7. Specifically, the p-nitrophenyl carbonate intermediate II was synthesized as previously described in Scheme 3. The Talazoparib derivative V was synthesized as previously described in Scheme 4.

Intermediates II and V were coupled to afford the bis-carbamate intermediate VI after removal of the t-butyl ester with TFA. Intermediate VI was esterified via treatment with tetrafluorophenol and DCC in DCM to afford crude Compounds 29 and 30, which were purified via preparative HPLC and lyophilized to yield the purified Compounds 29 and 30 as off-white powder.

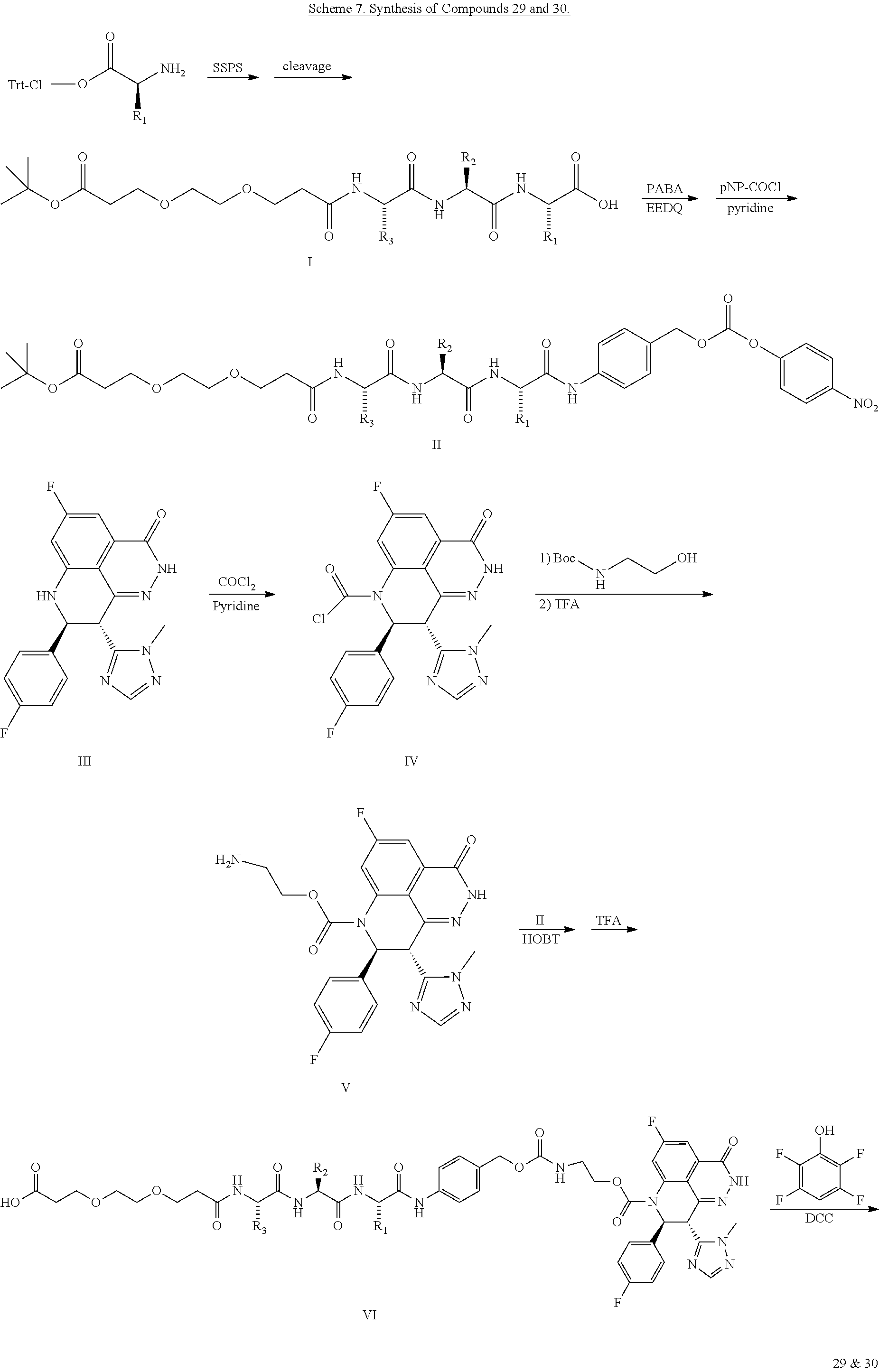

Compounds 31 and 32 were synthesized using a procedure similar to the procedures described for Compounds 27-30.

### Example I-6: Synthesis of Compounds 33-36

The synthesis of Compounds 33-36 with a terminal dibromomaleimide (DBM) moiety is analogous to the synthesis of similar compounds with maleimide linkers except a dibromo maleimide analog was used in lieu of the corresponding maleimide analog during the SPPS process. The synthesis of the dibromomaleimide-PEG2 acid derivative used in the synthesis of Compounds 33-36 is shown in Scheme 8A.

Compound 33 was synthesized using the procedure as shown in Scheme 8B. Specifically, the Ala-Lys dipeptide linker was synthesized on a chlorotrityl resin as described previously. The DBM-PEG-acid intermediate III was coupled to the Ala-Lys dipeptide linker and the resulting intermediate IV was cleaved from the resin and purified by HPLC. Coupling of intermediate IV with p-amino benzyl alcohol followed by activation with p-nitrophenyl chloroformate afforded p-nitrophenyl carbonate intermediate V after purification on silica gel eluted with 10:1 DCM/acetone. Coupling of intermediate V with exatecan followed by removal of the BOC protecting group afforded Compound 33 in a 7% overall yield (Scheme 8).

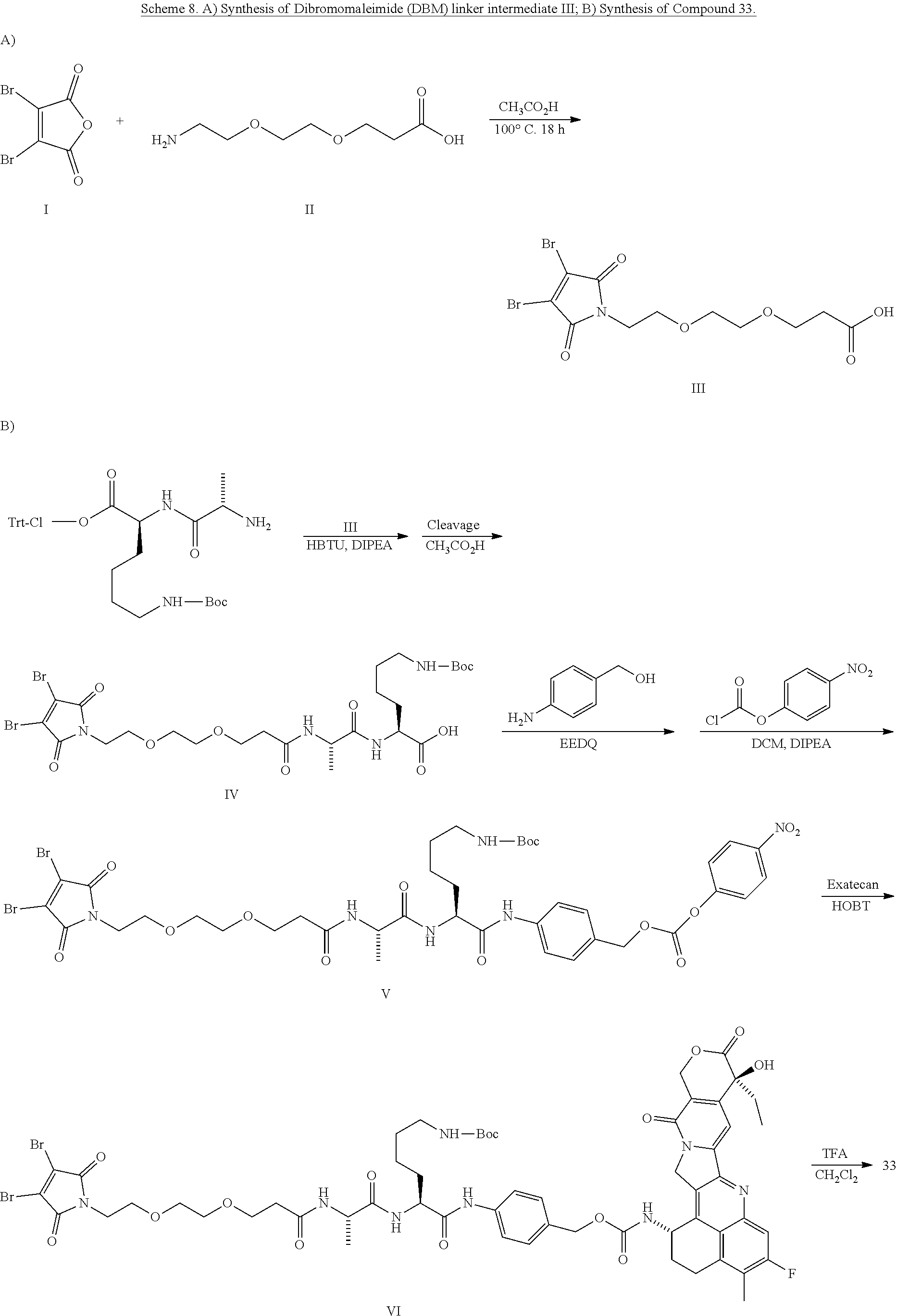

Compounds 34-36 were synthesized using a procedure similar to the procedures described for Compound 33.

### Example I-7: Synthesis of Compounds 37 and 38

Compounds 37 and 38 were synthesized using the procedure similar to the procedure described above for Compounds 15-18.

The characterization data of certain exemplary compounds synthesized as described above (e.g., LC-MS) are provided in Table 7 below.

TABLE 7

| Compound | | Analytical |
|---|---|---|
| # | Structure | Data |
| Compound 1 | 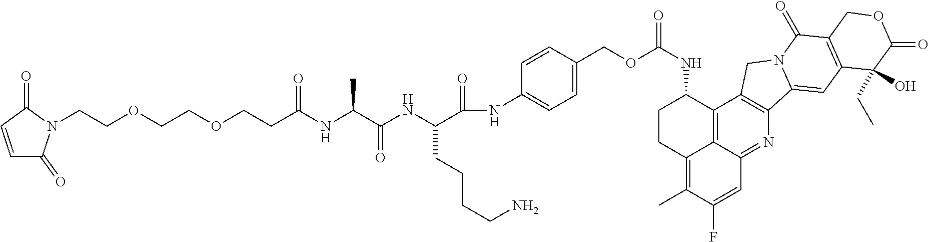 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCCN)NC(=O)[C@H](C)NC(=O)CCOCCOCCN2C(=O)C=CC2=O)cc1)CC3] | Retention time: 11.164 min; LC- MS m/z 1023.4 [M + 1]⁺. |
| Compound 2 | 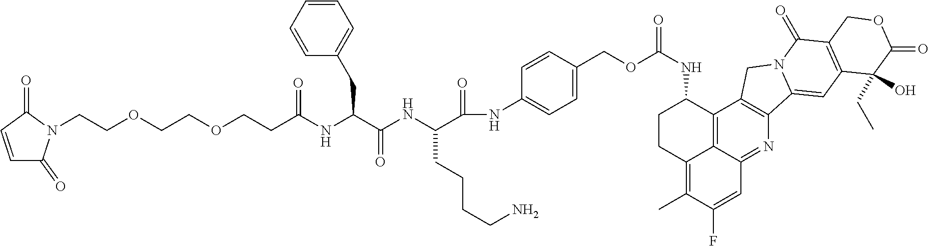 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCCN)NC(=O)[C@H](Cc2ccccc2)NC(=O)CCOCCOCCN2C(=O)C=CC2=O)cc1)CC3] | Retention time: 4.233 min; LC- MS m/z 1121.5 [M + 1]⁺. |
| Compound 3 | 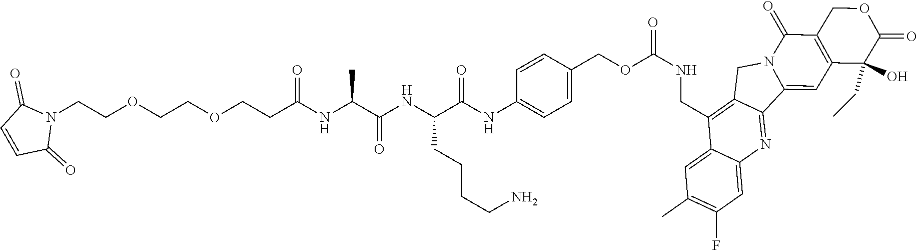 [SMILES: CC[C@@]1(O)C(=O)OCc2c1cc1n(c2=O)Cc2c-1nc1cc(F)c(C)cc1c2CNC(=O)OCc1ccc(NC(=O)[C@H](CCCCN)NC(=O)[C@H](C)NC(=O)CCOCCOCCN2C(=O)C=CC2=O)cc1] | Retention time: 2.171 min; LC- MS m/z 997.3 [M + 1]⁺. |
| Compound 4 | 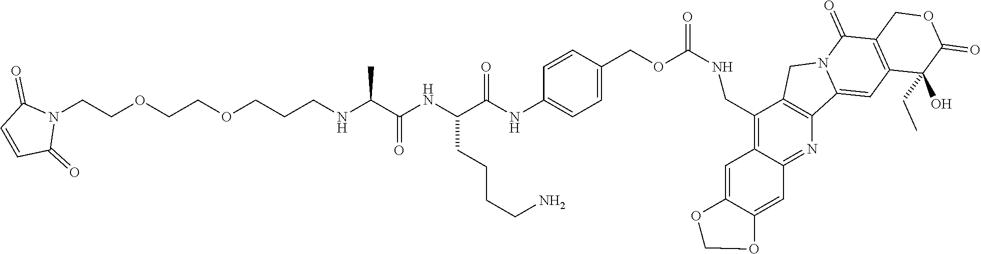 | Retention time: 7.795 min; LC- MS m/z 1009.4 [M + 1]⁺. |
| Compound 6 | 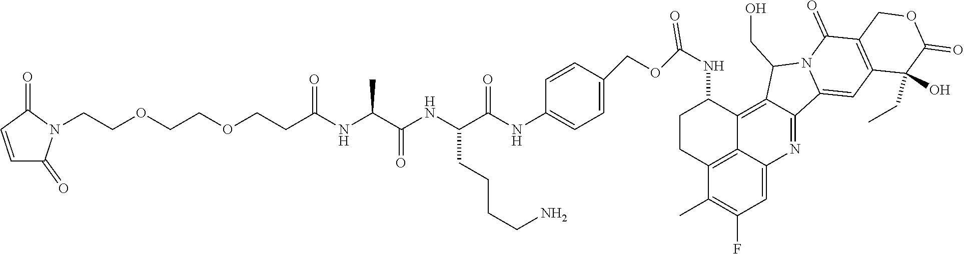 | Retention time: 0.733 min; LC- MS m/z 1068.5 [M + 2]. |
| Compound 7 | 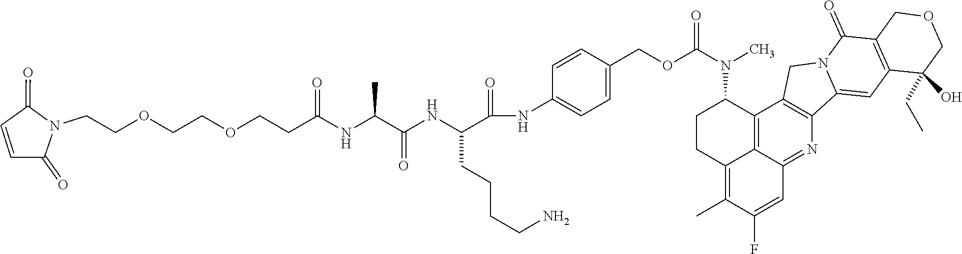 | Retention time: 0.743 min; LC- MS m/z 1038.9 [M + 1]⁺. |
| Compound 8 | 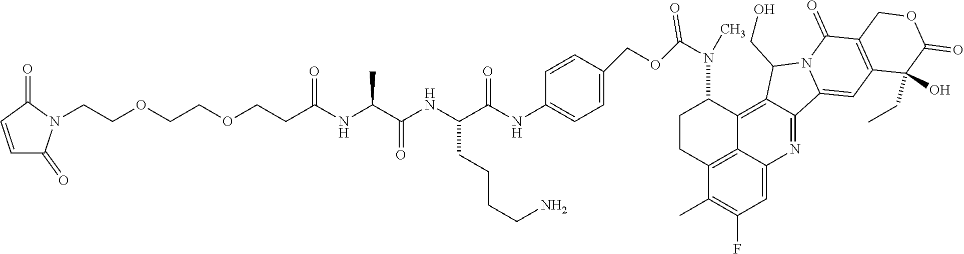 | Retention time: 0.733 min; LC- MS m/z 1068.5 [M + 1]⁺. |
| Compound 9 | 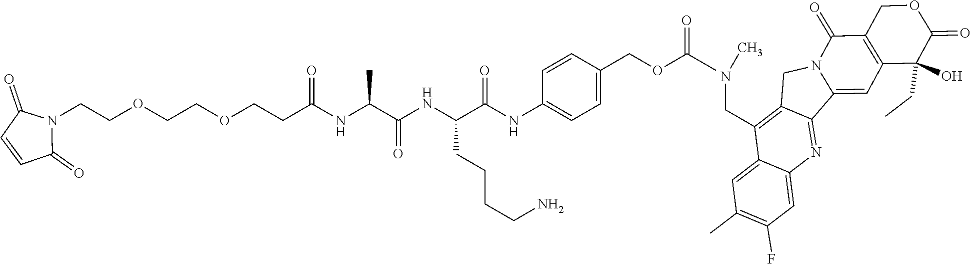 | Retention time: 2.164 min; LC- MS m/z 1068.5 [M + 1]⁺. |
| Compound 10 | 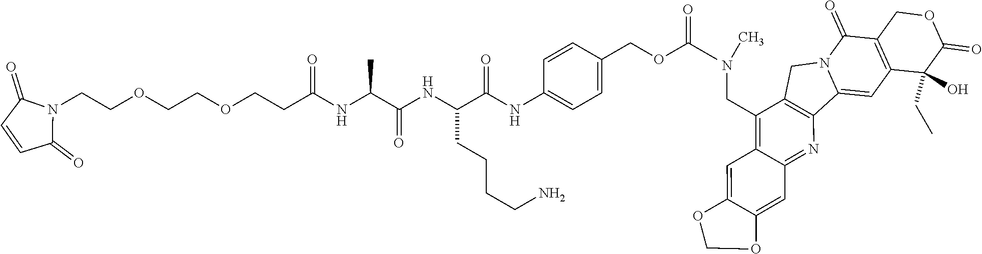 | Retention time: 2.259 min; LC- MS m/z 1011.3 [M + 1]⁺. |
| Compound 11 | 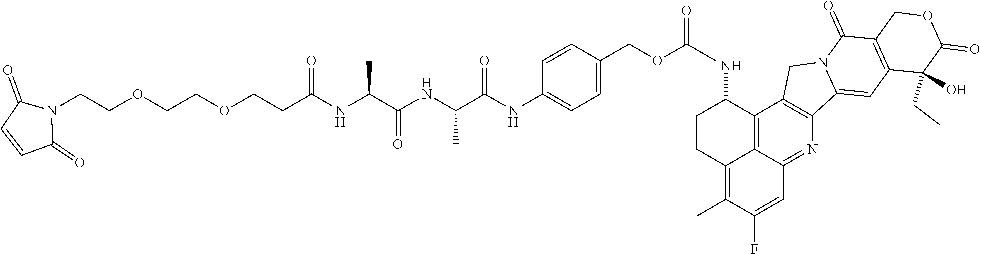 | Retention time: 1.618 min; LC- MS m/z 966.5 [M + 1]⁺. |
| Compound 12 | 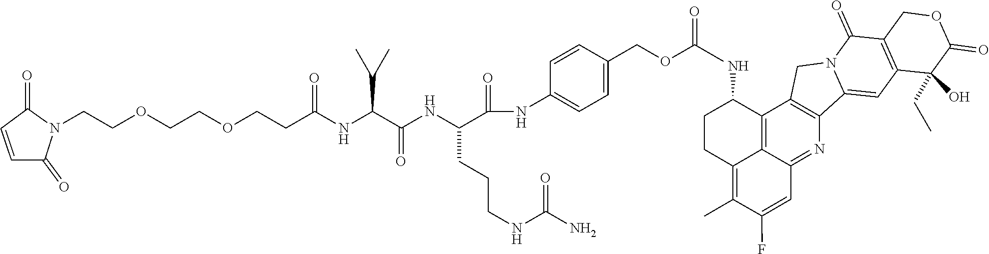 | Retention time: 4.736 min; LC- MS m/z 1034.4 [M + 1]⁺. |
| Compound 15 | 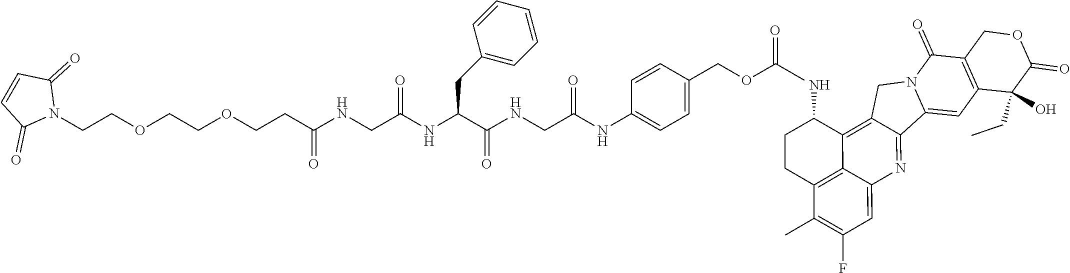 | Retention time: 2.946 min; LC- MS m/z 1085.4 [M + 1]⁺. |
| Compound 16 | 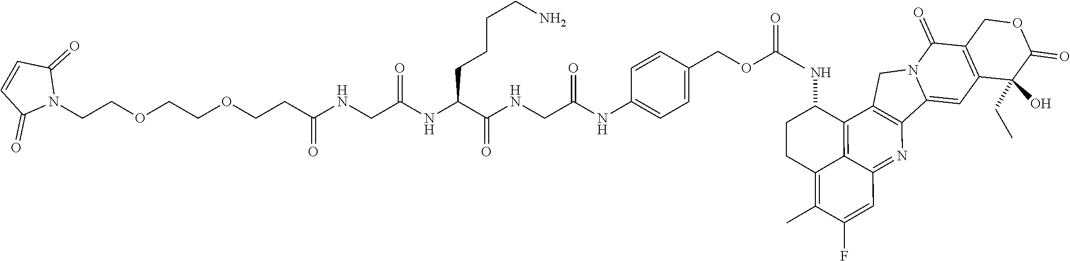 | Retention time: 3.939 min; LC- MS m/z 1088.5 [M + 1]⁺. |
| Compound 18 | 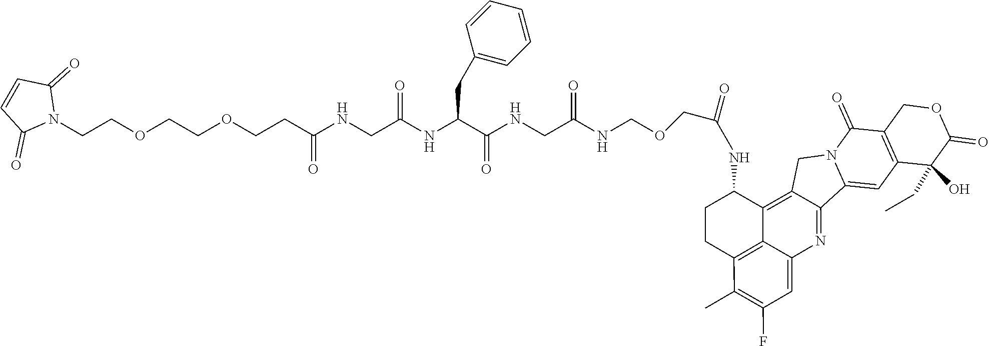 | Retention time: 3.762 min; LC- MS m/z 1023.3 [M + 1]⁺. |
| Compound 19 | 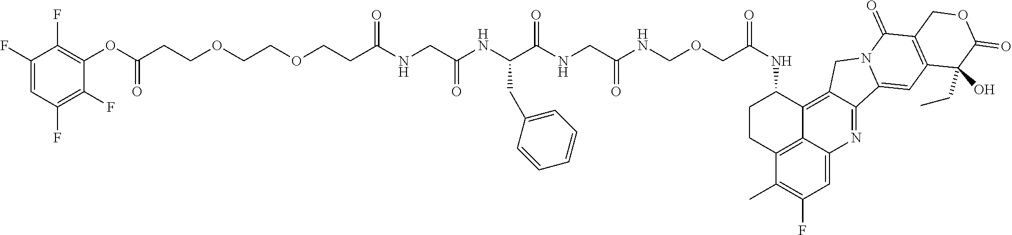 | Retention time: 3.248 min; LC- MS m/z 1117.4 [M + 1]⁺. |
| Compound 20 | 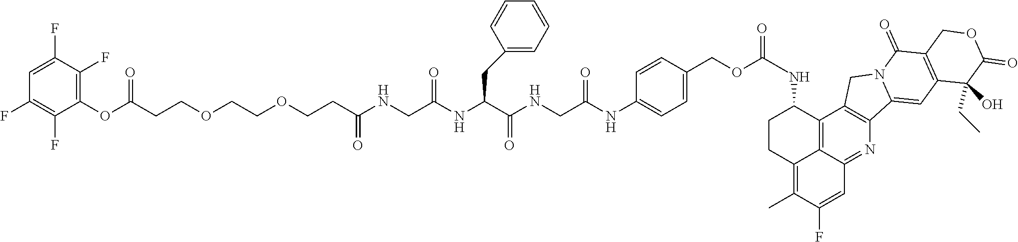 | Retention time: 4.048 min; LC- MS m/z 1182.5 [M + 1]⁺. |
| Compound 21 | 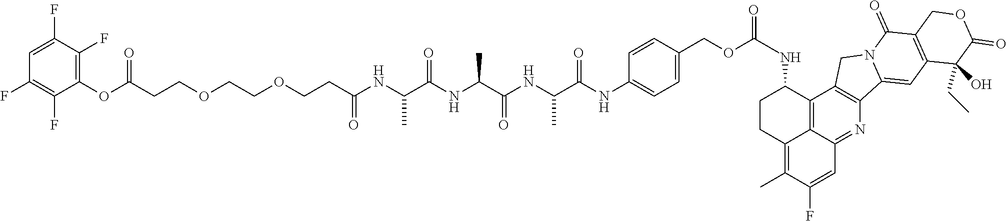 | Retention time: 1.15 min; LC- MS m/z 1134.4 [M + 1]⁺. |
| Compound 26 | 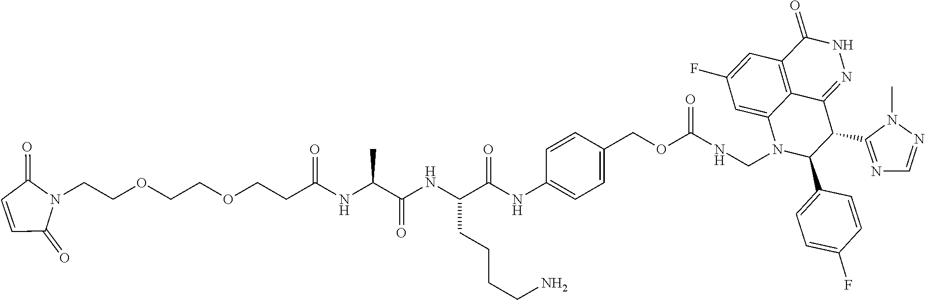 [SMILES: C[C@H](NC(=O)CCOCCOCCN1C(=O)C=CC1=O)C(=O)N[C@@H](CCCCN)C(=O)Nc1ccc(COC(=O)NCN2c3cc(F)cc4c(=O)[nH]nc(c34)[C@H](c3ncnn3C)[C@H]2c2ccc(F)cc2)cc1] | Retention time: 9.421 min; LC- MS m/z 997.80 [M + 1]⁺. |
| Compound 28 | 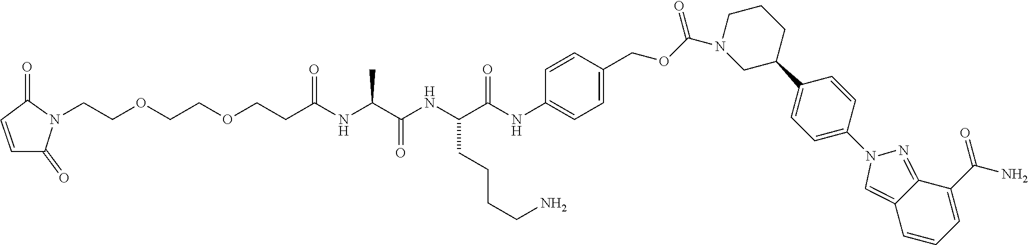 [SMILES: C[C@H](NC(=O)CCOCCOCCN1C(=O)C=CC1=O)C(=O)N[C@@H](CCCCN)C(=O)Nc1ccc(COC(=O)N2CCC[C@@H](c3ccc(-n4cc5cccc(C(N)=O)c5n4)cc3)C2)cc1] | Retention time: 2.843 min; LC- MS m/z 908.3 [M + 1]⁺. |
| Compound 29 | 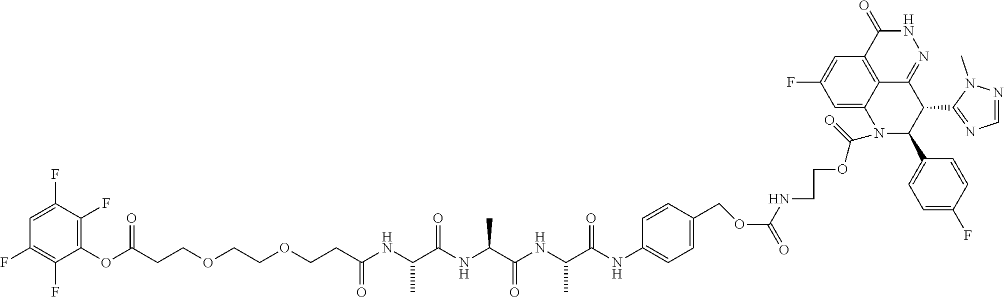 [SMILES: C[C@H](NC(=O)CCOCCOCCC(=O)Oc1c(F)c(F)cc(F)c1F)C(=O)N[C@@H](C)C(=O)N[C@@H](C)C(=O)Nc1ccc(COC(=O)NCCOC(=O)N2c3cc(F)cc4c(=O)[nH]nc(c34)[C@H](c3ncnn3C)[C@H]2c2ccc(F)cc2)cc1] | Retention time: 10.461 min; LC- MS m/z 1166.4 [M + 1]⁺. |
| Compound 31 | 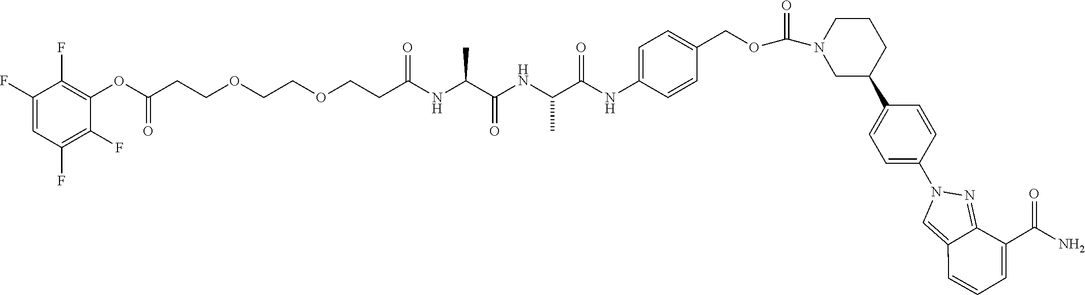 [SMILES: C[C@H](NC(=O)CCOCCOCCC(=O)Oc1c(F)c(F)cc(F)c1F)C(=O)N[C@@H](C)C(=O)Nc1ccc(COC(=O)N2CCC[C@@H](c3ccc(-n4cc5cccc(C(N)=O)c5n4)cc3)C2)cc1] | Retention time: 3.642 min; LC- MS m/z 948.3 [M + 1]⁺. |
| Compound 32 | 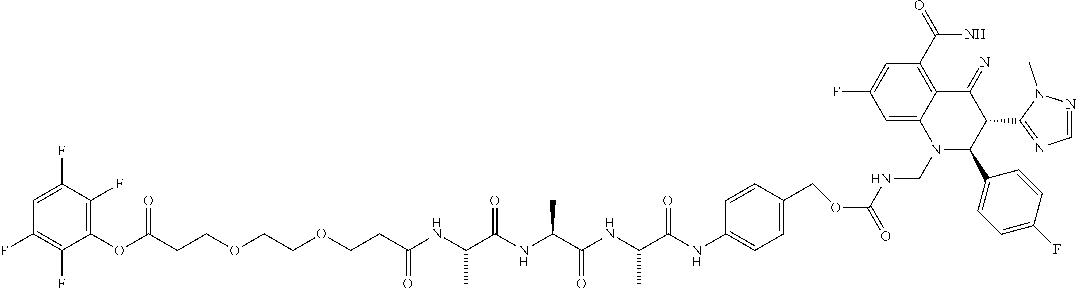 [SMILES: C[C@H](NC(=O)CCOCCOCCC(=O)Oc1c(F)c(F)cc(F)c1F)C(=O)N[C@@H](C)C(=O)N[C@@H](C)C(=O)Nc1ccc(COC(=O)NCN2c3cc(F)cc(C(N)=O)c3C(=N)[C@H](c3ncnn3C)[C@H]2c2ccc(F)cc2)cc1] | Retention time: 11.184 min; LC- MS m/z 1108.5 [M + 1]⁺. |
| Compound 33 | 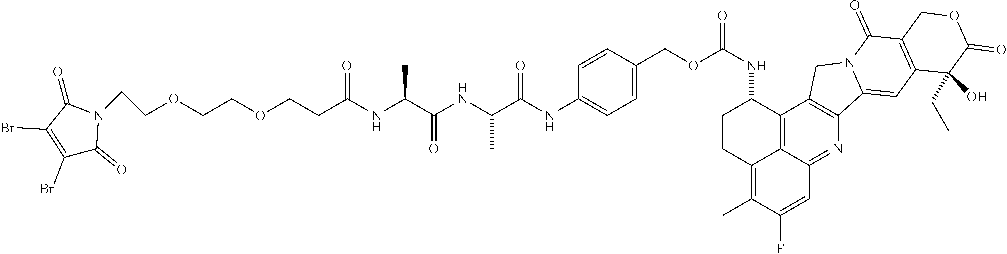 | Retention time: 10.285 min; LC- MS m/z 1124.1 [M + 1]⁺. |
| Compound 38 | 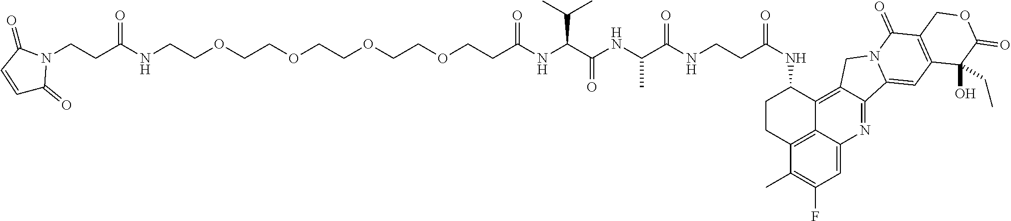 [SMILES: CC[C@@]1(O)C(=O)OCc2c1cc1n(c2=O)Cc2c-1nc1cc(F)c(C)c3c1c2[C@@H](NC(=O)CCNC(=O)[C@H](C)NC(=O)[C@@H](NC(=O)CCOCCOCCOCCOCCNC(=O)CCN1C(=O)C=CC1=O)C(C)C)CC3] | Retention time: 2.735 min; LC- MS m/z 1075.4 [M + 1]⁺. |
| Compound 40 | 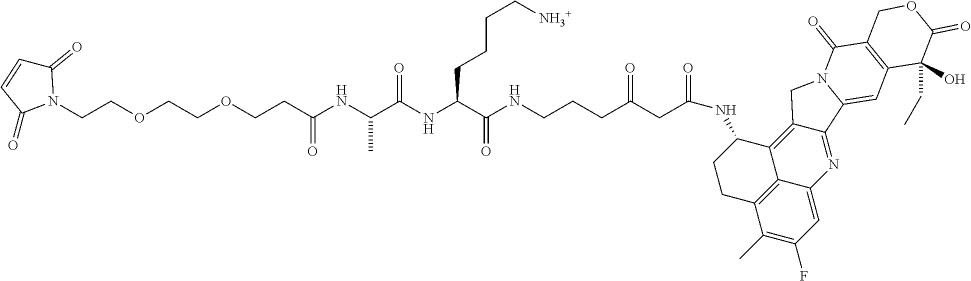 | Retention time: 2.617 min; LC- MS m/z 959.4 [M + 1]⁺. |
| Compound 41 | 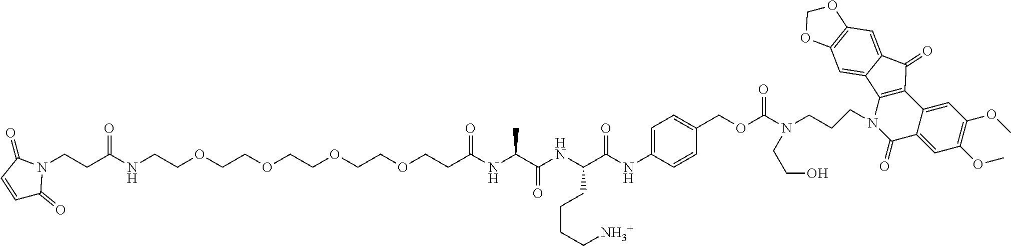 [SMILES: COc1cc2c3c(n(CCCN(CCO)C(=O)OCc4ccc(NC(=O)[C@H](CCCC[NH3+])NC(=O)[C@H](C)NC(=O)CCOCCOCCOCCOCCNC(=O)CCN5C(=O)C=CC5=O)cc4)c(=O)c2cc1OC)-c1cc2c(cc1C3=O)OCO2] | Retention time: 10.7 min; LC- MS m/z 1199.8 [M + 1]⁺. |
| Compound 42 | 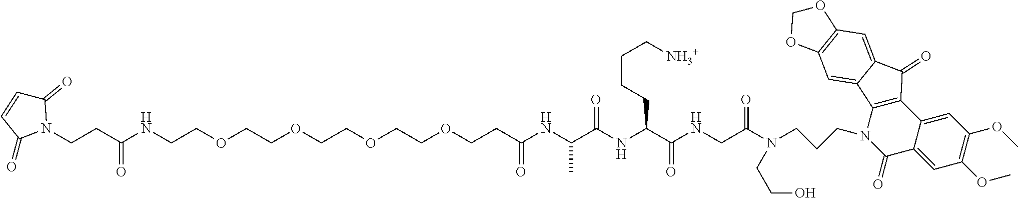 [SMILES: COc1cc2c3c(n(CCCN(CCO)C(=O)CNC(=O)[C@H](CCCC[NH3+])NC(=O)[C@H](C)NC(=O)CCOCCOCCOCCOCCNC(=O)CCN4C(=O)C=CC4=O)c(=O)c2cc1OC)-c1cc2c(cc1C3=O)OCO2] | Retention time: 9.6 min; LC- MS m/z 1107.7 [M + 1]⁺. |
| Compound 43 | 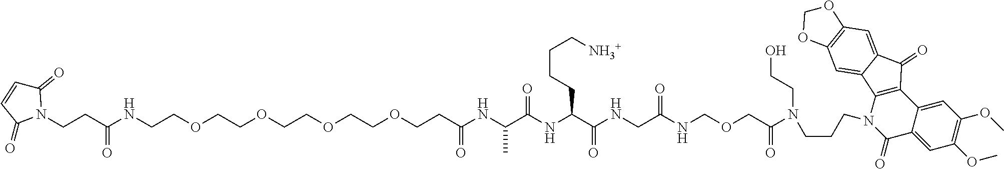 [SMILES: COc1cc2c3c(n(CCCN(CCO)C(=O)COCNC(=O)CNC(=O)[C@H](CCCC[NH3+])NC(=O)[C@H](C)NC(=O)CCOCCOCCOCCOCCNC(=O)CCN4C(=O)C=CC4=O)c(=O)c2cc1OC)-c1cc2c(cc1C3=O)OCO2] | Retention time: 9.5 min; LC- MS m/z 1194.7 [M + 1]⁺. |
| Compound 61 | 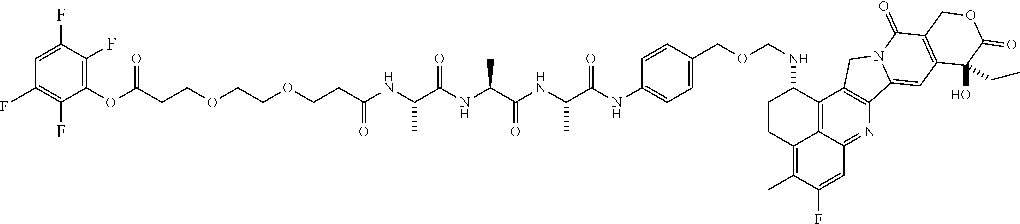 | Retention time: 9.564 min; LC- MS m/z 1135.4 [M + 1]⁺. |
| Compound 63 | 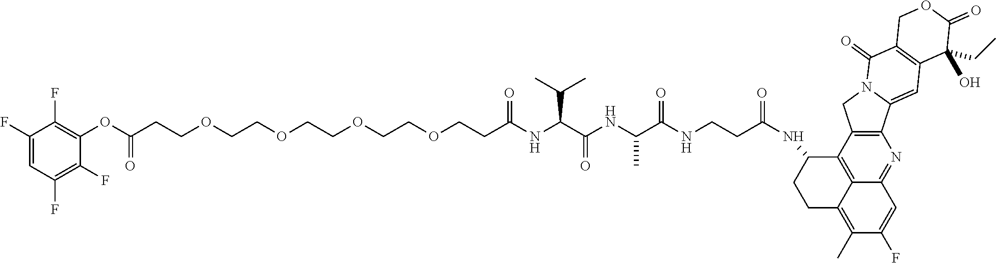 | Retention time: 9.8 min; LC- MS m/z 1101.8 [M + 1]⁺. |
| Compound 64 | 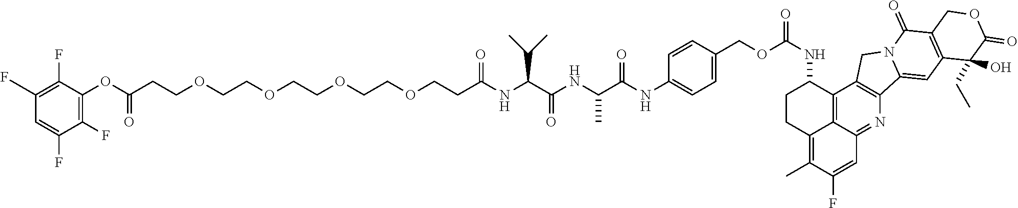 | Retention time: 3.575 min; LC- MS m/z 1153.7 [M + 1]⁺. |
| Compound 70 | 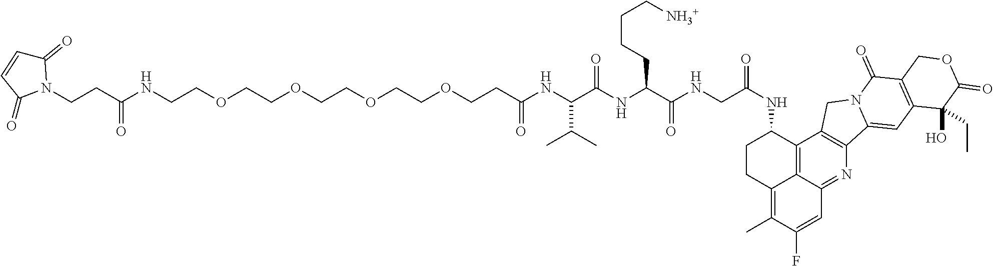 [SMILES: CC[C@@]1(O)C(=O)OCc2c1cc1n(c2=O)Cc2c-1nc1cc(F)c(C)c3c1c2[C@@H](NC(=O)CNC(=O)[C@H](CCCC[NH3+])NC(=O)[C@@H](NC(=O)CCOCCOCCOCCOCCNC(=O)CCN1C(=O)C=CC1=O)C(C)C)CC3] | Retention time: 0.783 min; LC- MS m/z 1119.0 [M + 1]⁺. |
| Compound 71 | 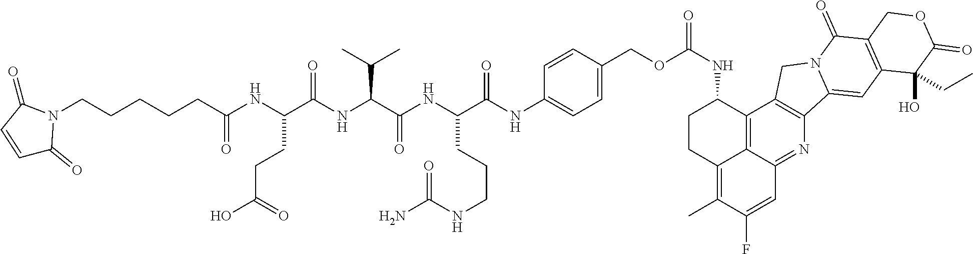 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)[C@H](CCC(=O)O)NC(=O)CCCCCN2C(=O)C=CC2=O)C(C)C)cc1)CC3] | Retention time: 7.181 min; LC- MS m/z 1163.7 [M + 1]⁺. |
| Compound 72 | 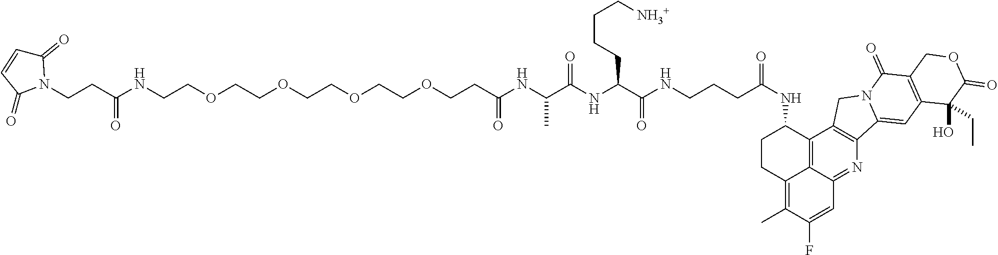 [SMILES: CC[C@@]1(O)C(=O)OCc2c1cc1n(c2=O)Cc2c-1nc1cc(F)c(C)c3c1c2[C@@H](NC(=O)CCCNC(=O)[C@H](CCCC[NH3+])NC(=O)[C@H](C)NC(=O)CCOCCOCCOCCOCCNC(=O)CCN1C(=O)C=CC1=O)CC3] | Retention time: 8.855 min; LC- MS m/z 1118.8 [M + 1]⁺. |
| Compound 75 | 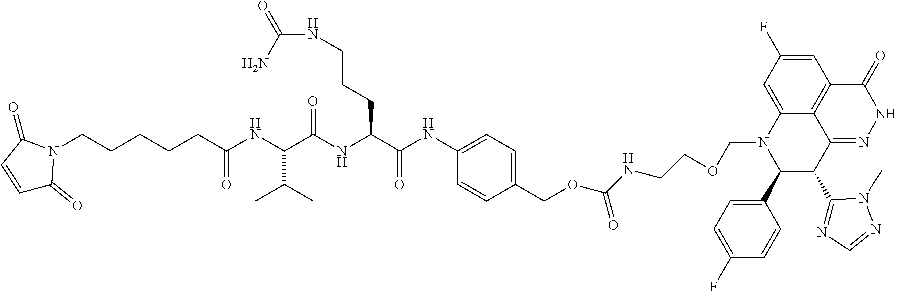 [SMILES: CC(C)[C@H](NC(=O)CCCCCN1C(=O)C=CC1=O)C(=O)N[C@@H](CCCNC(N)=O)C(=O)Nc1ccc(COC(=O)NCCOCN2c3cc(F)cc4c(=O)[nH]nc(c34)[C@H](c3ncnn3C)[C@H]2c2ccc(F)cc2)cc1] | Retention time: 3.748 min; LC- MS m/z 1052.6 [M + 1]⁺. |
| Compound 76 | 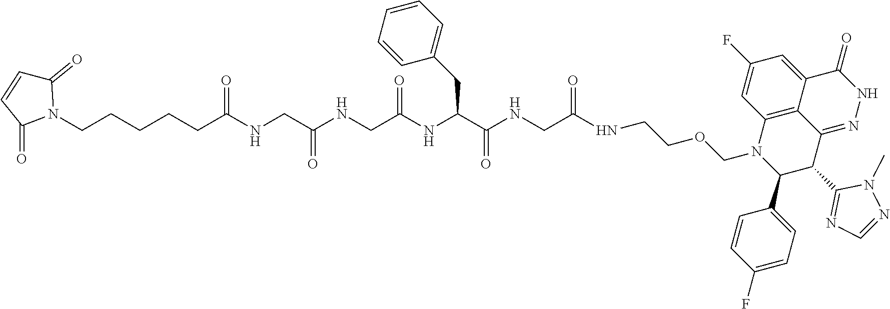 [SMILES: Cn1ncnc1[C@H]1c2n[nH]c(=O)c3cc(F)cc(c23)N(COCCNC(=O)CNC(=O)[C@H](Cc2ccccc2)NC(=O)CNC(=O)CNC(=O)CCCCCN2C(=O)C=CC2=O)[C@@H]1c1ccc(F)cc1] | Retention time: 3.006 min; LC- MS m/z 965.4 [M + 1]⁺. |
| Compound 86 | 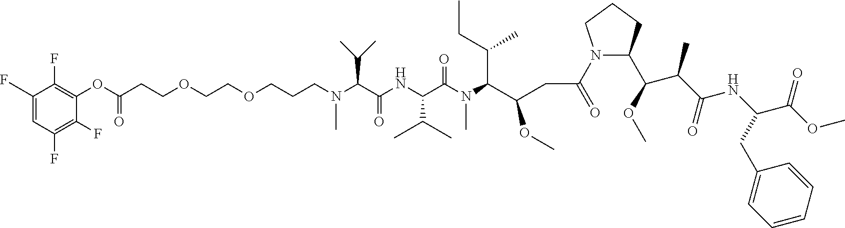 [SMILES: CC[C@H](C)[C@@H]([C@@H](CC(=O)N1CCC[C@H]1[C@H](OC)[C@@H](C)C(=O)N[C@@H](Cc1ccccc1)C(=O)OC)OC)N(C)C(=O)[C@@H](NC(=O)[C@H](C(C)C)N(C)CCCOCCOCCC(=O)Oc1c(F)c(F)cc(F)c1F)C(C)C] | Retention time: 3.932 min; LC- MS m/z 1068.5 [M + 1]⁺. |
| Compound 87 | 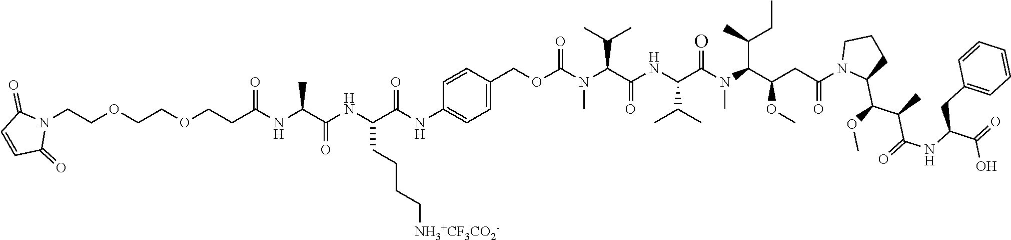 | Retention time: 2.920 min; LC- MS m/z 1319.6 [M + 1]⁺. |
| Compound 89 | 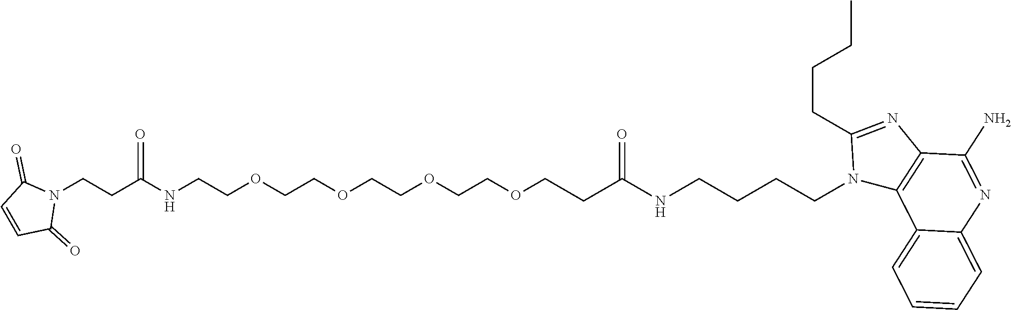 [SMILES: CCCCc1nc2c(N)nc3ccccc3c2n1CCCCNC(=O)CCOCCOCCOCCOCCNC(=O)CCN1C(=O)C=CC1=O] | Retention time: 1.213 min; LC- MS m/z 710.4 [M + 1]⁺. |
| Compound 90 | 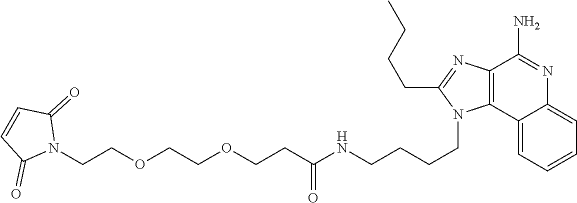 [SMILES: CCCCc1nc2c(N)nc3ccccc3c2n1CCCCNC(=O)CCOCCOCCN1C(=O)C=CC1=O] | Retention time: 3.036 min; LC- MS m/z 551.4 [M + 1]⁺. |
| Compound 91 | 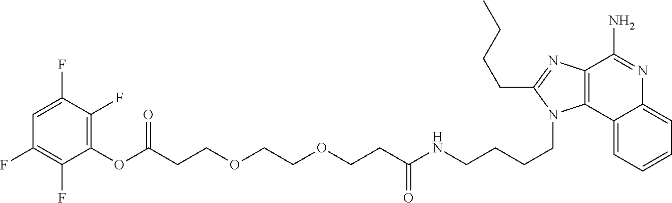 [SMILES: CCCCc1nc2c(N)nc3ccccc3c2n1CCCCNC(=O)CCOCCOCCC(=O)Oc1c(F)c(F)cc(F)c1F] | Retention time: 2.831 min; LC- MS m/z 648.3 [M + 1]⁺. |
| Compound 93 | 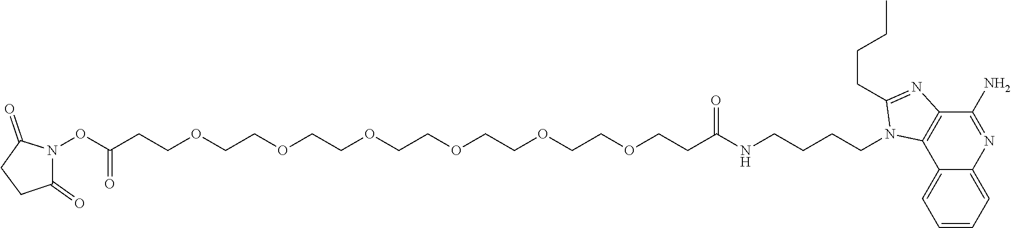 [SMILES: CCCCc1nc2c(N)nc3ccccc3c2n1CCCCNC(=O)CCOCCOCCOCCOCCOCCOCCC(=O)ON1C(=O)CCC1=O] | Retention time: 3.766 min; LC- MS m/z 773.3 [M + 1]⁺. |
| Compound 94 | 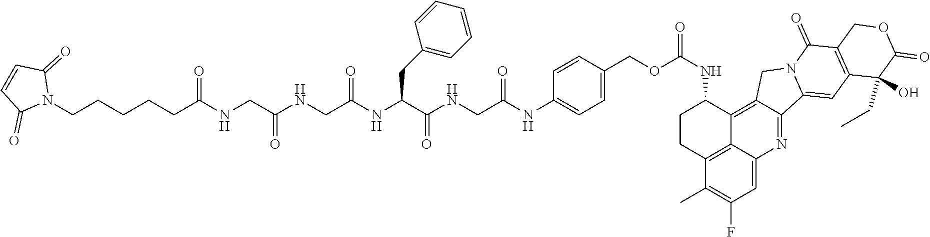 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)CNC(=O)[C@H](Cc2ccccc2)NC(=O)CNC(=O)CNC(=O)CCCCCN2C(=O)C=CC2=O)cc1)CC3] | Retention time: 3.049 min; LC- MS m/z 1096.4 [M + 1]⁺. |
| Compound 95 | 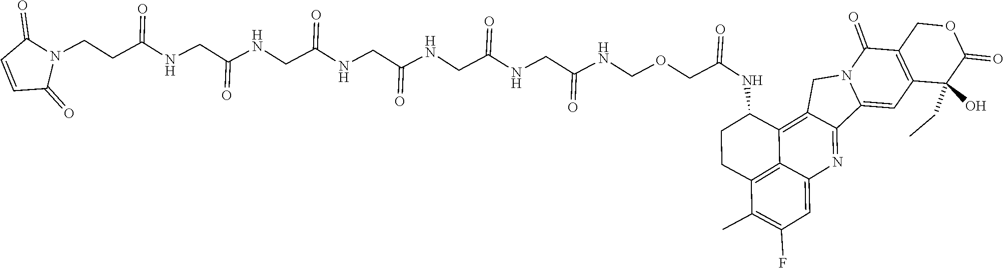 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)CNC(=O)CNC(=O)CNC(=O)CNC(=O)CCN1C(=O)C=CC1=O)CC3] | Retention time: 2.229 min; LC- MS m/z 959.4 [M + 1]⁺. |
| Compound 96 | 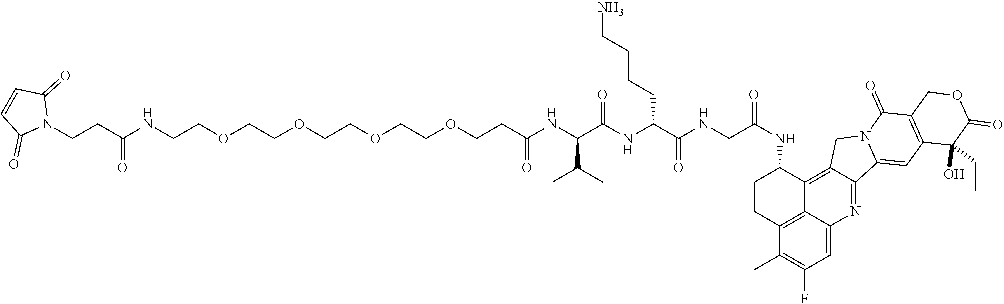 [SMILES: CC[C@@]1(O)C(=O)OCc2c1cc1n(c2=O)Cc2c-1nc1cc(F)c(C)c3c1c2[C@@H](NC(=O)CNC(=O)[C@@H](CCCC[NH3+])NC(=O)[C@H](NC(=O)CCOCCOCCOCCOCCNC(=O)CCN1C(=O)C=CC1=O)C(C)C)CC3] | Retention time: 0.783 min; LC- MS m/z 1119.0 [M + 1]⁺. |
| Compound 97 | 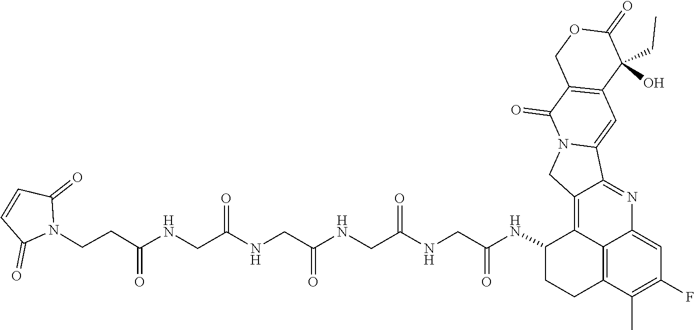 [SMILES: CC[C@@]1(O)C(=O)OCc2c1cc1n(c2=O)Cc2c-1nc1cc(F)c(C)c3c1c2[C@@H](NC(=O)CNC(=O)CNC(=O)CNC(=O)CNC(=O)CCN1C(=O)C=CC1=O)CC3] | Retention time: 2.389 min; LC- MS m/z 815.3 [M + 1]⁺. |
| Compound 98 | 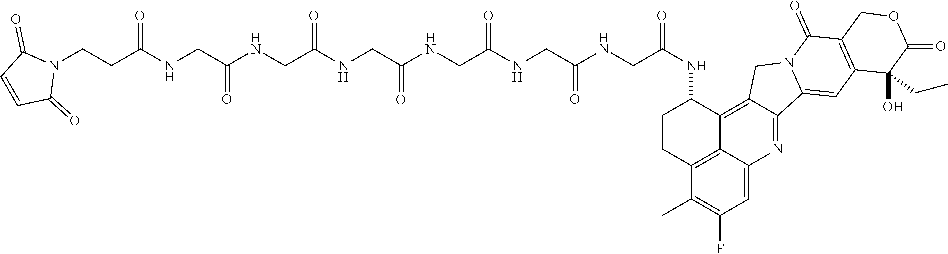 [SMILES: CC[C@@]1(O)C(=O)OCc2c1cc1n(c2=O)Cc2c-1nc1cc(F)c(C)c3c1c2[C@@H](NC(=O)CNC(=O)CNC(=O)CNC(=O)CNC(=O)CNC(=O)CNC(=O)CCN1C(=O)C=CC1=O)CC3] | Retention time: 2.051 min; LC- MS m/z 929.3 [M + 1]⁺. |
| Compound 99 | 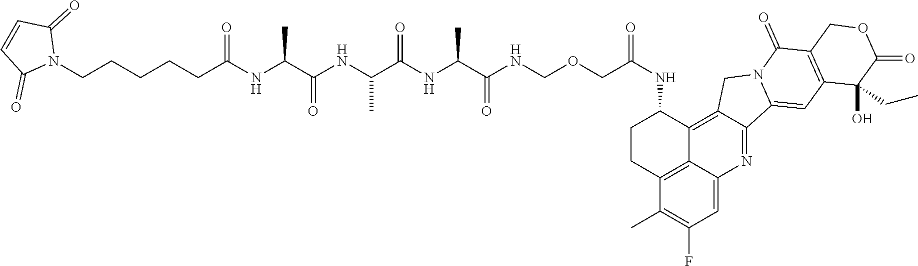 [SMILES: CC[C@@]1(O)C(=O)OCc2c1cc1n(c2=O)Cc2c-1nc1cc(F)c(C)c3c1c2[C@@H](NC(=O)COCNC(=O)[C@H](C)NC(=O)[C@H](C)NC(=O)[C@H](C)NC(=O)CCCCCN1C(=O)C=CC1=O)CC3] | Retention time: 2.543 min; LC- MS m/z 929.5 [M + 1]⁺. |
| Compound 100 | 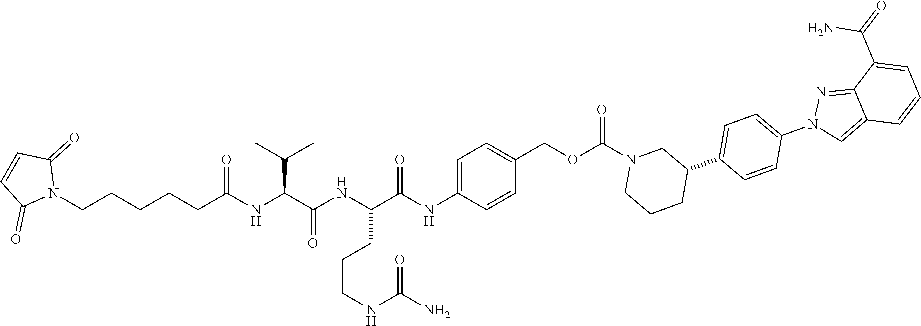 [SMILES: CC(C)[C@H](NC(=O)CCCCCN1C(=O)C=CC1=O)C(=O)N[C@@H](CCCNC(N)=O)C(=O)Nc1ccc(COC(=O)N2CCC[C@@H](c3ccc(-n4cc5cccc(C(N)=O)c5n4)cc3)C2)cc1] | Retention time: 0.985 min; LC- MS m/z 919.4 [M + 1]⁺. |
| Compound 101 | 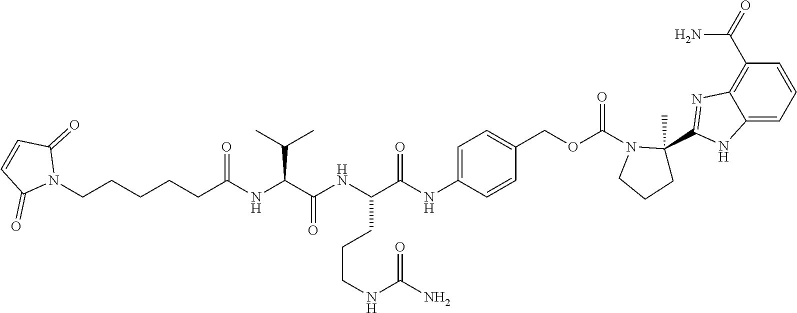 [SMILES: CC(C)[C@H](NC(=O)CCCCCN1C(=O)C=CC1=O)C(=O)N[C@@H](CCCNC(N)=O)C(=O)Nc1ccc(COC(=O)N2CCC[C@]2(C)c2nc3c(C(N)=O)cccc3[nH]2)cc1] | Retention time: 1.925 min; LC- MS m/z 865.5 [M + 1]⁺. |
| Compound 102 | 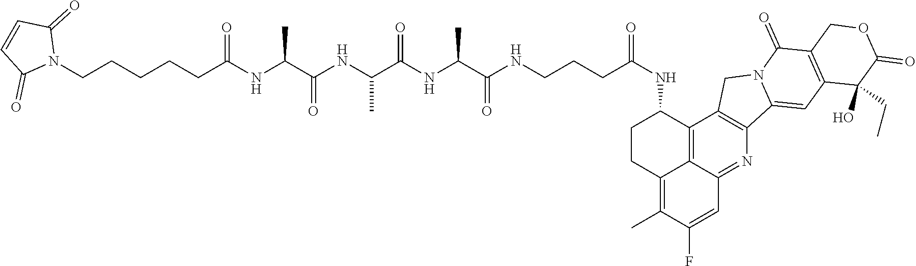 [SMILES: CC[C@@]1(O)C(=O)OCc2c1cc1n(c2=O)Cc2c-1nc1cc(F)c(C)c3c1c2[C@@H](NC(=O)CCCNC(=O)[C@H](C)NC(=O)[C@H](C)NC(=O)[C@H](C)NC(=O)CCCCCN1C(=O)C=CC1=O)CC3] | Retention time: 5.8 min; LC- MS m/z 929.7 [M + 1]⁺. |
| Compound 103 | 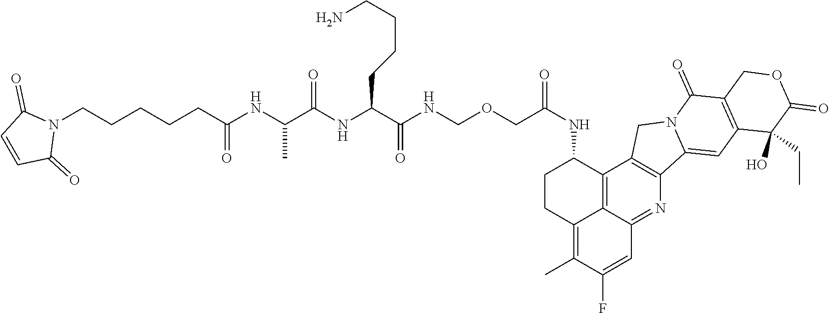 [SMILES: CC[C@@]1(O)C(=O)OCc2c1cc1n(c2=O)Cc2c-1nc1cc(F)c(C)c3c1c2[C@@H](NC(=O)COCNC(=O)[C@H](CCCCN)NC(=O)[C@H](C)NC(=O)CCCCCN1C(=O)C=CC1=O)CC3] | Retention time: 9.485 min; LC- MS m/z 915.9 [M + 1]⁺. |
| Compound 104 | 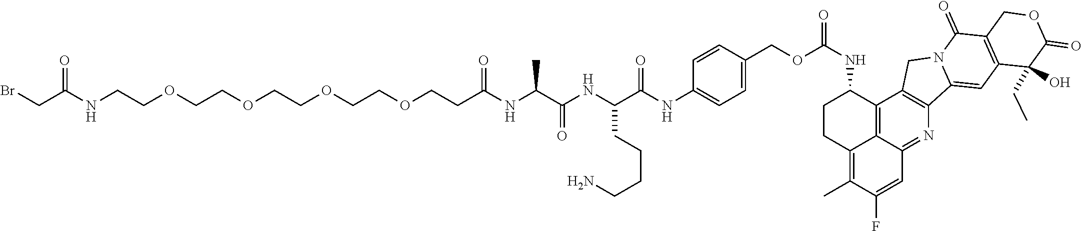 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCCN)NC(=O)[C@H](C)NC(=O)CCOCCOCCOCCOCCNC(=O)CBr)cc1)CC3] | Retention time: 10.6 min; LC- MS m/z 1153.4 [M + 1]⁺. |

### II. Conjugations Examples

### Example II-1: Conjugation of Link-Payload Compounds to Anti-ICAM1 or Anti-EphA2 Antibodies

Different methods were used for conjugation of cysteine-specific compounds (e.g., compounds with a terminal maleimide or dibromomaleimide reactor group depending on the desired DAR and homogeneity (see Schemes 9A-9C). All lysine specific compounds (e.g., compounds with a terminal tetrafluorophenyl ester reactor group) were conjugated using the procedure as shown in Scheme 10.

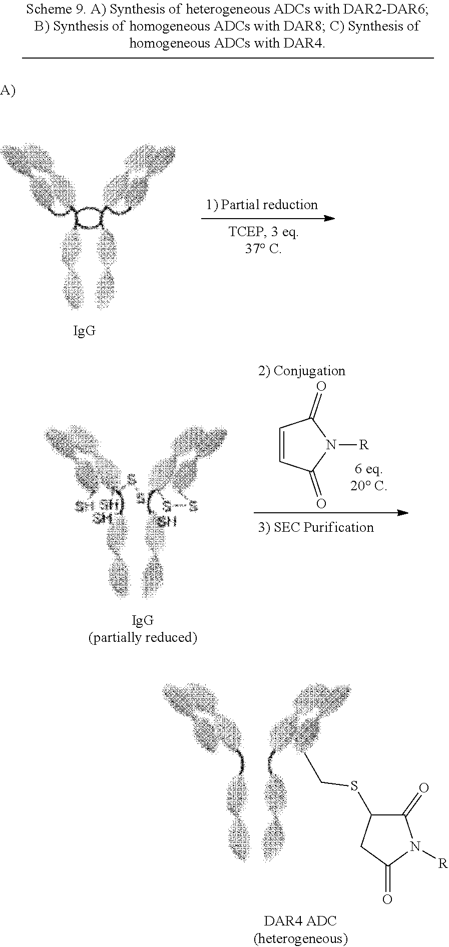

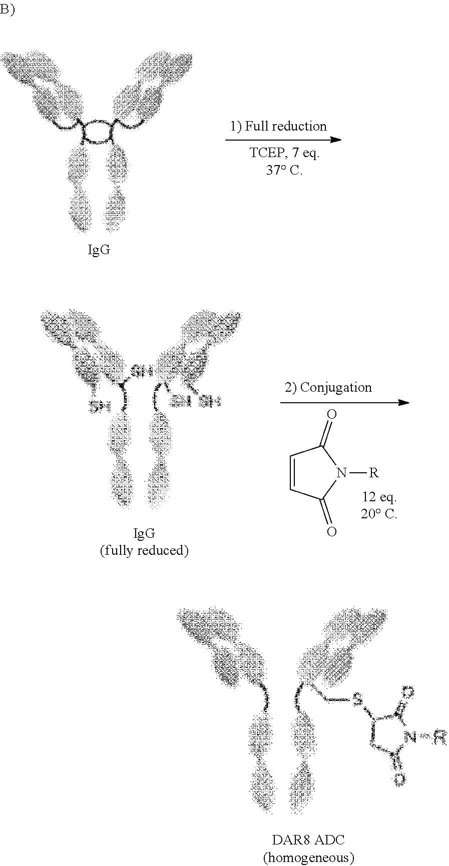

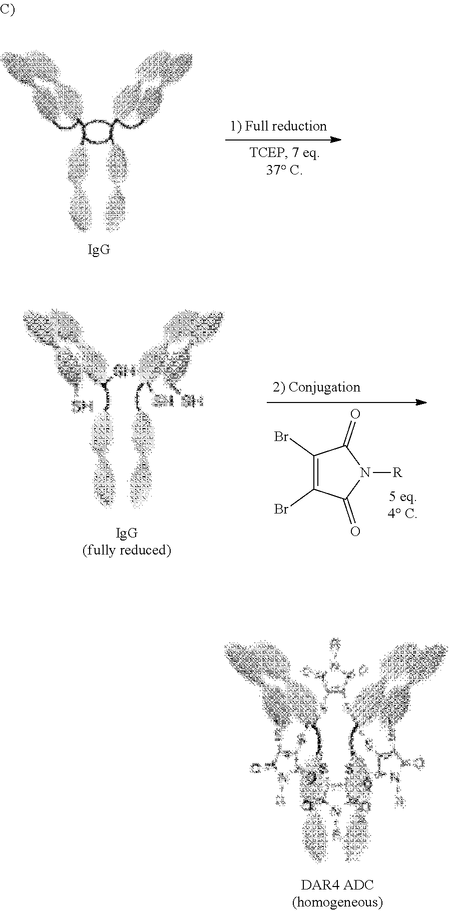

Conjugation with Cysteine Residues on Antibody

### A) Synthesis of Heterogeneous DAR4 Antibody-Drug-Conjugates (ADCs) Using Compounds 1-18 and 23-28.

All Buffers should be degassed & sterilized via filtration through a 0.2 micron filter prior to use. The antibody was buffer exchanged into PBS (pH 7.2) using a GE PD10 column (2.5 mL) and diluted to final concentration of 5 mg/mL (~33 μM) in PBS. The antibody solution was warmed to 37° C. in a heat block or water bath. A 20 mM stock solution of TCEP in water was prepared fresh and 3 molar equivalents (relative to the IgG) was added to the warmed antibody. The mixture was incubated for 2 hr at 37° C., removed from heat and allowed to cool to room temperature (~20° C.). A 5 mM stock solution of the maleimide linker-payload compound (e.g., Compounds 1-18 or 23-28) in DMSO (or DMA) was prepared and 6 molar equiv. of the maleimide linker-payload compound was added to the partially reduced antibody. After incubation at 20° C. for 1 hr, the resulting Antibody-Drug-Conjugate (ADC) was buffer exchanged into PBS using a disposable GE PD10 spin column (2.5 mL) and stored at −20° C. until needed. Monomeric purity of the ADC was determined via analytical SEC and the DAR was determined via LC/MS.

### B) Synthesis of Homogeneous DAR8 Antibody-Drug-Conjugates (ADCs) Using Compounds 1-18 and 23-28.

This protocol B) is similar to the protocol A) except that excess reagents are used to conjugate all 8 available cysteines in the antibody with the maleimide linker-payload compound. Specifically, the antibody was buffer exchanged into PBS (pH 7.2) using a GE PD10 column (2.5 mL) and diluted to final concentration of 5 mg/mL in PBS. The antibody solution was warmed to 37° C. in a heat block or water bath. A 50 mM stock solution of TCEP in water was prepared fresh and 7 molar equivalents (relative to the IgG) was added to the warmed antibody. The mixture was incubated at 37° C. for 2 hr, removed from heat and allowed to cool to room temperature (~20° C.). A 5 mM stock solution of the maleimide linker-payload compound (e.g., Compounds 1-18 or 23-28) in DMSO (or DMA) was prepared and 12 molar eq of the payload was added to the fully reduced antibody. After incubation at 20° C. for 1 hr, the resulting ADC was buffer exchanged into PBS using a disposable GE PD10 spin column (2.5 mL) and stored at −20° C. until needed. Monomeric purity of the ADC was determined via analytical SEC and the DAR was determined via LC/MS. The homogeneity and DAR 8 were confirmed via HIC analysis. The overall yield is typically 50% or higher.

### C) Synthesis of Homogeneous DAR4 Antibody-Drug-Conjugates (ADCs) Using Compounds 33-36.

The IgG1 antibody (trastuzumab) was buffer exchanged into PBS (pH 7.2-7.4) with a GE PD10 column (2.5 mL) using the gravity-based method. The antibody concentration was determined via A280 nm and diluted or concentrated to a final concentration of 5 mg/mL in PBS. The antibody was then warmed to 37° C. in a heat block or water bath. A 50 mM stock solution of TCEP was freshly prepared in water 7 molar equivalents of TCEP (relative to antibody) was added to the antibody. The mixture was incubated at 37° C. for 2-3 hrs. The reduced IgG was removed from the heat and cooled to 4° C. A fresh 5 mM stock solution of the DBM linker-payload compound (e.g., Compounds 33-36) in DMSO was prepared and 5 molar equiv. of the DBM linker-payload compound was added to the reduced antibody and incubated at 4° C. for 1 hr. The resulting ADC was buffer exchanged into PBS using a GE PD10 spin column (2.5 mL). The ADC purity (% monomer) was determined via SEC and DAR was determined via LC/MS and confirmed with HIC. The purified ADC was aliquoted, frozen and stored at −20° C. or lower until needed.

### D) Synthesis of Antibody-Drug-Conjugates (ADCs) Using Compounds 19-22 and 29-32.

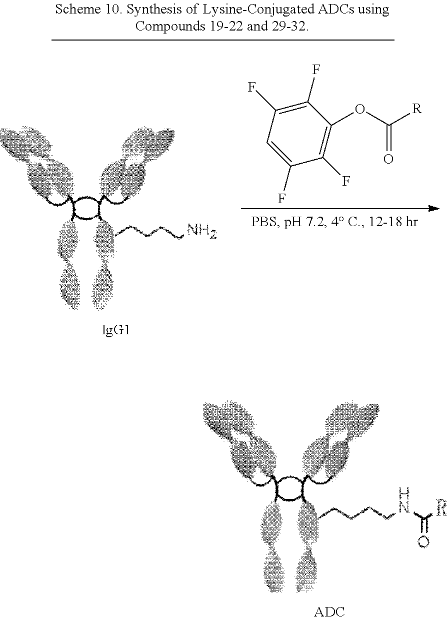

The IgG1 antibody was buffer exchanged into PBS (pH 7.2-7.4) using a GE PD10-spin column (2.5 mL) and diluted to a final concentration of 5 mg/mL in PBS. The antibody stock solution was then filtered through a 0.2 μm sterile syringe filter and stored at 4° C. until needed. The TFP linker-payload compound (e.g., Compounds 19-22 or 29-32) was dissolved in anhydrous DMSO to make a 20 mM stock solution. Eight molar equivalents of the TFP linker-payload compound (relative to IgG) is added to 10 mg of antibody. The tube is mixed gently and incubated overnight at 4° C. The conjugation reaction is monitored via LC/MS until desired DAR is reached.

The resulting ADC was buffer exchanged (GE PD10 spin column) to remove low molecular weight impurities. The purity and % monomer were determined by SEC (Agilent 1100 HPLC system with a BEH200 SEC 1.7 μm, 4.6×300 mm column equipped with a BEH SEC Guard column 4.6×30 mM (Waters)). The mobile phase consisted of 100 mM sodium phosphate, 100 mM sodium sulfate, and 10% IPA, pH 6.8. 5 μg of sample was injected and run at a flow rate of 0.3 mL/min at room temperature for 15 minutes. The molecular and DAR was determined via LC/MS. Samples were deglycosylated for 5 hr at 37° C. with PNGase prior to analysis.

### Example II-2: Synthesis of Conjugate IC-1 (DAR5 and DAR8)

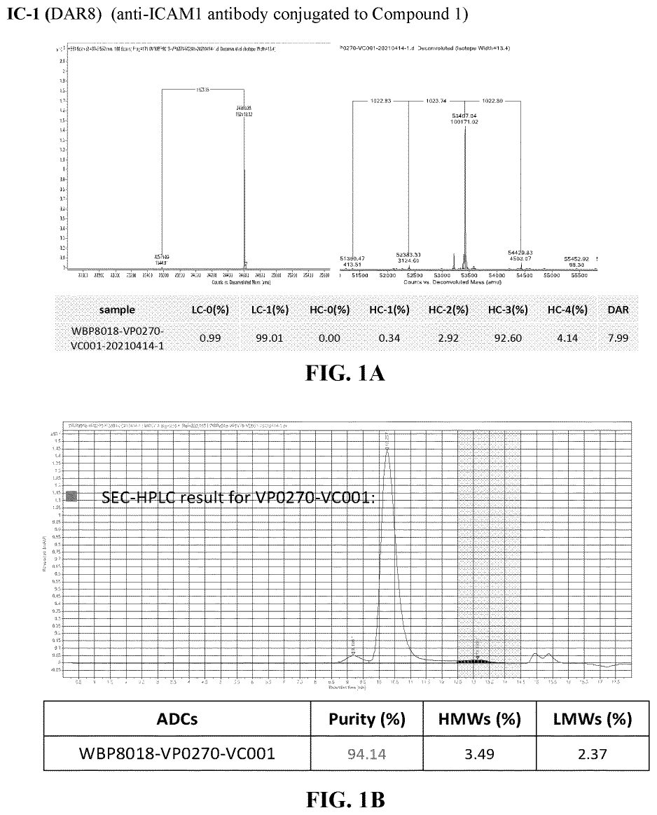

Conjugate IC-1 (DAR8) was synthesized according to Protocol B) above in Example II-1 using Compound 1 and ICAM1 specific IgG1 antibody VP0270. The afforded Conjugate IC-1 (DAR8) was analyzed using LC/MS to calculate the DAR and using SEC-HPLC for purity analysis, as shown in FIGS. 1A-1B.

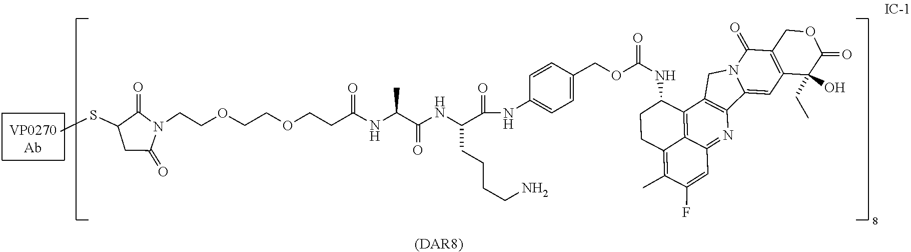

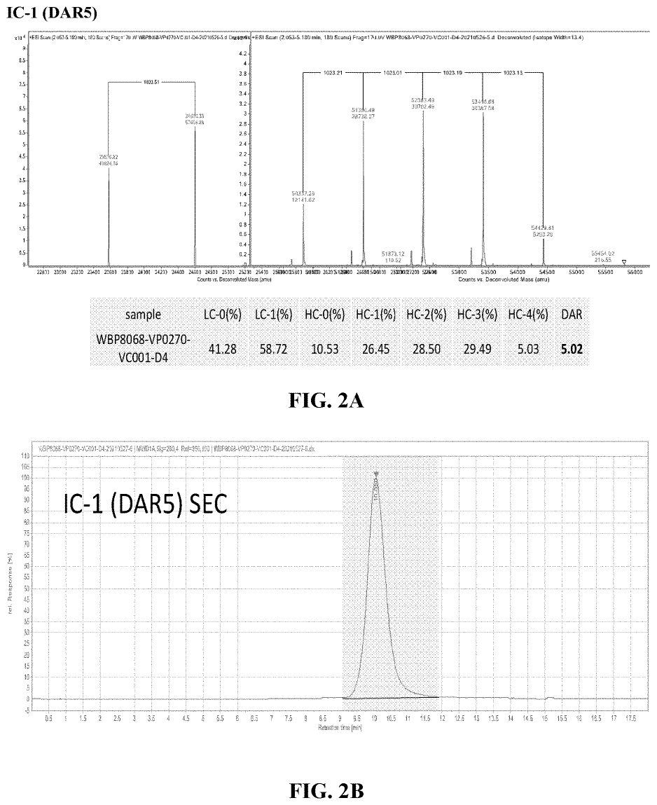

Conjugate IC-1 (DAR5) was synthesized according to Protocol A) above in Example II-1 using Compound 1 and ICAM1 specific IgG1 antibody VP0270. The afforded Conjugate IC-1 (DAR5) was analyzed using LC/MS to calculate the DAR and using SEC-HPLC for purity analysis, as shown in FIGS. 2A-2B.

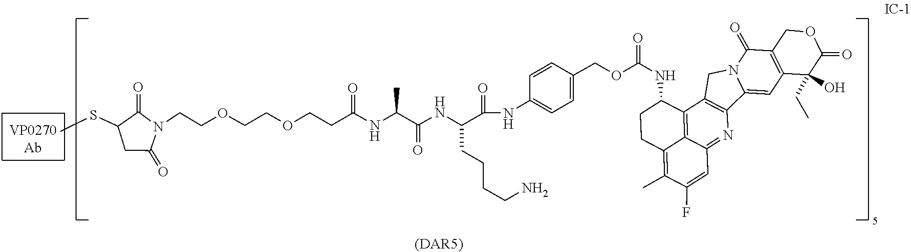

### Example II-3: Synthesis of Conjugate IC-3

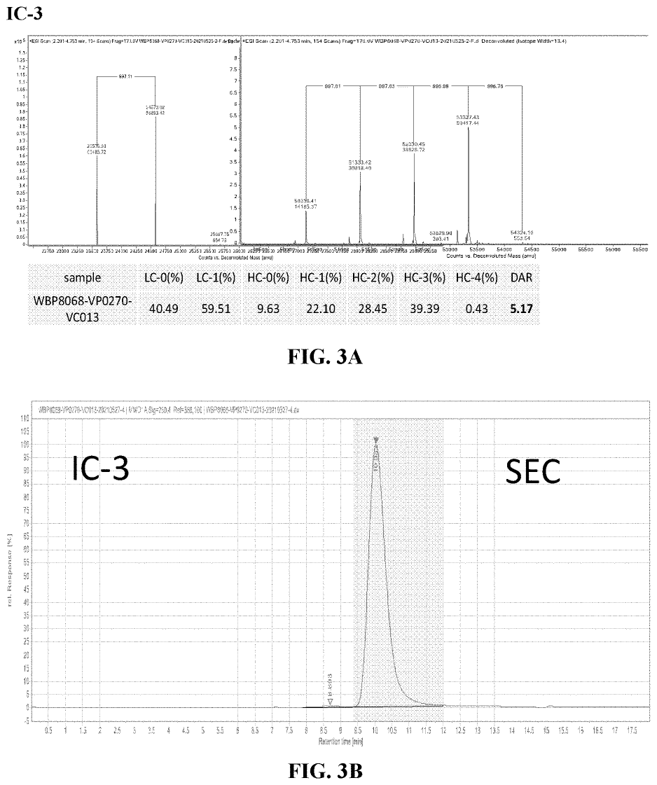

Conjugate IC-3 was synthesized according to Protocol A) above in Example II-1 using Compound 3 and ICAM1 specific IgG1 antibody VP0270. The afforded Conjugate IC-3 was analyzed using LC/MS to calculate the DAR and using SEC-HPLC for purity analysis, as shown in FIGS. 3A-3B.

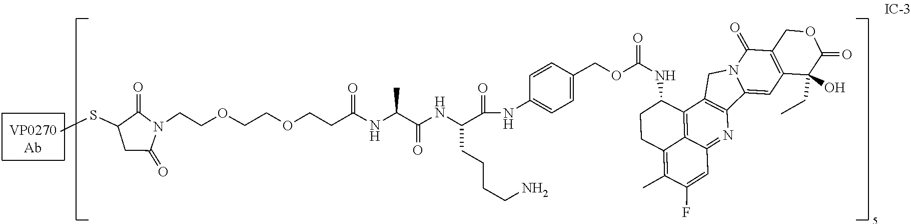

### Example II-4: Synthesis of Conjugate IC-4

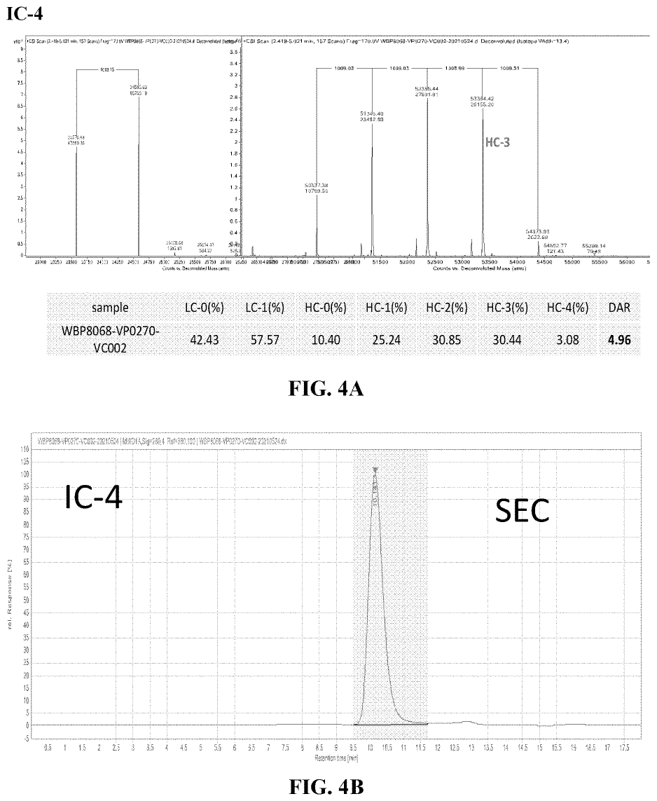

Conjugate IC-4 was synthesized according to Protocol A) above in Example II-1 using Compound 4 and ICAM1 specific IgG1 antibody VP0270. The afforded Conjugate IC-4 was analyzed using LC/MS to calculate the DAR and using SEC-HPLC for purity analysis, as shown in FIGS. 4A-4B.

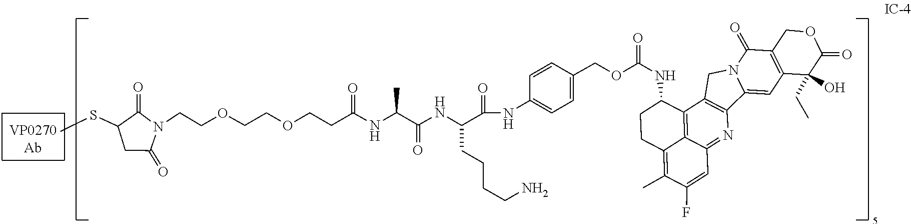

### Example II-5: Synthesis of Conjugate IC-7

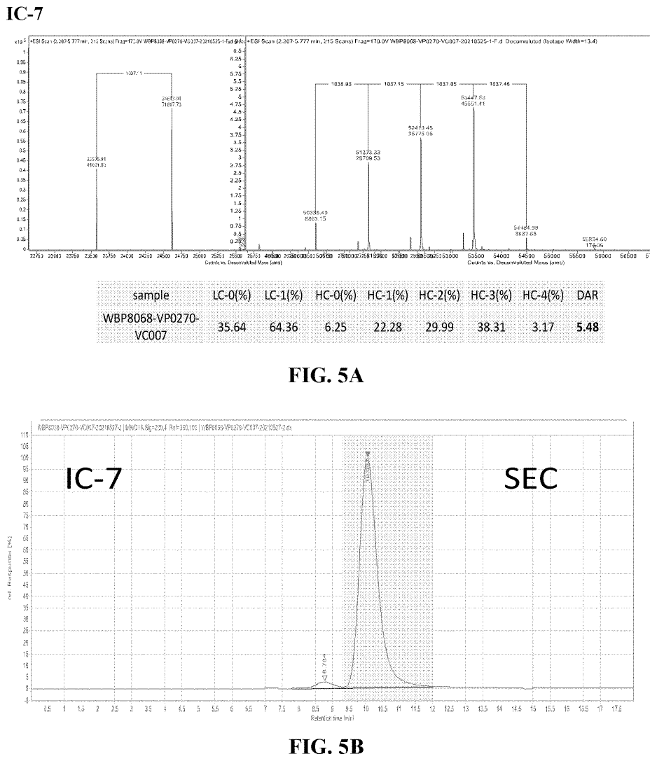

Conjugate IC-7 was synthesized according to Protocol A) above in Example II-1 using Compound 7 and ICAM1 specific IgG1 antibody VP0270. The afforded Conjugate IC-7 was analyzed using LC/MS to calculate the DAR and using SEC-HPLC for purity analysis, as shown in FIGS. 5A-5B.

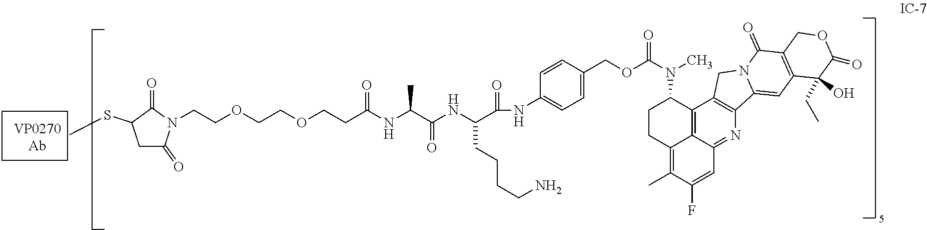

### Example II-6: Synthesis of Conjugate IC-9

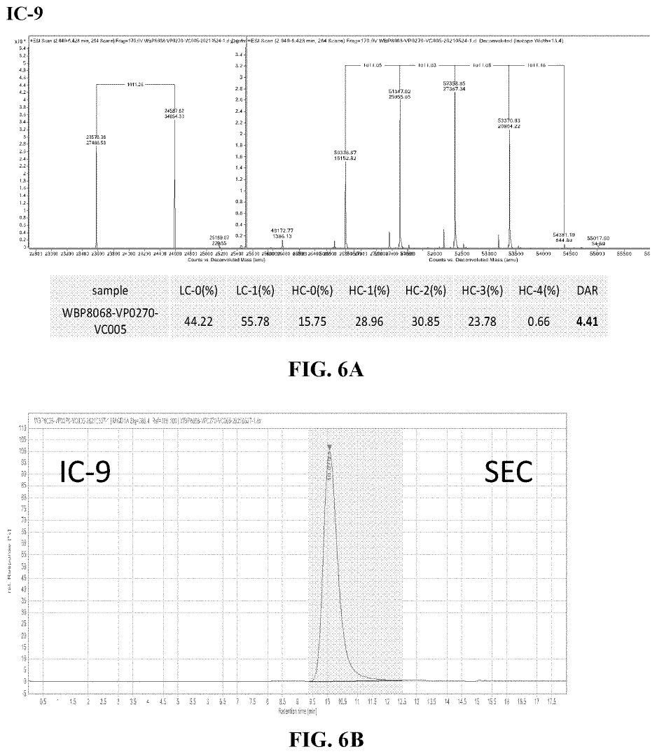

Conjugate IC-9 was synthesized according to Protocol A) above in Example II-1 using Compound 9 and ICAM1 specific IgG1 antibody VP0270. The afforded Conjugate IC-9 was analyzed using LC/MS to calculate the DAR and using SEC-HPLC for purity analysis, as shown in FIGS. 6A-6B.

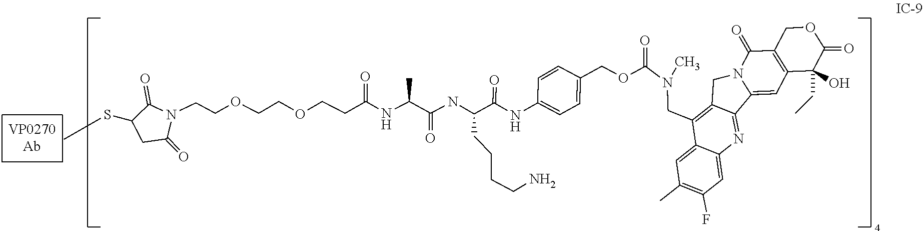

### Example II-7: Synthesis of Conjugate IC-12

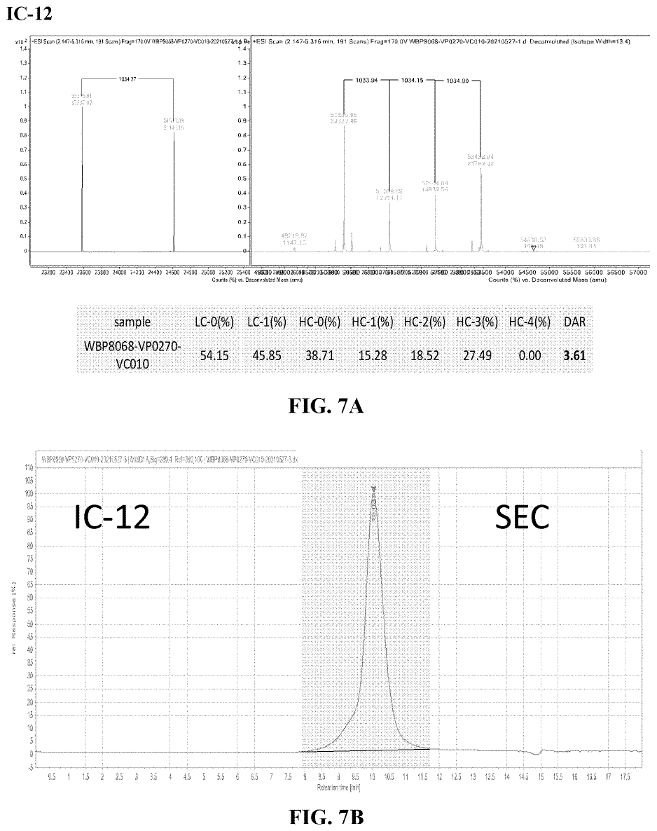

Conjugate IC-12 was synthesized according to Protocol A) above in Example II-1 using Compound 12 and ICAM1 specific IgG1 antibody VP0270. The afforded Conjugate IC-12 was analyzed using LC/MS to calculate the DAR and using SEC-HPLC for purity analysis, as shown in FIGS. 7A-7B.

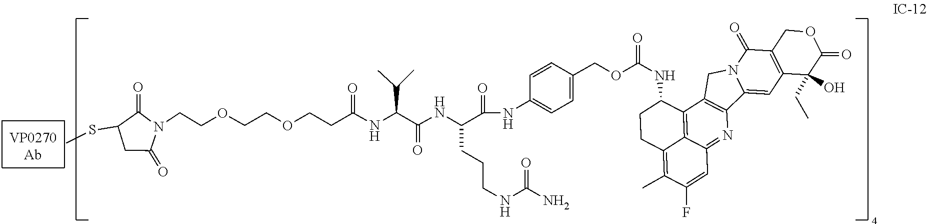

### Example II-8: Synthesis of Conjugate AC-1 (DAR8)

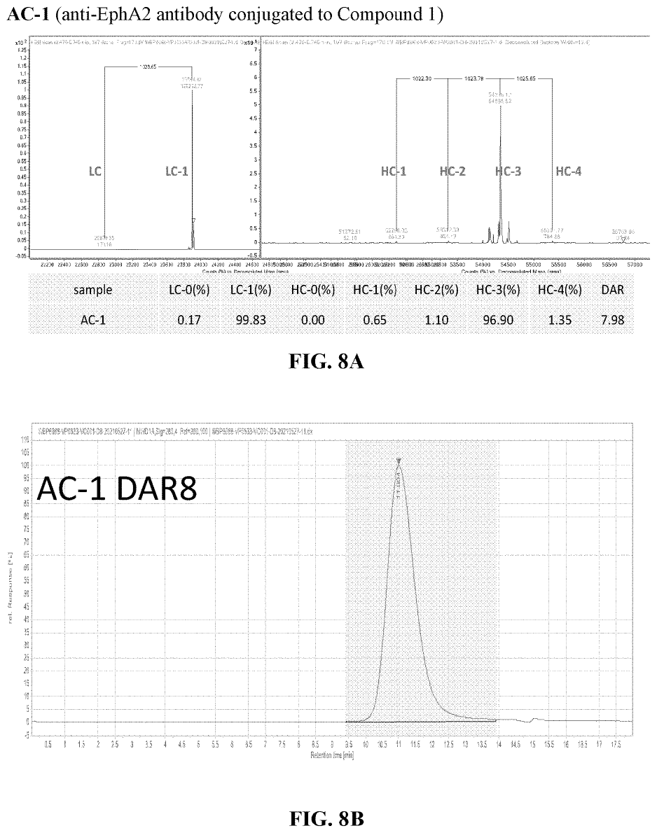

Conjugate AC-1 (VP0633-Compound 1) (DAR8) was synthesized according to Protocol B) above in Example II-1 using Compound 1 and EphA2 specific IgG1 antibody VP0633. The afforded Conjugate AC-1 was analyzed using LC/MS to calculate the DAR and using SEC-HPLC for purity analysis, as shown in FIGS. 8A-8B.

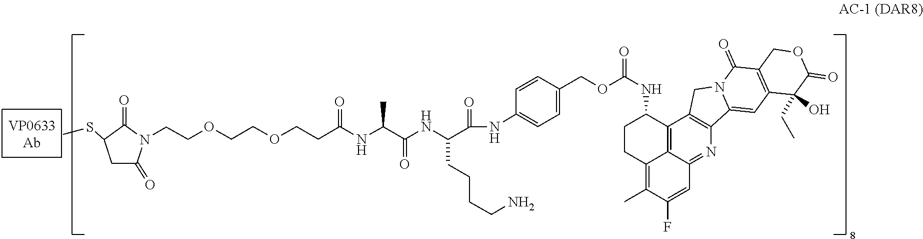

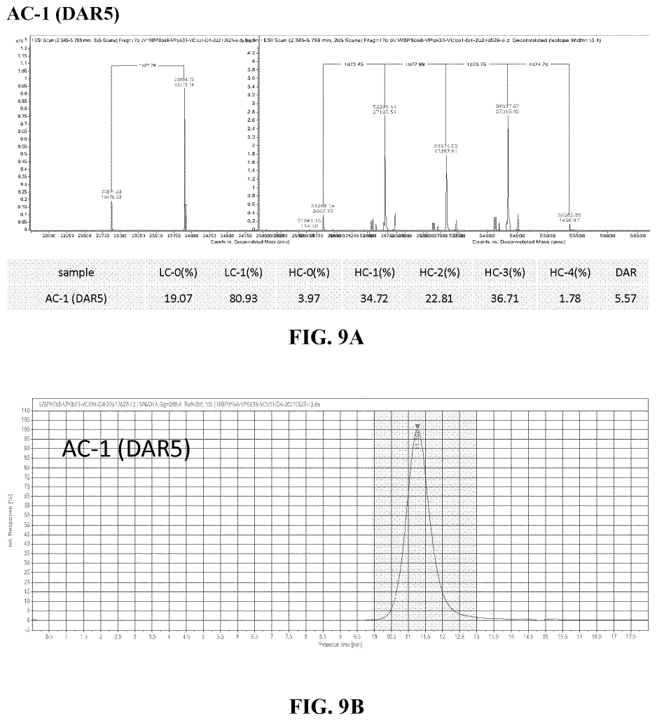

Conjugate AC-1 (DAR5) was synthesized according to Protocol A) above in Example II-1 using Compound 1 and EphA2 specific IgG1 antibody VP0633. The afforded Conjugate AC-1 (DAR5) was analyzed using LC/MS to calculate the DAR and using SEC-HPLC for purity analysis, as shown in FIGS. 9A-9B.

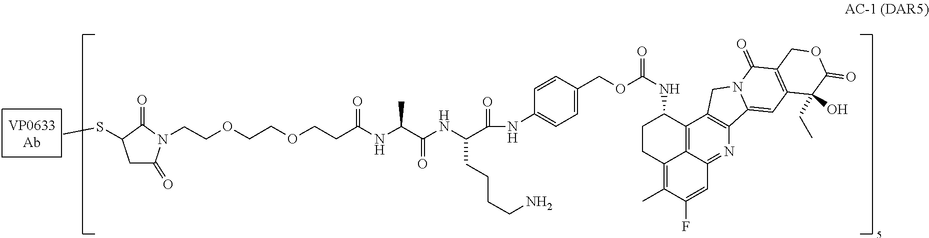

### Example II-9: Synthesis of Conjugate AC-3 (DAR5)

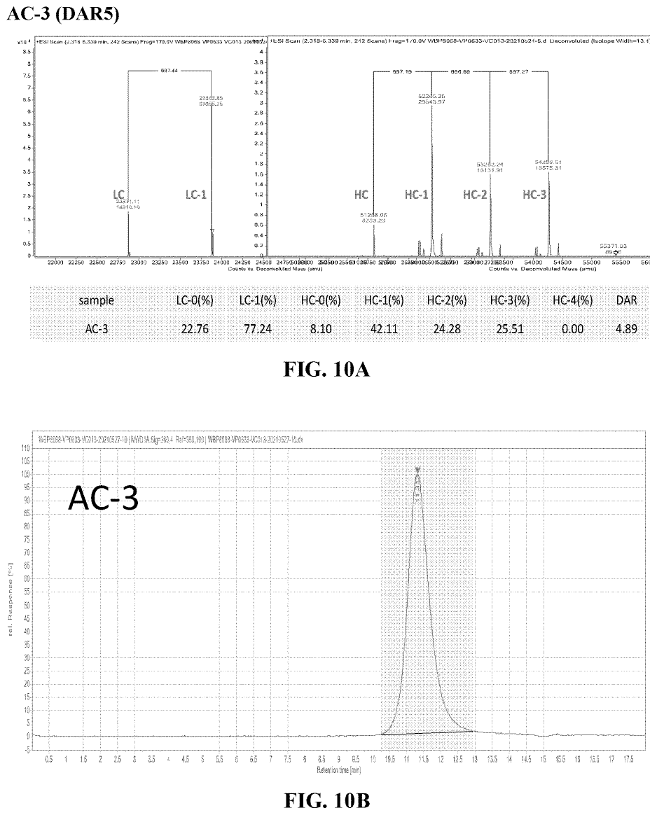

Conjugate AC-3 (DAR5) was synthesized according to Protocol A) above in Example II-1 using Compound 3 and EphA2 specific IgG1 antibody VP0633. The afforded Conjugate AC-3 (DAR5) was analyzed using LC/MS to calculate the DAR and using SEC-HPLC for purity analysis, as shown in FIGS. 10A-10B.

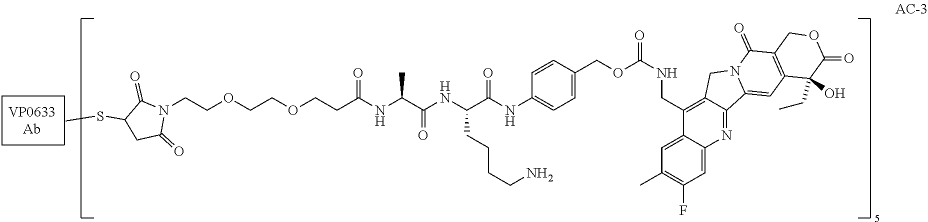

### Example II-10: Synthesis of Conjugate AC-4 (DAR4)

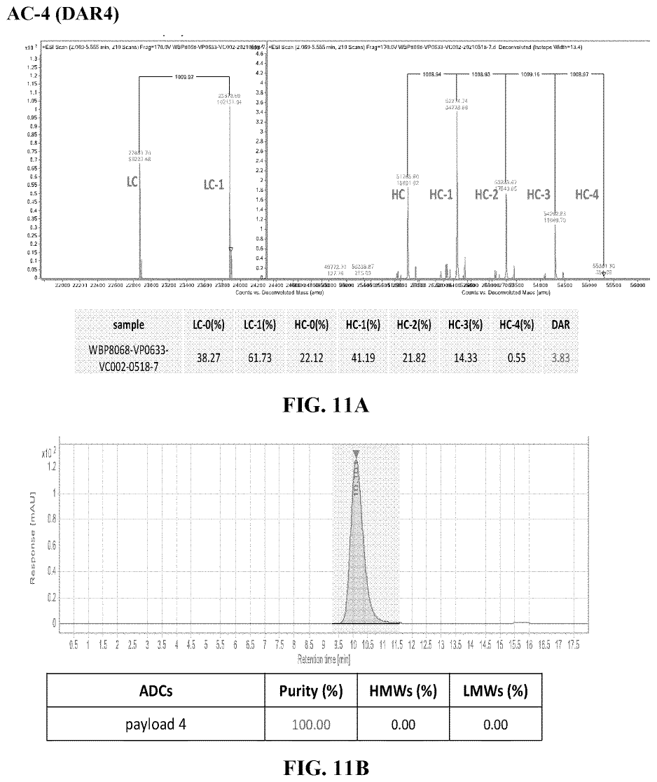

Conjugate AC-4 (DAR4) was synthesized according to Protocol A) above in Example II-1 using Compound 4 and EphA2 specific IgG1 antibody VP0633. The afforded Conjugate AC-4 (DAR4) was analyzed using LC/MS to calculate the DAR and using SEC-HPLC for purity analysis, as shown in FIGS. 11A-11B.

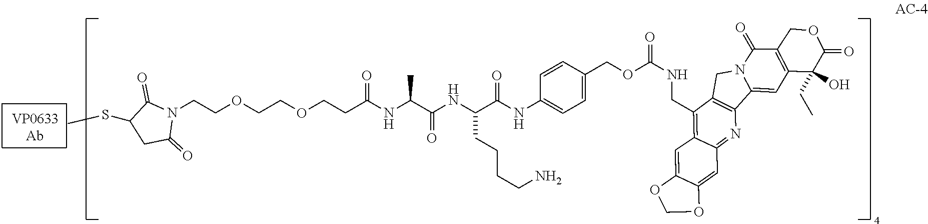

### Example II-11: Synthesis of Conjugate AC-7 (DAR4)

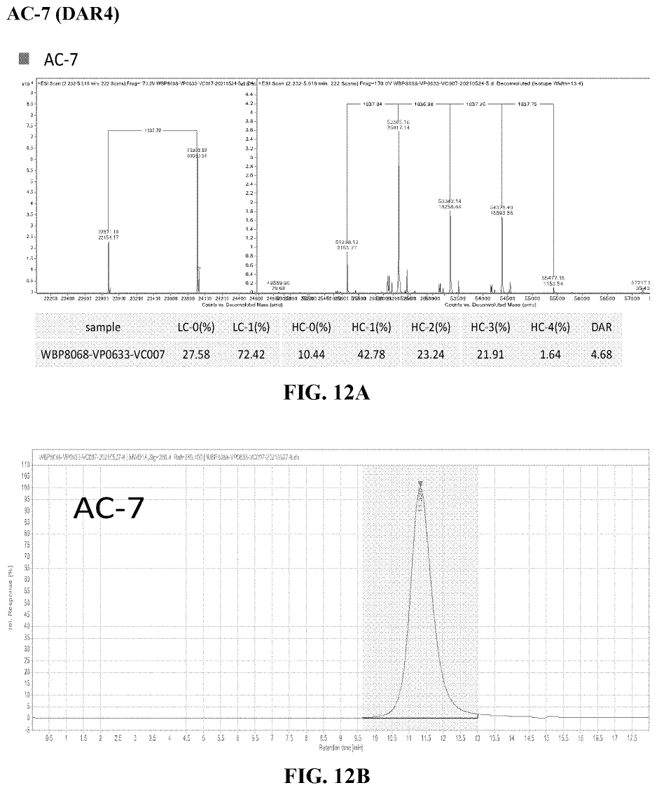

Conjugate AC-7 (DAR4) was synthesized according to Protocol A) above in Example II-1 using Compound 7 and EphA2 specific IgG1 antibody VP0633. The afforded Conjugate AC-7 (DAR4) was analyzed using LC/MS to calculate the DAR and using SEC-HPLC for purity analysis, as shown in FIGS. 12A-12B.

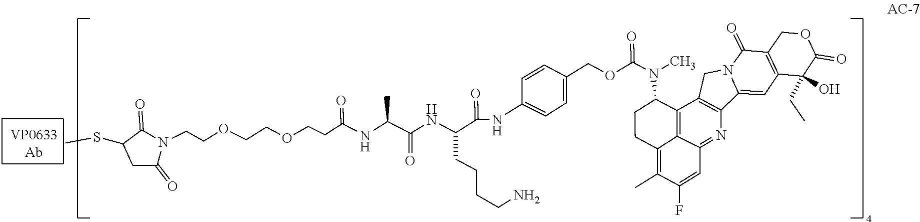

### Example II-12: Synthesis of Conjugate AC-9 (DAR4)

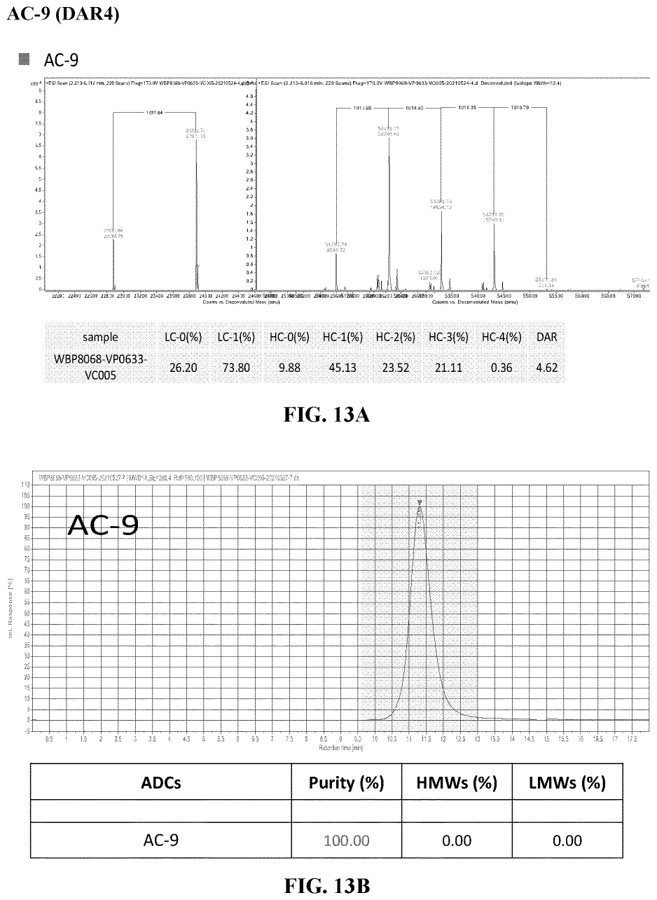

Conjugate AC-9 (DAR4) was synthesized according to Protocol A) above in Example II-1 using Compound 9 and EphA2 specific IgG1 antibody VP0633. The afforded Conjugate AC-9 (DAR4) was analyzed using LC/MS to calculate the DAR and using SEC-HPLC for purity analysis, as shown in FIGS. 13A-13B.

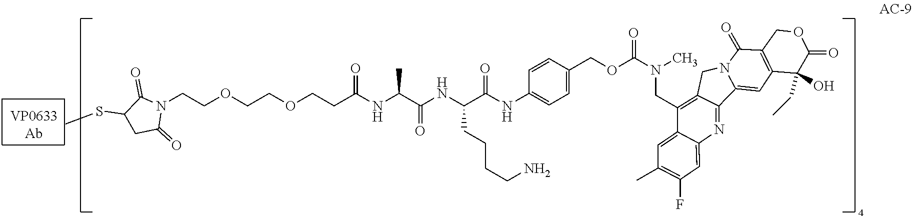

### Example II-13: Synthesis of Conjugate AC-12 (DAR4)

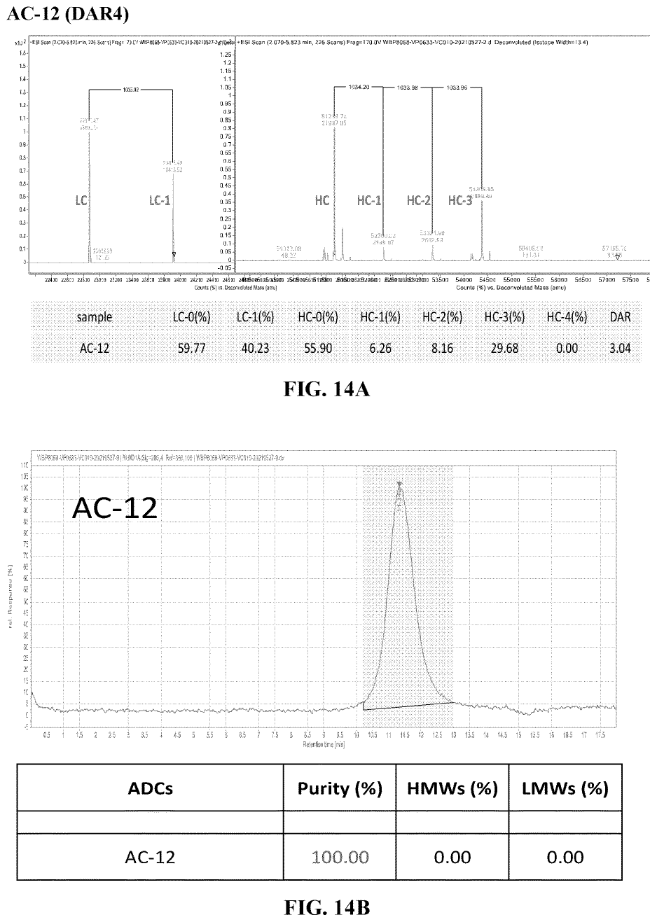

Conjugate AC-12 (DAR4) was synthesized according to Protocol A) above in Example II-1 using Compound 12 and EphA2 specific IgG1 antibody VP0633. The afforded Conjugate AC-12 (DAR4) was analyzed using LC/MS to calculate the DAR and using SEC-HPLC for purity analysis, as shown in FIGS. 14A-14B.

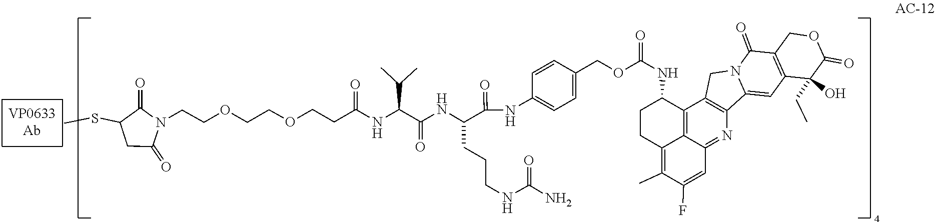

### Example II-14: Synthesis of Conjugate IC-40 (DAR8)

Conjugate IC-40 (VP0270-Compound 40) (DAR8) was synthesized similarly to IC-1 (according to Protocol B) above in Example II-1) using Compound 40 and ICAM1 specific IgG1 antibody VP0270. The afforded Conjugate IC-40 (DAR8) was analyzed using LC/MS to calculate the DAR and using SEC-HPLC for purity analysis.

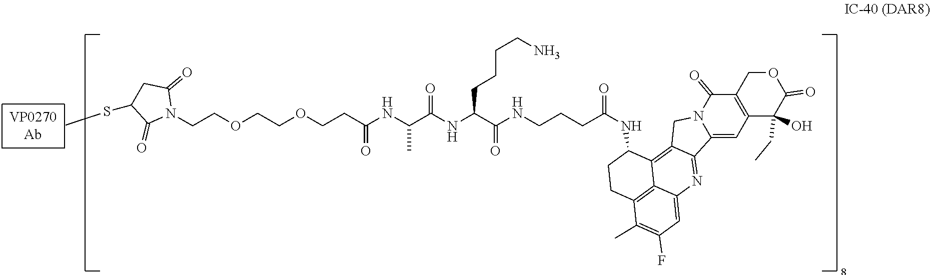

### Example II-15: Synthesis of Conjugate IC-105 (DAR8)

Conjugate IC-105 (VP0270-Compound 105) (DAR8) was synthesized similarly to IC-1 (according to Protocol B) above in Example II-1) using Compound 105 and ICAM1 specific IgG1 antibody VP0270. The afforded Conjugate IC-105 (DAR8) was analyzed using LC/MS to calculate the DAR and using SEC-HPLC for purity analysis.

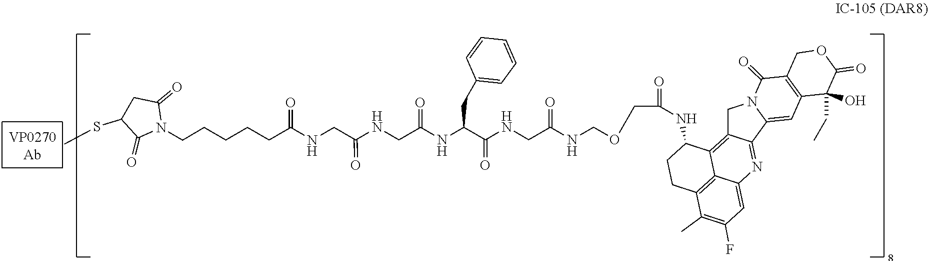

### Example II-16: Synthesis of Conjugate IC-106 (DAR8)

Conjugate IC-106 (VP0270-Compound 106) (DAR8) was synthesized similarly to IC-1 (according to Protocol B) above in Example II-1) using Compound 106 and ICAM1 specific IgG1 antibody VP0270. The afforded Conjugate IC-106 (DAR8) was analyzed using LC/MS to calculate the DAR and using SEC-HPLC for purity analysis.

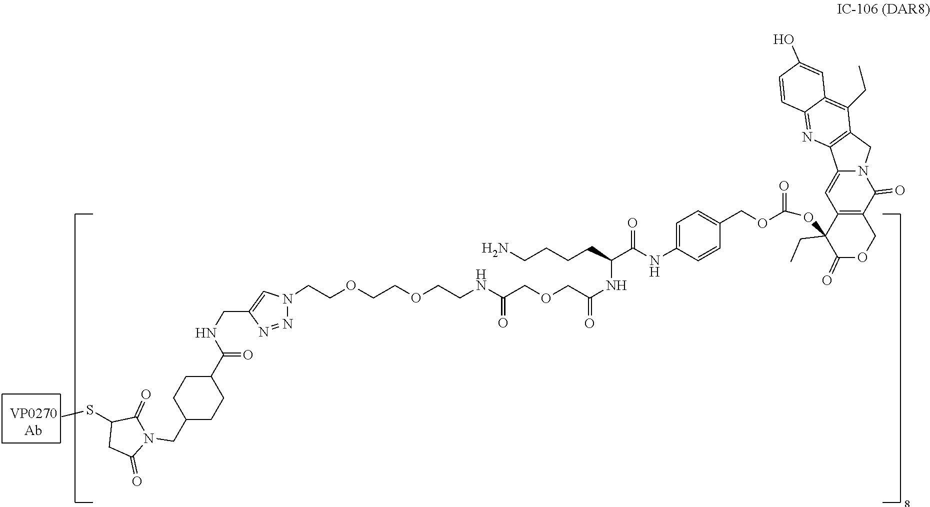

### Example II-17: Synthesis of Conjugate IC-A1 (DAR8)

Conjugate IC-A1 (VP1157-Compound 1) (DAR8) was synthesized similarly to IC-1 (according to Protocol B) above in Example II-1) using Compound 1 and ICAM1 specific IgG1 antibody VP1157. The afforded Conjugate IC-A1 (DAR8) was analyzed using LC/MS to calculate the DAR and using SEC-HPLC for purity analysis.

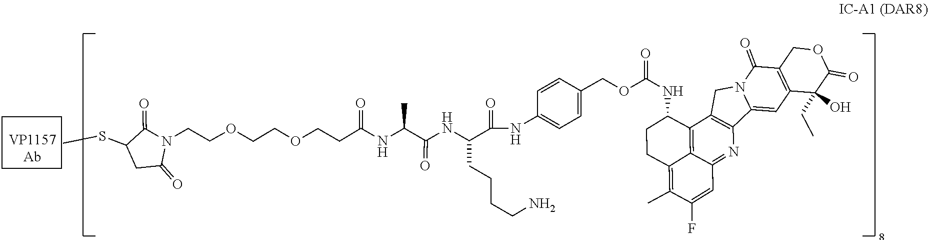

### Example II-18: Synthesis of Conjugate IC-A40 (DAR8)

Conjugate IC-A40 (VP1157-Compound 40) (DAR8) was synthesized similarly to IC-1 (according to Protocol B) above in Example II-1) using Compound 40 and ICAM1 specific IgG1 antibody VP1157. The afforded Conjugate IC-A40 (DAR8) was analyzed using LC/MS to calculate the DAR and using SEC-HPLC for purity analysis.

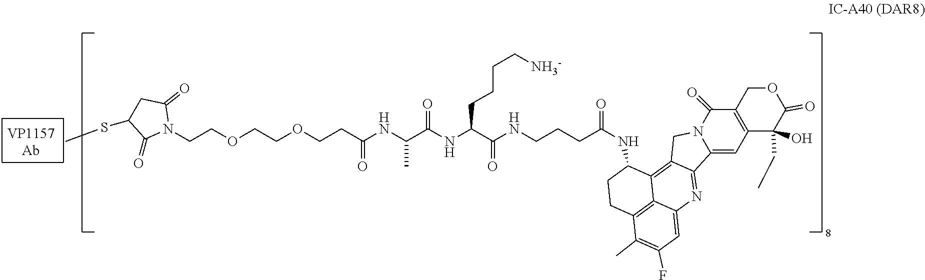

### Example II-19: Synthesis of Conjugate IC-A105 (DAR8)

Conjugate IC-A105 (VP1157-Compound 105) (DAR8) was synthesized similarly to IC-1 (according to Protocol B) above in Example II-1) using Compound 105 and ICAM1 specific IgG1 antibody VP1157. The afforded Conjugate IC-A105 (DAR8) was analyzed using LC/MS to calculate the DAR and using SEC-HPLC for purity analysis.

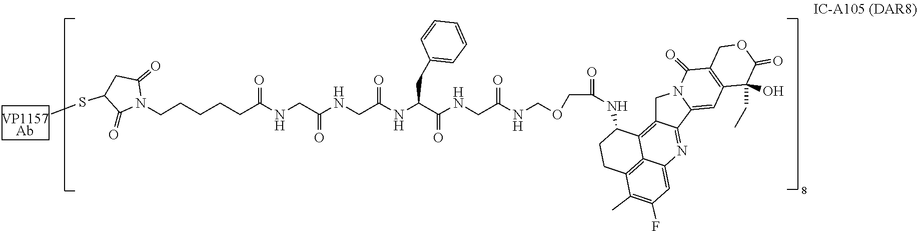

### Example II-20: Synthesis of Conjugate AC-40 (DAR8)

Conjugate AC-40 (VP0633-Compound 40) (DAR8) was synthesized similarly to IC-1 (according to Protocol B) above in Example II-1) using Compound 40 and EphA2 specific IgG1 antibody VP0633. The afforded Conjugate AC-40 (DAR8) was analyzed using LC/MS to calculate the DAR and using SEC-HPLC for purity analysis.

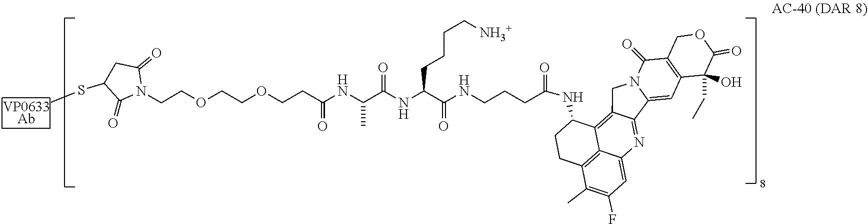

### Example II-21: Synthesis of Conjugate AC-105 (DAR8)

Conjugate AC-105 (VP0633-Compound 105) (DAR8) was synthesized similarly to IC-1 (according to Protocol B) above in Example II-1) using Compound 105 and EphA2 specific IgG1 antibody VP0633. The afforded Conjugate AC-105 (DAR8) was analyzed using LC/MS to calculate the DAR and using SEC-HPLC for purity analysis.

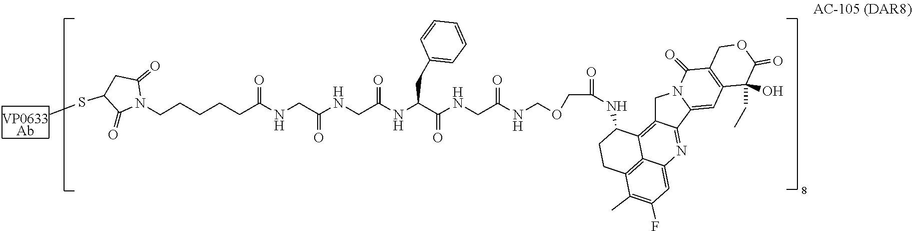

### Example II-22: Synthesis of Conjugate AC-A1 (DAR8)

Conjugate AC-A1 (DAR8) was synthesized similarly to AC-1 (according to Protocol B) above in Example II-1) using Compound 1 and EphA2 specific IgG1 antibody VP1127. The afforded Conjugate AC-A1 (DAR8) was analyzed using LC/MS to calculate the DAR and using SEC-HPLC for purity analysis.

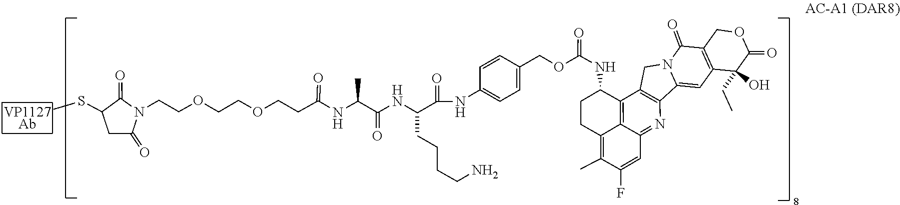

### Example II-23: Synthesis of Conjugate AC-B1 (DAR8)

Conjugate AC-B1 (VP1342-Compound 1) (DAR8) was synthesized similarly to AC-1 (according to Protocol B) above in Example II-1) using Compound 1 and EphA2 specific IgG1 antibody VP1342. The afforded Conjugate AC-B1 (DAR8) was analyzed using LC/MS to calculate the DAR and using SEC-HPLC for purity analysis.

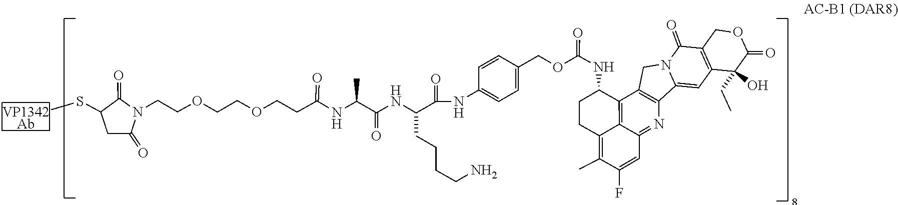

### Example II-24: Synthesis of Conjugate AC-C1 (DAR8)

Conjugate AC-C1 (VP0253-Compound 1) (DAR8) was synthesized similarly to AC-1 (according to Protocol B) above in Example II-1) using Compound 1 and EphA2 specific IgG1 antibody VP0253. The afforded Conjugate AC-C1 (DAR8) was analyzed using LC/MS to calculate the DAR and using SEC-HPLC for purity analysis.

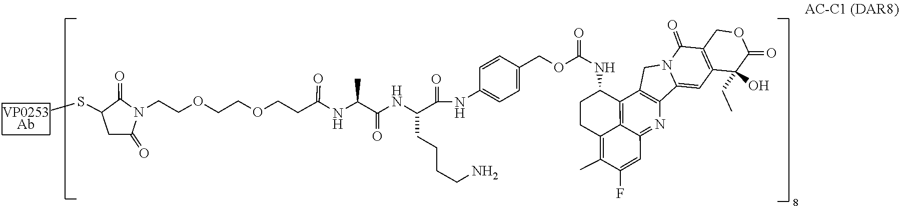

### III. Biological Evaluation

### Example III-1: In Vitro Activity of ADCs—Inhibition of Tumor Cell Growth In Vitro

The synthesized ADCs were tested were tested for their ability to inhibit growth of tumor cells in cell culture media after 6 days of treatment (including, e.g., MDA-MB-436 breast tumor cells, which express ICAM1 and EphA2 on the cell surface). All cell lines were obtained from ATCC (American Type Culture Collection).

A) Anti-ICAM1 and Anti-EphA2 ADCs Conjugated with Topoisomerase Inhibitors Inhibited Growth of MDA-MB-436 Breast Cancer Cells

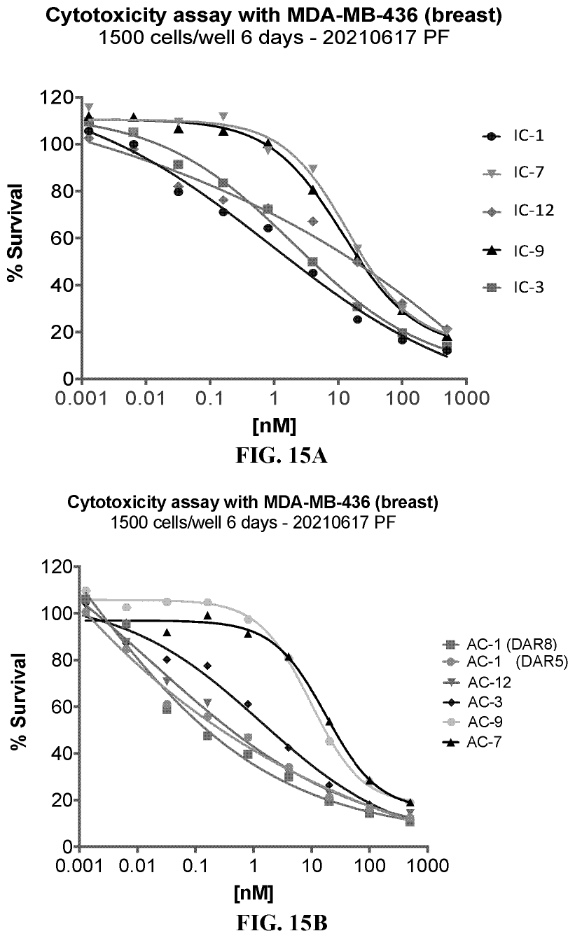

MDA-MB-436 cells, which express both ICAM1 and EphA2, were plated in 96-well plates at 1500 cells/well (140 μL/well) in complete culture medium (RPMI1640+10% FBS+10 mg/mL human insulin) and incubated at 37° C., 5% CO₂ overnight. Serial-diluted antibody drug conjudtaes (ADCs) were prepared in culture medium and were added to the well at L/well. Treated cells were incubated for another 6 days and cell viability was measured by CellTiter-Glo (Promega). Cell survival was calculated using GraphPad Prism software. As shown in FIGS. 15A and 15B, anti-ICAM1 antibody VP0270 and anti-EphA2 antibody VP0633 conjugated to topoisomerase inhibitors inhibited growth of MDA-MB-436 cells.

B) Anti-EphA2 ADCs Conjugated with Topoisomerase Inhibitors Preferentially Killed Target Expressing Cells

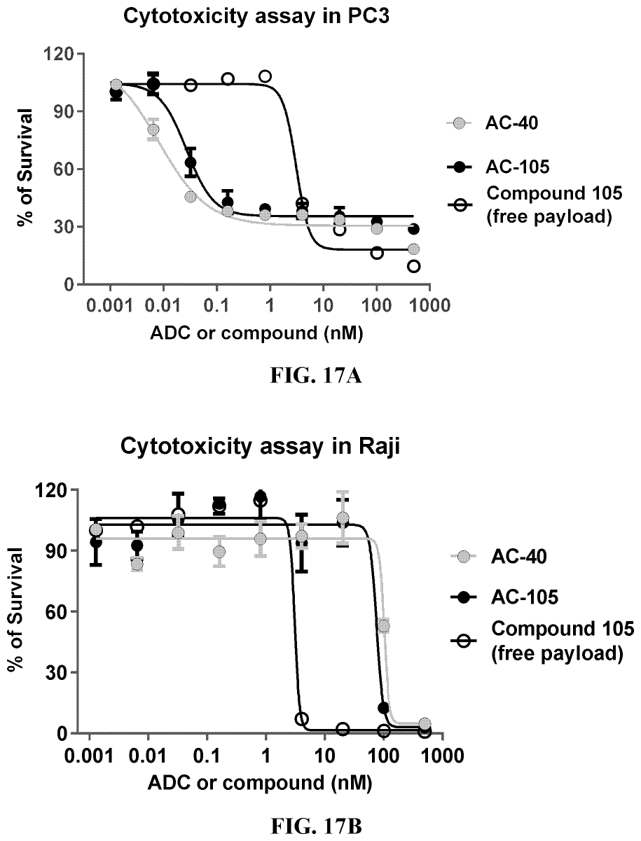

Additional in vitro cytotoxicity assays were performed in PC3 prostate cancer (seeded at 800 cells/well in Ham's F-12K medium+10% FBS) and Raji lymphoma cell lines (seeded at 1000 cells/well in RPMI1640+10% FBS) using anti-EphA2 ADCs, AC-105 (DAR8) (VP0633-Compound 105 (conjugated to reference payload DXd)) and AC-40 (DAR8) (VP0633-Compound 40), in comparison to the payload DXd only (not conjugated). The PC3 cell line expresses the targeting antigen (EphA2) while Raji does not express the targeting antigen. As shown in FIGS. 17A and 17B, both conjugates AC-105 (VP0633-Compound 105) and AC-40 (VP-0633-Compound 40) have higher inhibitory activity in PC3 cell line than in Raji cell line demonstrating target mediated killing by the ADCs. Also as shown in FIGS. 17A and 17B, AC-40 has comparable activity when compared to the antibody conjugate AC-105, which contains a reference payload DXd.

C) Additional Examples of Anti-EphA2 and Anti-ICAM1 ADCs Conjugated with Topoisomerase Inhibitors Inhibited Growth of Tumor Cells In Vitro

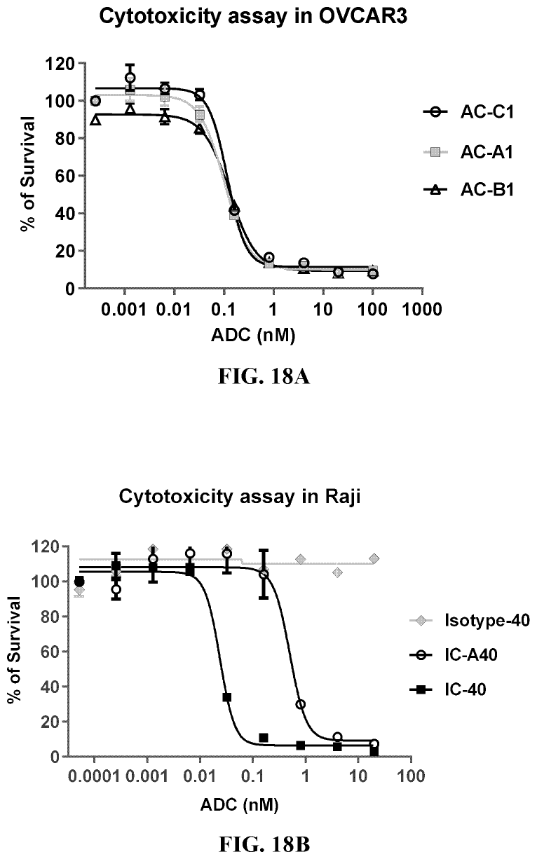

In vitro cytotoxicities of anti-EphA2 and anti-ICAM1 ADCs were further evaluated in OVCAR3 ovarian cancer cells (seeded at 2000 cells/well in RPMI1640+20% FBS+10 mg/mL bovine insulin) and Raji lymphoma cells, respectively. As shown in FIG. 18A, three anti-EphA2 antibody conjugates AC-C1 (DAR8) (VP0253-Compound 1), AC-A1 (DAR8) (VP1127-Compound 1) and AC-B1 (DAR8) (VP1342-Compound 1) have comparable cytotoxicity in OVCAR3 cells. For anti-ICAM1 ADCs, FIG. 18B shows that IC-40 (DAR8) (VP0270-Compound 40) killed Raji cells more effectively than IC-A40 (DAR8) (VP1157-Compound 40). In contrast, an isotype control antibody conjugated to Compound 40 (Isotype-Compound 40) did not kill Raji cells even at the highest concentration. This data demonstrated that cell killing by anti-ICAM1 ADCs is target mediated.

### Example III-2: In Vivo Activity of ADCs—Tumor Xenograft Therapeutic Model

In vivo anti-tumor activities of the synthesized anti-ICAM1 ADCs and anti-EphA2 ADCs were evaluated in several cancer cell line xenograft models as described below. All cell lines were obtained from ATCC (American Type Culture Collection).

A) Anti-ICAM1 ADCs Conjugated with Topoisomerase Inhibitor Inhibited Growth of HCC827 Non-Small Cell Lung Cancer Xenograft Model.

Specifically, HCC827 tumor cells were cultured in RPMI-1640 medium supplemented with 10% heat-inactivated FBS at 37° C. in an atmosphere of 5% CO₂ in air. Tumor cells growing in the exponential growth phase were harvested for tumor inoculation. Female Balb/c nude mice of 6-8 weeks in age were inoculated subcutaneously at the right flank with 10⁷ cells in 0.1 mL of PBS supplemented with equal volume of Matrigel for tumor development.

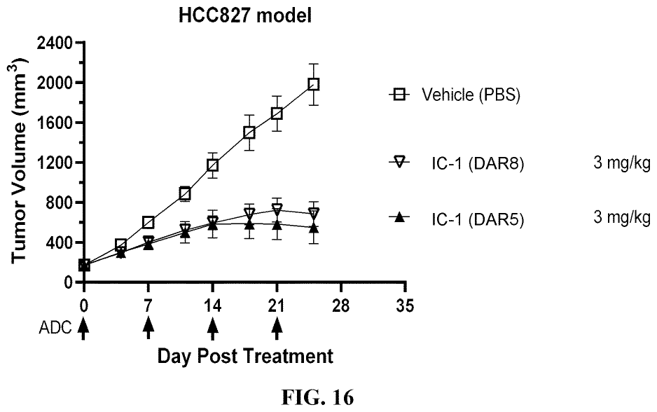

Tumor volume was measured using a caliper device and calculated with the following formula: Tumor volume=(length×width²)/2. On day 6 post tumor inoculation when the mean tumor volume reached approximately 170 mm³, animals were randomized by tumor volumes into 6 animals per group. The tested anti-ICAM1-ADCs (IC-1 (DAR5) and IC-1 (DAR8), respectively) were administered through bolus tail vein injection at a dose of 3 mg/kg, once per week for four consecutive weeks. Tumor volume was measured twice weekly. As shown in FIG. 16, both Conjugates IC-1 (DAR5) and IC-1 (DAR8), which comprise anti-ICAM1 antibody VP0270 conjugated to Compound 1, inhibited HCC827 tumor growth in vivo in xenograft model.

B) Anti-EphA2 ADCs Conjugated with Topoisomerase Inhibitors Inhibited Growth of OVCAR3 Ovarian Cancer Xenograft Model.

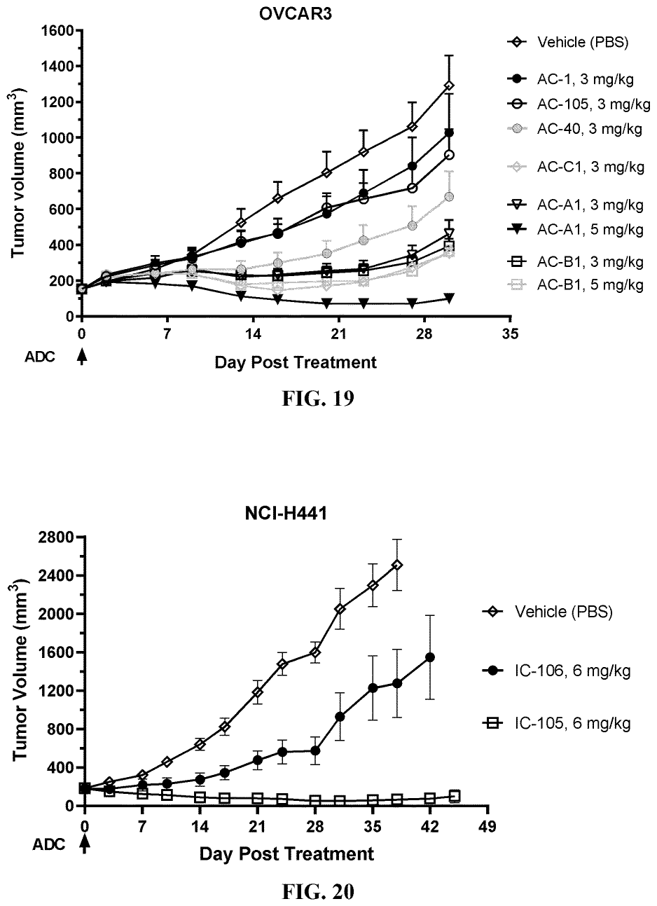

To evaluate anti-tumor activities of anti-EphA2 ADCs, female Balb/c nude mice of 6-8 weeks in age were inoculated subcutaneously at the right flank with 10⁷ OVCAR3 cells in 0.1 mL of PBS supplemented with equal volume of Matrigel for tumor development. Animals were randomized into groups of 6 mice per group for treatment when the mean tumor volume reached 150-200 mm³. A single injection of anti-EphA2 ADCs (DAR8) was administered through bolus tail vein injection at a dose of either 3 or 5 mg/kg. As shown in FIG. 19, anti-EphA2 antibody VP0633 conjugated to Compound 1 (AC-1) or conjugated to Compound 105 (which contains the reference payload DXd) was not effective in inhibiting OVCAR3 tumor growth. In contrast, AC-40 (VP0633-Compound 40), AC-C1 (VP0253-Compound 1), AC-A1 (VP1127-Compound 1) and AC-B1 (VP1342-Compound 1) effectively inhibited OVCAR3 tumor growth at various extent.

C) Anti-ICAM1 ADCs Conjugated with Reference Topoisomerase Inhibitors Inhibited Growth of NCI-11441 Non-Small Cell Lung Cancer Xenograft Model.

The anti-tumor activities of ICAM1 antibody VP0270 conjugated to linker payload Compound 105 (which comprises reference payload DXd) and Compound 106 (which comprises reference payload SN38) were evaluated in NCI-H441 non-small cell lung cancer model. For tumor development, 5×10⁶ cells (cultured in RPMI-1640 medium+10% heat-inactivated FBS) in 0.1 mL PBS were inoculated subcutaneously at the right flank of 6-8 weeks old female Balb/c nude mice. Once the mean tumor volume reached 150-200 mm³, animals were randomized into groups of 6 mice per group for treatment. As shown in FIG. 20, a single bolus tail vein injection of 6 mg/kg IC-105 (VP0270-Compound 105) led to tumor regression whereas IC-106 (VP0270-Compound 106) only slowed tumor growth.

D) Anti-ICAM1 ADCs Conjugated with Compound 1 Topoisomerase Inhibitor Induced Tumor Regression in Hep3B2.1-7 Hepatocellular Carcinoma Xenograft Model.

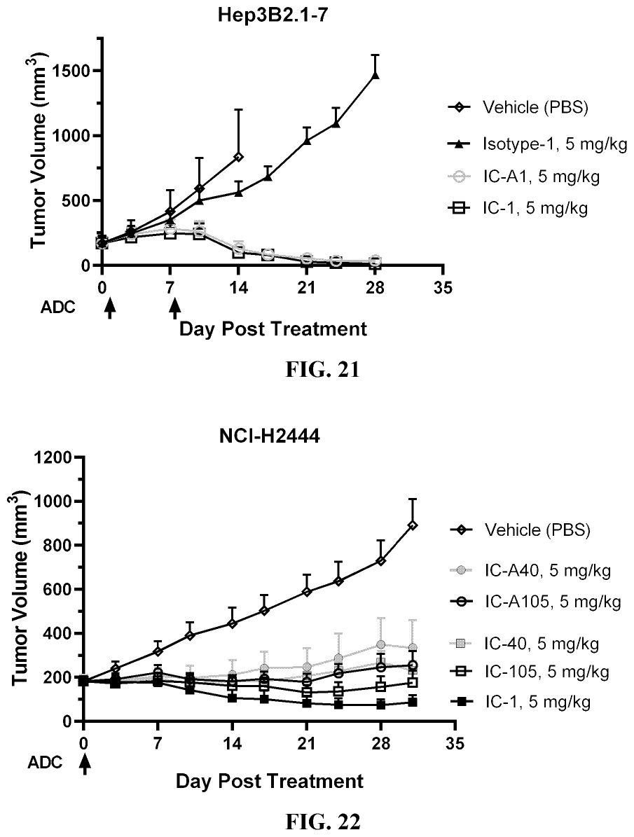

Hep3B2.1-7 cells were cultured in EMEM medium supplemented with 10% heat inactivated FBS. Female Balb/c nude mice of 6-8 weeks in age were inoculated subcutaneously at the right flank with 8×10⁶ cells in 0.1 mL of PBS for tumor development. Once the mean tumor volume reached 150-200 mm³, animals were randomized into groups of 6 mice per group for treatment. As shown in FIG. 21, two bolus tail vein injection of 5 mg/kg of anti-ICAM1 conjugates IC-A1 (DAR8) (VP1157-Compound 1) or IC-1 (DAR8) (VP0270-Compound 1) given one week apart effectively drove tumor regression. In contrast, isotype antibody conjugated to Compound 1 (Isotype-Compound 1) had little effect in controlling tumor growth.

E) Anti-ICAM1 ADCs Conjugated with Topoisomerase Inhibitors Inhibited Tumor Progression in NCI-112444 Non-Small Cell Lung Cancer Xenograft Model.

NCI-H2444 cells were cultured in RPMI-1640 medium supplemented with 10% heat inactivated FBS. Female Balb/c nude mice of 6-8 weeks in age were inoculated subcutaneously at the right flank with 5×10⁶ cells in 0.1 mL of PBS supplemented with equal volume of Matrigel for tumor development. When the mean tumor volume reached 180-200 mm³, animals were randomized into groups of 6 mice each for treatment. All anti-ICAM1 ADCs tested were conjugated with payloads of Compound 1, Compound 40, or Compound 105 at a DAR of 8: IC-1 (VP0270-Compound 1), IC-40 (VP0270-Compound 40), IC-105 (VP0270-Compound 105), IC-A40 (VP1157-Compound 40), and IC-A105 (VP1157-Compound 105). As shown in FIG. 22, a single bolus tail vein injection of 5 mg/kg of all tested anti-ICAM1 ADCs was sufficient to significantly delay tumor growth. In addition, IC-1 (VP0270-Compound 1) has similar anti-tumor activity as the antibody conjugated with a reference payload, IC-105 (VP0270-Compound 105), in the NCI-H2444 model.

### DESCRIPTION OF THE SEQUENCES

TABLE 8
Sequence Information.

| SEQ ID | | |
|---|---|---|
| NO. | DESCRIPTION | SEQUENCE |
| 1 | VP0270 heavy chain | GAGGTGCAGGTGCTGGAGAGCGGAGGAGGACTGGTG |
| | DNA sequence | CAGCCCGGAGGGTCACTGAGACTGTCATGTGCCGTGT |
| | | CAGGATTTTCACTGAGCTCCCACGCTATGGGCTGGGTG |
| | | CGGCAGGCACCAGGCAAGGGCCTGGAGTGGATCGGC |
| | | ATCATCGGCTCTAGCGACCGGACCTACTATGCCAGCT |
| | | GGGCCAAGGGCAGATTCACCATCTCCAAGGATAACTC |
| | | TAAGAATACAGTGTACCTGCAGATCAACTCCCCCAGG |
| | | GCCGAGGACACAGCCGTGTACTATTGCGTGCGCGATC |
| | | CTTATGATTCTTATGATGAGGGCTACCGACTGTGGGGG |
| | | CAGGGGACACTGGTGACCGTCTCCTCAGCTAGCACCA |
| | | AAGGTCCCAGCGTGTTCCCACTCGCCCCGAGTTCAAA |
| | | ATCAACTTCTGGAGGCACCGCCGCCCTGGGTTGCCTG |
| | | GTAAAGGACTACTTCCCAGAGCCCGTGACCGTGAGCT |
| | | GGAACTCCGGGGCACTGACATCTGGCGTTCATACTTTC |
| | | CCGGCCGTGCTCCAGTCTTCAGGTCTGTATAGTCTCTC |
| | | CTCTGTGGTCACTGTCCCATCTAGCTCTCTGGGCACCC |
| | | AAACCTACATATGCAACGTTAATCACAAGCCGAGCAA |
| | | TACTAAAGTTGACAAAAAGGTGGAACCCAAGTCTTGT |
| | | GACAAGACCCACACGTGTCCCCCCTGCCCGGCTCCTG |
| | | AGCTGCTTGGCGGCCCCAGCGTCTTTCTCTTTCCCCCA |
| | | AAGCCAAAAGATACCTTGATGATCAGCAGAACTCCCG |
| | | AGGTGACATGCGTCGTCGTGGACGTAAGCCATGAAGA |
| | | TCCCGAGGTTAAGTTCAACTGGTATGTCGATGGCGTG |
| | | GAAGTCCATAATGCTAAGACTAAACCTCGCGAAGAGC |
| | | AGTACAATTCAACTTACCGGGTCGTTTCCGTTCTGACC |
| | | GTGCTGCATCAGGACTGGCTGAATGGTAAAGAGTACA |
| | | AGTGCAAAGTGTCTAACAAGGCACTCCCCGCCCCAAT |
| | | TGAGAAGACTATCTCCAAAGCTAAAGGGCAACCAAGA |
| | | GAGCCCCAGGTCTACACCCTGCCCCCCTCAAGGGATG |
| | | AGCTTACTAAGAACCAGGTTAGTCTCACCTGCTTGGTT |
| | | AAAGGATTTTATCCAAGCGATATTGCTGTGGAGTGGG |
| | | AGTCCAACGGCCAGCCTGAGAACAATTATAAAACCAC |
| | | CCCCCCTGTTCTTGACAGTGACGGTAGTTTCTTCCTGT |
| | | ATTCCAAACTGACCGTCGATAAGAGCAGATGGCAACA |
| | | GGGAAATGTGTTCAGCTGCTCCGTGATGCATGAGGCG |
| | | CTCCATAATCATTACACACAAAAAAGTTTGTCCCTGAG |
| | | CCCAGGCAAG |
| 2 | VP0270 heavy chain | EVQVLESGGGLVQPGGSLRLSCAVSGFSLSSHAMGWVR |
| | amino acid sequence | QAPGKGLEWIGIIGSSDRTYYASWAKGRFTISKDNSKNT |
| | | VYLQINSPRAEDTAVYYCVRDPYDSYDEGYRLWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSSS |
| | | LGTQTYICNVNHKPSNTKVDKKVEPKSCDKTHTCPPCPA |
| | | PELLGGPSVFLFPPKPKDTLMISRTPEVTCVVVDVSHEDP |
| | | EVKFNWYVDGVEVHNAKTKPREEQYNSTYRVVSVLTV |
| | | LHQDWLNGKEYKCKVSNKALPAPIEKTISKAKGQPREPQ |
| | | VYTLPPSRDELTKNQVSLTCLVKGFYPSDIA VEWESNGQ |
| | | PENNYKTTPPVLDSDGSFFLYSKLTVDKSRWQQGNVFSC |
| | | SVMHEALHNHYTQKSLSLSPGK |
| 3 | VP0270 light chain | GATATTCAGATGACCCAGAGCCCCAGCACACTGAGCG |
| | DNA sequence | CAAGCGTCGGAGATAGAGTGACCATTACCTGCCAGGC |
| | | CAGCCAGAGCATTTACAGATACCTGTCTTGGTATCAGC |
| | | AGAAGCCCGGCAAGGCCCCTAAGTTCCTGATCTATGA |
| | | CGCCTCTAAGCTGGCCAGCGGCGTGCCATCCAGATTCT |
| | | CTGGCAGCGGCTCCGGCACCGAGTTTACCCTGACAAT |
| | | CAGCTCCGTGCAGCCAGACGATTTCGCAACATACTATT |
| | | GCCAGCAGGCTTACAGCAGCGGCAGCATTGACAACGC |
| | | ATTCGGAGGAGGGACTAAAGTGGAGATCAAGCGTACG |
| | | GTGGCAGCTCCATCAGTTTTTATCTTCCCACCAAGCGA |
| | | CGAGCAATTGAAGTCCGGCACTGCCTCTGTGGTGTGC |
| | | CTTCTGAACAACTTCTATCCAAGGGAGGCCAAGGTCC |
| | | AGTGGAAGGTCGATAATGCGCTGCAGAGCGGGAACA |
| | | GCCAAGAGTCAGTGACCGAGCAGGACTCAAAAGATA |
| | | GCACATACTCTCTGAGTTCCACCCTGACCCTGTCAAAG |
| | | GCTGACTACGAAAAGCATAAGGTATACGCATGCGAAG |
| | | TGACCCATCAGGGTCTCTCATCTCCCGTAACCAAATCT |
| | | TTTAATAGAGGAGAATGC |
| 4 | VP0270 light chain | DIQMTQSPSTLSASVGDRVTITCQASQSIYRYLSWYQQKP |
| | amino acid sequence | GKAPKFLIYDASKLASGVPSRFSGSGSGTEFTLTISSVQPD |
| | | DFATYYCQQAYSSGSIDNAFGGGTKVEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 5 | VP0633 heavy chain | GAGGTGCAGCTGGTGGAGAGCGGCGGCGGCCTGGTGC |
| | DNA sequence | AGCCCGGCGGCAGCCTGAGACTGAGCTGCGCCGCCAG |
| | | CGGCTTCACCTTCAGCAGCTACAGCATGAACTGGGTG |
| | | AGACAGGCCCCCGGCAAGGGCCTGGAGTGGGTGAGCT |
| | | ACATCAGCAGCAGCAGCAGCACCATCTACTACGCCGA |
| | | CAGCGTGAAGGGCAGATTCACCATCAGCAGAGACAAC |
| | | GCCAAGAACAGCCTGTACCTGCAGATGAACAGCCTGA |
| | | GAGCCGAGGACACCGCCGTGTACTACTGCGCCAGATA |
| | | CAGACTGCCCAACTTCTGGAGCGGCTACCCCAACTAC |
| | | GGCACCGACGTGTGGGGCCAGGGCACCACCGTGACCG |
| | | TGAGCAGCGCTAGCACCAAGGGCCCATCGGTCTTCCC |
| | | CCTGGCACCCTCCTCCAAGAGCACCTCTGGGGGCACA |
| | | GCGGCCCTGGGCTGCCTGGTCAAGGACTACTTCCCCG |
| | | AACCGGTGACGGTGTCGTGGAACTCAGGCGCCCTGAC |
| | | CAGCGGCGTGCACACCTTCCCGGCTGTCCTACAGTCCT |
| | | CAGGACTCTACTCCCTCAGCAGCGTGGTGACCGTGCC |
| | | CTCCAGCAGCTTGGGCACCCAGACCTACATCTGCAAC |
| | | GTGAATCACAAGCCCAGCAACACCAAGGTGGACAAG |
| | | AAAGTTGAGCCCAAATCTTGTGACAAAACTCACACAT |
| | | GCCCACCGTGCCCAGCACCTGAACTCCTGGGGGGACC |
| | | GTCAGTCTTCCTCTTCCCCCCAAAACCCAAGGACACCC |
| | | TCATGATCTCCCGGACCCCCGAGGTCACATGCGTGGT |
| | | GGTGGACGTGAGCCACGAAGACCCTGAGGTCAAGTTC |
| | | AACTGGTACGTGGACGGCGTGGAGGTGCATAATGCCA |
| | | AGACAAAGCCGCGGGAGGAGCAGTACAACAGCACGT |
| | | ACCGTGTGGTCAGCGTCCTCACCGTCCTGCACCAGGA |
| | | CTGGCTGAATGGCAAGGAGTACAAGTGCAAGGTCTCC |
| | | AACAAAGCCCTCCCAGCCCCCATCGAGAAAACCATCT |
| | | CCAAAGCCAAAGGGCAGCCCCGAGAACCACAGGTGT |
| | | ACACCCTGCCCCCATCCCGGGAGGAGATGACCAAGAA |
| | | CCAGGTCAGCCTGACCTGCCTGGTCAAAGGCTTCTATC |
| | | CCAGCGACATCGCCGTGGAGTGGGAGAGCAATGGGCA |
| | | GCCGGAGAACAACTACAAGACCACGCCTCCCGTGCTG |
| | | GACTCCGACGGCTCCTTCTTCCTCTACAGCAAGCTCAC |
| | | CGTGGACAAGAGCAGGTGGCAGCAGGGGAACGTCTTC |
| | | TCATGCTCCGTGATGCATGAGGCTCTGCACAACCACTA |
| | | CACGCAGAAGAGCCTCTCCCTGTCTCCGGGTAAA |
| 6 | VP0633 heavy chain | EVQLVESGGGLVQPGGSLRLSCAASGFTFSSYSMNWVR |
| | amino acid sequence | QAPGKGLEWVSYISSSSSTIYYADSVKGRFTISRDNAKNS |
| | | LYLQMNSLRAEDTAVYYCARYRLPNFWSGYPNYGTDV |
| | | WGQGTTVTVSSASTKGPSVFPLAPSSKSTSGGTAALGCL |
| | | VKDYFPEPVTVSWNSGALTSGVHTFPAVLQSSGLYSLSS |
| | | VVTVPSSSLGTQTYICNVNHKPSNTKVDKKVEPKSCDKT |
| | | HTCPPCPAPELLGGPSVFLFPPKPKDTLMISRTPEVTCVV |
| | | VDVSHEDPEVKFNWYVDGVEVHNAKTKPREEQYNSTY |
| | | RVVSVLTVLHQDWLNGKEYKCKVSNKALPAPIEKTISKA |
| | | KGQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| 7 | VP0633 light chain | CAGTCTGTGCTGACACAGCCACCCAGCGTGAGCGGAG |
| | DNA sequence | CCCCTGGACAGAGAGTGACCATCAGCTGCACCGGAAG |
| | | CAGCAGCAATATCGGAGCCGGATATGACGTGCATTGG |
| | | TACCAGCAGCTGCCCGGCACCGCCCCAAAACTGCTGA |
| | | TCTACGGAAACAGCAACAGACCCAGCGGCGTGCCCGA |
| | | CAGATTCAGCGGCAGCAAGAGCGGCACAAGCGCCAGT |
| | | CTGGCTATCACCGGCCTGCAGGCCGAGGACGAAGCCG |
| | | ACTACTACTGCCAGAGCTACGACAGCAGCCTGAGCGG |
| | | CCACGTGGTGTTTGGAGGAGGAACCAAGCTGACCGTG |
| | | CTGGGTCAGCCCAAGGCCAACCCCACTGTCACTCTGTT |
| | | CCCGCCCTCCTCTGAGGAGCTCCAAGCCAACAAGGCC |
| | | ACACTAGTGTGTCTGATCAGTGACTTCTACCCGGGAGC |
| | | TGTGACAGTGGCCTGGAAGGCAGATGGCAGCCCCGTC |
| | | AAGGCGGGAGTGGAGACCACCAAACCCTCCAAACAG |
| | | AGCAACAACAAGTACGCGGCCAGCAGCTACCTGAGCC |
| | | TGACGCCCGAGCAGTGGAAGTCCCACAGAAGCTACAG |
| | | CTGCCAGGTCACGCATGAAGGGAGCACCGTGGAGAAG |
| | | ACAGTGGCCCCTACAGAATGTTCA |
| 8 | VP0633 light chain | QSVLTQPPSVSGAPGQRVTISCTGSSSNIGAGYDVHWYQ |
| | amino acid sequence | QLPGTAPKLLIYGNSNRPSGVPDRFSGSKSGTSASLAITG |
| | | LQAEDEADYYCQSYDSSLSGHVVFGGGTKLTVLGQPKA |
| | | NPTVTLFPPSSEELQANKATLVCLISDFYPGAVTVAWKA |
| | | DGSPVKAGVETTKPSKQSNNKYAASSYLSLTPEQWKSH |
| | | RSYSCQVTHEGSTVEKTVAPTECS |
| 9 | VP0253 heavy chain | CAGGTGCAGCTGCAGGAGAGCGGCGGCGGACTGGTGC |
| | DNA sequence | AGCCAGGAGGAAGCCTGAGACTGTCTTGCGCTGCTTC |
| | | CGGCTTCACATTCAGCAGCTACAGCATGAACTGGGTG |
| | | AGACAGGCCCCCGGAAAAGGACTGGAGTGGGTGAGC |
| | | TACATCAGCAGCAGTAGTAGCACCATCTATTACGCTG |
| | | ATTCCGTGAAAGGCAGATTCACAATCTCCCGGGACAA |
| | | CGCCAAGAACAGCCTGTACCTGCAGATGAACAGCCTG |
| | | AGAGCCGAGGACACCGCCGTGTACTACTGCGCCAGGT |
| | | ACCGGCTGCCCGATTTCTGGTCCGGCTACCCCAACTAC |
| | | GGCATGGATGTGTGGGGCCAGGGCACCACCGTGACAG |
| | | TGAGCAGCGCTAGCACCAAGGGCCCATCGGTCTTCCC |
| | | CCTGGCACCCTCCTCCAAGAGCACCTCTGGGGGCACA |
| | | GCGGCCCTGGGCTGCCTGGTCAAGGACTACTTCCCCG |
| | | AACCGGTGACGGTGTCGTGGAACTCAGGCGCCCTGAC |
| | | CAGCGGCGTGCACACCTTCCCGGCTGTCCTACAGTCCT |
| | | CAGGACTCTACTCCCTCAGCAGCGTGGTGACCGTGCC |
| | | CTCCAGCAGCTTGGGCACCCAGACCTACATCTGCAAC |
| | | GTGAATCACAAGCCCAGCAACACCAAGGTGGACAAG |
| | | AAAGTTGAGCCCAAATCTTGTGACAAAACTCACACAT |
| | | GCCCACCGTGCCCAGCACCTGAACTCCTGGGGGGACC |
| | | GTCAGTCTTCCTCTTCCCCCCAAAACCCAAGGACACCC |
| | | TCATGATCTCCCGGACCCCCGAGGTCACATGCGTGGT |
| | | GGTGGACGTGAGCCACGAAGACCCTGAGGTCAAGTTC |
| | | AACTGGTACGTGGACGGCGTGGAGGTGCATAATGCCA |
| | | AGACAAAGCCGCGGGAGGAGCAGTACAACAGCACGT |
| | | ACCGTGTGGTCAGCGTCCTCACCGTCCTGCACCAGGA |
| | | CTGGCTGAATGGCAAGGAGTACAAGTGCAAGGTCTCC |
| | | AACAAAGCCCTCCCAGCCCCCATCGAGAAAACCATCT |
| | | CCAAAGCCAAAGGGCAGCCCCGAGAACCACAGGTGT |
| | | ACACCCTGCCCCCATCCCGGGAGGAGATGACCAAGAA |
| | | CCAGGTCAGCCTGACCTGCCTGGTCAAAGGCTTCTATC |
| | | CCAGCGACATCGCCGTGGAGTGGGAGAGCAATGGGCA |
| | | GCCGGAGAACAACTACAAGACCACGCCTCCCGTGCTG |
| | | GACTCCGACGGCTCCTTCTTCCTCTACAGCAAGCTCAC |
| | | CGTGGACAAGAGCAGGTGGCAGCAGGGGAACGTCTTC |
| | | TCATGCTCCGTGATGCATGAGGCTCTGCACAACCACTA |
| | | CACGCAGAAGAGCCTCTCCCTGTCTCCGGGTAAA |
| 10 | VP0253 heavy chain | QVQLQESGGGLVQPGGSLRLSCAASGFTFSSYSMNWVR |
| | amino acid sequence | QAPGKGLEWVSYISSSSSTIYYADSVKGRFTISRDNAKNS |
| | | LYLQMNSLRAEDTAVYYCARYRLPDFWSGYPNYGMDV |
| | | WGQGTTVTVSSASTKGPSVFPLAPSSKSTSGGTAALGCL |
| | | VKDYFPEPVTVSWNSGALTSGVHTFPAVLQSSGLYSLSS |
| | | VVTVPSSSLGTQTYICNVNHKPSNTKVDKKVEPKSCDKT |
| | | HTCPPCPAPELLGGPSVFLFPPKPKDTLMISRTPEVTCVV |
| | | VDVSHEDPEVKFNWYVDGVEVHNAKTKPREEQYNSTY |
| | | RVVSVLTVLHQDWLNGKEYKCKVSNKALPAPIEKTISKA |
| | | KGQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| 11 | VP0253 light chain | CAGTCTGTGCTGACACAGCCACCCAGCGTGAGCGGAG |
| | DNA sequence | CCCCTGGACAGAGAGTGACCATCAGCTGCACCGGAAG |
| | | CAGCAGCAATATCGGAGCCGGATATGACGTGCATTGG |
| | | TACCAGCAGCTGCCCGGCACCGCCCCAAAACTGCTGA |
| | | TCTACGGAAACAGCAACAGACCCAGCGGCGTGCCCGA |
| | | CAGATTCAGCGGCAGCAAGAGCGGCACAAGCGCCAGT |
| | | CTGGCTATCACCGGCCTGCAGGCCGAGGACGAAGCCG |
| | | ACTACTACTGCCAGAGCTACGACAGCAGCCTGAGCGG |
| | | CCACGTGGTGTTTGGAGGAGGAACCAAGCTGACCGTG |
| | | CTGGGTCAGCCCAAGGCCAACCCCACTGTCACTCTGTT |
| | | CCCGCCCTCCTCTGAGGAGCTCCAAGCCAACAAGGCC |
| | | ACACTAGTGTGTCTGATCAGTGACTTCTACCCGGGAGC |
| | | TGTGACAGTGGCCTGGAAGGCAGATGGCAGCCCCGTC |
| | | AAGGCGGGAGTGGAGACCACCAAACCCTCCAAACAG |
| | | AGCAACAACAAGTACGCGGCCAGCAGCTACCTGAGCC |
| | | TGACGCCCGAGCAGTGGAAGTCCCACAGAAGCTACAG |
| | | CTGCCAGGTCACGCATGAAGGGAGCACCGTGGAGAAG |
| | | ACAGTGGCCCCTACAGAATGTTCA |
| 12 | VP0253 light chain | QSVLTQPPSVSGAPGQRVTISCTGSSSNIGAGYDVHWYQ |
| | amino acid sequence | QLPGTAPKLLIYGNSNRPSGVPDRFSGSKSGTSASLAITG |
| | | LQAEDEADYYCQSYDSSLSGHVVFGGGTKLTVLGQPKA |
| | | NPTVTLFPPSSEELQANKATLVCLISDFYPGAVTVAWKA |
| | | DGSPVKAGVETTKPSKQSNNKYAASSYLSLTPEQWKSH |
| | | RSYSCQVTHEGSTVEKTVAPTECS |
| 13 | VP1157 heavy chain | GAGGTGCAGCTGCTGGAGAGCGGGGGGGGACTGGTG |
| | DNA sequence | CAGCCAGGAGGAAGCCTGAGACTGAGCTGTGCCGCCA |
| | | GCGGGTTTAGCCTGAGCAGCCACGCCATGGGATGGGT |
| | | GAGGCAGGCCCCTGGCAAAGGCCTGGAGTGGGTGAGC |
| | | ATTATTGGCAGCAGTGACAGAACCTACTACGCCGACA |
| | | GCGTGAAAGGGCGGTTCACCATCAGCAGAGATAACAG |
| | | CAAGAACACACTGTACCTGCAGATGAACAGCCTGAGA |
| | | GCCGAGGACACCGCTGTGTACTACTGCGCCAAGGACC |
| | | CATACGATAGTTACGACGAAGGATACAGACTGTGGGG |
| | | GCAGGGAACCCTGGTGACCGTGAGCAGCGCTAGCACC |
| | | AAAGGTCCCAGCGTGTTCCCACTCGCCCCGAGTTCAA |
| | | AATCAACTTCTGGAGGCACCGCCGCCCTGGGTTGCCT |
| | | GGTAAAGGACTACTTCCCAGAGCCCGTGACCGTGAGC |
| | | TGGAACTCCGGGGCACTGACATCTGGCGTTCATACTTT |
| | | CCCGGCCGTGCTCCAGTCTTCAGGTCTGTATAGTCTCT |
| | | CCTCTGTGGTCACTGTCCCATCTAGCTCTCTGGGCACC |
| | | CAAACCTACATATGCAACGTTAATCACAAGCCGAGCA |
| | | ATACTAAAGTTGACAAAAAGGTGGAACCCAAGTCTTG |
| | | TGACAAGACCCACACGTGTCCCCCCTGCCCGGCTCCTG |
| | | AGCTGCTTGGCGGCCCCAGCGTCTTTCTCTTTCCCCCA |
| | | AAGCCAAAAGATACCTTGATGATCAGCAGAACTCCCG |
| | | AGGTGACATGCGTCGTCGTGGACGTAAGCCATGAAGA |
| | | TCCCGAGGTTAAGTTCAACTGGTATGTCGATGGCGTG |
| | | GAAGTCCATAATGCTAAGACTAAACCTCGCGAAGAGC |
| | | AGTACAATTCAACTTACCGGGTCGTTTCCGTTCTGACC |
| | | GTGCTGCATCAGGACTGGCTGAATGGTAAAGAGTACA |
| | | AGTGCAAAGTGTCTAACAAGGCACTCCCCGCCCCAAT |
| | | TGAGAAGACTATCTCCAAAGCTAAAGGGCAACCAAGA |
| | | GAGCCCCAGGTCTACACCCTGCCCCCCTCAAGGGATG |
| | | AGCTTACTAAGAACCAGGTTAGTCTCACCTGCTTGGTT |
| | | AAAGGATTTTATCCAAGCGATATTGCTGTGGAGTGGG |
| | | AGTCCAACGGCCAGCCTGAGAACAATTATAAAACCAC |
| | | CCCCCCTGTTCTTGACAGTGACGGTAGTTTCTTCCTGT |
| | | ATTCCAAACTGACCGTCGATAAGAGCAGATGGCAACA |
| | | GGGAAATGTGTTCAGCTGCTCCGTGATGCATGAGGCG |
| | | CTCCATAATCATTACACACAAAAAAGTTTGTCCCTGAG |
| | | CCCAGGCAAG |
| 14 | VP1157 heavy chain | EVQLLESGGGLVQPGGSLRLSCAASGFSLSSHAMGWVR |
| | amino acid sequence | QAPGKGLEWVSIIGSSDRTYYADSVKGRFTISRDNSKNTL |
| | | YLQMNSLRAEDTAVYYCAKDPYDSYDEGYRLWGQGTL |
| | | VTVSSASTKGPSVFPLAPSSKSTSGGTAALGCLVKDYFPE |
| | | PVTVSWNSGALTSGVHTFPAVLQSSGLYSLSSVVTVPSSS |
| | | LGTQTYICNVNHKPSNTKVDKKVEPKSCDKTHTCPPCPA |
| | | PELLGGPSVFLFPPKPKDTLMISRTPEVTCVVVDVSHEDP |
| | | EVKFNWYVDGVEVHNAKTKPREEQYNSTYRVVSVLTV |
| | | LHQDWLNGKEYKCKVSNKALPAPIEKTISKAKGQPREPQ |
| | | VYTLPPSRDELTKNQVSLTCLVKGFYPSDIAVEWESNGQ |
| | | PENNYKTTPPVLDSDGSFFLYSKLTVDKSRWQQGNVFSC |
| | | SVMHEALHNHYTQKSLSLSPGK |
| 15 | VP1157 light chain | GACATTCAGATGACCCAGAGCCCCAGCACCCTGAGCG |
| | DNA sequence | CCAGCGTGGGAGACAGAGTGACCATCACATGCCAGGC |
| | | TAGCCAGAGCATCTACAGATATTTGAGCTGGTACCAG |
| | | CAGAAACCCGGCAAAGCCCCCAAACTGCTGATCTACG |
| | | ACGCCAGTAAGCTGGCCAGCGGAGTGCCCAGCAGATT |
| | | CAGCGGCAGCGGCAGCGGAACCGAGTTCACCCTGACC |
| | | ATCAGTAGCCTGCAGCCCGACGACTTCGCCACCTACT |
| | | ACTGCCAGCAGGCCTACTCATCCGGAAGCATTGACAA |
| | | CGCCTTCGGCGGCGGGACAAAGGTGGAGATTAAGCGT |
| | | ACGGTGGCAGCTCCATCAGTTTTTATCTTCCCACCAAG |
| | | CGACGAGCAATTGAAGTCCGGCACTGCCTCTGTGGTG |
| | | TGCCTTCTGAACAACTTCTATCCAAGGGAGGCCAAGG |
| | | TCCAGTGGAAGGTCGATAATGCGCTGCAGAGCGGGAA |
| | | CAGCCAAGAGTCAGTGACCGAGCAGGACTCAAAAGAT |
| | | AGCACATACTCTCTGAGTTCCACCCTGACCCTGTCAAA |
| | | GGCTGACTACGAAAAGCATAAGGTATACGCATGCGAA |
| | | GTGACCCATCAGGGTCTCTCATCTCCCGTAACCAAATC |
| | | TTTTAATAGAGGAGAATGC |
| 16 | VP1157 light chain | DIQMTQSPSTLSASVGDRVTITCQASQSIYRYLSWYQQKP |
| | amino acid sequence | GKAPKLLIYDASKLASGVPSRFSGSGSGTEFTLTISSLQPD |
| | | DFATYYCQQAYSSGSIDNAFGGGTKVEIKRTVAAPSVFIF |
| | | PPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |
| 17 | VP1127 heavy chain | GAGGTGCAGCTGCAGGAGAGCGGGGGGGGACTGGTG |
| | DNA sequence | CAGCCAGGAGGAAGCCTGAGACTGAGCTGTGCCGCCA |
| | | GCGGATTTACATTTAGTAGTTATAGTATGAACTGGGTG |
| | | AGGCAGGCCCCCGGAAAAGGCCTGGAGTGGGTGAGCT |
| | | ACATTAGCAGCAGTAGCAGCACCATCTATTACGCTGA |
| | | CTCCGTGAAGGGCAGATTCACCATCTCCAGAGACAAC |
| | | GCCAAAAACAGCCTGTACCTGCAGATGAACAGCCTGA |
| | | GAGCCGAGGACACAGCCGTGTACTACTGCGCCAGATA |
| | | CAGACTGCCCGATTTCTGGAGCGGCTACCCCAACTAC |
| | | GGAATGGATGTGTGGGGCCAGGGCACCACCGTGACAG |
| | | TGAGCAGTGCTAGCACCAAGGGCCCATCGGTCTTCCC |
| | | CCTGGCACCCTCCTCCAAGAGCACCTCTGGGGGCACA |
| | | GCGGCCCTGGGCTGCCTGGTCAAGGACTACTTCCCCG |
| | | AACCGGTGACGGTGTCGTGGAACTCAGGCGCCCTGAC |
| | | CAGCGGCGTGCACACCTTCCCGGCTGTCCTACAGTCCT |
| | | CAGGACTCTACTCCCTCAGCAGCGTGGTGACCGTGCC |
| | | CTCCAGCAGCTTGGGCACCCAGACCTACATCTGCAAC |
| | | GTGAATCACAAGCCCAGCAACACCAAGGTGGACAAG |
| | | AAAGTTGAGCCCAAATCTTGTGACAAAACTCACACAT |
| | | GCCCACCGTGCCCAGCACCTGAACTCCTGGGGGGACC |
| | | GTCAGTCTTCCTCTTCCCCCCAAAACCCAAGGACACCC |
| | | TCATGATCTCCCGGACCCCCGAGGTCACATGCGTGGT |
| | | GGTGGACGTGAGCCACGAAGACCCTGAGGTCAAGTTC |
| | | AACTGGTACGTGGACGGCGTGGAGGTGCATAATGCCA |
| | | AGACAAAGCCGCGGGAGGAGCAGTACAACAGCACGT |
| | | ACCGTGTGGTCAGCGTCCTCACCGTCCTGCACCAGGA |
| | | CTGGCTGAATGGCAAGGAGTACAAGTGCAAGGTCTCC |
| | | AACAAAGCCCTCCCAGCCCCCATCGAGAAAACCATCT |
| | | CCAAAGCCAAAGGGCAGCCCCGAGAACCACAGGTGT |
| | | ACACCCTGCCCCCATCCCGGGAGGAGATGACCAAGAA |
| | | CCAGGTCAGCCTGACCTGCCTGGTCAAAGGCTTCTATC |
| | | CCAGCGACATCGCCGTGGAGTGGGAGAGCAATGGGCA |
| | | GCCGGAGAACAACTACAAGACCACGCCTCCCGTGCTG |
| | | GACTCCGACGGCTCCTTCTTCCTCTACAGCAAGCTCAC |
| | | CGTGGACAAGAGCAGGTGGCAGCAGGGGAACGTCTTC |
| | | TCATGCTCCGTGATGCATGAGGCTCTGCACAACCACTA |
| | | CACGCAGAAGAGCCTCTCCCTGTCTCCGGGTAAA |
| 18 | VP1127 heavy chain | EVQLQESGGGLVQPGGSLRLSCAASGFTFSSYSMNWVR |
| | amino acid sequence | QAPGKGLEWVSYISSSSSTIYYADSVKGRFTISRDNAKNS |
| | | LYLQMNSLRAEDTAVYYCARYRLPDFWSGYPNYGMDV |
| | | WGQGTTVTVSSASTKGPSVFPLAPSSKSTSGGTAALGCL |
| | | VKDYFPEPVTVSWNSGALTSGVHTFPAVLQSSGLYSLSS |
| | | VVTVPSSSLGTQTYICNVNHKPSNTKVDKKVEPKSCDKT |
| | | HTCPPCPAPELLGGPSVFLFPPKPKDTLMISRTPEVTCVV |
| | | VDVSHEDPEVKFNWYVDGVEVHNAKTKPREEQYNSTY |
| | | RVVSVLTVLHQDWLNGKEYKCKVSNKALPAPIEKTISKA |
| | | KGQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| 19 | VP1127 light chain | CAGAGCGTGCTGACCCAGCCCCCCAGCGTGAGCGGAG |
| | DNA sequence | CTCCAGGACAGAGAGTGACAATCAGCTGCACCGGCAG |
| | | CAGCAGCAACATCGGCGCCGGCTACGACGTGCACTGG |
| | | TACCAGCAGCTGCCTGGCACAGCCCCCAAACTGCTGA |
| | | TCTACGGCCAGAGCAACAGACCCAGCGGCGTGCCCGA |
| | | CAGGTTCAGCGGAAGCAAGTCAGGAACCAGCGCCTCC |
| | | CTGGCCATCACCGGACTGCAGGCCGAAGATGAAGCTG |
| | | ACTACTACTGCCAGAGCTACGACAGCAGCCTGAGTGG |
| | | CCACGTGGTGTTTGGCGGAGGAACCAAGCTGACCGTG |
| | | CTGGGTCAGCCCAAGGCCAACCCCACTGTCACTCTGTT |
| | | CCCGCCCTCCTCTGAGGAGCTCCAAGCCAACAAGGCC |
| | | ACACTAGTGTGTCTGATCAGTGACTTCTACCCGGGAGC |
| | | TGTGACAGTGGCCTGGAAGGCAGATGGCAGCCCCGTC |
| | | AAGGCGGGAGTGGAGACCACCAAACCCTCCAAACAG |
| | | AGCAACAACAAGTACGCGGCCAGCAGCTACCTGAGCC |
| | | TGACGCCCGAGCAGTGGAAGTCCCACAGAAGCTACAG |
| | | CTGCCAGGTCACGCATGAAGGGAGCACCGTGGAGAAG |
| | | ACAGTGGCCCCTACAGAATGTTCA |
| 20 | VP1127 light chain | QSVLTQPPSVSGAPGQRVTISCTGSSSNIGAGYDVHWYQ |
| | amino acid sequence | QLPGTAPKLLIYGQSNRPSGVPDRFSGSKSGTSASLAITG |
| | | LQAEDEADYYCQSYDSSLSGHVVFGGGTKLTVLGQPKA |
| | | NPTVTLFPPSSEELQANKATLVCLISDFYPGAVTVAWKA |
| | | DGSPVKAGVETTKPSKQSNNKYAASSYLSLTPEQWKSH |
| | | RSYSCQVTHEGSTVEKTVAPTECS |
| 21 | VP1342 heavy chain | GAGGTGCAGCTGCAGGAGAGCGGGGGGGGACTGGTG |
| | DNA sequence | CAGCCAGGAGGAAGCCTGAGACTGAGCTGTGCCGCCA |
| | | GCGGATTTACATTTAGTAGTTATAGTATGAACTGGGTG |
| | | AGGCAGGCCCCCGGAAAAGGCCTGGAGTGGGTGAGCT |
| | | ACATTAGCAGCAGTAGCAGCACCATCTATTACGCTGA |
| | | CTCCGTGAAGGGCAGATTCACCATCTCCAGAGACAAC |
| | | GCCAAAAACAGCCTGTACCTGCAGATGAACAGCCTGA |
| | | GAGCCGAGGACACAGCCGTGTACTACTGCGCCAGATA |
| | | CAGACTGCCCGATTTCTGGAGCGGCTACCCCAACTAC |
| | | GGAATGGATGTGTGGGGCCAGGGCACCACCGTGACAG |
| | | TGAGCAGTGCTAGCACCAAGGGCCCATCGGTCTTCCC |
| | | CCTGGCACCCTCCTCCAAGAGCACCTCTGGGGGCACA |
| | | GCGGCCCTGGGCTGCCTGGTCAAGGACTACTTCCCCG |
| | | AACCGGTGACGGTGTCGTGGAACTCAGGCGCCCTGAC |
| | | CAGCGGCGTGCACACCTTCCCGGCTGTCCTACAGTCCT |
| | | CAGGACTCTACTCCCTCAGCAGCGTGGTGACCGTGCC |
| | | CTCCAGCAGCTTGGGCACCCAGACCTACATCTGCAAC |
| | | GTGAATCACAAGCCCAGCAACACCAAGGTGGACAAG |
| | | AAAGTTGAGCCCAAATCTTGTGACAAAACTCACACAT |
| | | GCCCACCGTGCCCAGCACCTGAACTCCTGGGGGGACC |
| | | GTCAGTCTTCCTCTTCCCCCCAAAACCCAAGGACACCC |
| | | TCATGATCTCCCGGACCCCCGAGGTCACATGCGTGGT |
| | | GGTGGACGTGAGCCACGAAGACCCTGAGGTCAAGTTC |
| | | AACTGGTACGTGGACGGCGTGGAGGTGCATAATGCCA |
| | | AGACAAAGCCGCGGGAGGAGCAGTACAACAGCACGT |
| | | ACCGTGTGGTCAGCGTCCTCACCGTCCTGCACCAGGA |
| | | CTGGCTGAATGGCAAGGAGTACAAGTGCAAGGTCTCC |
| | | AACAAAGCCCTCCCAGCCCCCATCGAGAAAACCATCT |
| | | CCAAAGCCAAAGGGCAGCCCCGAGAACCACAGGTGT |
| | | ACACCCTGCCCCCATCCCGGGAGGAGATGACCAAGAA |
| | | CCAGGTCAGCCTGACCTGCCTGGTCAAAGGCTTCTATC |
| | | CCAGCGACATCGCCGTGGAGTGGGAGAGCAATGGGCA |
| | | GCCGGAGAACAACTACAAGACCACGCCTCCCGTGCTG |
| | | GACTCCGACGGCTCCTTCTTCCTCTACAGCAAGCTCAC |
| | | CGTGGACAAGAGCAGGTGGCAGCAGGGGAACGTCTTC |
| | | TCATGCTCCGTGATGCATGAGGCTCTGCACAACCACTA |
| | | CACGCAGAAGAGCCTCTCCCTGTCTCCGGGTAAA |
| 22 | VP1342 heavy chain | EVQLQESGGGLVQPGGSLRLSCAASGFTFSSYSMNWVR |
| | amino acid sequence | QAPGKGLEWVSYISSSSSTIYYADSVKGRFTISRDNAKNS |
| | | LYLQMNSLRAEDTAVYYCARYRLPDFWSGYPNYGMDV |
| | | WGQGTTVTVSSASTKGPSVFPLAPSSKSTSGGTAALGCL |
| | | VKDYFPEPVTVSWNSGALTSGVHTFPAVLQSSGLYSLSS |
| | | VVTVPSSSLGTQTYICNVNHKPSNTKVDKKVEPKSCDKT |
| | | HTCPPCPAPELLGGPSVFLFPPKPKDTLMISRTPEVTCVV |
| | | VDVSHEDPEVKFNWYVDGVEVHNAKTKPREEQYNSTY |
| | | RVVSVLTVLHQDWLNGKEYKCKVSNKALPAPIEKTISKA |
| | | KGQPREPQVYTLPPSREEMTKNQVSLTCLVKGFYPSDIA |
| | | VEWESNGQPENNYKTTPPVLDSDGSFFLYSKLTVDKSR |
| | | WQQGNVFSCSVMHEALHNHYTQKSLSLSPGK |
| 23 | VP1342 light chain | CAGTCCGTCCTCACCCAGCCCCCATCCGTCTCCGGCGC |
| | DNA sequence | ACCAGGACAGCGGGTCACCATCTCCTGCACCGGCTCC |
| | | TCCTCCAACATCGGCGCCGGCTACGACGTCCACTGGT |
| | | ACCAGCAGCTCCCAGGCACCGCCCCCAAACTCCTGAT |
| | | CTACGGCCAGAGCAACAGACCCTCCGGCGTCCCCGAC |
| | | AGATTCTCCGGCTCCAAGTCTGGAACATCCGCCTCCCT |
| | | CGCTATCACCGGCCTCCAGGCAGAAGATGAAGCTGAC |
| | | TACTACTGCCAGTCCTACTCCTCCTCCCTCAGCGGTCA |
| | | CGTGGTGTTCGGCGGGGGCACCAAGCTGACAGTGCTG |
| | | GGTCAGCCCAAGGCCAACCCCACTGTCACTCTGTTCCC |
| | | GCCCTCCTCTGAGGAGCTCCAAGCCAACAAGGCCACA |
| | | CTAGTGTGTCTGATCAGTGACTTCTACCCGGGAGCTGT |
| | | GACAGTGGCCTGGAAGGCAGATGGCAGCCCCGTCAAG |
| | | GCGGGAGTGGAGACCACCAAACCCTCCAAACAGAGC |
| | | AACAACAAGTACGCGGCCAGCAGCTACCTGAGCCTGA |
| | | CGCCCGAGCAGTGGAAGTCCCACAGAAGCTACAGCTG |
| | | CCAGGTCACGCATGAAGGGAGCACCGTGGAGAAGAC |
| | | AGTGGCCCCTACAGAATGTTCA |
| 24 | VP1342 light chain | QSVLTQPPSVSGAPGQRVTISCTGSSSNIGAGYDVHWYQ |
| | amino acid sequence | QLPGTAPKLLIYGQSNRPSGVPDRFSGSKSGTSASLAITG |
| | | LQAEDEADYYCQSYSSSLSGHVVFGGGTKLTVLGQPKA |
| | | NPTVTLFPPSSEELQANKATLVCLISDFYPGAVTVAWKA |
| | | DGSPVKAGVETTKPSKQSNNKYAASSYLSLTPEQWKSH |
| | | RSYSCQVTHEGSTVEKTVAPTECS |

Table 9 and Table 10 provide the amino acid sequences for the CDRs referenced above. CDRs were determined by Kabat numbering system.

TABLE 9
Antibody Heavy Chain CDR sequences

| | | | SEQ | | SEQ | | SEQ |
|---|---|---|---|---|---|---|---|
| Antibody | Target | | ID | | ID | | ID |
| ID | Antigen | HC-CDR-1 | NO: | HC-CDR-2 | NO: | HC-CDR-3 | NO: |
| VP0270 | ICAM1 | SHAMG | 25 | IIGSSDRTYYAS | 26 | DPYDSYDEGYRL | 27 |
| | | | | WAKG | | | |
| VP1157 | ICAM1 | SHAMG | 28 | IIGSSDRTYYA | 29 | DPYDSYDEGYRL | 30 |
| | | | | DSVKG | | | |
| VP0633 | EphA2 | SYSMN | 31 | YISSSSSTIYYA | 32 | YRLPNFWSGYPN | 33 |
| | | | | DSVKG | | YGTDV | |
| VP0253 | EphA2 | SYSMN | 34 | YISSSSSTIYYA | 35 | YRLPDFWSGYPN | 36 |
| | | | | DSVKG | | YGMDV | |
| VP1127 | EphA2 | SYSMN | 37 | YISSSSSTIYYA | 38 | YRLPDFWSGYPN | 39 |
| | | | | DSVKG | | YGMDV | |
| VP1342 | EphA2 | SYSMN | 40 | YISSSSSTIYYA | 41 | YRLPDFWSGYPN | 42 |
| | | | | DSVKG | | YGMDV | |

TABLE 10
Antibody Light Chain CDR sequences

| | | | SEQ | | SEQ | | SEQ |
|---|---|---|---|---|---|---|---|
| Antibody | Target | | ID | | ID | | ID |
| ID | Antigen | LC-CDR-1 | NO: | LC-CDR-2 | NO: | LC-CDR-3 | NO: |
| VP0270 | ICAM1 | QASQSIYRY | 43 | DASKLAS | 44 | QQAYSSGSID | 45 |
| | | LS | | | | NA | |
| VP1157 | ICAM1 | QASQSIYRY | 46 | DASKLAS | 47 | QQAYSSGSID | 48 |
| | | LS | | | | NA | |
| VP0633 | EphA2 | TGSSSNIGA | 49 | GNSNRPS | 50 | QSYDSSLSGH | 51 |
| | | GYDVH | | | | VV | |
| VP0253 | EphA2 | TGSSSNIGA | 52 | GNSNRPS | 53 | QSYDSSLSGH | 54 |
| | | GYDVH | | | | VV | |
| VP1127 | EphA2 | TGSSSNIGA | 55 | GQSNRPS | 56 | QSYDSSLSGH | 57 |
| | | GYDVH | | | | VV | |
| VP1342 | EphA2 | TGSSSNIGA | 58 | GQSNRPS | 59 | QSYSSSLSGH | 60 |
| | | GYDVH | | | | VV | |

TABLE 11
Antibody Heavy Chain Variable Domain Sequences

| Anti- | Target | | SEQ |
|---|---|---|---|
| body | Anti- | | ID |
| ID | gen | Heavy Chain Variable Domain | NO: |
| VP0270 | ICAM1 | EVQVLESGGGLVQPGGSLRLSCAVSGFSLSS | 61 |
| | | HAMGWVRQAPGKGLEWIGIIGSSDRTYYAS | |
| | | WAKGRFTISKDNSKNTVYLQINSPRAEDTAV | |
| | | YYCVRDPYDSYDEGYRLWGQGTLVTVSS | |
| VP1157 | ICAM1 | EVQLLESGGGLVQPGGSLRLSCAASGFSLSS | 62 |
| | | HAMGWVRQAPGKGLEWVSIIGSSDRTYYAD | |
| | | SVKGRFTISRDNSKNTLYLQMNSLRAEDTAV | |
| | | YYCAKDPYDSYDEGYRLWGQGTLVTVSS | |
| VP0633 | EphA2 | EVQLVESGGGLVQPGGSLRLSCAASGFTFSS | 63 |
| | | YSMNWVRQAPGKGLEWVSYISSSSSTIYYA | |
| | | DSVKGRFTISRDNAKNSLYLQMNSLRAEDT | |
| | | AVYYCARYRLPNFWSGYPNYGTDVWGQGT | |
| | | TVTVSS | |
| VP0253 | EphA2 | QVQLQESGGGLVQPGGSLRLSCAASGFTFSS | 64 |
| | | YSMNWVRQAPGKGLEWVSYISSSSSTIYYA | |
| | | DSVKGRFTISRDNAKNSLYLQMNSLRAEDT | |
| | | AVYYCARYRLPDFWSGYPNYGMDVWGQG | |
| | | TTVTVSS | |
| VP1127 | EphA2 | EVQLQESGGGLVQPGGSLRLSCAASGFTFSS | 65 |
| | | YSMNWVRQAPGKGLEWVSYISSSSSTIYYA | |
| | | DSVKGRFTISRDNAKNSLYLQMNSLRAEDT | |
| | | AVYYCARYRLPDFWSGYPNYGMDVWGQG | |
| | | TTVTVSS | |
| VP1342 | EphA2 | EVQLQESGGGLVQPGGSLRLSCAASGFTFSS | 66 |
| | | YSMNWVRQAPGKGLEWVSYISSSSSTIYYA | |
| | | DSVKGRFTISRDNAKNSLYLQMNSLRAEDT | |
| | | AVYYCARYRLPDFWSGYPNYGMDVWGQG | |
| | | TTVTVSS | |

TABLE 12
Antibody Light Chain Variable Domain Sequences

| | | | SEQ |
|---|---|---|---|
| Antibody | Target | | ID |
| ID | Antigen | Light Chain Variable Domain | NO: |
| VP0270 | ICAM1 | DIQMTQSPSTLSASVGDRVTITCQASQSIYRYLS | 67 |
| | | WYQQKPGKAPKFLIYDASKLASGVPSRFSGSGS | |
| | | GTEFTLTISSVQPDDFATYYCQQAYSSGSIDNAF | |
| | | GGGTKVEIK | |
| VP1157 | ICAM1 | DIQMTQSPSTLSASVGDRVTITCQASQSIYRYLS | 68 |
| | | WYQQKPGKAPKLLIYDASKLASGVPSRFSGSG | |
| | | SGTEFTLTISSLQPDDFATYYCQQAYSSGSIDNA | |
| | | FGGGTKVEIK | |
| VP0633 | EphA2 | QSVLTQPPSVSGAPGQRVTISCTGSSSNIGAGYD | 69 |
| | | VHWYQQLPGTAPKLLIYGNSNRPSGVPDRFSG | |
| | | SKSGTSASLAITGLQAEDEADYYCQSYDSSLSG | |
| | | HVVFGGGTKLTVL | |
| VP0253 | EphA2 | QSVLTQPPSVSGAPGQRVTISCTGSSSNIGAGYD | 70 |
| | | VHWYQQLPGTAPKLLIYGNSNRPSGVPDRFSG | |
| | | SKSGTSASLAITGLQAEDEADYYCQSYDSSLSG | |
| | | HVVFGGGTKLTVL | |
| VP1127 | EphA2 | QSVLTQPPSVSGAPGQRVTISCTGSSSNIGAGYD | 71 |
| | | VHWYQQLPGTAPKLLIYGQSNRPSGVPDRFSG | |
| | | SKSGTSASLAITGLQAEDEADYYCQSYDSSLSG | |
| | | HVVFGGGTKLTVL | |
| VP1342 | EphA2 | QSVLTQPPSVSGAPGQRVTISCTGSSSNIGAGYD | 72 |
| | | VHWYQQLPGTAPKLLIYGQSNRPSGVPDRFSG | |
| | | SKSGTSASLAITGLQAEDEADYYCQSYSSSLSG | |
| | | HVVFGGGTKLTVL | |

### Embodiments

Embodiment A1 comprises an antibody that comprises a heavy chain variable domain that comprises complementarity determining regions (CDR)s: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise the amino acid sequences according to HC-CDR1: SEQ ID NO: 28, HC-CDR2: SEQ ID NO: 29, HC-CDR3: SEQ ID NO: 30, LC-CDR1: SEQ ID NO: 46, LC-CDR2: SEQ ID NO: 47, LC-CDR3: SEQ ID NO: 48.

Embodiment A2 comprises the antibody according to embodiment A1, wherein the antibody comprises an antibody format selected from IgG, Fab, Fab′, scFv, and (Fab′)₂.

Embodiment A3 comprises the antibody according to any one of embodiments A1-A2, wherein the heavy chain variable domain is fused to a human IgG1 constant region.

Embodiment A4 comprises the antibody according to any one of embodiments A1-A2, wherein the heavy chain variable domain is fused to a human IgG4 constant region.

Embodiment A5 comprises the antibody according to any one of embodiments A1-A4, wherein the light chain variable domain is fused to a human Kappa constant region.

Embodiment A6 comprises the antibody according to any one of embodiments A1-A5, wherein the heavy chain variable domain comprises a variable domain of an IgG1, IgG2, IgG3, or IgG4 heavy chain.

Embodiment A7 comprises the antibody according to any one of embodiments A1-A6, wherein the light chain variable domain comprises a variable domain of a Kappa or Lambda light chain.

Embodiment A8 comprises the antibody according to any one of embodiments A1-A7, wherein the heavy chain variable domain comprises an amino acid sequence with at least 90%, 95%, 96%, 97%, 98%, 99%, or 100% identity to SEQ ID NO:62.

Embodiment A9 comprises the antibody according to any one of embodiments A1-A8, wherein the light chain variable domain comprises an amino acid sequence with at least 90%, 95%, 96%, 97%, 98%, 99%, or 100% identity to SEQ ID NO: 68.

Embodiment A10 comprises the antibody according to any one of embodiments A1-A9, wherein the antibody comprises an amino acid sequence with at least 90%, 95%, 96%, 97%, 98%, 99%, or 100% identity to SEQ ID NOs: 14.

Embodiment A11 comprises the antibody according to any one of embodiments A1-A10, wherein the antibody comprises an amino acid sequence with at least 90%, 95%, 96%, 97%, 98%, 99%, or 100% identity to SEQ ID NOs: 16.

Embodiment A12 comprises the antibody according to any one of embodiments A1-A11, wherein the antibody is conjugated to a payload.

Embodiment A13 comprises a nucleic acid molecule encoding the antibody of any one of embodiments A1-A11.

Embodiment A14 comprises a vector comprising the nucleic acid molecule of embodiment A13.

Embodiment A15 comprises a pharmaceutical composition comprising the antibody of any one of embodiments A1-A12.

Embodiment A16 comprises the pharmaceutical composition of embodiment A15, further comprising a pharmaceutically acceptable carrier, an excipient, or any combinations thereof.

Embodiment A17 comprises a method of treating a subject having cancer, the method comprising: administering to the subject the antibody of any one of embodiments A1-A12.

Embodiment A18 comprises the method of embodiment A17 wherein the cancer comprises cancer cells that express ICAM1.

Embodiment B1 comprises an antibody that comprises a heavy chain variable domain that comprises complementarity determining regions (CDR)s: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise the amino acid sequences according to HC-CDR1: SEQ ID NO: 37, HC-CDR2: SEQ ID NO: 38, HC-CDR3: SEQ ID NO: 39, LC-CDR1: SEQ ID NO: 55, LC-CDR2: SEQ ID NO: 56, LC-CDR3: SEQ ID NO: 57.

Embodiment B2 comprises the antibody according to embodiment B1, wherein the antibody comprises an antibody format selected from IgG, Fab, Fab′, scFv, and (Fab′)₂.

Embodiment B3 comprises the antibody according to any one of embodiments B1-B2, wherein the heavy chain variable domain is fused to a human IgG1 constant region.

Embodiment B4 comprises the antibody according to any one of embodiments B1-B2, wherein the heavy chain variable domain is fused to a human IgG4 constant region.

Embodiment B5 comprises the antibody according to any one of embodiments B1-B4, wherein the light chain variable domain is fused to a human Lambda constant region.

Embodiment B6 comprises the antibody according to any one of embodiments B1-B5, wherein the heavy chain variable domain comprises a variable domain of an IgG1, IgG2, IgG3, or IgG4 heavy chain.

Embodiment B7 comprises the antibody according to any one of embodiments B1-B6, wherein the light chain variable domain comprises a variable domain of a Kappa or Lambda light chain.

Embodiment B8 comprises the antibody according to any one of embodiments B1-B7, wherein the heavy chain variable domain comprises an amino acid sequence with at least 90%, 95%, 96%, 97%, 98%, 99%, or 100% identity to SEQ ID NO: 65.

Embodiment B9 comprises the antibody according to any one of embodiments B1-B8, wherein the light chain variable domain comprises an amino acid sequence with at least 90%, 95%, 96%, 97%, 98%, 99%, or 100% identity to SEQ ID NO: 71.

Embodiment B10 comprises the antibody according to any one of embodiments B1-B9, wherein the antibody comprises an amino acid sequence with at least 90%, 95%, 96%, 97%, 98%, 99%, or 100% identity to SEQ ID NOs: 18.

Embodiment B11 comprises the antibody according to any one of embodiments B1-B10, wherein the antibody comprises an amino acid sequence with at least 90%, 95%, 96%, 97%, 98%, 99%, or 100% identity to SEQ ID NOs: 20.

Embodiment B12 comprises the antibody according to any one of embodiments B1-B11, wherein the antibody is conjugated to a payload.

Embodiment B13 comprises a nucleic acid molecule encoding the antibody of any one of embodiments B1-B11.

Embodiment B14 comprises a vector comprising the nucleic acid molecule of embodiment B13.

Embodiment B15 comprises a pharmaceutical composition comprising the antibody of any one of embodiments B1-B12.

Embodiment B16 comprises the pharmaceutical composition of embodiment B15, further comprising a pharmaceutically acceptable carrier, an excipient, or any combinations thereof.

Embodiment B17 comprises a method of treating a subject having cancer, the method comprising: administering to the subject the antibody of any one of embodiments B1-B12.

Embodiment B18 comprises the method of embodiment B17 wherein the cancer comprises cancer cells that express EphA2.

Embodiment C1 comprises an antibody that comprises a heavy chain variable domain that comprises complementarity determining regions (CDR)s: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise the amino acid sequences according to HC-CDR1: SEQ ID NO: 40, HC-CDR2: SEQ ID NO: 41, HC-CDR3: SEQ ID NO: 42, LC-CDR1: SEQ ID NO: 58, LC-CDR2: SEQ ID NO: 59, LC-CDR3: SEQ ID NO: 60.

Embodiment C2 comprises the antibody according to embodiment C1, wherein the antibody comprises an antibody format selected from IgG, Fab, Fab′, scFv, and (Fab′)₂.

Embodiment C3 comprises the antibody according to any one of embodiments C1-C2, wherein the heavy chain variable domain is fused to a human IgG1 constant region.

Embodiment C4 comprises the antibody according to any one of embodiments C1-C2, wherein the heavy chain variable domain is fused to a human IgG4 constant region.

Embodiment C5 comprises the antibody according to any one of embodiments C₁-C₄, wherein the light chain variable domain is fused to a human Lambda constant region.

Embodiment C6 comprises the antibody according to any one of embodiments C₁-C₅, wherein the heavy chain variable domain comprises a variable domain of an IgG1, IgG2, IgG3, or IgG4 heavy chain.

Embodiment C7 comprises the antibody according to any one of embodiments C1-C6, wherein the light chain variable domain comprises a variable domain of a Kappa or Lambda light chain.

Embodiment C8 comprises the antibody according to any one of embodiments C1-C7, wherein the heavy chain variable domain comprises an amino acid sequence with at least 90%, 95%, 96%, 97%, 98%, 99%, or 100% identity to SEQ ID NO: 66.

Embodiment C9 comprises the antibody according to any one of embodiments C1-C8, wherein the light chain variable domain comprises an amino acid sequence with at least 90%, 95%, 96%, 97%, 98%, 99%, or 100% identity to SEQ ID NO: 72.

Embodiment C10 comprises the antibody according to any one of embodiments C1-C9, wherein the antibody comprises an amino acid sequence with at least 90%, 95%, 96%, 97%, 98%, 99%, or 100% identity to SEQ ID NOs: 22.

Embodiment C11 comprises the antibody according to any one of embodiments C1-C10, wherein the antibody comprises an amino acid sequence with at least 90%, 95%, 96%, 97%, 98%, 99%, or 100% identity to SEQ ID NOs: 24.

Embodiment C12 comprises the antibody according to any one of embodiments C1-C11, wherein the antibody is conjugated to a payload.

Embodiment C13 comprises a nucleic acid molecule encoding the antibody of any one of embodiments C1-C11.

Embodiment C14 comprises a vector comprising the nucleic acid molecule of embodiment C13.

Embodiment C15 comprises a pharmaceutical composition comprising the antibody of any one of embodiments C1-C12.

Embodiment C16 comprises the pharmaceutical composition of embodiment C15, further comprising a pharmaceutically acceptable carrier, an excipient, or any combinations thereof.

Embodiment C17 comprises a method of treating a subject having cancer, the method comprising: administering to the subject the antibody of any one of embodiments C1-C12.

Embodiment C18 comprises the method of embodiment C17 wherein the cancer comprises cancer cells that express EphA2.

Embodiment D1 comprises an antibody that comprises a heavy chain variable domain that comprises complementarity determining regions (CDR)s: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise the amino acid sequences according to HC-CDR1: SEQ ID NO: 25, HC-CDR2: SEQ ID NO: 26, HC-CDR3: SEQ ID NO: 27, LC-CDR1: SEQ ID NO: 43, LC-CDR2: SEQ ID NO: 44, LC-CDR3: SEQ ID NO: 45.

Embodiment D2 comprises the antibody according to embodiment D1, wherein the antibody comprises an antibody format selected from IgG, Fab, Fab′, scFv, and (Fab′)₂.

Embodiment D3 comprises the antibody according to any one of embodiments D1-D2, wherein the heavy chain variable domain is fused to a human IgG1 constant region.

Embodiment D4 comprises the antibody according to any one of embodiments D1-D2, wherein the heavy chain variable domain is fused to a human IgG4 constant region.

Embodiment D5 comprises the antibody according to any one of embodiments D1-D4, wherein the light chain variable domain is fused to a human Kappa constant region.

Embodiment D6 comprises the antibody according to any one of embodiments D1-D5, wherein the heavy chain variable domain comprises a variable domain of an IgG1, IgG2, IgG3, or IgG4 heavy chain.

Embodiment D7 comprises the antibody according to any one of embodiments D1-D6, wherein the light chain variable domain comprises a variable domain of a Kappa or Lambda light chain.

Embodiment D8 comprises the antibody according to any one of embodiments D1-D7, wherein the heavy chain variable domain comprises an amino acid sequence with at least 90%, 95%, 96%, 97%, 98%, 99%, or 100% identity to SEQ ID NO:61.

Embodiment D9 comprises the antibody according to any one of embodiments D1-D8, wherein the light chain variable domain comprises an amino acid sequence with at least 90%, 95%, 96%, 97%, 98%, 99%, or 100% identity to SEQ ID NO: 67.

Embodiment D10 comprises the antibody according to any one of embodiments D1-D9, wherein the antibody comprises an amino acid sequence with at least 90%, 95%, 96%, 97%, 98%, 99%, or 100% identity to SEQ ID NOs: 2.

Embodiment D11 comprises the antibody according to any one of embodiments D1-D10, wherein the antibody comprises an amino acid sequence with at least 90%, 95%, 96%, 97%, 98%, 99%, or 100% identity to SEQ ID NOs: 4.

Embodiment D12 comprises the antibody according to any one of embodiments D1-D11, wherein the antibody is conjugated to a payload.

Embodiment D13 comprises a nucleic acid molecule encoding the antibody of any one of embodiments D1-D11.

Embodiment D14 comprises a vector comprising the nucleic acid molecule of embodiment D13.

Embodiment D15 comprises a pharmaceutical composition comprising the antibody of any one of embodiments D1-D12.

Embodiment D16 comprises the pharmaceutical composition of embodiment D15, further comprising a pharmaceutically acceptable carrier, an excipient, or any combinations thereof.

Embodiment D17 comprises a method of treating a subject having cancer, the method comprising: administering to the subject the antibody of any one of embodiments D1-D12.

Embodiment D18 comprises the method of embodiment D17 wherein the cancer comprises cancer cells that express ICAM1.

Embodiment E1 comprises an antibody that comprises a heavy chain variable domain that comprises complementarity determining regions (CDR)s: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise the amino acid sequences according to HC-CDR1: SEQ ID NO: 31, HC-CDR2: SEQ ID NO: 32, HC-CDR3: SEQ ID NO: 33, LC-CDR1: SEQ ID NO: 49, LC-CDR2: SEQ ID NO: 50, LC-CDR3: SEQ ID NO: 51.

Embodiment E2 comprises the antibody according to embodiment E1, wherein the antibody comprises an antibody format selected from IgG, Fab, Fab′, scFv, and (Fab′)₂.

Embodiment E3 comprises the antibody according to any one of embodiments E1-E2, wherein the heavy chain variable domain is fused to a human IgG1 constant region.

Embodiment E4 comprises the antibody according to any one of embodiments E1-E2, wherein the heavy chain variable domain is fused to a human IgG4 constant region.

Embodiment E5 comprises the antibody according to any one of embodiments E1-E4, wherein the light chain variable domain is fused to a human Lambda constant region.

Embodiment E6 comprises the antibody according to any one of embodiments E1-E5, wherein the heavy chain variable domain comprises a variable domain of an IgG1, IgG2, IgG3, or IgG4 heavy chain.

Embodiment E7 comprises the antibody according to any one of embodiments E1-E6, wherein the light chain variable domain comprises a variable domain of a Kappa or Lambda light chain.

Embodiment E8 comprises the antibody according to any one of embodiments E1-E7, wherein the heavy chain variable domain comprises an amino acid sequence with at least 90%, 95%, 96%, 97%, 98%, 99%, or 100% identity to SEQ ID NO: 63.

Embodiment E9 comprises the antibody according to any one of embodiments E1-E8, wherein the light chain variable domain comprises an amino acid sequence with at least 90%, 95%, 96%, 97%, 98%, 99%, or 100% identity to SEQ ID NO: 69.

Embodiment E10 comprises the antibody according to any one of embodiments E1-E9, wherein the antibody comprises an amino acid sequence with at least 90%, 95%, 96%, 97%, 98%, 99%, or 100% identity to SEQ ID NOs: 6.

Embodiment E11 comprises the antibody according to any one of embodiments E1-E10, wherein the antibody comprises an amino acid sequence with at least 90%, 95%, 96%, 97%, 98%, 99%, or 100% identity to SEQ ID NOs: 8.

Embodiment E12 comprises the antibody according to any one of embodiments E1-E11, wherein the antibody is conjugated to a payload.

Embodiment E13 comprises a nucleic acid molecule encoding the antibody of any one of embodiments E1-E11.

Embodiment E14 comprises a vector comprising the nucleic acid molecule of embodiment E13.

Embodiment E15 comprises a pharmaceutical composition comprising the antibody of any one of embodiments E1-E12.

Embodiment E16 comprises the pharmaceutical composition of embodiment E15, further comprising a pharmaceutically acceptable carrier, an excipient, or any combinations thereof.

Embodiment E17 comprises a method of treating a subject having cancer, the method comprising: administering to the subject the antibody of any one of embodiments E1-E12.

Embodiment E18 comprises the method of embodiment E17 wherein the cancer comprises cancer cells that express EphA2.

Embodiment F1 comprises an antibody that comprises a heavy chain variable domain that comprises complementarity determining regions (CDR)s: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise the amino acid sequences according to HC-CDR1: SEQ ID NO: 34, HC-CDR2: SEQ ID NO: 35, HC-CDR3: SEQ ID NO: 36, LC-CDR1: SEQ ID NO: 52, LC-CDR2: SEQ ID NO: 53, LC-CDR3: SEQ ID NO: 54.

Embodiment F2 comprises the antibody according to embodiment F1, wherein the antibody comprises an antibody format selected from IgG, Fab, Fab′, scFv, and (Fab′)₂.

Embodiment F3 comprises the antibody according to any one of embodiments F1-F2, wherein the heavy chain variable domain is fused to a human IgG1 constant region.

Embodiment F4 comprises the antibody according to any one of embodiments F1-F2, wherein the heavy chain variable domain is fused to a human IgG4 constant region.

Embodiment F5 comprises the antibody according to any one of embodiments F1-F4, wherein the light chain variable domain is fused to a human Lambda constant region.

Embodiment F6 comprises the antibody according to any one of embodiments F1-F5, wherein the heavy chain variable domain comprises a variable domain of an IgG1, IgG2, IgG3, or IgG4 heavy chain.

Embodiment F7 comprises the antibody according to any one of embodiments F1-F6, wherein the light chain variable domain comprises a variable domain of a Kappa or Lambda light chain.

Embodiment F8 comprises the antibody according to any one of embodiments F1-F7, wherein the heavy chain variable domain comprises an amino acid sequence with at least 90%, 95%, 96%, 97%, 98%, 99%, or 100% identity to SEQ ID NO: 64.

Embodiment F9 comprises the antibody according to any one of embodiments F1-F8, wherein the light chain variable domain comprises an amino acid sequence with at least 90%, 95%, 96%, 97%, 98%, 99%, or 100% identity to SEQ ID NO: 70.

Embodiment F10 comprises the antibody according to any one of embodiments F1-F9, wherein the antibody comprises an amino acid sequence with at least 90%, 95%, 96%, 97%, 98%, 99%, or 100% identity to SEQ ID NOs: 10.

Embodiment F11 comprises the antibody according to any one of embodiments F1-F10, wherein the antibody comprises an amino acid sequence with at least 90%, 95%, 96%, 97%, 98%, 99%, or 100% identity to SEQ ID NOs: 12.

Embodiment F12 comprises the antibody according to any one of embodiments F1-F11, wherein the antibody is conjugated to a payload.

Embodiment F13 comprises a nucleic acid molecule encoding the antibody of any one of embodiments F1-F11.

Embodiment F14 comprises a vector comprising the nucleic acid molecule of embodiment F13.

Embodiment F15 comprises a pharmaceutical composition comprising the antibody of any one of embodiments F1-F12.

Embodiment F16 comprises the pharmaceutical composition of embodiment F15, further comprising a pharmaceutically acceptable carrier, an excipient, or any combinations thereof.

Embodiment F17 comprises a method of treating a subject having cancer, the method comprising: administering to the subject the antibody of any one of embodiments F1-F12.

Embodiment F18 comprises the method of embodiment F17 wherein the cancer comprises cancer cells that express EphA2.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound of Formula (I): 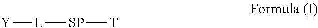
wherein,
T is a moiety derived from a compound capable of inhibiting topoisomerase or poly (ADP-ribose) polymerase (PARP);
SP is absent, a di- or tri-peptide linking moiety having L bonded to the N-terminus and T bonded to the C-terminus, or a linking moiety capable of self-immolation at pH less than 8;
L is a di- or tri-peptide linking moiety having Y bonded to the N-terminus and SP bonded to the C-terminus; and
Y is a conjugation moiety capable of forming a covalent bond with a nitrogen atom of a lysine residue or a sulfur atom of a cysteine residue;
or a salt or a hydrate thereof.

2. The compound of claim 1, wherein Y is a conjugation moiety capable of forming a covalent bond with a nitrogen atom.

3. The compound of claim 1, wherein Y is a conjugation moiety capable of forming a covalent bond with a sulfur atom.

4. The compound of claim 2, wherein the conjugation moiety comprises an activated carbonyl group.

5. The compound of claim 4, wherein the activated carbonyl group is

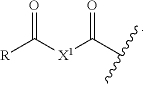

6. The compound of claim 5, wherein X¹ is selected from bond, —(CH₂)ₙ—, —(CH₂)ₙ—(OCH₂CH₂)ₘ—, —(CH₂)ₙ—O—(CH₂)ₘ—, and —(CH₂)ₙ—NH—(CH₂)ₘ—, wherein n is an integer from 1 to 5, and m is an integer from 1 to 8.

7. The compound of claim 5, wherein R is selected from the group consisting of: 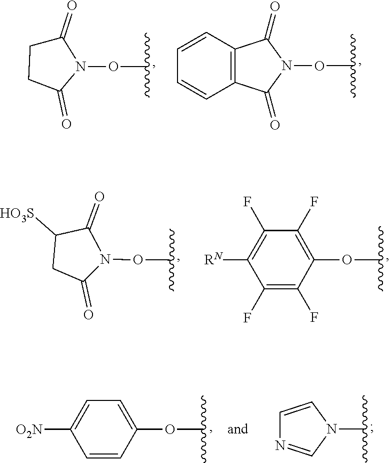
wherein R^{N} is hydrogen or fluoro.

8. The compound of claim 3, wherein the conjugation moiety comprises an acyl halide group or a Michael acceptor group.

9. The compound of claim 8, wherein the conjugation moiety is an acyl halide group.

10. The compound of claim 9, wherein the acyl halide is:

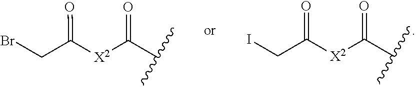

11. The compound of claim 10, wherein X² is selected from bond, —(CH₂)ₙ—, —(CH₂)ₙ—(OCH₂CH₂)ₚ—, —(CH₂)ₙ—O—(CH₂)ₚ—, and —(CH₂)ₙ—NH—(CH₂)ₚ—, wherein n is an integer from 1 to 5, and p is an integer from 1 to 8.

12. The compound of claim 8, wherein the conjugation moiety is a Michael acceptor group.

13. The compound of claim 12, wherein the Michael acceptor group is: 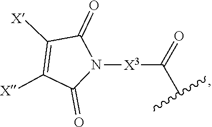
wherein X³ is selected from bond, —(CH₂)ₙ—, —(CH₂)ₙ—(OCH₂CH₂)ₚ—, —(CH₂)ₙ—O—(CH₂)ₚ—, —(CH₂)ₙ—NH—(CH₂)ₚ—, wherein n is an integer from 1 to 5, and p is an integer from 1 to 8; wherein x′ is H, Cl, Br, I, 2-thiopyridyl, or 4-cyanophenoxy; and x″ is H, Cl, Br, I, 2-thiopyridyl, or 4-cyanophenoxy.

14. The compound of claim 12, wherein x′ and x″ are both H or both Br.

15. The compound of any one of claims 1 to 14, wherein L is a di-peptide linking moiety having the structure of: 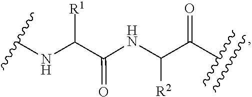
wherein R¹ and R² are each independently selected from H, —CH₂CH₂CH₂NHCONH₂, or a side chain of a naturally occurring amino acid, and wherein [symbol] represents the point of attachment to Y and [symbol] represents the point of attachment to SP.

16. The compound of claim 15, wherein R¹ and R² are each independently selected from the group consisting of hydrogen, —CH₃, —CH(CH₃)₂, —CH₂CH₂CH₂CH₂NH₂, —CH₂C₆H₅, —CH₂C₆H₄OH, —CH₂CH₂CH₂NH(NH)NH₂, —CH₂CH₂CH₂NHCONH₂, and —CH₂CH₂CO₂H.

17. The compound of claim 16, wherein R¹ is selected from the group consisting of —CH₃, —CH(CH₃)₂, —CH₂CH₂CH₂CH₂NH₂, and —CH₂C₆H₅.

18. The compound of claim 16 or 17, wherein R² is selected from the group consisting of —CH₃, —CH₂CH₂CH₂CH₂NH₂, —CH₂C₆H₅, and —CH₂CH₂CH₂NHCONH₂.

19. The compound of claim 16 or 17, wherein R¹ is —CH₃ and R² is —CH₂CH₂CH₂CH₂NH₂.

20. The compound of claim 16 or 17, wherein R¹ is —CH₃ and R² is —CH₃.

21. The compound of claim 16 or 17, wherein R¹ is —CH(CH₃)₂ and R² is —CH₂CH₂CH₂NHCONH₂.

22. The compound of claim 16 or 17, wherein R¹ is —CH₂C₆H₅ and R² is —CH₂CH₂CH₂CH₂NH₂.

23. The compound of claim 16 or 17, wherein R¹ is —CH₃ and R² is —CH₂C₆H₅.

24. The compound of claim 16 or 17, wherein R¹ is —CH₂CH₂CH₂CH₂NH₂ and R² is —CH₃.

25. The compound of any one of claims 1 to 14, wherein L is a tri-peptide linking moiety having the structure of: 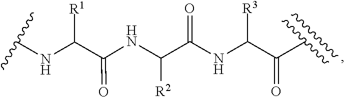
wherein R¹, R² and R³ are each independently H, —CH₂CH₂CH₂NHCONH₂, or a side chain of a naturally occurring amino acid, and wherein [symbol] represents the point of attachment to Y and [symbol] represents the point of attachment to SP.

26. The compound of claim 25, wherein R¹, R², and R³ are each independently selected from the group consisting of hydrogen, CH₃, —CH(CH₃)₂, —CH₂CH₂CH₂CH₂NH₂, —CH₂C₆H₅, CH₂C₆H₄OH, —CH₂CH₂CH₂NH(NH)NH₂, —CH₂CH₂CH₂NHCONH₂, and —CH₂CH₂CO₂H.

27. The compound of claim 26, wherein R¹ is H or CH₃.

28. The compound of claim 26 or 27, wherein R² is —CH₂CH₂CH₂CH₂NH₂, —CH₂C₆H₅, or CH₃.

29. The compound of any one of claims 26 to 28, wherein R³ is H or CH₃.

30. The compound of any one of claims 25 to 29, wherein R¹ is H, R² is —CH₂C₆H₅, and R³ is H.

31. The compound of any one of claims 25 to 29, wherein R¹ is H, R² is —CH₂CH₂CH₂CH₂NH₂, and R³ is H.

32. The compound of any one of claims 25 to 29, wherein R¹ is CH₃, R² is CH₃, and R³ is CH₃.

33. The compound of any one of the preceding claims, wherein SP is absent.

34. The compound of any one of claims 1-32, wherein SP is a di- or tri-peptide linking moiety having L bonded to the N-terminus, and T bonded to the C-terminus.

35. The compound of claim 34, wherein SP is a di-peptide linking moiety.

36. The compound of claim 35, wherein the di-peptide linking moiety is 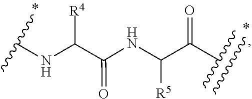
wherein R⁴ and R⁵ are each independently H or —CH₃, wherein [symbol] represents the point of attachment to L and [symbol] represents the point of attachment to T.

37. The compound of claim 36, wherein R⁴ is H, and R⁵ is H.

38. The compound of claim 34, wherein SP is a tri-peptide linking moiety.

39. The compound of claim 38, wherein the tri-peptide linking moiety is 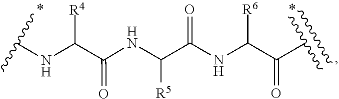
wherein R⁴, R⁵ and R⁶ are each independently H or —CH₃, wherein [symbol] represents the point of attachment to L and [symbol] represents the point of attachment to T.

40. The compound of claim 39, wherein R⁴ is H; R⁵ is H; and R⁶ is H.

41. The compound of any one of claims 1-32, wherein SP is a linking moiety capable of self-immolation at pH less than 8.

42. The compound of claim 41, wherein SP is selected from:

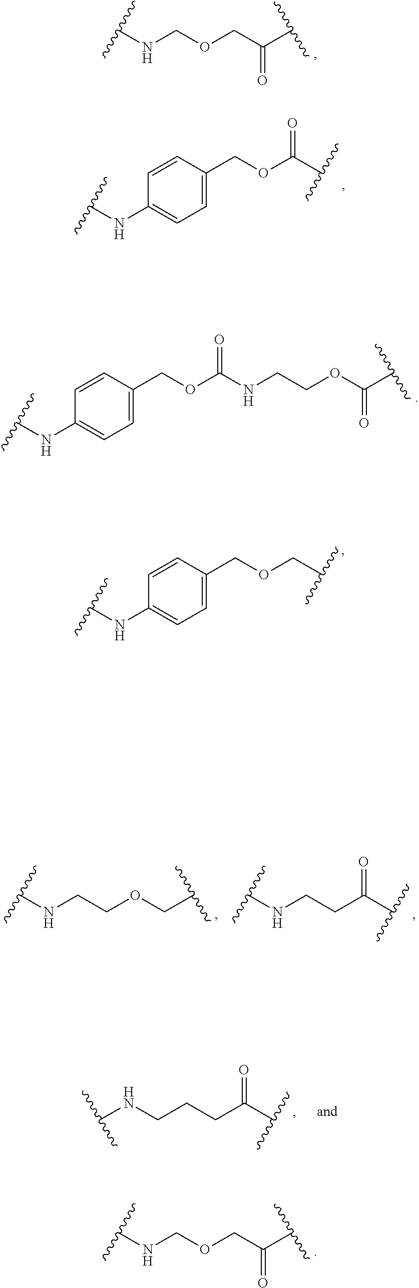

43. The compound of any one of the preceding claims, wherein T is a moiety derived from a compound capable of inhibiting topoisomerase.

44. The compound of claim 43, wherein T is selected from: 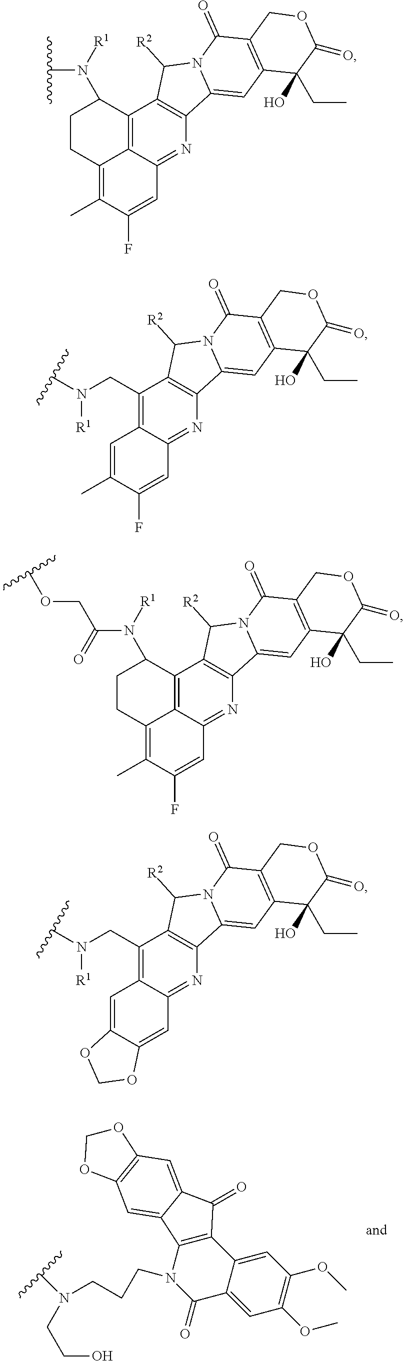 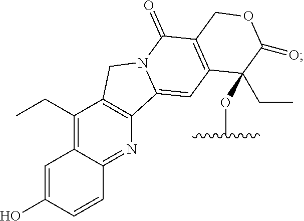
wherein R¹ is H, CH₃ or SO₂CH₃; and R² is H or CH₂OH.

45. The compound of any one of claims 1-42, wherein T is a moiety derived from a compound capable of inhibiting poly (ADP-ribose) polymerase (PARP).

46. The compound of claim 45, wherein the moiety derived from a compound capable of inhibiting PARP is selected from:

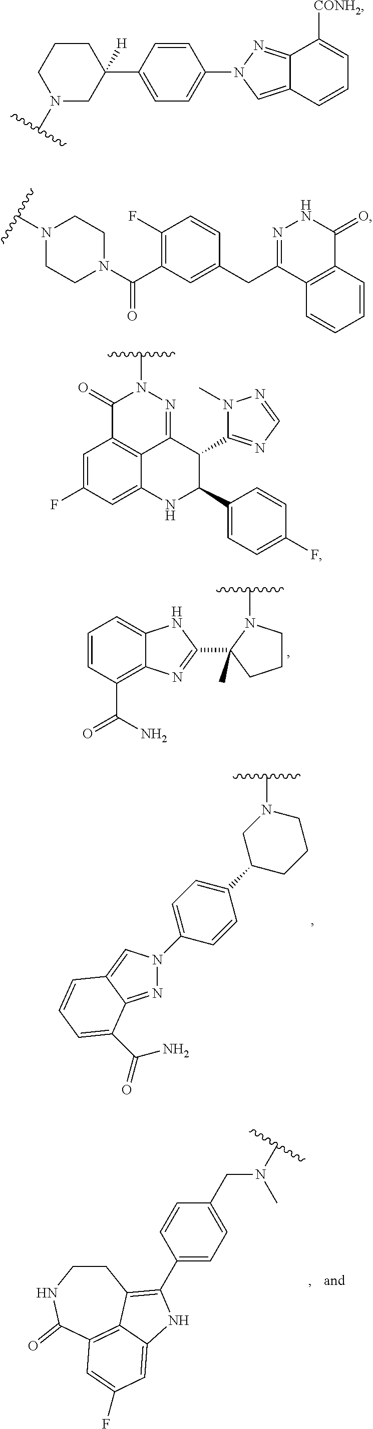

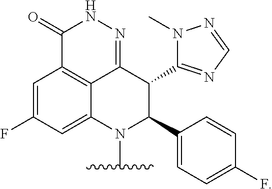 [SMILES: *N1c2cc(F)cc3c(=O)[nH]nc(c23)[C@H](c2ncnn2C)[C@H]1c1ccc(F)cc1]

47. The compound of claim 1, wherein the compound is a compound listed in Table 1, Table 2, Table 3, or Table 4.

48. A conjugate of Formula (II): 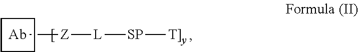
wherein:
T is a moiety derived from a compound capable of inhibiting topoisomerase enzyme or poly (ADP-ribose) polymerase (PARP);
SP is absent, a di- or tri-peptide linking moiety having L bonded to the N-terminus and T bonded to the C-terminus, or a linking moiety capable of self-immolation at pH less than 8;
L is a di- or tri-peptide linking moiety having Y bonded to the N-terminus and SP bonded to the C-terminus; [symbol]
is an antibody;
Z is a residual moiety resulting from the covalent linkage of Y to [symbol] ; and Y is a conjugation moiety capable of forming a covalent bond with a nitrogen atom of a lysine residue or a sulfur atom of a cysteine reside; and
wherein y is an integer from 1 to 20.

49. The conjugate of claim 48, wherein the conjugate is a conjugate of Formula (IIa): 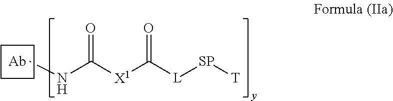
wherein,
X¹ is selected from bond, —(CH₂)ₙ—, —(CH₂)ₙ—(OCH₂CH₂)ₘ—, —(CH₂)ₙ—O—(CH₂)ₘ—, —(CH₂)ₙ—NH—(CH₂)ₘ—, wherein n is 1 to 5, and m is 1 to 8;
T is a moiety derived from a compound capable of inhibiting topoisomerase or poly (ADP-ribose) polymerase (PARP);
SP is absent, a di- or tri-peptide linking moiety having L bonded to the N-terminus and T bonded to the C-terminus, or a linking moiety capable of self-immolation at pH less than 8;
L is a di- or tri-peptide linking moiety having SP bonded to the C-terminus; [symbol]
is an anti-ICAM1 or anti-EphA2 antibody; and
wherein y is an integer from 1 to 20.

50. The conjugate of claim 48, wherein the conjugate is a conjugate of Formula (IIb): 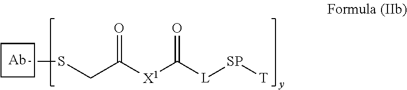
wherein,
X¹ is selected from bond, —(CH₂)ₙ—, —(CH₂)ₙ—(OCH₂CH₂)ₘ—, —(CH₂)ₙ—O—(CH₂)ₘ—, —(CH₂)ₙ—NH—(CH₂)ₘ—, wherein n is 1 to 5, and m is 1 to 8;
T is a moiety derived from a compound capable of inhibiting topoisomerase or poly (ADP-ribose) polymerase (PARP);
SP is absent, a di- or tri-peptide linking moiety having L bonded to the N-terminus and T bonded to the C-terminus, or a linking moiety capable of self-immolation at pH less than 8;
L is a di- or tripeptide linking moiety having SP bonded to the C-terminus; [symbol]
is an anti-ICAM1 or anti-EphA2 antibody;
wherein y is an integer from 1 to 8.

51. The conjugate of claim 48, wherein the conjugate is a conjugate of Formula (IIc), 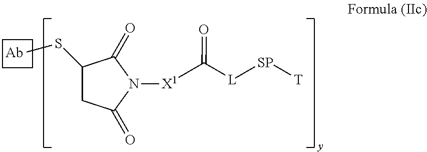
wherein,
X¹ is selected from bond, —(CH₂)ₙ—, —(CH₂)ₙ—(OCH₂CH₂)ₘ—, —(CH₂)ₙ—O—(CH₂)ₘ—, —(CH₂)ₙ—NH—(CH₂)ₘ—, wherein n is 1 to 5, and m is 1 to 8;
T is a moiety derived from a compound capable of inhibiting topoisomerase or poly (ADP-ribose) polymerase (PARP);
SP is absent, a di- or tri-peptide linking moiety having L bonded to the N-terminus and T bonded to the C-terminus, or a linking moiety capable of self-immolation at pH less than 8;
L is a di- or tripeptide linking moiety SP bonded to the C-terminus;
Ab is an anti-ICAM1 or anti-EphA2 antibody; and
wherein y is an integer from 1 to 8.

52. The conjugate of claim 48, wherein the conjugate is a conjugate of Formula (IId): 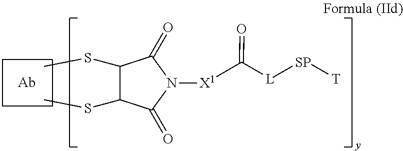
wherein,
X¹ is selected from bond, —(CH₂)ₙ—, —(CH₂)ₙ—(OCH₂CH₂)ₘ—, —(CH₂)ₙ—O—(CH₂)ₘ—, —(CH₂)ₙ—NH—(CH₂)ₘ—, wherein n is 1 to 5, and m is 1 to 8;
T is a moiety derived from a compound capable of inhibiting topoisomerase or poly (ADP-ribose) polymerase (PARP);
SP is absent, a di- or tri-peptide linking moiety having L bonded to the N-terminus and T bonded to the C-terminus, or a linking moiety capable of self-immolation at pH less than 8;
L is a di- or tri-peptide linking moiety having SP bonded to the C-terminus;
Ab is an anti-ICAM1 or anti-EphA2 antibody; and
wherein y is an integer from 1 to 4.

53. The conjugate of any one of claims 48-52, wherein L is a dipeptide linking moiety having the structure of: 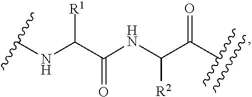
wherein R¹ and R² are each independently selected from H, —CH₂CH₂CH₂NHCONH₂, or a side chain of a naturally occurring amino acid, and wherein [symbol] represents the point of attachment to Y and [symbol] represents the point of attachment to SP.

54. The conjugate of claim 53, wherein R¹ and R² are each independently selected from the group consisting of hydrogen, —CH₃, —CH(CH₃)₂, —CH₂CH₂CH₂CH₂NH₂, —CH₂C₆H₅, —CH₂C₆H₄OH, —CH₂CH₂CH₂NH(NH)NH₂, —CH₂CH₂CH₂NHCONH₂, and —CH₂CH₂CO₂H.

55. The conjugate of claim 54, wherein R¹ is selected from the group consisting of —CH₃, —CH(CH₃)₂, —CH₂CH₂CH₂CH₂NH₂, or —CH₂C₆H₅.

56. The conjugate of claim 54 or 55, wherein R² is selected from the group consisting of —CH₃, —CH₂CH₂CH₂CH₂NH₂, —CH₂C₆H₅, or —CH₂CH₂CH₂NHCONH₂.

57. The conjugate of claim 54 or 55, wherein R¹ is —CH₃ and R² is —CH₂CH₂CH₂CH₂NH₂.

58. The conjugate of claim 54 or 55, wherein R¹ is —CH₃ and R² is —CH₃.

59. The conjugate of claim 54 or 55, wherein R¹ is —CH(CH₃)₂ and R² is —CH₂CH₂CH₂NHCONH₂.

60. The conjugate of claim 54 or 55, wherein R¹ is —CH₂C₆H₅ and R² is —CH₂CH₂CH₂CH₂NH₂.

61. The conjugate of claim 52 or 53, wherein R¹ is —CH₃ and R² is —CH₂C₆H₅.

62. The conjugate of claim 52 or 53, wherein R¹ is —CH₂CH₂CH₂CH₂NH₂ and R² is —CH₃.

63. The conjugate of any one of claims 48-52, wherein L is a tri-peptide linking moiety having the structure of: 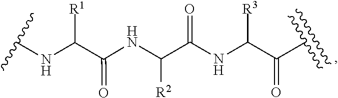
wherein R¹, R² and R³ are each independently H, —CH₂CH₂CH₂NHCONH₂, or a side chain of a naturally occurring amino acid, and wherein [symbol] represents the point of attachment to Y and [symbol] represents the point of attachment to SP.

64. The conjugate of claim 63, wherein R¹, R², and R³ are each independently selected from the group consisting of hydrogen, CH₃, —CH(CH₃)₂, —CH₂CH₂CH₂CH₂NH₂, —CH₂C₆H₅, CH₂C₆H₄OH, —CH₂CH₂CH₂NH(NH)NH₂, —CH₂CH₂CH₂NHCONH₂, and —CH₂CH₂CO₂H.

65. The conjugate of claim 64, wherein R¹ is H or CH₃.

66. The conjugate of claim 64 or 65, wherein R² is —CH₂CH₂CH₂CH₂NH₂, —CH₂C₆H₅, or CH₃.

67. The conjugate of any one of claims 64 to 66, wherein R³ is H or CH₃.

68. The conjugate of any one of claims 63 to 67, wherein R¹ is H, R² is —CH₂C₆H₅, and R³ is H.

69. The conjugate of any one of claims 63 to 67, wherein R¹ is H, R² is —CH₂CH₂CH₂CH₂NH₂, and R³ is H.

70. The conjugate of any one of claims 63 to 67, wherein R¹ is CH₃, R² is CH₃, and R³ is CH₃.

71. The conjugate of any one of claims 48-70, wherein SP is absent.

72. The conjugate of any one of claims 48-70, wherein SP is a di- or tri-peptide linking moiety having L bonded to the N-terminus, and T bonded to the C-terminus.

73. The conjugate of claim 72, wherein SP is a di-peptide.

74. The conjugate of claim 73, wherein the di-peptide linking moiety is 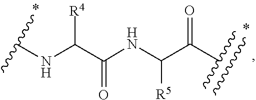
wherein R⁴ and R⁵ are each independently H or —CH₃, wherein [symbol] represents the point of attachment to L and [symbol] represents the point of attachment to T.

75. The conjugate of claim 74, wherein R⁴ is H, and R⁵ is H.

76. The conjugate of claim 72, wherein SP is a tri-peptide.

77. The conjugate of claim 76, wherein the tri-peptide linking moiety is 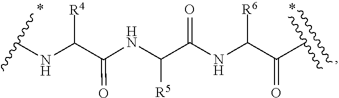
wherein R⁴, R⁵ and R⁶ are each independently H or —CH₃, wherein [symbol] represents the point of attachment to L and [symbol] represents the point of attachment to T.

78. The conjugate of claim 77, wherein R⁴ is H; R⁵ is H; and R⁶ is H.

79. The conjugate of any one of claims 48-70, wherein SP is a linking moiety capable of self-immolation at pH less than 8.

80. The conjugate of claim 79, wherein SP is selected from:

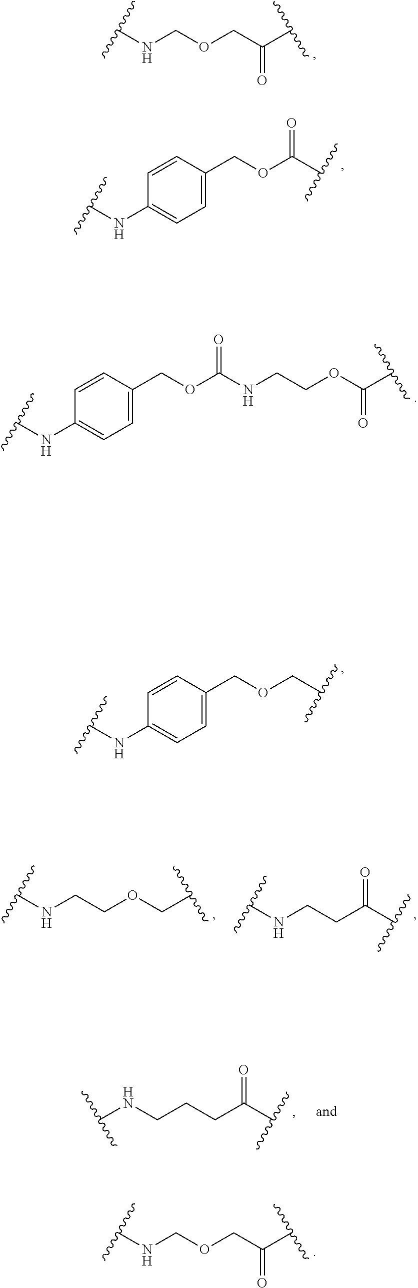

81. The conjugate of any one of claims 48-80, wherein T is a moiety derived from a compound capable of inhibiting topoisomerase.

82. The conjugate of claim 79, wherein T is: 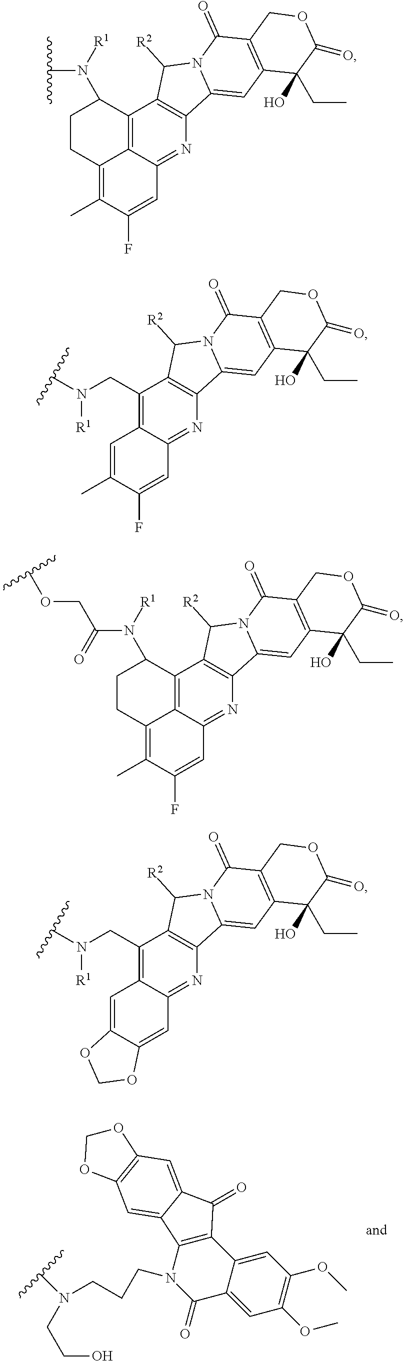 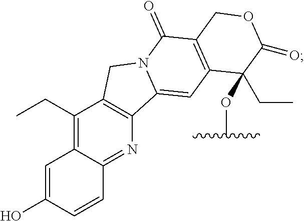
wherein R¹ is H, CH₃ or SO₂Me; and R² is H or CH₂OH.

83. The conjugate of any one of claims 48-80, wherein T is a moiety derived from a compound capable of inhibiting poly (ADP-ribose) polymerase (PARP).

84. The conjugate of claim 83, wherein T is selected from:

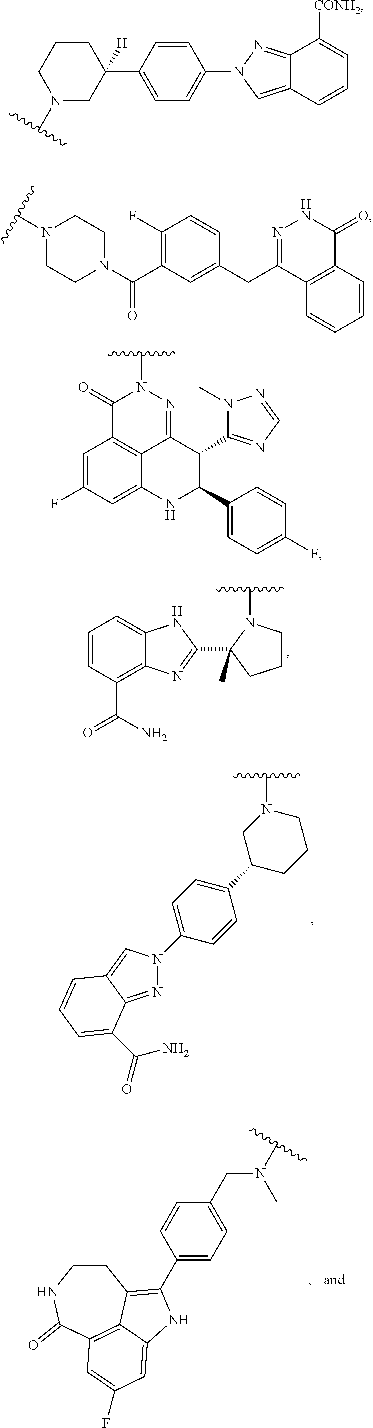

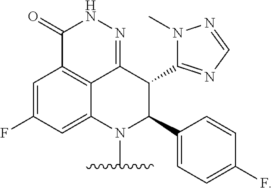 [SMILES: *N1c2cc(F)cc3c(=O)[nH]nc(c23)[C@H](c2ncnn2C)[C@H]1c1ccc(F)cc1]

85. The conjugate of any one of claims 48-84, wherein the antibody comprises an anti-ICAM1 antibody.

86. The conjugate of any one of claims 48-84, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise the amino acid sequences according to HC-CDR1: SEQ ID NO: 25, HC-CDR2: SEQ ID NO: 26, HC-CDR3: SEQ ID NO: 27, LC-CDR1: SEQ ID NO: 43, LC-CDR2: SEQ ID NO: 44, LC-CDR3: SEQ ID NO: 45.

87. The conjugate of any one of claims 48-84, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise the amino acid sequences according to HC-CDR1: SEQ ID NO: 28, HC-CDR2: SEQ ID NO: 29, HC-CDR3: SEQ ID NO: 30, LC-CDR1: SEQ ID NO: 46, LC-CDR2: SEQ ID NO: 47, LC-CDR3: SEQ ID NO: 48.

88. The conjugate of any one of claims 48-84, wherein the antibody comprises an antibody format selected from IgG, Fab, Fab′, scFv, and (Fab′)₂.

89. The conjugate of any one of claims 48-84, wherein the heavy chain variable domain is fused to a human IgG1 constant region.

90. The conjugate of any one of claims 48-84, wherein the heavy chain variable domain is fused to a human IgG4 constant region.

91. The conjugate of any one of claims 48-84, wherein the light chain variable domain is fused to a human Kappa constant region.

92. The conjugate of any one of claims 48-84, wherein the heavy chain variable domain comprises a variable domain of an IgG1, IgG2, IgG3, or IgG4 heavy chain.

93. The conjugate of any one of claims 48-84, wherein the light chain variable domain comprises a variable domain of a Kappa or Lambda light chain.

94. The conjugate of any one of claims 48-84, wherein the heavy chain variable domain comprises an amino acid sequence with at least 90%, 95%, 96%, 97%, 98%, 99%, or 100% identity to any one of SEQ ID NOs: 61 or 62.

95. The conjugate of any one of claims 48-84, wherein the light chain variable domain comprises an amino acid sequence with at least 90%, 95%, 96%, 97%, 98%, 99%, or 100% identity to any one of SEQ ID NOs: 67 or 68.

96. The conjugate of any one of claims 48-84, wherein the antibody comprises an amino acid sequence with at least 90%, 95%, 96%, 97%, 98%, 99%, or 100% identity to SEQ ID NO: 2 and an amino acid sequence with at least 90%, 95%, 96%, 97%, 98%, 99%, or 100% identity to SEQ ID NO: 4.

97. The conjugate of any one of claims 48-84, wherein the antibody comprises an amino acid sequence with at least 90%, 95%, 96%, 97%, 98%, 99%, or 100% identity to SEQ ID NO: 14 and an amino acid sequence with at least 90%, 95%, 96%, 97%, 98%, 99%, or 100% identity to SEQ ID NO: 16.

98. The conjugate of any one of claims 48-84, wherein the antibody is an anti-ICAM1 antibody.

99. The conjugate of claim 98, wherein the anti-ICAM1 antibody is VP0270, comprising a heavy chain of an amino acid sequence of SEQ ID NO:2, and a light chain of an amino acid sequence of SEQ ID NO:4.

100. The conjugate of any one of claims 48-84, wherein the antibody is an anti-EphA2 antibody.

101. The conjugate of claim 100, wherein the anti-EphA2 antibody is VP0633, comprising a heavy chain of an amino acid sequence of SEQ ID NO:6, and a light chain of an amino acid sequence of SEQ ID NO:8.

102. The conjugate of claim 100, wherein the anti-EphA2 antibody is VP0253, comprising a heavy chain of an amino acid sequence of SEQ ID NO:10, and a light chain of an amino acid sequence of SEQ ID NO:12.

103. The conjugate of any one of claims 48-84, wherein the antibody comprises an anti-EphA2 antibody.

104. The conjugate of any one of claims 48-84, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise the amino acid sequences according to HC-CDR1: SEQ ID NO: 31, HC-CDR2: SEQ ID NO: 32, HC-CDR3: SEQ ID NO: 33, LC-CDR1: SEQ ID NO: 49, LC-CDR2: SEQ ID NO: 50, LC-CDR3: SEQ ID NO: 51.

105. The conjugate of any one of claims 48-84, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise the amino acid sequences according to HC-CDR1: SEQ ID NO: 34, HC-CDR2: SEQ ID NO: 35, HC-CDR3: SEQ ID NO: 36, LC-CDR1: SEQ ID NO: 52, LC-CDR2: SEQ ID NO:
53, LC-CDR3: SEQ ID NO: 54.

106. The conjugate of any one of claims 48-84, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise the amino acid sequences according to HC-CDR1: SEQ ID NO: 37, HC-CDR2: SEQ ID NO: 38, HC-CDR3: SEQ ID NO: 39, LC-CDR1: SEQ ID NO: 55, LC-CDR2: SEQ ID NO: 56, LC-CDR3: SEQ ID NO: 57.

107. The conjugate of any one of claims 48-84, wherein the antibody comprises a heavy chain variable domain that comprises CDRs: HC-CDR1, HC-CDR2, HC-CDR3, and a light chain variable domain that comprises CDRs: LC-CDR1, LC-CDR2, LC-CDR3, wherein the HC-CDR1, HC-CDR2, HC-CDR3, LC-CDR1, LC-CDR2, LC-CDR3 comprise the amino acid sequences according to HC-CDR1: SEQ ID NO: 40, HC-CDR2: SEQ ID NO: 41, HC-CDR3: SEQ ID NO: 42, LC-CDR1: SEQ ID NO: 58, LC-CDR2: SEQ ID NO: 59, LC-CDR3: SEQ ID NO: 60.

108. The conjugate of any one of claims 48-84, wherein the antibody comprises an antibody format selected from IgG, Fab, Fab′, scFv, and (Fab′)₂.

109. The conjugate of any one of claims 48-84, wherein the heavy chain variable domain is fused to a human IgG1 constant region.

110. The conjugate of any one of claims 48-84, wherein the heavy chain variable domain is fused to a human IgG4 constant region.

111. The conjugate of any one of claims 48-84, wherein the light chain variable domain is fused to a human Lambda constant region.

112. The conjugate of any one of claims 48-84, wherein the heavy chain variable domain comprises a variable domain of an IgG1, IgG2, IgG3, or IgG4 heavy chain.

113. The conjugate of any one of claims 48-84, wherein the light chain variable domain comprises a variable domain of a Kappa or Lambda light chain.

114. The conjugate of any one of claims 48-84, wherein the heavy chain variable domain comprises an amino acid sequence with at least 90%, 95%, 96%, 97%, 98%, 99%, or 100% identity to any one of SEQ ID NOs: 63, 64, 65, or 66.

115. The conjugate of any one of claims 48-84, wherein the light chain variable domain comprises an amino acid sequence with at least 90%, 95%, 96%, 97%, 98%, 99%, or 100% identity to any one of SEQ ID NOs: 69, 70, 71, or 72.

116. The conjugate of any one of claims 48-84, wherein the antibody comprises an amino acid sequence with at least 90%, 95%, 96%, 97%, 98%, 99%, or 100% identity to any one of SEQ ID NOs: 6, 10, 18, or 22.

117. The conjugate of any one of claims 48-84, wherein the antibody comprises an amino acid sequence with at least 90%, 95%, 96%, 97%, 98%, 99%, or 100% identity to any one of SEQ ID NOs: 8, 12, 20, or 24.

118. The conjugate of any one of claims 48-84, wherein the antibody comprises an amino acid sequence with at least 90%, 95%, 96%, 97%, 98%, 99%, or 100% identity to SEQ ID NO: 6 and an amino acid sequence with at least 90%, 95%, 96%, 97%, 98%, 99%, or 100% identity to SEQ ID NO: 8.

119. The conjugate of any one of claims 48-84, wherein the antibody comprises an amino acid sequence with at least 90%, 95%, 96%, 97%, 98%, 99%, or 100% identity to SEQ ID NO: 10 and an amino acid sequence with at least 90%, 95%, 96%, 97%, 98%, 99%, or 100% identity to SEQ ID NO: 12.

120. The conjugate of any one of claims 48-84, wherein the antibody comprises an amino acid sequence with at least 90%, 95%, 96%, 97%, 98%, 99%, or 100% identity to SEQ ID NO: 18 and an amino acid sequence with at least 90%, 95%, 96%, 97%, 98%, 99%, or 100% identity to SEQ ID NO: 20.

121. The conjugate of any one of claims 48-84, wherein the antibody comprises an amino acid sequence with at least 90%, 95%, 96%, 97%, 98%, 99%, or 100% identity to SEQ ID NO: 22 and an amino acid sequence with at least 90%, 95%, 96%, 97%, 98%, 99%, or 100% identity to SEQ ID NO: 24.

122. The conjugate of any one of claims 48-52, wherein the conjugate is a conjugated listed in Table 5 or Table 6.

123. The conjugate of any one of claims 48-90, wherein the topoisomerase is topoisomerase I.

124. A pharmaceutical composition comprising a conjugate of any one of claims 48-123, and at least one pharmaceutically acceptable carrier.

125. A method of treating a disease or disorder in a patient in need thereof, comprising administering to the patient the conjugate of any one of claims 48-123 or the pharmaceutical composition of claim 124.

126. The method of claim 125, wherein the disease or disorder is cancer.

127. The method of claim 126, wherein the cancer is a solid tumor.

128. The method of claim 125, wherein the cancer is selected from the group consisting of ovarian cancer, head and neck cancer, thyroid cancer, gastric cancer, bladder cancer, cholangiocarcinoma, endometrial cancer, hepatocellular carcinoma, kidney cancer, melanoma, lung cancer (e.g., non-small cell lung cancer), colorectal cancer, prostate cancer, pancreatic cancer, and Ewing's sarcoma.

129. The method of claim 128, wherein the cancer is lung cancer.

130. The method of claim 129, wherein the cancer is non-small cell lung cancer.

131. The method of claim 128, wherein the cancer is colorectal cancer.

132. The method of claim 128, wherein the cancer is prostate cancer.

133. The method of claim 128, wherein the cancer is pancreatic cancer.

134. The method of claim 128, wherein the cancer is hepatocellular carcinoma.

135. The method of claim 126, wherein the cancer is a hematological malignancy.

136. The method of claim 135, wherein the hematological malignancy is multiple myeloma (MM).

137. The method of claim 135, wherein the hematological malignancy is non-hodgkin lymphoma (NHL).
